(19) United States
(12) Patent Application Publication
(10) Pub. No.: US 2025/0289903 A1
(43) Pub. Date: Sep. 18, 2025

(54) METHODS AND COMPOSITIONS FOR TREATING EOSINOPHIL DRIVEN DISEASES AND DISORDERS

(71) Applicant: Purinnomia Biotech, Inc., Woburn, MA (US)

(72) Inventors: Yan Wu Shang, North Reading, MA (US); Haitao Zhao, Beijing (CN)

(21) Appl. No.: 18/860,380

(22) PCT Filed: Apr. 14, 2023
(86) PCT No.: PCT/US23/65762

§ 371 Date: Oct. 25, 2024

Related U.S. Application Data
(60) Provisional application No. 63/336,418, filed on Apr. 29, 2022.

(51) Int. Cl. C07K 16/28; A61K 39/395; A61K 45/06; A61P 11/02; A61P 11/06; A61P 17/00

(52) U.S. Cl. CPC C07K 16/2896; A61K 39/3955; A61K 45/06; A61P 11/02; A61P 11/06; A61P 17/00

(57) ABSTRACT

The present disclosure relates, in part, to compositions comprising anti-CD39 antibodies for use in methods for reducing eosinophil cells or function and/or treating diseases or conditions associated with unwanted eosinophil activity.

### CROSS-REFERENCE TO RELATED APPLICATIONS

This application claims the benefit of priority to U.S. Provisional Application Ser. No. 63/336,418, filed on 29 Apr. 2022; the entire contents of said application are incorporated herein in their entirety by this reference.

### SEQUENCE LISTING

The present specification contains a Sequence Listing which has been submitted electronically in XML format. The Sequence Listing XML is incorporated herein by reference. Said XML file, created on Apr. 7, 2023, is named PNC-00325_SL.xml and is 86,881 bytes in size.

### BACKGROUND OF THE INVENTION

Recent research has shown that eosinophils are also involved in several homeostatic processes, including metabolism, tissue remodeling and development, neuronal regulation, epithelial and microbiome regulation, and immunoregulation, indicating that these cells may play a crucial role in metabolic regulation and organ function in healthy humans.

Eosinophils play a homeostatic role in the body's immune responses. These cells are involved in combating some parasitic, bacterial, and viral infections and certain cancers and have pathologic roles in inflammatory disorders and diseases including asthma, rhinosinusitis, eosinophilic gastrointestinal disorders, and hypereosinophilic syndromes.

Treatment of eosinophilic diseases has traditionally been through nonspecific eosinophil attenuation by use of glucocorticoids. However, several novel biologic therapies targeting eosinophil maturation factors, such as interleukin (IL)-5 and the IL-5 receptor or IL-4/IL-13, have recently been approved for clinical use. Despite the success of biologic therapies, some patients with eosinophilic inflammatory disease may not achieve adequate symptom control, underlining the need to further investigate the contribution of patient characteristics, such as comorbidities and other processes, in driving ongoing disease activity.

For instance, esophageal inflammation disorders such as eosinophilic esophagitis (EoE), a disease characterized by high levels of eosinophils in the esophagus, as well as basal zonal hyperplasia, is increasingly being diagnosed in children and adults. Many aspects of the disease remain unclear including its etiology, natural history, and optimal therapy. EoE affects all age groups but most frequently individuals between 20 and 50 years of age. Symptoms of EoE often mimic those of gastroesophageal reflux disease (GERD) and include vomiting, dysphagia, pain and food impaction. The disease is painful, leads to difficulty swallowing, and predisposes patients to other complications. EoE is often misdiagnosed for GERD, causing delay in adequate treatment for EoE patients. Currently, no topically administered anti-inflammatory medications are approved for the treatment of conditions associated with inflammation of the upper portion of the gastrointestinal tract, particularly the inflammatory conditions of the esophagus, such as EoE. Although systemic treatments with corticosteroids such as prednisolone are effective, these therapeutics are associated with significant adverse effects such as suppression of the hypothalamic-pituitary-adrenal (HPA) axis as reflected in salivary cortisol levels, generalized suppression of immune function, and particularly in children, troubling side-effects from long term systemic exposure include growth retardation.

In addition, some drugs have been reported to cause eosinophilia-so called “drug-induced eosinophilia”. For instance, immune checkpoint inhibitors (ICIs) are a new standard of care in several cancers. However, ICIs are also associated with frequent and potentially organ- or life-threatening immune-related adverse events (irAE), which generally mimic autoimmune or inflammatory conditions including eosinophilic asthma and hypereosinophilic disorders. Other drugs associated with drug-induced eosinophilia that are reported to produce serious reactions include, but are not limited to, antimalarials (e.g., pyrimethamine and dapson), penicillins, glycopeptides, cephalosporins, sulphonamides, tetracyclines (especially minocycline), nitrofurantoin, anti-tuberculous therapies, ACE inhibitors, tryptophan, anticonvulsants (e.g., phenytoin, carbamazepine, and phenobarbitone), NSAIDs, gold, H2-receptor antagonists, proton pump inhibitors, aminosalicylates, and chlorpropamide.

### SUMMARY OF THE INVENTION

Eosinophils can regulate local immune and inflammatory responses, and their accumulation in the blood and tissue is associated with several inflammatory and infectious diseases. Thus, therapies that target eosinophils may help control diverse diseases, including atopic disorders such as asthma and allergy, as well as diseases that are not primarily associated with eosinophils, such as autoimmunity and malignancy. The present invention relates to eosinophil-targeted therapeutic agents.

The present invention is based on the observation that eosinophils express CD39 and can be targeted with CD39-targeted eosinophil-depleting agents, such as ADCC (antibody-dependent cellular cytotoxicity)-competent and/or ADCP (antibody-dependent cellular phagocytosis)-competent anti-CD39 antibodies or CD39-targeted cytotoxic drug conjugates, in order to reduce the level of eosinophils and/or eosinophilic function either systemically or locally (or both). These CD39-targeted eosinophil-depleting agents thus have utilities ranging from use as part of a treatment for diseases in which aberrant activation or overabundance of eosinophils is part of the pathology, including, for example, inflammatory or autoimmune diseases and systemic or localized eosinophilia or drug-induced eosinophilia. Use as part of treatment measures for preventing tissue graft (autologous or allogeneic) rejection is also contemplated.

With the exception of the PEO22 antibody described herein, the current clinical use of anti-CD39 antibodies is in oncology and has focused on inhibiting the ectonucleotidase activity of CD39, that is, have been targeted to produce a decrease in the intratumoral level of the enzymatic activity associated with that protein, and, in doing so, reduce the intratumoral levels of the immunosuppressive agent, adenosine. Those antibodies have been chosen to not have any CD39-dependent cell depletion activity, i.e., the antibodies are purposefully selected to not include an ADCC and/or ADCP function. Again, the purpose of these prior art antibodies has been in the ability to inhibit the enzymatic activity of CD39 not to kill cells which express CD39, and in the formats those antibodies are used would not have the level of eosinophil depletion of the antibodies contemplated for use in the present methods and formulations.

However, in the context of the current invention, those versions of the inventive methods and formulations utilizing anti-CD39 antibodies that retain ADCC and/or ADCP function-such that when bound to eosinophils results in the ADCC-mediated and/or ADCP-mediated clearance of those cells. Such anti-CD39 antibodies can be, to illustrate, monovalent or multivalent (including bivalent) for CD39. However, the invention also provides for bispecific antibodies which include, in addition to one or more CD39 binding moieties, additional binding moieties that bind to one or more cell surface epitopes expressed by eosinophils.

Numerous embodiments are provided that may be applied to any aspect encompassed by the present invention and/or combined with any other embodiment described herein. For example, in one aspect, an anti-CD39 antibody, or antigen-binding fragment thereof, comprising (i) at least one antigen binding domain that binds ectonucleoside triphosphate diphosphohydrolase-1 (CD39) at a site such that the anti-CD39 antibody forms a stable immune complex, and (ii) an FcγRIIIa binding moiety that binds FcγRIIIa receptor and confers ADCC and/or ADCP activity against CD39+ cells to the anti-CD39 antibody, is provided.

Numerous embodiments are further provided that may be applied to any aspect of the present invention and/or combined with any other embodiment described herein. For example, in one embodiment, the eosinophil cells are CD39+eosinophil cells. In some embodiments, the CD39+eosinophil cells (i) co-express one or more of the cell surface markers selected from the group consisting of CD45, CD11b, Siglec-8, the a subunit of IL-5 receptor (IL-5Rα or CD125), the a subunit of IL-3 receptor (IL-3Rα or CD123), IL-4R, IL-9R, IL-13R, IL-14R, ST2 (IL-33R), paired immunoglobulin-lie receptor A (PIRA), paired immunoglobulin-lie receptor B (PIRB), L-selectin, EGF-like module containing mucin-like hormone receptor (EMR) 1, CCR3 (CD193), and the chemoattractant receptor expressed on type 2 helper T cells (CRTh2, also called DP2, the type 2 prostaglandin D2 receptor or CD294); (ii) are CD45⁺CD11b⁺eosinophil cells; and/or (iii) are CD45⁺CD11b⁺Siglec-8+eosinophil cells. In some embodiments, the anti-CD39 antibody, or antigen-binding fragment thereof, promotes: (i) stable immune complex formation when incubated with HCC1739BL cells as characterized by loss of less than 40% of the immune complex after 24 hours, or less than 35%, less than 30%, less than 25%, less than 20%, less than 15% or even less than 10% after 24 hours, optionally wherein the immune complex formation is detected by fluorescence intensity using a fluorescently labeled secondary antibody (e.g., merely to illustrate, the stability of an immune complex formed with an anti-CD39 antibody can be determined by incubating anti-CD39 monoclonal antibodies (mAbs) (e.g., at 2 μg/mL or greater) with HCC1739BL cells for different times and then detecting the presence of immune complex by fluorescent conjugated secondary antibody); (ii) depletion of CD39+eosinophils; (iii) binding to a CD39 epitope having a sequence selected from the group of CD39 amino acid epitope sequences listed in FIG. 30; and/or (iv) binding to CD39 in a manner that is non-competitive or only partially competitive with monoclonal antibody Clone A1 binding to CD39. In some embodiments, the anti-CD39 antibody, or antigen-binding fragment thereof, promotes depletion of CD39+eosinophils via ADCC-mediated killing and/or ADCP-mediated killing. In some embodiments, the anti-CD39 antibody, or antigen-binding fragment thereof, promotes depletion of CD39+eosinophils in the form of an antibody-drug conjugate that is taken up by and is toxic to the CD39+eosinophils.

In another embodiment, the FcγRIIIa binding moiety is selected from the group consisting of an Fc domain, an antibody or fragment thereof that binds to FcγRIIIa, and an FcγRIIIa binding peptide.

In still another embodiment, the antigen binding domain is selected from the group consisting of a Fab, Fab′, F(ab′)₂, Fv or single chain Fv (scFv), Fav, dsFv, sc (Fv)₂, Fde, sdFv, single domain antibody (dAb), and diabodies fragments and/or wherein the anti-CD39 antibody, or antigen-binding fragment, is monoclonal. In some embodiments, the antigen-binding domain is an scFV comprising the sequence of SEQ ID NO: 40.

In another embodiment, the anti-CD39 antibody, or antigen-binding fragment thereof, has a VH domain with an amino acid sequence that can be encoded by the nucleic acid sequence of or a nucleic acid that hybridizes under stringent conditions to the nucleic acid of SEQ ID No. 1 and a VL domain with an amino acid sequence that can be encoded by the nucleic acid sequence of or a nucleic acid that hybridizes under stringent conditions to the nucleic acid of SEQ ID No. 3 (such as hybridization under 6× sodium chloride/sodium citrate (SSC) at 45° C., and washing in 0.2×SSC/0.1% SDS at 50-65° C.). In still another embodiment, the anti-CD39 antibody, or antigen-binding fragment thereof, comprises a heavy chain having CDRs at least 60% identical (e.g., at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater) to the CDRs of SEQ ID No. 2, 6, 10, 14, 18, 22, 26, 42, 46, 50, or 54, and a light chain having CDRs at least 60% identical (e.g., at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater) to the CDRs of SEQ ID No. 4, 8, 12, 16, 20, 24, 28, 44, 48, 52, or 56. In yet another embodiment, the anti-CD39 antibody, or antigen-binding fragment thereof, comprises a variable heavy (VH) chain at least 60% identical (e.g., at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 2, 6, 10, 14, 18, 22, 26, 42, 46, 50, or 54, and a variable light (VL) chain at least 60% identical (e.g., at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 4, 8, 12, 16, 20, 24, 28, 44, 48, 52, or 56. In another embodiment, the anti-CD39 antibody, or antigen-binding fragment thereof, comprises: (i) a heavy chain having a CDR1 amino acid sequence at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 29, a CDR2 amino acid sequence at least 80% (e.g., at least 81%, 82%, 83%, 84%, 85%, 86%, 87%, 88%, 89%, 90%, 91%, 92%, 93%, 94%, 95%, 96%, 97%, 98%, 99%, or greater) identical to SEQ ID No. 30, and a CDR3 amino acid sequence at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 31; and (ii) a light chain having a CDR1 amino acid sequence at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 32, a CDR2 amino acid sequence at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 33, and a CDR3 amino acid sequence at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or greater) to SEQ ID No. 34. In still another embodiment, the anti-CD39 antibody, or antigen-binding fragment thereof, comprises a heavy chain having CDRs selected from the group consisting of CDRs of SEQ ID No. 6, 10, 14, 18, 22, 26, 42, 46, 50, and 54, a light chain having CDRs selected from the group consisting of CDRs of SEQ ID No. 8, 12, 16, 20, 24, 28, 44, 48, 52, and 56, and human framework sequences to form humanized heavy and light chains with an antigen binding site able to specifically bind human CD39. In still another embodiment, the anti-CD39 antibody, or antigen-binding fragment thereof, comprising (i) a heavy chain variable domain comprising a CDRH1 having the sequence of SEQ ID NO: 29, a CDRH2 having the amino acid sequence of SEQ ID NO: 30, and a CDRH3 having the sequence of SEQ ID NO: 31; and (ii) a light chain variable domain comprising a CDRL1 having the sequence of SEQ ID NO: 32, a CDRL2 having the sequence of SEQ ID NO: 33, and a CDRL3 having the sequence of SEQ ID NO: 34. In another embodiment, the anti-CD39 antibody, or antigen-binding fragment thereof, comprises a heavy chain having CDRs selected from the group consisting of CDRs of SEQ ID NO. 6, 10, 14, 18, 22, 26, 42, 46, 50, and 54, and a light chain having CDRs selected from the group consisting of CDRs of SEQ ID NO. 8, 12, 16, 20, 24, 28, 44, 48, 52, and 56, and human framework sequences to form humanized heavy and light chains with an antigen binding site able to specifically bind human CD39.

In yet another embodiment, the anti-CD39 antibody, or antigen-binding fragment thereof, comprises an Fc domain of an IgG1 or IgG3 isotype, optionally wherein the Fc domain is human. In another embodiment, the anti-CD39 antibody, or antigen-binding fragment thereof, is hypo-fucosylated or afucosylated.

In still another embodiment, the anti-CD39 antibody, or antigen-binding fragment thereof, is human or is humanized.

In yet another embodiment, the anti-CD39 antibody, or antigen-binding fragment thereof, is a bispecific including at least one additional antigen binding site for an eosinophil antigen.

In certain embodiments, the present invention provides bispecific antibodies that bind to both CD39 and an eosinophil cell surface marker selected from the group consisting of Siglec-8, the a subunit of IL-5 receptor (IL-5Rα or CD125), the a subunit of IL-3 receptor (IL-3Rα or CD123), IL-4R, IL-9R, IL-13R, IL-14R, ST2 (IL-33R), PIRA, PIRB, L-selectin, EMR1, CCR3 (CD193), and CRTh2 (CD294), and preferably cause eosinophil depletion-such as by ADCC-mediated and/or ADCP-mediated killing or in the form of an antibody-drug conjugate that is preferentially taken up by and toxic to eosinophils.

In other embodiments, the bispecific is generated with binding domains against antigens that are upregulated on activated eosinophils (or eosinophils found in lesions), with the avidity of the bispecific for different antigens (CD39 and a second antigen) both expressed on eosinophils providing for the selectivity. Exemplary antigens for generation of bispecifics using the CD39 binders of the present invention include CD3, CD4, γSTCR, CD9, CD28, CD29, CD40, CD44, CD45, CD45RO, CD48, CD58, CD63 (lysosome-associated membrane protein 3), CD66b (CEACAM8), CD66e (CEACAM5), CD67, CD69, CD80, CD86, C5aR (CD88), CD101, CD122, CD137 (tumor necrosis factor receptor superfamily member 9, induced by lymphocyte activation, 4-1BB), CD274 (programmed death ligand 1), amb integrin (CD41), α2 integrin (CD49b), α4 integrin (CD49d), aL integrin (CD11a), aM integrin (CD11b), αX integrin (CD11c), αD integrin, β2 integrin (CD18), Aminopeptidase N (CD13), FcαRI (CD89), FcγRIII (CD16), FcγRII (CD32), Fc∈RII (CD23), Granulocyte monocyte-colony stimulating factorRα (CD116), HLA-DR, Intercellular adhesion molecule-1 (CD54), Interleukin (IL)-2Rα (CD25), IL-17RA, IL-17RB, Galectin-3, Neuropeptide S receptor, P-selectin glycoprotein ligand-1 (CD162), Semaphorin 7A (CD108), Thymic stromal lymphopoietin protein receptor (TSLPR), activated aM integrin, activated β1 integrin (CD29), activated β2 integrin, activated FcγRII, and activated CRTh2 (CD294). Such bispecifics bind to and preferably cause eosinophil depletion-such as by ADCC-mediated and/or ADCP-mediated killing or in the form of an antibody-drug conjugate that is preferentially taken up by and toxic to eosinophils. In some embodiments, the anti-CD39 antibody, or antigen-binding fragment thereof, reduces eosinophils. In some embodiments, the anti-CD39 antibody, or antigen-binding fragment thereof, reduces CD39^{high} inducible and/or activated eosinophils, optionally wherein the CD39^{high} inducible and/or activated eosinophils are i) presented in a pathological condition such as asthma, vasculitis, dermatitis, or rhinosinusitis; and/or ii) located in a space selected from the group consisting of blood, bone marrow, lesions, and/or a combination thereof. In some embodiments, an eosinophil-associated disease amenable to therapy with the anti-CD39 antibody is determined according to the presence of CD39^{high} inducible and/or activated eosinophils in a space selected from the group consisting of blood, bone marrow, lesions, and/or a combination thereof.

In another aspect, a pharmaceutical preparation comprising a therapeutically effective amount of at least one anti-CD39 antibody, or antigen-binding fragment thereof, described herein, and one or more pharmaceutically acceptable excipients, buffers or solutions, is provided. For example, the pharmaceutical preparation can be for reducing eosinophil levels, activity, and/or function and suitable for administration to a subject having an inflammatory condition or tissue graft (merely to illustrate) comprising an effective amount of the anti-CD39 antibody, or antigen-binding fragment thereof, and one or more pharmaceutically acceptable excipients, buffers or solutions, wherein administration of the anti-CD39 antibody to the subject results in a reduction in numbers and/or function of CD39+eosinophil cells.

In another aspect, a method for eosinophil participation in a disease or condition by depleting CD39+eosinophil cells, comprising administering to a subject having an unwanted eosinophilic condition an effective amount of a pharmaceutical composition of an anti-CD39 antibody, or antigen-binding fragment thereof, described herein, wherein administration of the anti-CD39 antibody, or antigen-binding fragment thereof, results in a reduction in numbers and/or function of CD39+eosinophil cells.

In certain embodiments, the CD39-targeted eosinophil-depleting agent can be used as part of a treatment for patients suffering from an inflammatory disorder or autoimmune disease characterized by fibrosis, including: pulmonary fibrosis, such as cystic fibrosis, idiopathic pulmonary fibrosis, progressive massive fibrosis; liver fibrosis, such as liver cirrhosis, primary biliary cirrhosis; heart disease, such as atrial fibrosis, endomyocardial fibrosis, old myocardial infarction; arthrofibrosis; Dupuytren's contracture; keloid fibrosis; mediastinal fibrosis; myelofibrosis; nephrogenic systemic fibrosis; retroperitoneal fibrosis; and scleroderma.

In some embodiments, the subject has a disease or condition that involves unwanted eosinophilic activity. The unwanted eosinophilic activity may be caused by aberrant activation or overabundance of eosinophils. In some embodiments, the disease or condition is an inflammatory disorder or an autoimmune disease, such as a gastrointestinal inflammatory disorder, such as eosinophilic esophagitis and/or Crohn's disease. In some embodiments, the method further comprises administering to the subject one or more agents selected from the group consisting of glucocorticosteroids, leukotriene antagonists, mast cell stabilizers, immunomodulators, and Proton Pump Inhibitors (PPIs). In some embodiments, the inflammatory disorder is a chronic inflammatory condition. In some embodiments, the chronic inflammatory condition is selected from the group consisting of rheumatoid arthritis (RA), autoimmune conditions, inflammatory bowel diseases, non-healing wounds, multiple sclerosis, cancer, atherosclerosis, vasculitis, Sjogren's disease, diabetes, lupus erythematosus, asthma, fibrotic diseases, UV damage, and psoriasis. In some embodiments, the fibrotic disease is selected from the group consisting of pulmonary fibrosis, liver fibrosis, heart disease, arthrofibrosis, Dupuytren's contracture, keloid fibrosis, mediastinal fibrosis, myelofibrosis, nephrogenic systemic fibrosis, retroperitoneal fibrosis, and scleroderma. In some embodiments, the pulmonary fibrosis is cystic fibrosis, idiopathic pulmonary fibrosis, or progressive massive fibrosis. In some embodiments, the liver fibrosis is liver fibrosis, liver cirrhosis, or primary biliary cirrhosis. In some embodiments, the heart disease is atrial fibrosis, endomyocardial fibrosis, or old myocardial infarction. In some embodiments, the inflammatory bowel disease is ulcerative colitis or Crohn's disease. In some embodiments, the disease or condition is an inflammatory or obstructive airways disease. In some embodiments, the inflammatory or obstructive airways disease is selected from the group consisting of asthma, acute lung injury (ALI), adult/acute respiratory distress syndrome (ARDS), chronic obstructive pulmonary, airways or lung disease (COPD, COAD or COLD), emphysema, exacerbation of airways hyperreactivity consequent to other drug therapy, bronchitis, and pneumoconiosis. In some embodiments, the disease or condition is an inflammatory or allergic condition of the skin. In some embodiments, the inflammatory or allergic condition of the skin is selected from the group consisting of psoriasis, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, and acne vulgaris. In some embodiments, the disease or condition is pulmonary inflammatory diseases, axial spondyloarthropathy, primary biliary cholangitis, allergic rhinitis, chronic pulmonary disease, allergy, or eosinophilia.

In certain embodiments, the CD39-targeted eosinophil-depleting agent can be used as part of a treatment for drug-induced eosinophilia, such as eosinophilic asthma and hypereosinophilic disorders that are secondary to ICI therapies and/or other drugs including but not limited to antimalarials (e.g., pyrimethamine and dapson), penicillins, glycopeptides, cephalosporins, sulphonamides, tetracyclines (especially minocycline), nitrofurantoin, anti-tuberculous therapies, ACE inhibitors, tryptophan, anticonvulsants (e.g., phenytoin, carbamazepine, and phenobarbitone), NSAIDs, gold, H2-receptor antagonists, proton pump inhibitors, aminosalicylates, and chlorpropamide.

In certain embodiments, the CD39-targeted eosinophil-depleting agent can be used as part of a treatment for inflammatory bowel diseases, such as ulcerative colitis and Crohn's disease.

In certain embodiments, the CD39-targeted eosinophil-depleting agent can be used as part of a treatment for atopic dermatitis, vasculitis, eosinophilic esophagitis, allergic rhinitis (including seasonal rhinitis), asthma, chronic pulmonary disease (including chronic obstructive pulmonary disease), and allergy (for example a peanut allergy), in particular asthma.

In some embodiments, the disease or condition relates to the respiratory, digestive, cardiovascular, endocrine, integumentary, skeletomuscular, or neurological system, or is a non-oncology hematologic disease. In some embodiments, the disease or condition is tissue graft rejection.

In some embodiments, the tissue graft is autologous or allogeneic. In some embodiments, the subject is a mammal, such as a human or a rodent. In some embodiments, the anti-CD39 antibody, or antigen-binding fragment thereof, is administered to the subject with one or more pharmaceutically acceptable excipients, buffers or solutions. In some embodiments, the anti-CD39 antibody, or antigen-binding fragment thereof, is administered to the subject at a dose of 0.01 to 10 mg/kg, optionally wherein the dosing is provided by a continuous slow-releasing delivery platform to avoid/limit antibody-mediated target cytosis (or antigenic modulation or antigen shaving) for optimum ADCC-mediated and/or ADCP-mediated eosinophil cell depletion efficacy. In some embodiments, the anti-CD39 antibody, or antigen-binding fragment thereof, is administered to the subject once or more daily, trice a week, twice a week, once a week, once every two weeks, once every three weeks, or once every four weeks, optionally wherein the administration is once a week. In some embodiments, the anti-CD39 antibody, or antigen-binding fragment thereof, is administered to the subject for a duration of at least 2 to 6 treatment cycles, or is administered to the subject monthly for life-long use. In some embodiments, the anti-CD39 antibody, or antigen-binding fragment thereof, is administered to the subject via parenteral administration, submucosal hydrogel administration, pulmonary, or topical application, wherein the parenteral administration is by subcutaneous, intravenous, or intramuscular administration.

### BRIEF DESCRIPTION OF THE FIGURES

The patent of application file contains at least one drawing executed in color. Copies of this patent or patent application publication with color drawing(s) will be provided by the Office upon request and payment of the necessary fee.

FIG. 1 shows that a fucosylated PEONP22 and PEOAF22 counterparts exhibit higher ADCC activity (NK cytotoxicity) toward CD39^{high} HCC1739BL cells than their parental fully-fucosylated clone PEOWT22. Two different afucosylation methods through chemical modifications were employed using the fully human anti-CD39 monoclonal antibody PEOWT22 to produce its ADCC-enhanced counterparts PEOAF22 and PEONP22, without genetic modifications. To evaluate their ADCC profiles, target cells (CFSE-labeled HCC1739BL, an Epstein-Barr virus (EBV)-transformed human B lymphoblastoid cell line) were incubated with serially diluted human IgG1 isotype control antibody (KLH-hIgG1) or PEOWT22, PEOAF22 or PEONP22 for 30 minutes at 37° C. in 5% CO₂. Cells were then co-cultured with NK-92-CD16 V/V effector cells (E:T=1:8) for 6 hours at 37° C. Target cell death was analyzed by flow cytometry and cytotoxicity was determined by the % of CFSE+P/I+ cells.

FIG. 2 shows that IgG1 Fc fraction confers ADCC activity to antibodies. The hCD39 Ref antibody shares the antigen binding sites with an antibody in the art. However, the Ref antibody used in the current examples was generated with an Fc portion specifically designed to have ADCC function. Therefore, both hCD39 Ref antibody and our PEOWT22 contain the same ADCC-competent human IgG1 Fc fraction. NK cytotoxicity toward HCC1739BL cells were performed as described in FIG. 1 and ADCC cell killing profiles of hCD39 Ref and PEOWT22 were determined. Note that hCD39 Ref and PEOWT22 exert similar ADCC activity profiles.

FIG. 3 shows that a further optimized afucosylated counterpart PEO22 exerts higher ADCC activity than PEONP22. PEO22 is a further ADCC-enhanced version by optimizing the afucosylation process for the parental clone PEOWT22. NK cytotoxicity toward HCC1739BL cells were performed as described in FIG. 1 and ADCC cell killing profiles of PEO22 and PEONP22 were determined.

FIG. 4 shows that PEOWT22 elicited ADCC activity is selective against cells highly expressing human CD39. Various Raji cell lines expressing different levels of human CD39 including Raji cells (Raji-hCD39neg), hCD39-transfected Raji cells that highly express human CD39 (Raji-hCD39hi), or hCD39-transfected Raji cells that express low level of human CD39 (Raji-hCD39lo) were used as target cells and were pre-incubated with serially diluted PEOWT22 for 30 minutes at 37° C. in 5% CO₂. Afterwards, effector cells (Jurkat cells stably expressing luciferase and hCD16a-158V) were added into the culture (E:T=6:1) and incubated for 6 hours. ADCC activity was indicated by an increase of luciferase activity over background (RLU). RLU: Relative Luminescence Unit.

FIG. 5 shows that PEOWT22 does not exert ADCC activity toward CD39-low normal endothelial cells (HUVEC), suggestive of its low potential for systemic side-effects. Both human melanoma cells (SK-MEL-28; endogenously expressing intermediate level of CD39) and human umbilical vein endothelial cells (HUVEC; endogenously expressing low level of CD39) were used as target cells and were pre-incubated with serially diluted PEOWT22 for 30 minutes at 37° C. in 5% CO₂. Afterwards, effector cells (i.e., Jurkat cells stably expressing luciferase and hCD16a-158V) were added into the culture (E:T=6:1) and incubated for 6 hours. ADCC activity was indicated by an increase of luciferase activity over background. RLU: Relative Luminescence Unit.

FIG. 6 shows that most of Human/Rabbit chimeric anti-human CD39 monoclonal antibodies target the same epitope as the reference anti-human CD39 monoclonal antibody Clone A1: Epitope competition assay using HCC1739BL cells. HCC1739BL cells were incubated with a panel of 18 anti-human CD39 monoclonal antibodies (Human/Rabbit chimeric clones; unconjugated, 2 μg/mL) at 4° C. for 30 minutes, followed by washing and staining with PE-conjugated mouse anti-human CD39 monoclonal antibody (Clone A1) for 30 minutes at 4° C. Cells were then analyzed by flow cytometry and PE median fluorescence intensity (MFI) was detected. Cells incubated with media instead of chimeric antibody were used as control.

FIG. 7 shows that human/rabbit chimeric antibodies which do not compete for the same epitope of Clone A1 have high ADCC activity. HCC1739BL cells were used as target cells and were pre-incubated with serially diluted chimeric antibodies for 30 minutes at 37° C. in 5% CO₂. Afterwards, effector cells (i.e., Jurkat cells stably expressing luciferase and hCD16a-158V) were added into the culture (E:T=6:1) and incubated for 6 hours. ADCC activity was indicated by an increase of luciferase activity over background. RLU: Relative Luminescence Unit. Note that among these high ADCC clones, only PEO23 antibody competes for the same epitope of Clone A1 (See FIG. 6).

FIG. 8 shows that human/rabbit chimeric antibodies which completely compete for the same epitope of Clone A1 have low or no ADCC activity. Luc-reporter ADCC assay was performed as described above in FIG. 7. PEO18 serves as a positive control.

FIG. 9 shows that antibodies with high ADCC activity form stable immune complex on cell membrane while antibodies with low or no ADCC activity do not. Exemplary anti-human CD39 antibodies presenting high, low and no ADCC activity (2 μg/mL) were incubated with HCC1739BL cells for 24 hours at 37° C. in 5% CO₂ or 20 minutes at 4° C., followed by secondary antibody staining (anti-human IgG (Fc specific), Alexa Fluor® 488) for 30 minutes at 4° C. Cells were then washed and analyzed by flow cytometry. The difference in AF488 MFI between 20 minutes and 24 hours treatment represents the loss of human CD39 on cell membrane that was calculated as described in Materials and Methods. Note that clone PEO26 (low-ADCC activity) and clones PEO27, PEO28 and PEO29 (no-ADCC activity) do not form a stable immune complex on cell membrane after 24 hours incubation (e.g., the loss of CD39 is greater than 40%), whereas clones PEONP22, PEO19, PEO20, PEO21 (high-ADCC activity) form a stable antibody-antigen immune complex (e.g., the loss of CD39 is lower than 30%). Hu/Ra: Human/Rabbit chimeric antibody; hIgG1: humanized rabbit antibody, IgG1 isotype.

FIG. 10 shows that percentages of granulocytes subpopulations in blood and bone marrow of healthy mice. Blood and bone marrow (BM) samples were collected from healthy h(uman) CD39KI mice and analyzed by flow cytometry. Granulocytes were gated as eosinophils (EO) (CD45⁺CD11b⁺SSC^{high}Siglec-F⁺), neutrophils (Neu) (CD45⁺CD11b⁺Ly-6G⁺), and basophils (Baso) (CD45⁺SSClowCD200R3+Fc∈R1α⁺). The percentage of each cell subtype was calculated and presented as % of CD45⁺CD11b⁺ cells. n=8 mouse per gender.

FIG. 11 shows that eosinophils are the granulocytes subpopulations that express the highest CD39 levels on their membrane in mice-CD39 is a highly selective phenotypic biomarker for eosinophils in mice granulocytes. Blood and BM of healthy hCD39KI mouse were analyzed for human CD39 (hCD39) expression on granulocytes cell surface by flow cytometry. hCD39 expression levels on each subpopulation were shown in comparison to the Isotype Control. Note that CD39 expression is much higher on eosinophils than neutrophils, while basophils barely express the protein-CD39 expression level ranking: eosinophils>neutrophils>basophils. n=8 mice.

FIG. 12 shows that ADCC-enhanced PEO22 presents a higher blood eosinophil-depletion effect compared with its lower-ADCC counterpart PEOWT22. Healthy hCD39KI mice were treated with saline or 1 mg/kg of PEO22 or PEOWT22 (ADCC activity ranking: PEO22>PEOWT22) i.p. twice (on days 0 and 2). Blood samples were collected and analyzed on day 0 (before treatment) and on day 3 at the terminal harvest for granulocytes by flow cytometry. Note that both PEO22 and PEOWT22 antibodies are capable of selectively depleting EO, without reducing Neu amount; and such in vivo EO depletion effects positively correlate with their in vitro ADCC activities-PEO22>PEOWT22. n=1 mouse per group.

FIG. 13 shows that PEO22 selectively depletes blood and bone marrow eosinophils in healthy hCD39KI mice. Healthy hCD39KI mice were treated with saline or PEO22 (1 mg/kg) i.p. every two days for a total of 4 doses. Blood and BM samples were collected one day after the last dosing and analyzed by flow cytometry. Note that PEO22 depletes eosinophils but not neutrophils, despite decreasing both neutrophils and eosinophils CD39 membrane expression. n=4-8 mice per group. ns: non-significant; * p<0.05; *** p<0.001 **** p<0.0001 in relation to control saline group (unpaired, t-test, one-tailed).

FIG. 14 shows that eosinophils are markedly increased in asthma mice. Blood and BM samples were collected from eosinophilic asthma hCD39KI mice one day after the last OVA (i.n.) challenge and analyzed by flow cytometry. Data obtained from healthy animals were used as the reference for comparison. n=8 mice per group. ** p<0.01 in relation to healthy animals (unpaired, t-test, one-tailed).

FIG. 15 shows that PEO22 selectively depletes eosinophils in asthmatic hCD39KI mice. Asthmatic hCD39KI mice were treated with saline or PEO22 (1 mg/kg) i.p. every two days for a total of four doses. Blood and BM samples were collected one day after the last dosing and analyzed by flow cytometry. Note that eosinophils CD39 expression is also drastically decreased post PEO22 treatment in both compartments. n=8 mice per group. *p<0.05; ** p<0.01; **** p<0.0001 in relation to saline control group (unpaired, t-test, one-tailed).

FIG. 16 shows that PEO22 selectively depletes the CD39^{high} eosinophils subpopulations in asthmatic hCD39KI mice. Asthmatic hCD39KI mice were treated with saline or PEO22 (1 mg/kg) i.p. every two days for a total of four doses. Blood and BM samples were collected one day after the last dosing and analyzed by flow cytometry. Data obtained from healthy mice were used as the reference for comparison. Note that most eosinophils are CD39^{high} in healthy mice that are “inducible”-responsive to daily environmental challenges; and PEO22 treatment restores the EO absolute number in asthma mice to equal levels present in healthy mice and tips the EO subpopulations from CD39^{high} to CD39low. n=8 mice per group. ***p<0.0001 in relation to saline control group (unpaired, t-test, one-tailed).

FIG. 17 shows that PEO22 markedly depletes eosinophils in lungs of asthmatic hCD39KI mice and improves disease severity. Asthmatic hCD39KI mice were treated with saline or PEO22 (1 mg/kg) i.p. every two days for a total of four doses. Lung samples were collected one day after the last dosing and analyzed by pathology. Note that PEO22 treated group showed a remarkable improvement of focal eosinophilic inflammation predominantly in peri bronchial and perivascular areas, which are characteristic signs of OVA-induced asthma presented in the saline control group. n=8 mice per group. Pictures were taken at 100×(bar 100 μm) and 400×(bar 10 μm). Representative images of one animal per group were shown. Solid asterisk highlights the perivascular areas infiltrated with eosinophils; white asterisk indicates the vascular wall areas infiltrated with eosinophils-significant reductions in lung-infiltrating eosinophils are seen in both areas in the PEO22 treated group.

FIG. 18 shows that PEO22 improves pulmonary eosinophilic vasculitis in asthmatic hCD39KI mice. Asthmatic hCD39KI mice were treated with saline or PEO22 (1 mg/kg) i.p. every two days for a total of four doses. Lung samples were collected one day after the last dosing and analyzed by pathology. Note that PEO22 treated group showed signs of decreased eosinophil numbers and activity within the vascular wall when compared to saline control group. n=8 mice per group. Pictures were taken at 100×(bar 100 μm) and 400×(bar 10 μm). Representative images of one animal per group were shown. Solid asterisk highlights the perivascular areas infiltrated with eosinophils; white asterisk indicates the vascular wall infiltrated with eosinophils.

FIG. 19 shows that CD39^{high} phenotypically and functionally defines activated eosinophils subpopulations in asthma mice. Blood and BM samples from asthmatic hCD39KI mice were collected one day after the last OVA (i.n.) challenge and analyzed by flow cytometry for eosinophil biomarkers including previously reported activation markers. Note that EO cells highly expressing CD39 (the CD39^{high} subtype) represent the vast majority of EO subpopulations in both blood and bone marrow of asthma mice. n=10 mice per group.

FIG. 20 shows that IL-5Rα is not a selective biomarker for eosinophils in mice granulocytes, as shown in both healthy and asthma mice. Blood and BM samples from asthmatic hCD39KI mice collected one day after the last OVA (i.n.) challenge were analyzed by flow cytometry for IL-5Rα expression on granulocytes. Data from healthy mice were used in parallel for comparison. Note that neutrophils, but not eosinophils, are the granulocytes subpopulations that highly express IL-5Rα in blood and BM of both healthy and asthmatic mice. n=8 mice per group.

FIG. 21 shows that blood eosinophils are markedly increased in mouse dermatitis. hCD39KI mice were subjected to atopic dermatitis model induction as detailed in Materials and Methods. Blood samples were collected two days after the last OVA challenge and analyzed by flow cytometry. Data from healthy animals were used as the reference for comparison. n=5-8 mice per group. ** p<0.01 in relation to healthy mice (unpaired, t-test, one-tailed).

FIG. 22 shows that PEO22 decreases skin tissue-infiltrating eosinophils and improves characteristic signs of dermatitis-epidermal hyperplasia, hyperkeratosis, necrotic keratinocytes and inflammatory infiltration. hCD39KI mice subjected to dermatitis model induction were treated with saline or PEO22 (1 mg/kg) i.p. for a total of three doses. Skin samples were collected two days after the last dosing and analyzed by pathology. n=8 mice per group in the dermatitis model. Pictures were taken at 400×(bar 10 μm). Representative images of one animal per group were shown. Skin pathology of healthy mice was illustrated in parallel as the reference for comparison. Solid asterisk indicates local inflammation areas infiltrated with eosinophils-marked reductions in skin tissue inflammation are noted in the PEO22 treated group.

FIG. 23 shows that PEO22 selectively depletes blood eosinophils in mouse eosinophilic rhinosinustis. hCD39KI mice subjected to eosinophilic rhinosinustis model induction, as detailed in Materials and Methods, were treated with saline or PEO22 (3 mg/kg) i.p. every two days for a total of four doses. Blood samples were collected one day after the last dosing and analyzed by flow cytometry. Data from healthy animals were used as the reference for comparison. Note that blood EO cells are markedly increased in the rhinosinustis model in contrast to healthy mice, which is reduced in the PEO22 treated group. n=5 mice per group in the rhinosinusitis model. ** p<0.01 in relation to respective group (One-way ANOVA, followed by Tukey's multiple comparisons test).

FIG. 24 shows that PEO22 decreases nasal cavity subepithelial infiltrating inflammatory cells that are dominated by eosinophils, the pathogenesis of eosinophilic rhinosinusitis, and improves local lesions. hCD39KI mice subjected to eosinophilic rhinosinustis model induction were treated with saline or PEO22 (3 mg/kg) i.p. every two days for a total of four doses. Nasal cavities samples were collected one day after the last dosing and analyzed by pathology. n=5 mice per group. Nasal cavities were consistently sampled at level II (through the incisive papilla rostral to the first palatal ridge), and pathology images were focused on the nasal septum. Pictures were taken at 100×(bar 100 μm) and 400×(bar 10 μm). Representative images of one animal per group were shown. Solid asterisk indicates subepithelial infiltrating inflammatory cells dominated with eosinophils.

FIG. 25 shows that percentages of granulocytes subpopulations in human blood. Healthy human blood samples were analyzed by flow cytometry. Granulocytes were gated as eosinophils (EO) (CD45⁺SSC^{high}Siglec-8⁺), neutrophils (Neu) (CD45⁺SSC^{high}Siglec-8⁻), and basophils (Baso) (CD45⁺SSCLOWHLA-DR-IL-3Rα⁺). The percentage of each cell subtype was calculated and presented as % of CD45⁺SSC^{high} (EO and Neu) and % of CD45⁺SSC^{low} (Baso).

FIG. 26 shows that eosinophils are the granulocytes subpopulations that express the highest CD39 levels on their membrane in humans-CD39 is a highly selective phenotypic biomarker for eosinophils in human granulocytes. Healthy human blood samples were analyzed for hCD39 expression on granulocytes cell surface by flow cytometry. hCD39 expression levels on each subtype were shown in comparison to the Isotype Control. Note that CD39 expression is much higher on eosinophils than neutrophils, while basophils barely express the protein-CD39 expression level ranking: eosinophils>neutrophils>basophils.

FIG. 27 shows that like healthy mice, most eosinophils are CD39^{high} in healthy humans. Blood eosinophils of healthy humans were further subdivided based on hCD39 expression analyzed by flow cytometry. Note that the vast majority of eosinophils are the CD39^{high} inducible subtype in healthy humans.

FIG. 28 shows that ADCC-enhanced PEO22 exhibits increased ADCC activity toward human eosinophils in comparison to its lower-ADCC counterpart PEOWT22. Isolated human EO target cells were incubated with serially diluted PEOWT22 or PEO22 for 30 minutes at 37° C. in 5% CO₂. Cells were then co-cultured with isolated and activated human NK effector cells (E:T=1:5) for 6 hours at 37° C. Target cell death was analyzed by flow cytometry and cytotoxicity was determined by the % of 7-AAD⁺EO cells.

FIG. 29 shows that PEO22 exhibits ADCP activity toward human eosinophils. Violet labeled human EO target cells were incubated with Isotype Control (10 μg/mL) or PEO22 (1 or 10 μg/mL) for 30 min at 37° C. in 5% CO₂. Cells were then co-cultured with human macrophages effector cells (E:T=3:1) for 6 hours at 37° C. Attached macrophages were collected and analyzed by flow cytometry and ADCP was determined by the % of Pacific Blue⁺ (EO-engulfed) macrophages cells. Macrophages incubated alone with ADCP media were used as the negative control in the phagocytosis assay.

FIG. 30 shows conformational epitope mapping and a list of main putative epitope candidates.

### DETAILED DESCRIPTION OF THE INVENTION

### I. Overview

Effective treatment of inflammatory diseases is often challenging owing to their heterogeneous pathophysiology. Understanding of the underlying disease mechanisms is improving and it is now clear that eosinophils play a complex pathophysiological role in a broad range of inflammatory diseases, including central roles in type 2 inflammatory diseases.

Eosinophils are a subset of differentiated granulocytes which circulate in peripheral blood and home in several body tissues. Along with their traditional relevance in helminth immunity and allergy, eosinophils have been progressively attributed important roles in a number of homeostatic and pathologic situations. For instance, in allergy, eosinophils are implicated in the pathogenesis of atopic dermatitis, allergic rhinitis, and asthma. They are also fundamentally involved in autoimmune disorders such as eosinophilic esophagitis, eosinophilic gastroenteritis, acute and chronic eosinophilic pneumonia, and eosinophilic granulomatosis with polyangiitis (a rare form of vasculitis). In addition, secondary (or reactive) eosinophilias can also be induced/caused by drugs such as ICIs.

The present invention is based at least in part on the discovery that certain CD39-targeted agents, such as ADCC-competent and/or ADCP-competent anti-CD39 antibodies are capable of selectively targeting and ablating CD39 expressing eosinophils, and as a consequence causing a decrease in the number of eosinophils systemically and/or located in a target tissue (such as a site of inflammation or lesions or the site of eosinophil production-bone marrow). The resulting reduction in numbers and/or function of CD39+eosinophil cells can lead to changes in the inflammatory phenotype of the tissue.

Accordingly, in some aspects, the present invention relates to CD39-targeted eosinophil-depleting agents. The term “CD39-targeted eosinophil-depleting agents” refers to any agent (e.g., antibodies, small molecules, aptamers, etc.) that specifically binds to CD39 on the surface of eosinophils and induces cell death of eosinophils. In some embodiments, the CD39-targeted eosinophil-depleting agent is anti-CD39 antibodies, such as ADCC-competent and/or ADCP-competent anti-CD39 antibodies.

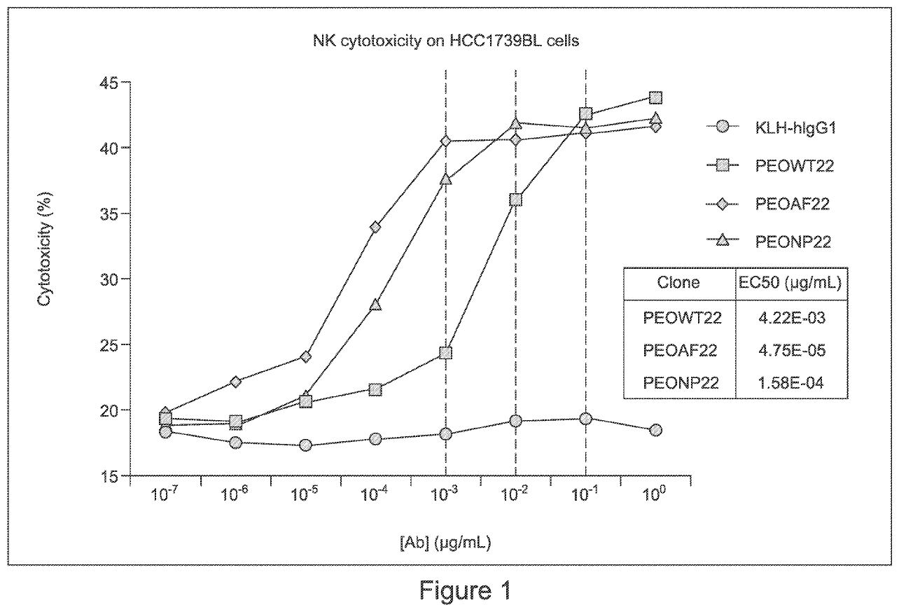

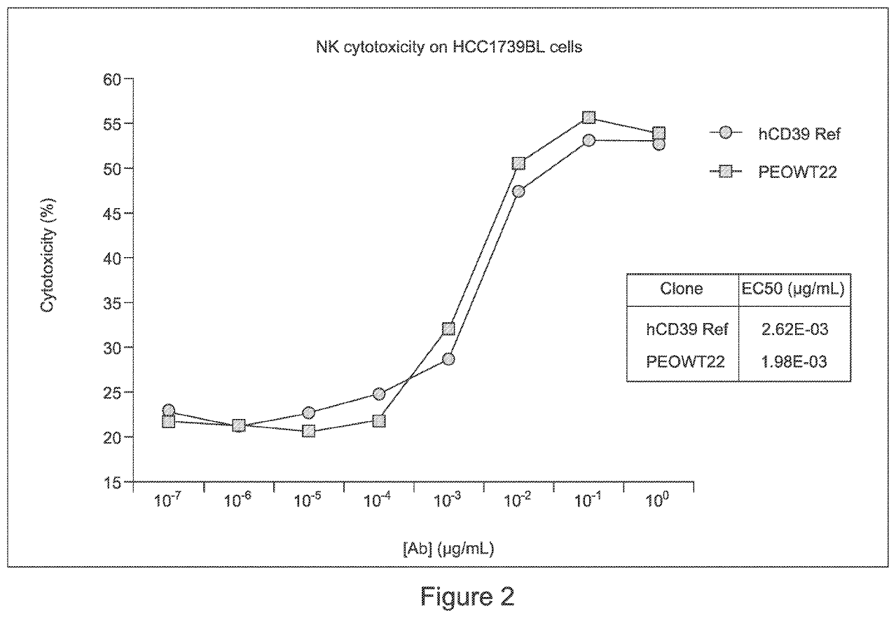

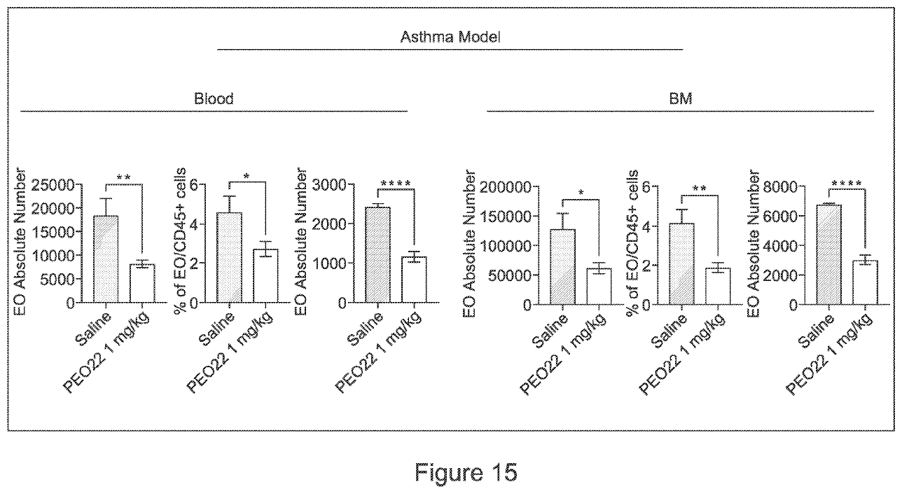

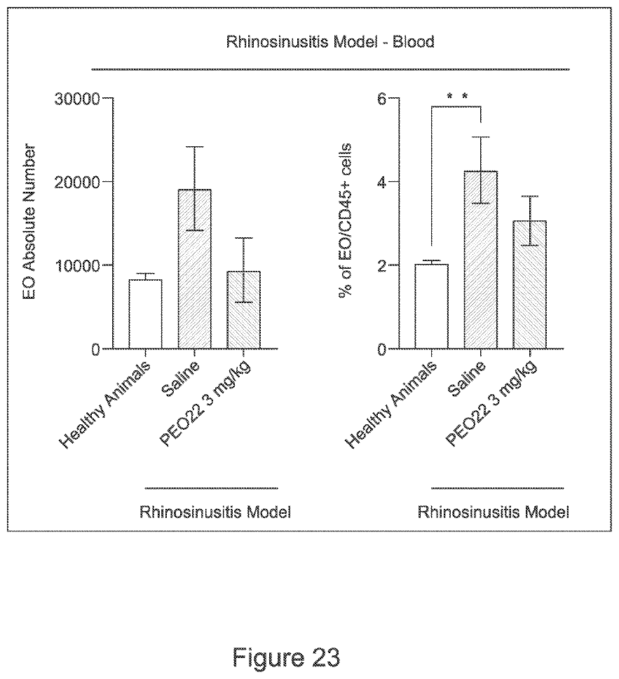

To illustrate the use of anti-CD39 antibodies as an example of CD39-targeted eosinophil-depleting agents, antibodies which can be selected for use in the methods of the present invention are capable of forming more stable immune complexes with CD39 in order to produce a more potent ADCC killing efficacy. Antibodies that are not able to form stable immune complexes with CD39, the inventors have observed, result in reduction of CD39 from the surface but through a mechanism of increased shedding of CD39 (antigenic modulation) or cytosis (internalization) and do not have the same efficacy in terms of being able to ablate the CD39 expressing cells by antibody-dependent cellular cytotoxicity. Without being bound by theory, it is further believed that the use of anti-CD39 antibodies as an example of CD39-targeted eosinophil-depleting agents, antibodies which can be selected for use in the methods of the present invention are capable of forming more stable immune complexes with CD39 in order to produce a more potent ADCP killing efficacy. ADCP is another major Fc effector function whose mechanism, by which antibody-opsonized target cells activate FcγRs (such as FcγRIIIa receptors described herein on cells, such as those expressed on NK cells and conferring NK cells' ADCC killing function) on the surface of macrophages to induce phagocytosis, resulting in internalization and degradation of the target cell and, ultimately, killing of target cells. Hypo-fucosylation or afucosylation of therapeutic monoclonal antibodies has been shown to result in enhanced FcγRIIIa receptor binding and subsequent ADCC-mediated target cell depletion activity by NK cells, as well as ADCP-mediated target cell depletion by macrophages (see FIGS. 1, 3, 12, 13, 15-18, 22-24, 28, and 29 herein; also Dagher et al., 2022, Eur. Respir. J. 59:2004306; wherein target cells in both experimental settings are eosinophils).

Exemplary features of certain preferred anti-CD39 monoclonal antibodies, features which are taught away from for use in therapeutic anti-CD39 antibodies described in the literature, are summarized as below.

The subject antibodies reduce CD39+eosinophil cell populations through FcγRIIIa receptor-dependent activity e.g. ADCC and/or ADCP.

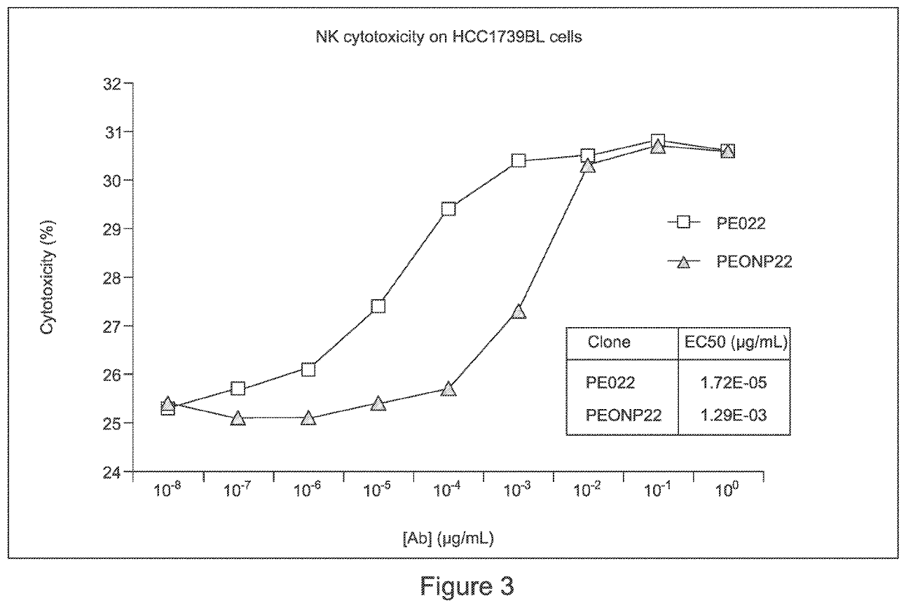

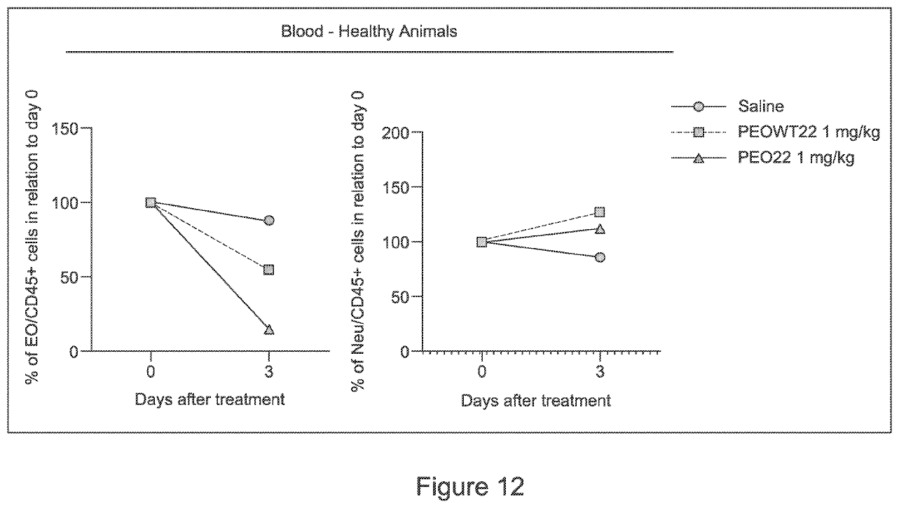

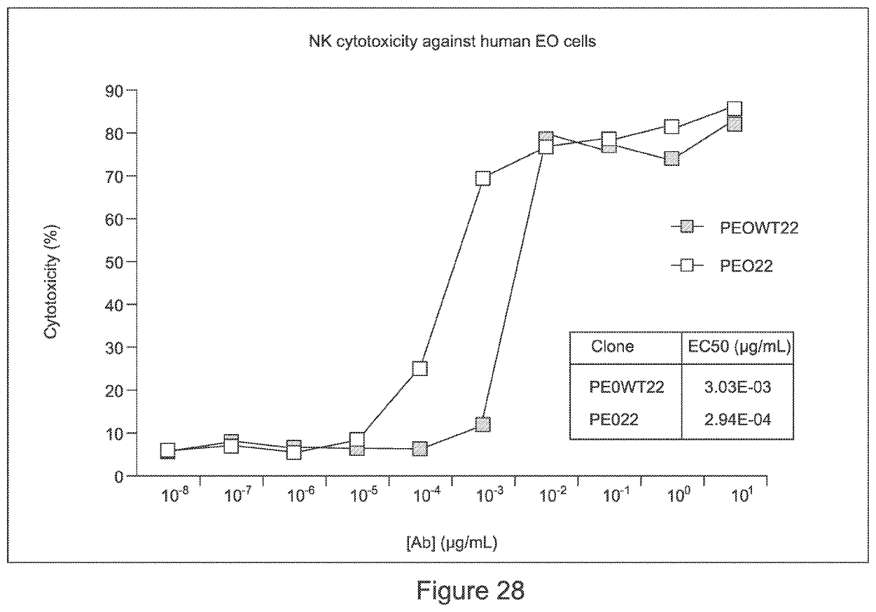

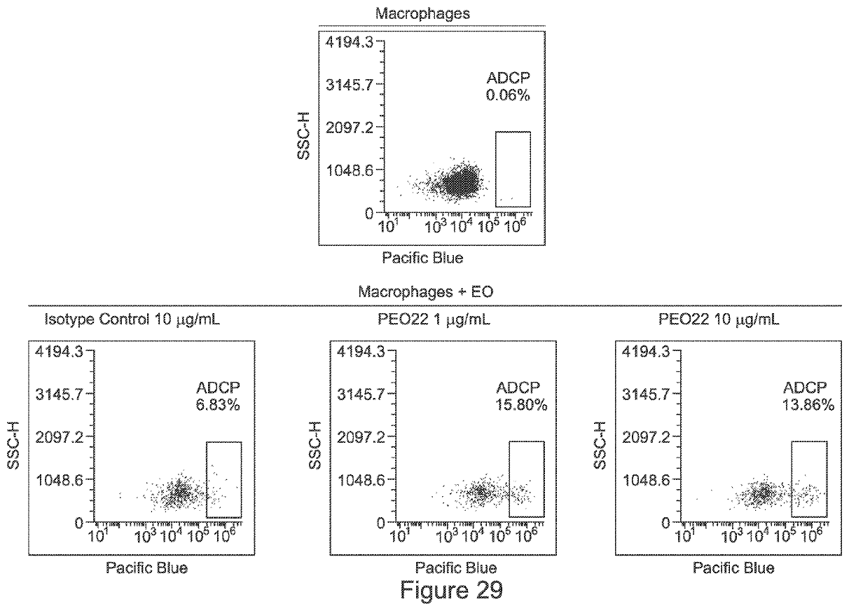

As examples, FIGS. 1 and 3 show that decreased fucosylation (a.k.a. hypo-fucosylation or afucosylation) of clone PEOWT22, a fully human anti-CD39 monoclonal antibody, either by using a fucosylation inhibitor (PEOAF22) or by optimizing the production process (PEONP22 and PEO22), dramatically boosts its ADCC activity against CD39+ cells in vitro-ADCC activity ranking: PEO22>PEONP22>PEOWT22. Such phenomenon is also valid when using human NK cells (as the effector cells) and human eosinophils (as the target cells) in an in-vitro ADCC assay, viz., PEO22 shows markedly enhanced NK cytotoxicity toward eosinophils (EC50=2.94E-04 μg/mL) in contrast to its fully-fucosylated counterpart PEOWT22 (EC50=3.03E-03 μg/mL) (FIG. 28). In addition, PEO22 also demonstrates ADCP activity against eosinophils in an in-vitro phagocytosis assay using human macrophages (as the effector cells) and human eosinophils (as the target cells) (FIG. 29). As expected, all these in vitro CD39-targeted eosinophil-depleting activities are concurrent with enhancement of anti-eosinophilic activity of the afucosylated antibody PEO22 in vivo (FIGS. 12, 13, 15-18, and 22-24).

CD39^{high} phenotypically and/or functionally defines inducible and/or activated eosinophil subpopulations.

Eosinophils are heterogeneous and different eosinophil subpopulations exist, i.e., resident eosinophils in various tissues and inducible eosinophils, which may have an impact on the efficacy of eosinophil-targeted therapy. Characterization of eosinophil subpopulations is an important aim of eosinophil research. Thus, it would be worthwhile to phenotype different eosinophil subpopulations by membrane surface markers, in order to distinguish homeostatic versus inflammatory eosinophils, for the development of safer, more selective and more effective therapies to treat diseases or conditions associated with unwanted eosinophilic activity, viz., therapeutic intervention with biological agents that totally deplete tissues and circulating eosinophils or, vice versa, maintain a minimal proportion of eosinophils, particularly those residents in tissues or homeostatic, could therefore have a very different impact, especially when considering the administration of these therapies for prolonged periods. Similarly, use of bona fide biomarkers of inducible eosinophils (e.g., circulating eosinophils, organ-specific eosinophils) can also help in to choose the best eosinophil-targeted approach among the increasing therapeutic armamentarium of biological agents. However, eosinophil subtypes have not been fully characterized in humans. (Messnil et al., 2016, J. Clin. Invest. 126:3279-3295; Kanda et al., 2021, Allergol. Int. 70 (1): 9-18; Lombardi et al., 2022, Curr. Res. Immunol. 3:42-53).

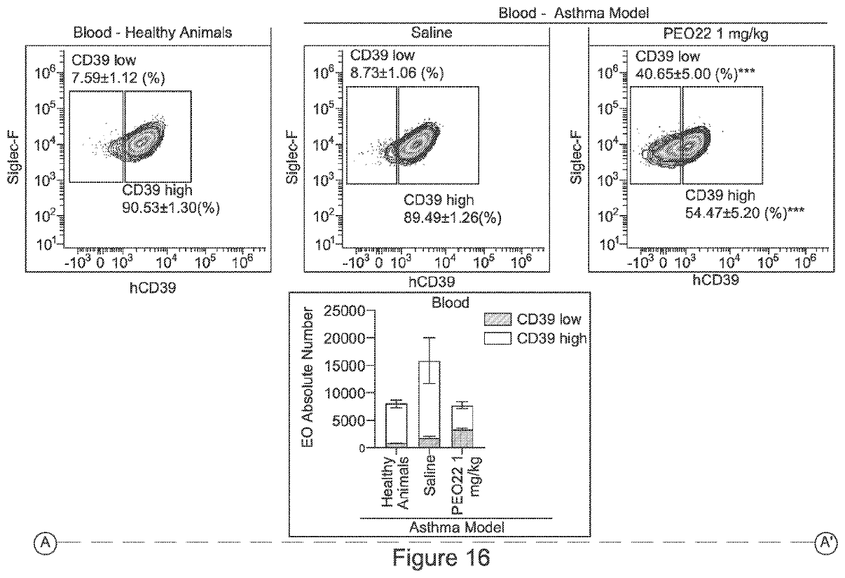

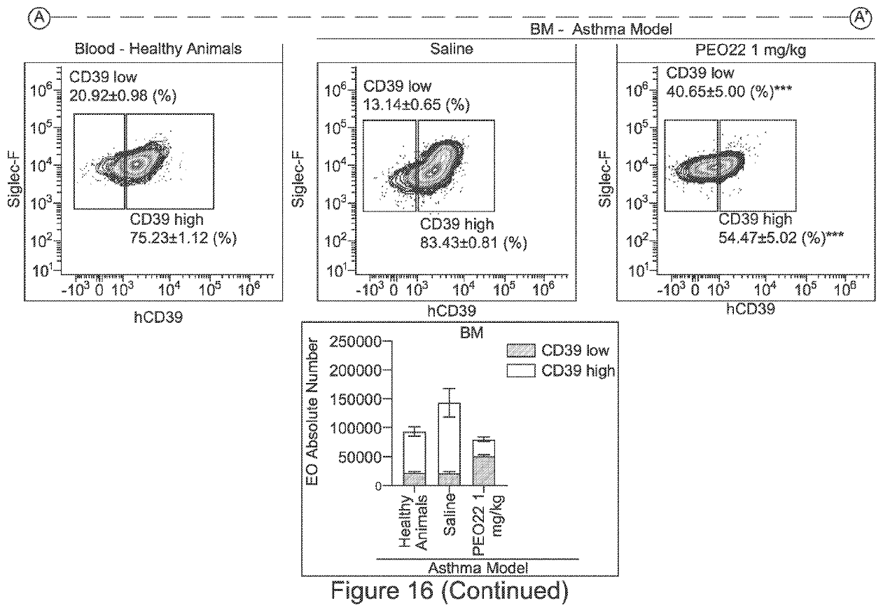

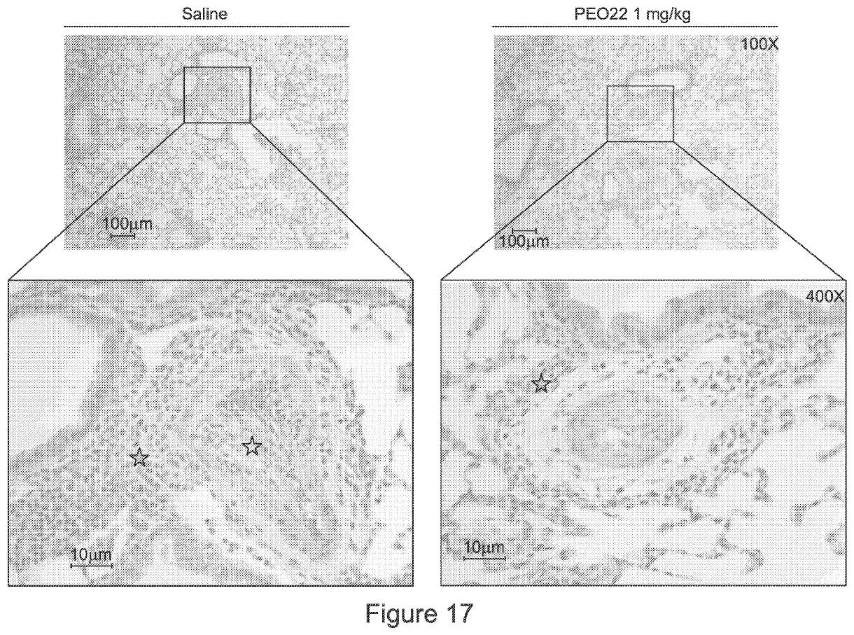

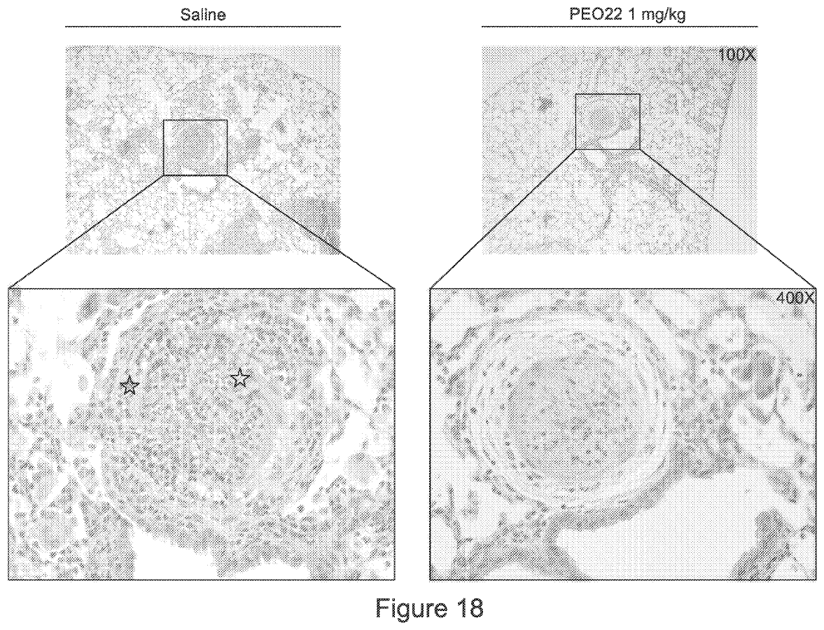

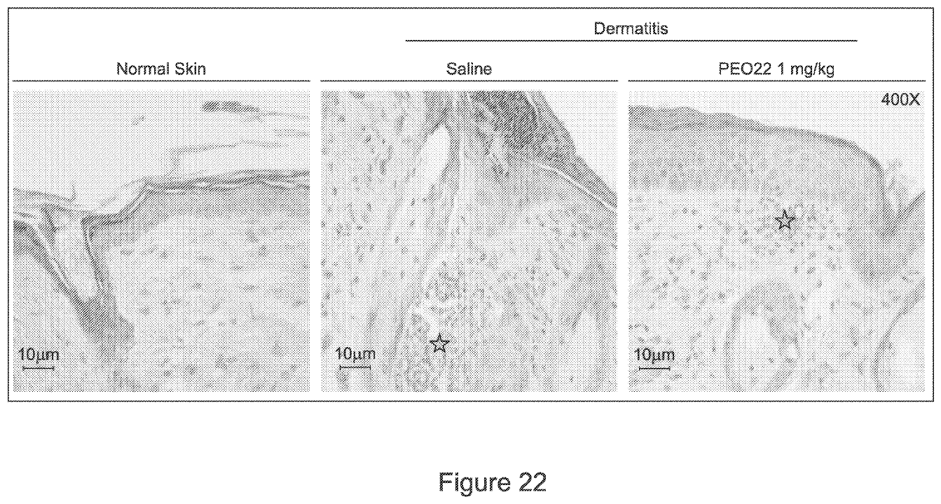

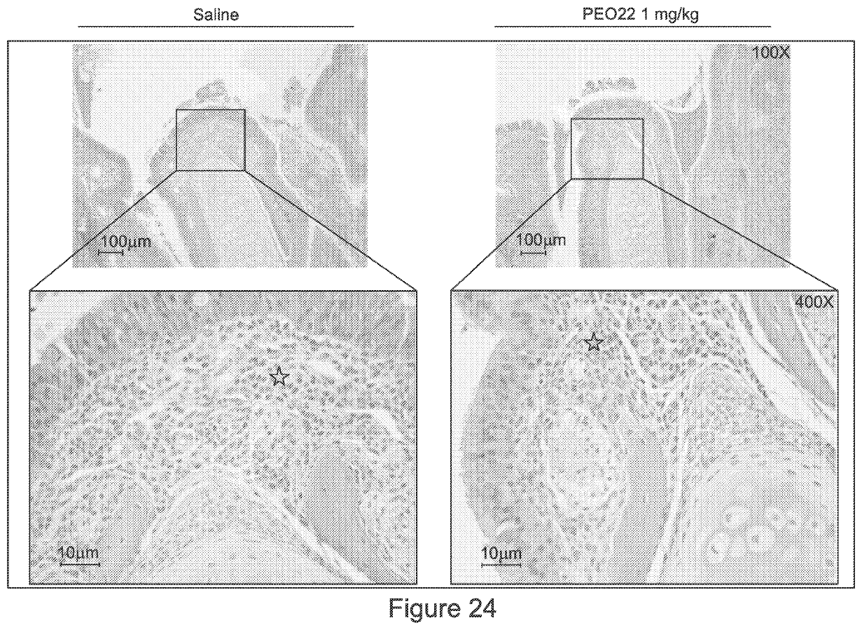

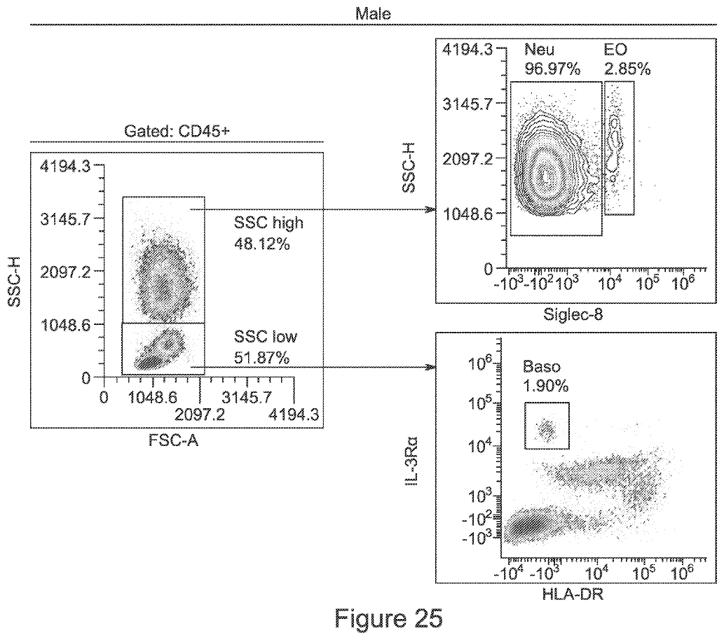

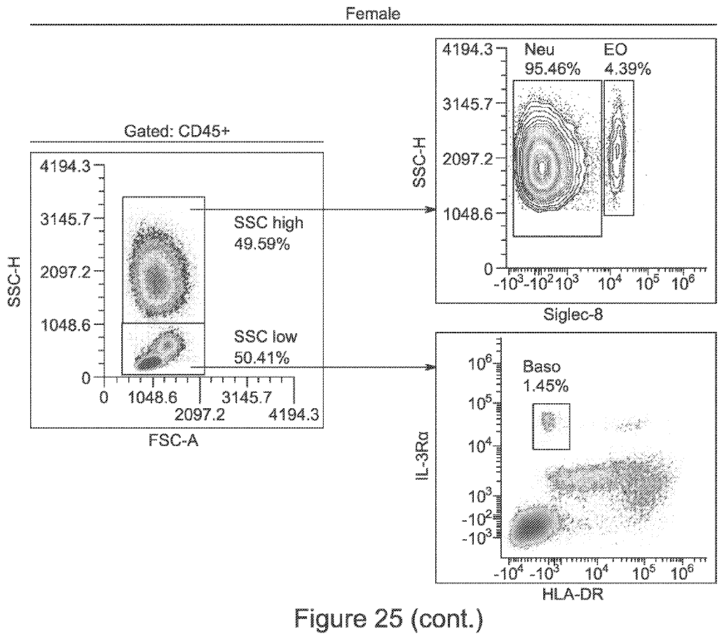

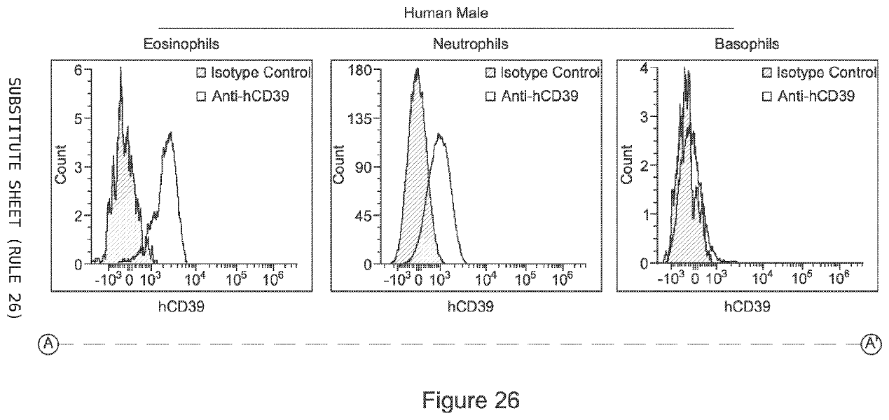

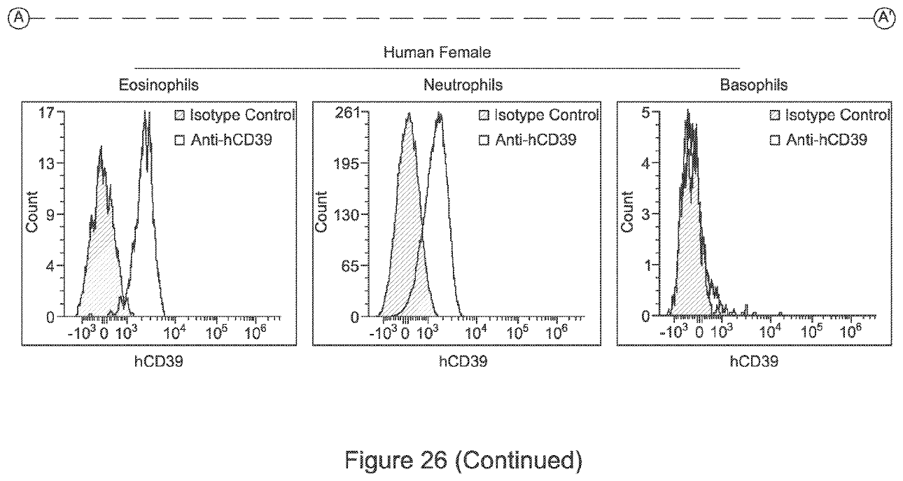

Herein, we show that 1) almost all eosinophils express CD39 (eosinophil cells are CD39⁺); and 2) CD39^{high} phenotypically and/or functionally defines inducible and/or activated eosinophils, which account for the vast majority of eosinophil cells, in both mice and humans. As examples, FIG. 16 shows that, based on cell surface CD39 expression levels analyzed by flow cytometry, eosinophils can be phenotypically classified into two distinct subpopulations in both healthy and asthma mice, that is CD39^{high} versus CD39low. Additionally, these CD39^{high} eosinophils demonstrate “inducible” functional traits—are responsive to and expand upon inflammation induction, i.e., increased numbers of CD39^{high} eosinophil subpopulations in the peripheral blood and bone marrow are noted in asthma mice (FIG. 16); concurrent with increased numbers of infiltrating eosinophils in the lungs (e.g., focal eosinophilic inflammation predominantly in peri bronchial and perivascular areas, accompanied with vasculitis (FIGS. 17 and 18); all are positively correlated with increased disease severity (FIGS. 17 and 18). Such phenomena are also seen in the atopic dermatitis and eosinophilic rhinosinusitis mouse models (FIGS. 22 and 24). More importantly, similar eosinophils' CD39 expression profiles are also observed in human blood (FIGS. 25-27)-implying the good clinical translatability of CD39-targeted eosinophil-depleting agents, such as the subject anti-CD39 antibodies in the present invention.

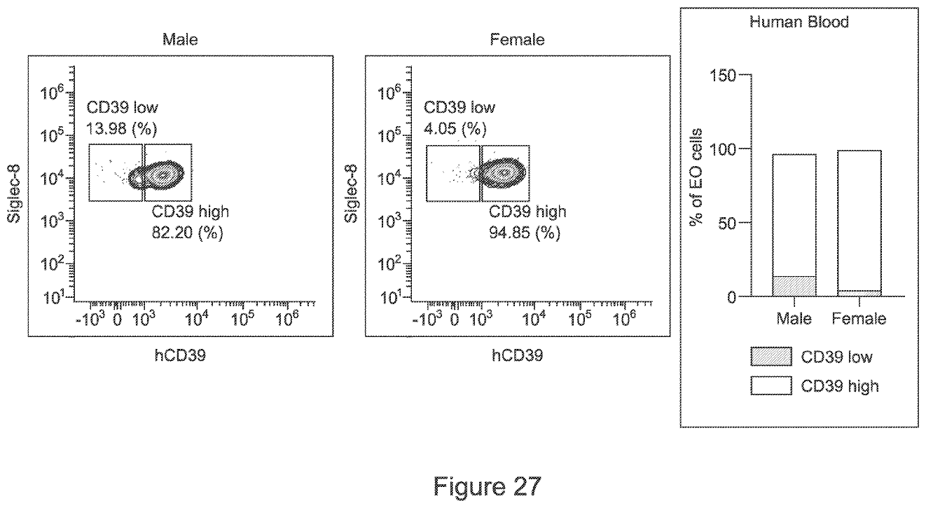

Notably, the vast majority of eosinophils are the CD39^{high} inducible subtypes under hemostatic conditions (such as healthy mice and healthy humans), i.e., 80-90% CD39^{high} versus 10-20% CD39low (FIGS. 16 and 27). In eosinophil-associated diseases (EADs) such as asthma, these CD39^{high} eosinophils are expanded and increased (FIG. 16)-suggesting CD39^{high} predisposes eosinophils to be “inducible” and responsive to inflammation induction; and CD39^{high} is a selective phenotypic and/or functional biomarker of “activated” eosinophils, which are the pathogenesis of EADs.

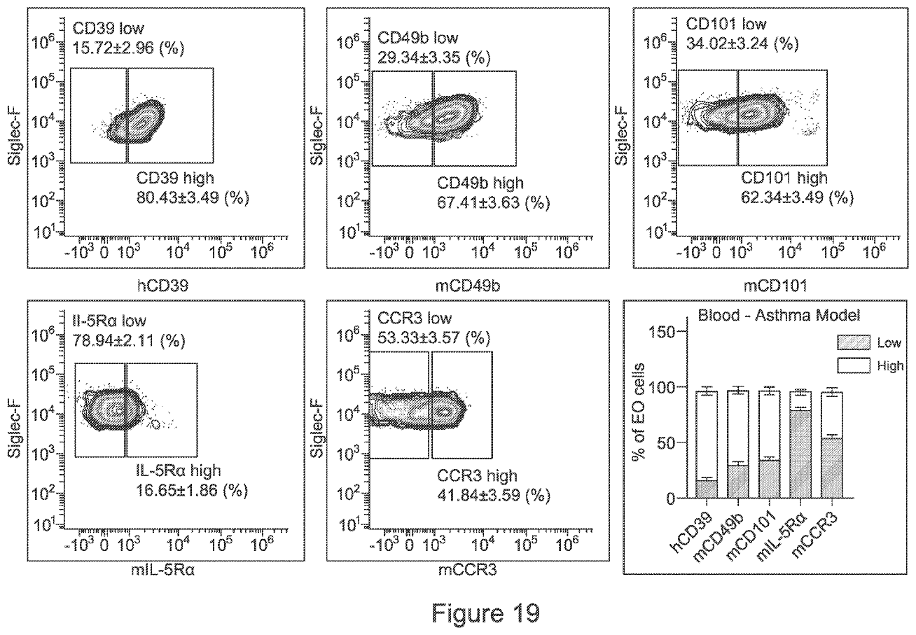

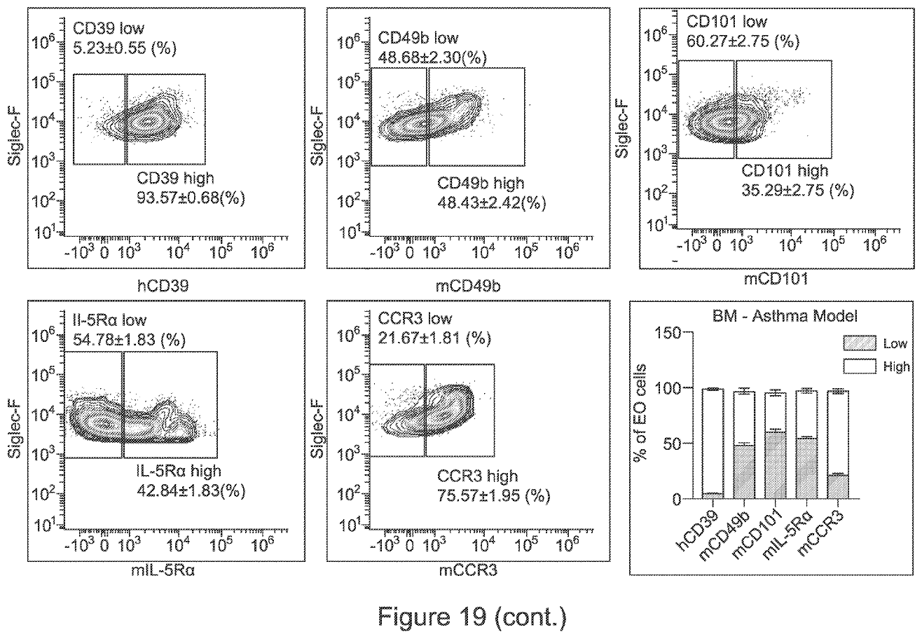

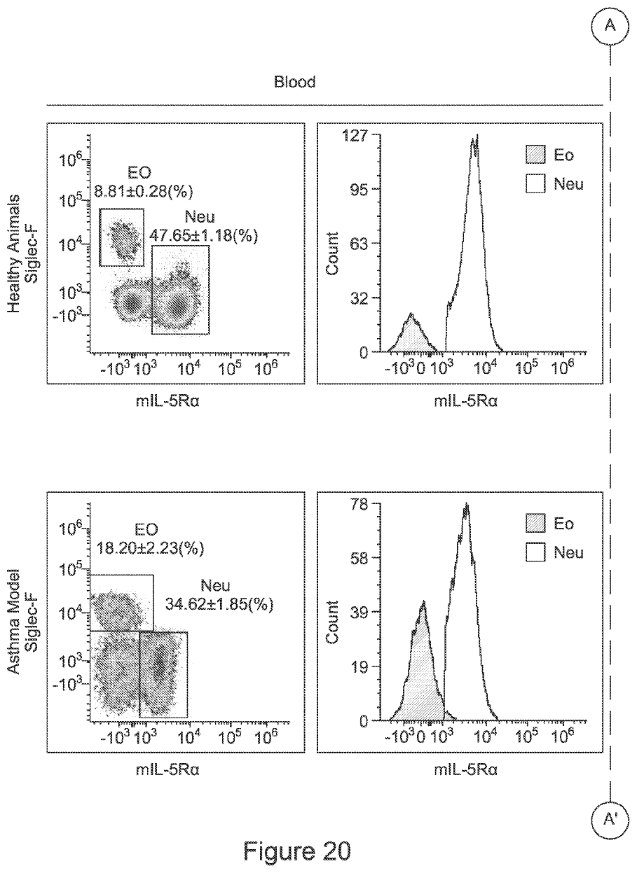

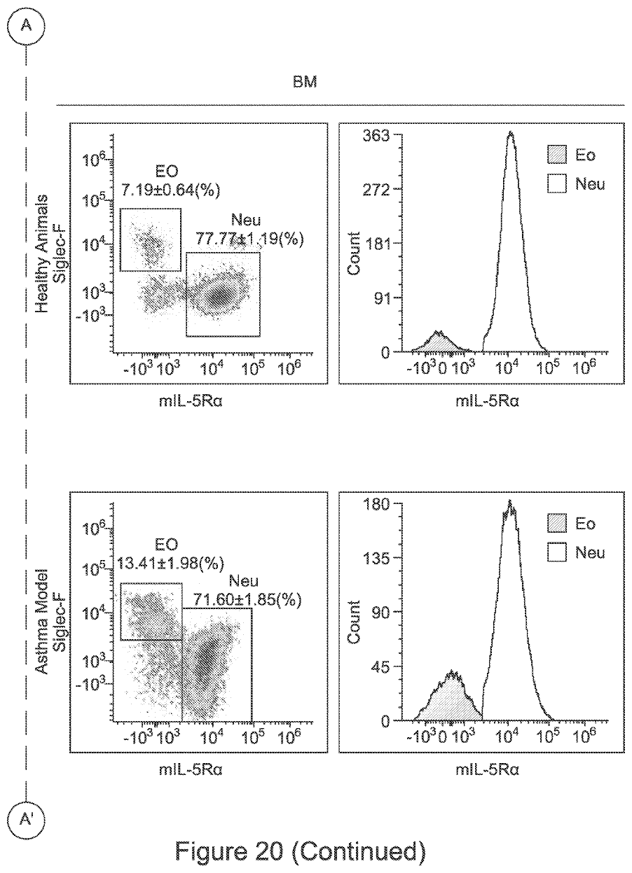

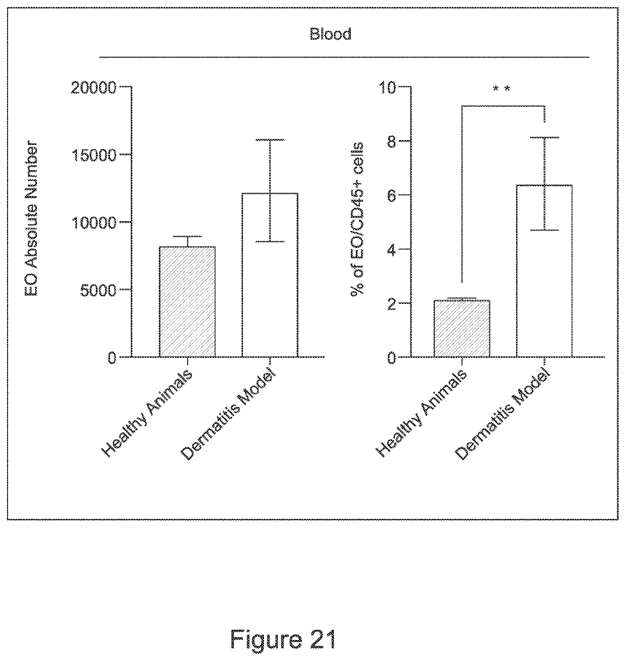

Unlike CD39, other previously reported phenotypic biomarkers of “activated” eosinophils in EADs, as examples, such as CD49b and CD101, being referred to as “upregulated” on eosinophils, are unable (or at least to a much lesser extent than CD39) to distinguish activated eosinophil subpopulations from their homeostatic counterparts (FIG. 19). As another example, FASENRA® (Benralizumab), a humanized IgG1 anti-IL-5Rx monoclonal antibody that is afucosylated for ADCC enhancement to deplete eosinophils and basophils, is the first and only anti-eosinophil biologic that binds directly at the surface of the eosinophil and approved for eosinophilic asthma (available on the World Wide Web at fasenrahcp.com). However, in eosinophilic asthma, the IL-5Rα^{high} eosinophil subpopulations only account for a small proportion of eosinophils, i.e., around 20% in blood and less than 50% in bone marrow (FIG. 19); and moreover, neutrophils indeed express much higher IL-5Rα levels than eosinophils (Kanda et al., 2021, Allergol. Int. 70 (1): 9-18-FIG. 3; and FIG. 20 herein). Therefore, the depleting activities of IL-5Rx-targeted eosinophil-depleting agents such as Benralizumab indeed lack eosinophil cell selectivity.

These data indicate that CD39^{high} serves as a bona fide phenotypic and/or functional biomarker for the inducible and/or activated eosinophil subtypes in EADs-providing further evidence of high selectivity and potentially high efficacy of CD39-targeted eosinophil-depleting agents to treat diseases or conditions associated with unwanted eosinophil activity. On another note, our data herein also suggest the potential clinical utility of CD39^{high} eosinophils as biomarkers to aid in stratification of patients with EADs.

ADCC activity of the subject anti-CD39 antibodies is selective toward CD39^{high} cells, e.g., CD39^{high} inducible and/or activated eosinophils.

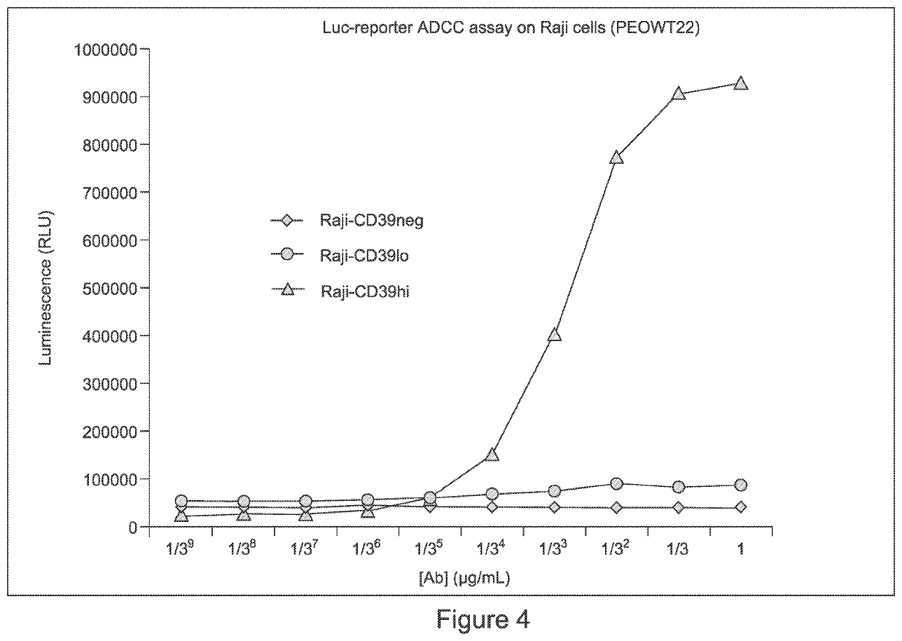

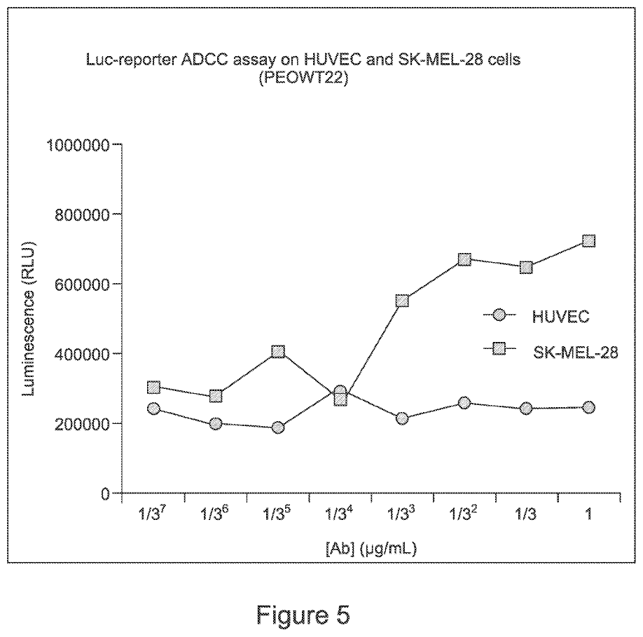

As examples, FIGS. 4 and 5 show that ADCC activity of PEOWT22 is selective against CD39^{high} cells in vitro (i.e., Raji-hCD39hi cells in FIG. 4 and SK-MEL-28 cells in FIG. 5).

As further examples shown in FIG. 16, PEO22 is capable of selectively targeting and ablating CD39^{high} inducible and activated eosinophils in vivo; and as a consequence causing a decrease in the number of these CD39^{high} eosinophils systemically in EADs such as eosinophilic asthma (FIG. 16). Concurrently, numbers of tissue-infiltrating eosinophils are also markedly reduced in the pathological lesions by PEO22 treatment in mouse models of asthma, vasculitis, atopic dermatitis, and eosinophilic rhinosinusitis (FIGS. 17, 18, 22, and 24). The resulting reduction in numbers and/or function of CD39^{high} eosinophil subpopulations lead to improvements in the inflammatory phenotype of the tissues as well as decreases in disease severity (FIGS. 17, 18, 22, and 24).

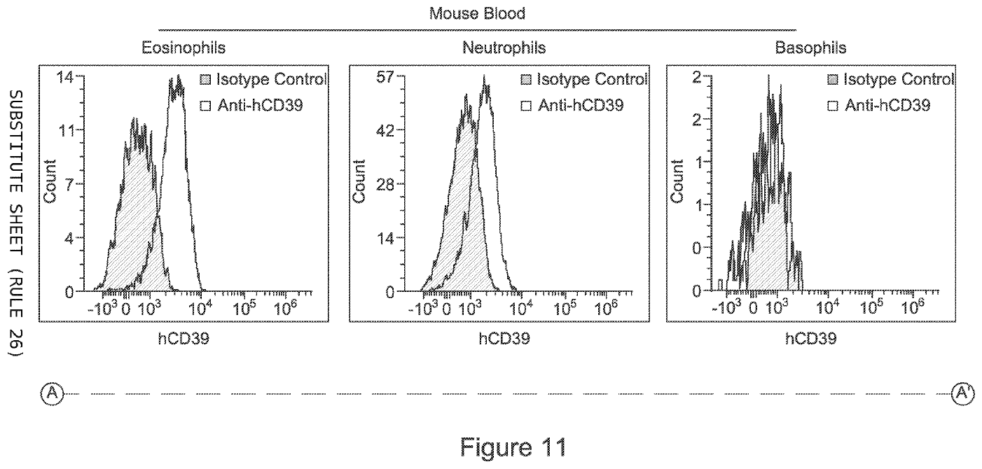

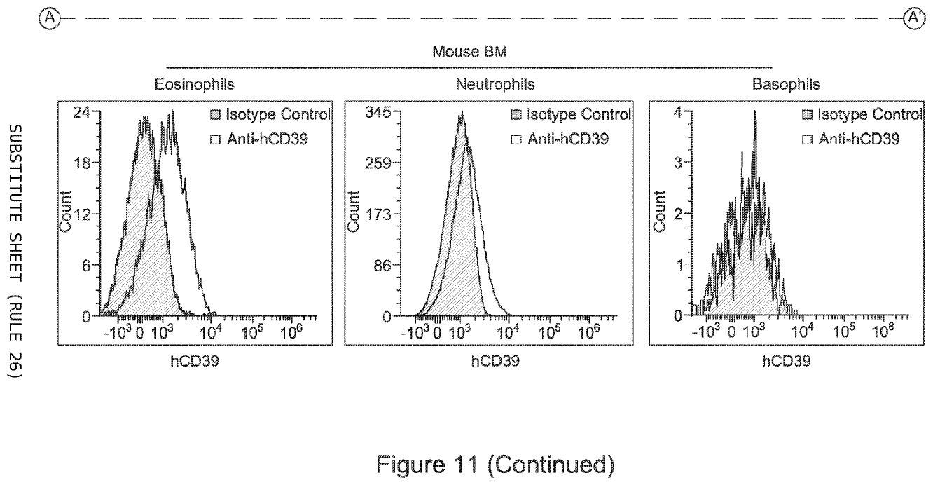

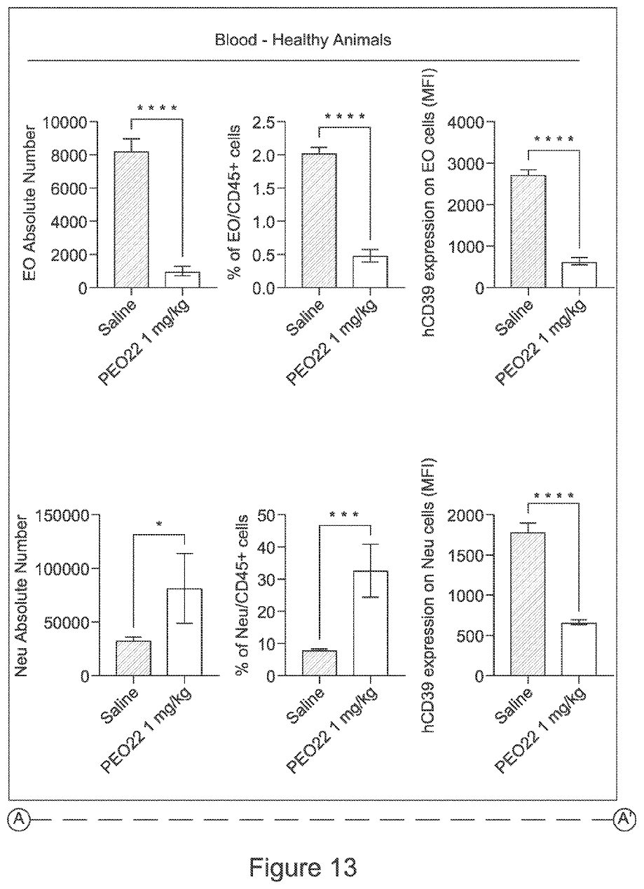

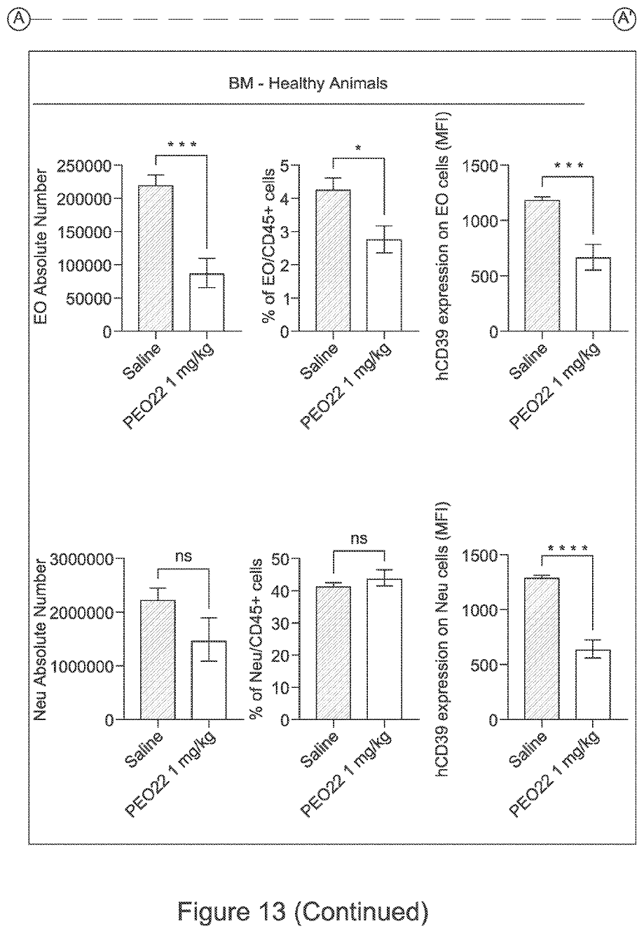

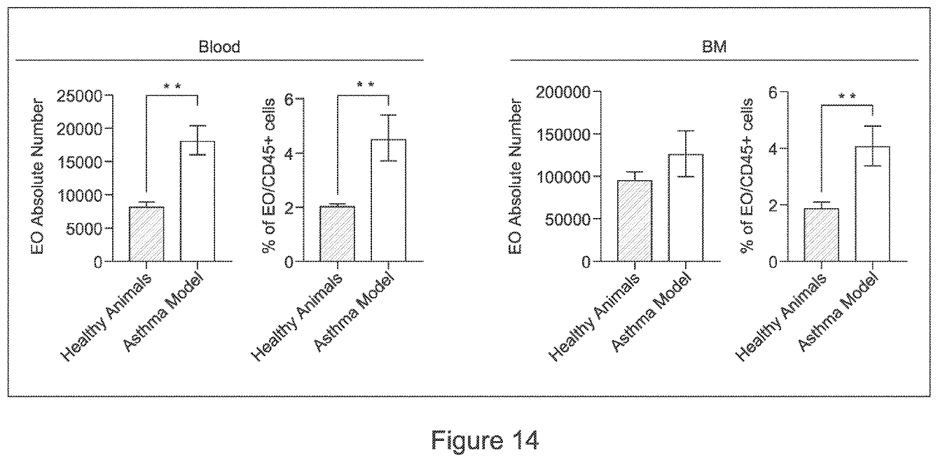

As additional examples shown in FIGS. 12 and 13, in contrast, neutrophils which express much lower CD39 levels than eosinophils (FIG. 11), are not depleted by in vivo PEO22 treatment.

In summary, this functional trait should confer specificity to the subject antibodies, avoiding systemic side-effects which should result in a safer, highly selective, and more effective anti-CD39 antibody to treat EADs—diseases or conditions associated with unwanted eosinophil activity.

Formation of a stable immune complex of anti-CD39 antibodies with the antigen on target cell membrane confers high ADCC activity to the antibodies.

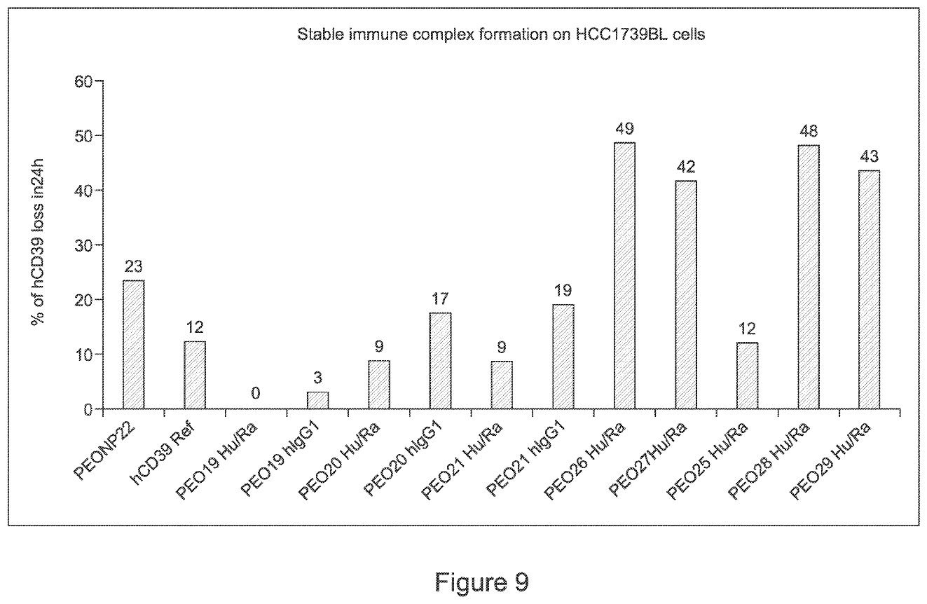

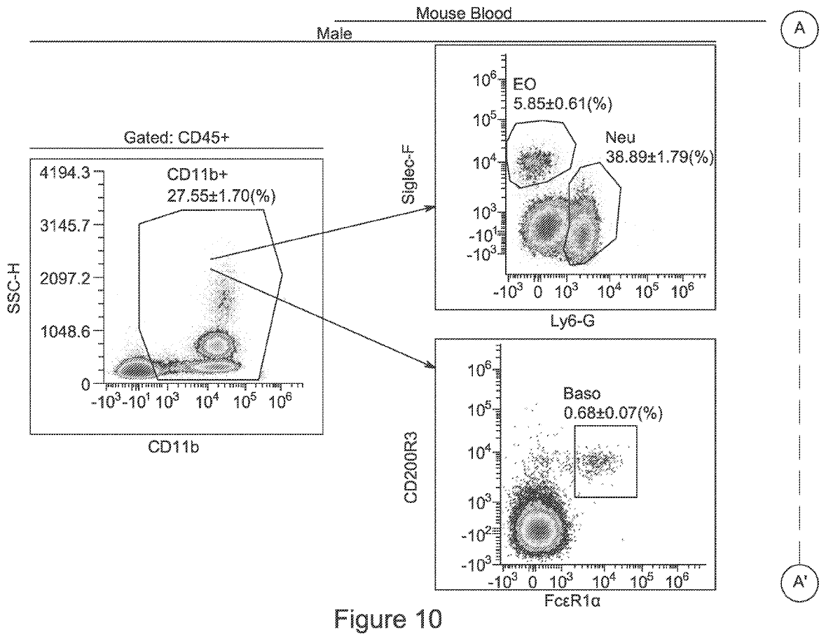

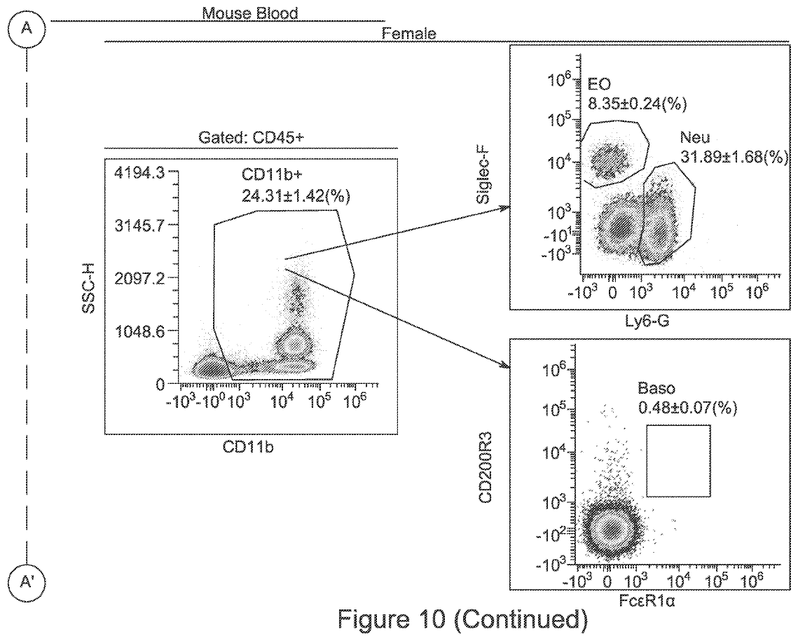

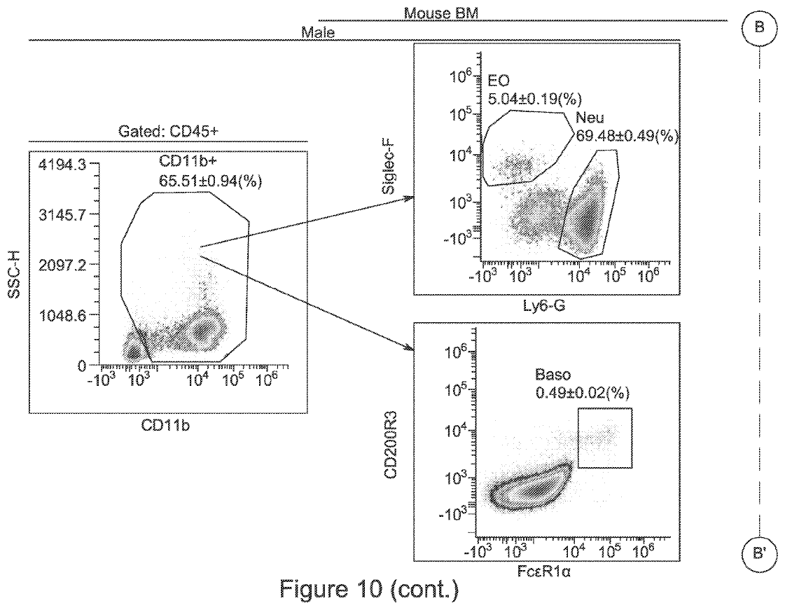

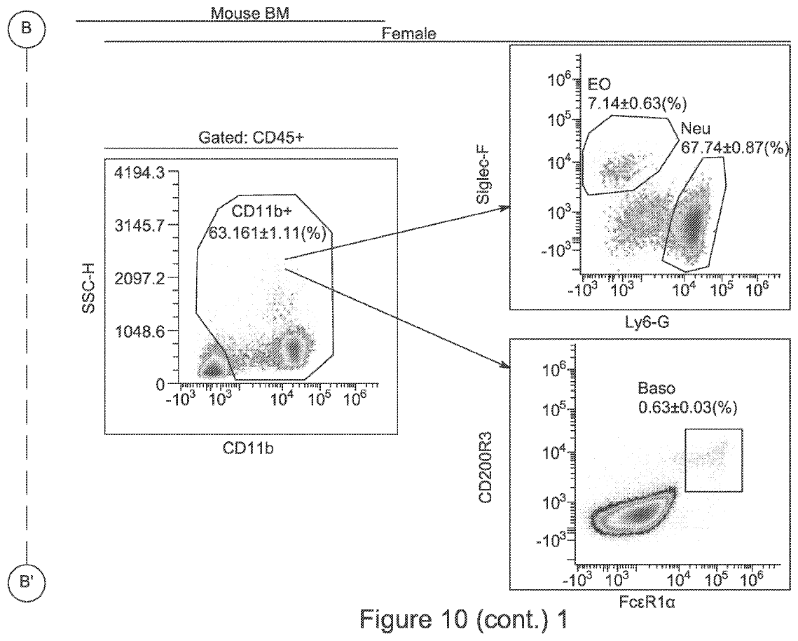

As an example shown in FIG. 9, the stability of the antibody-antigen immune complex on target cell surface was examined using antibodies selected from three groups: ADCC-high (i.e. PEONP22, hCD39 Ref, and Human/Rabbit chimeric clones PEO19, PEO20, PEO21 and PEO25), ADCC-low (Human/Rabbit chimeric clone PEO26), or ADCC-negative (Human/Rabbit chimeric clones PEO27, PEO28 and PEO29). A strong and positive correlation between the stability of such immune complex and the antibody's ADCC activity is clearly seen, viz., the more stable the antibody-antigen immune complex is, the higher the antibody's ADCC activity.

Different epitopes of anti-CD39 antibodies directly link to antibodies' ADCC activity.

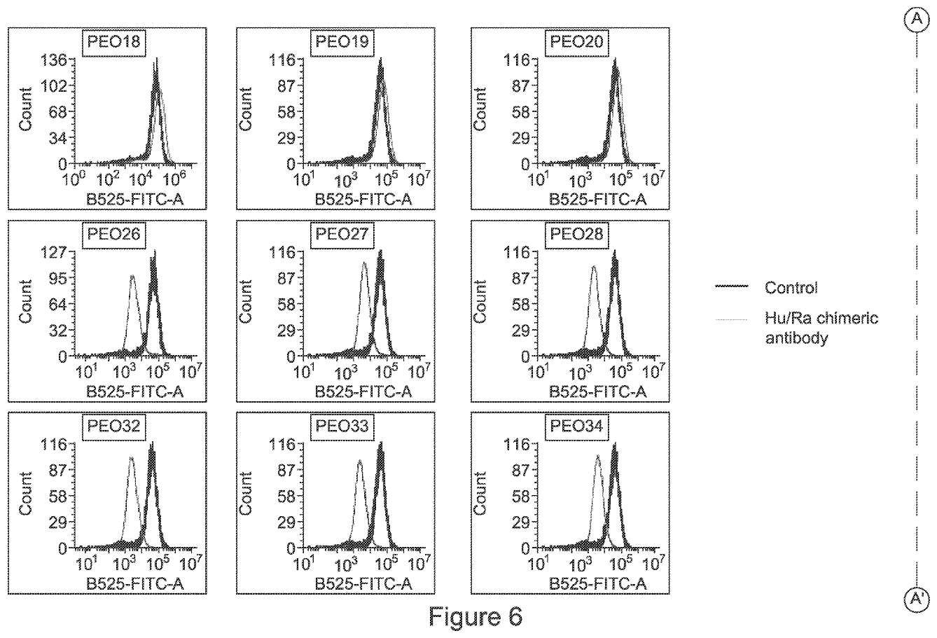

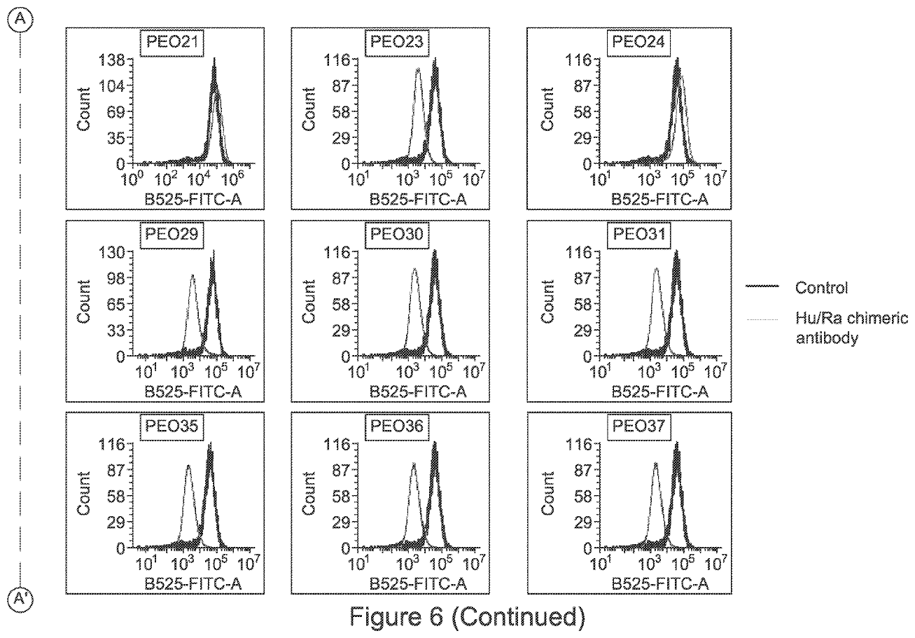

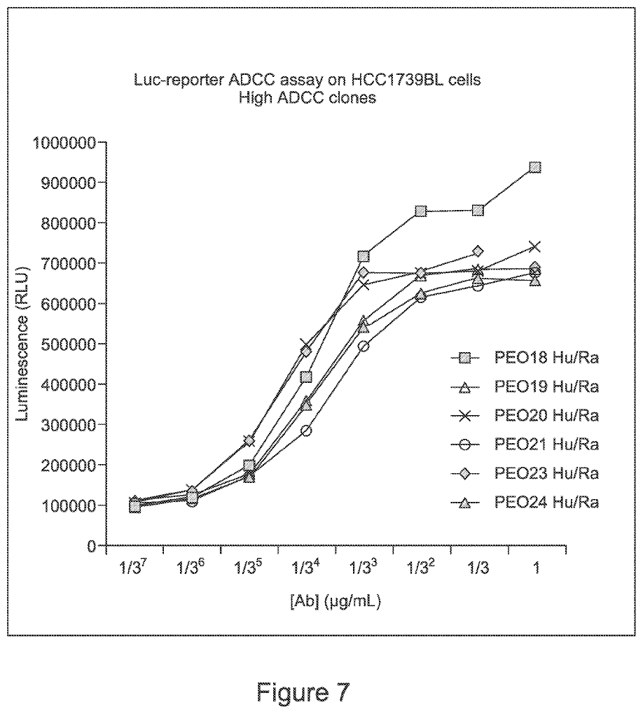

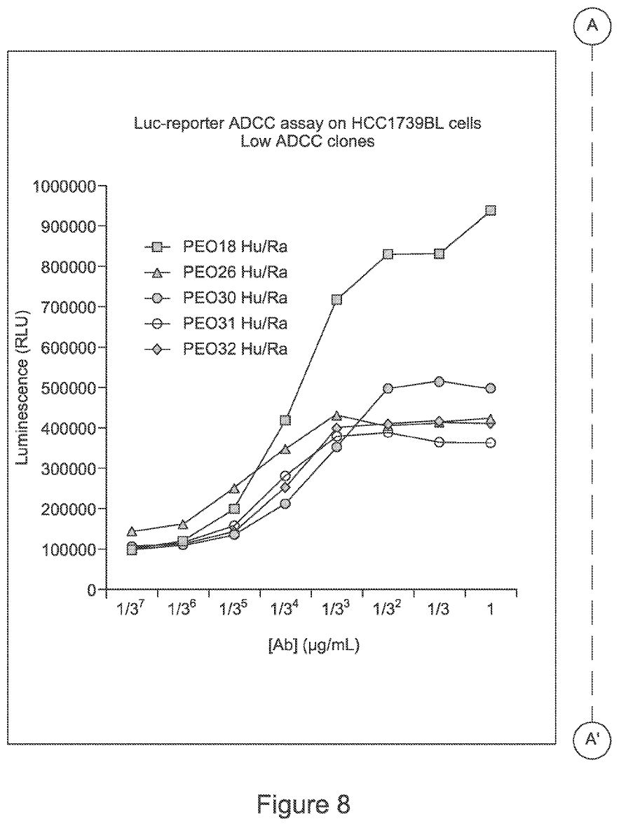

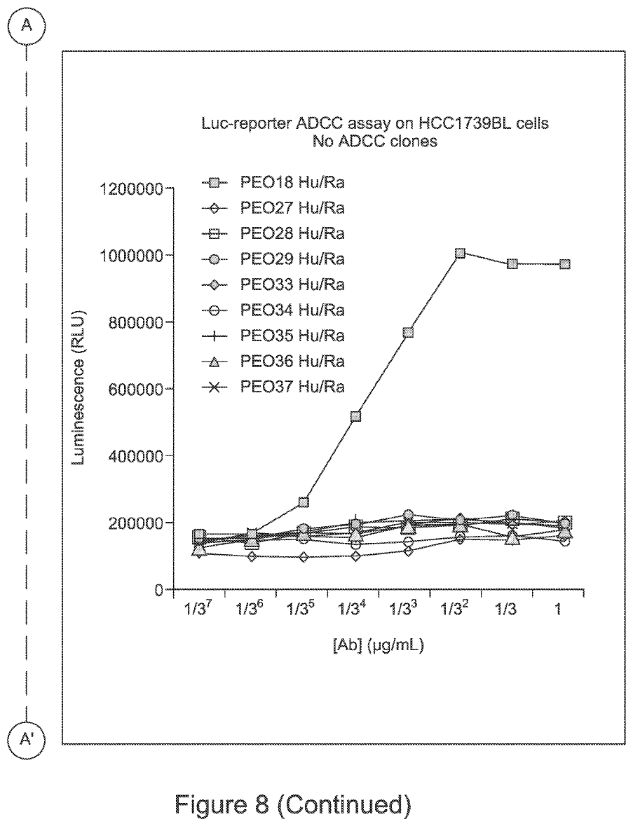

As an example, by comparing epitopes of the subject Human/Rabbit chimeric anti-hCD39 antibodies against the commercially available anti-hCD39 monoclonal antibody Clone A1, FIGS. 6-8 show anti-CD39 antibodies that bind to CD39 in a manner that is non-competitive or only partially competitive with Clone A1 binding to CD39 have a high likelihood of containing high ADCC activity, e.g., five (PEO18, PEO19, PEO20, PEO21 and PEO24,) out of six ADCC-high antibodies, except PEO23, display such trait (FIGS. 6 and 7). In contrast, all antibodies in the ADCC-low (PEO26, PEO30, PEO31 and PEO32) and ADCC-negative (PEO27, PEO28, PEO29, PEO33, PEO34, PEO35, PEO36 and PEO37) groups display epitopes that completely overlap with Clone A1's (FIGS. 6 and 8).

In certain embodiments, rather than be focused on direct inhibition of CD39 NTPase activity, e.g., as is the focus for anti-CD39 therapeutic antibodies in the oncological use of the prior art (Perrot et al., 2019, Cell Reports 27:2411-2425; Li et al., 2019, Cancer Discovery 9 (12): 1754-1773; and WO/2017/089334), anti-CD39 antibodies useful in the present invention (whether or not inhibitory of the NTPase activity) can be specifically designed to have human constant regions with an IgG1 Fc domain. This design confers FcγRIIIa receptor-dependent cellular activities, e.g., antibody-dependent cellular cytotoxicity (ADCC), antibody-dependent cellular phagocytosis (ADCP) and (optionally) complement dependent cytotoxicity (CDC) against CD39+ cells. Consequently, such cellular activities result in ablation and reduction of CD39+eosinophils.

In certain embodiments, certain antibodies encompassed by the present invention have been shown to bind to epitopes on CD39 that are non-competitive with or only partially competitive with the binding of the monoclonal antibody Clone A1 to CD39.

### II. Definitions

To facilitate an understanding of the present invention, a number of terms and phrases are defined below.

“CD39”, also referred to as “Cluster of Differentiation 39”, “ectonucleoside triphosphate diphosphohydrolase-1” or (gene) “ENTPD1” and (protein) “NTPDasel” is a cell surface-located ectonucleotidase with an extracellularly facing catalytic site that catalyses the hydrolysis of γ- and β-phosphate residues of triphospho- and diphosphonucleosides to the monophosphonucleoside derivative (ENZYME entry: EC 3.6.1.5), such as to hydrolyze P2 receptor ligands such as ATP, ADP, UTP and UDP (Junger et al., 2011, Nat. Rev. Immunol. 11:201-212). A representative human NTPDasel protein sequence is provided in the UniProtKB entry “P49961 (ENTP1_HUMAN)”, and a representative human coding sequence for the enzyme is provided in GenBank Accession S73813.

Representative human CD39 cDNA and protein sequences are well-known in the art and are publicly available from the National Center for Biotechnology Information (NCBI). For example, at least seven human CD39 transcript variants are known encoding six different human CD39 isoforms. Human CD39 isoform 1 is available under accession numbers NM_001776.5 and NP_001767.3. The transcript variant represents the longest transcript and encodes isoform 1. Human CD39 isoform 2, available under accession numbers NM_001098175.1 and NP_001091645.1, uses an alternate 5′ exon than transcript variant 1 that results in a distinct 5′ untranslated region (UTR) and causes translation initiation at an alternate start codon leading to a longer and distinct N-terminus. Human CD39 isoform 3, available under accession numbers NM_001164178.1 and NP_001157650.1, uses an alternate 5′ exon than transcript variant 1 that results in a distinct 5′ UTR and causes translation initiation at an alternate start codon leading to a longer and distinct N-terminus. Human CD39 isoform 4, available under accession numbers NM_001164179.1 and NP_001157651.1, uses an alternate in-frame splice site as compared with transcript variant 1 resulting in a shorter isoform. Human CD39 isoform 5, available under accession numbers NM_001164181.1 and NP_001157653.1, uses an alternate exon in the 5′ region that results in a distinct 5′ UTR and translation initiation at a downstream start codon relative to transcript variant 1 resulting in a shorter isoform. Human CD39 isoform 6, available under accession numbers NM_001164182.1 and NP_001157654.1, lacks an alternate exon that results in a distinct 5′ UTR and causes translation initiation at a downstream start codon relative to transcript variant 1 resulting in a shorter isoform. Human CD39 isoform 6 is also encoded by another transcript variant, available under accession numbers NM_001164183.1 and NP_001157655.1, which lacks two alternate internal exons that results in a distinct 5′ UTR and causes translation initiation at a downstream start codon relative to transcript variant 1 resulting in a shorter isoform.

Nucleic acid and polypeptide sequences of CD39 orthologs in organisms other than humans are well known and include, for example, mouse CD39 (NM_009848.3 and NP_033978.1), rat CD39 (NM_022587.1 and NP_072109.1), cow CD39 (NM_174536.2 and NP_776961.1), frog CD39 (NM_001006795.1 and NP_001006796.1), and zebrafish CD39 (NM_001003545.1 and NP_001003545.1).

The extensive glycosylation of CD39 is associated with its cell surface expression and activity such that deletion of glycosylated residues or mutations to non-glycosylatable residues results in significantly reduced CD39 activity (see, for example, deletion or mutation of glycosylatable residues 73 at the N terminus, 333 in the middle, and/or 429 and/or 458 at the C terminus of rat CD39 or corresponding residues in orthologs thereof (Wu et al., 2005, Mol. Biol. Cell. 16:1661-1672). Similarly, mutations of conserved residues in the apyrase conserved region (ACR) of any one or more of ACRs 1-5 causes a reduction in CD39 activity (Schulte am Esch et al., 1999, Biochem. 38:2248-2258; Yang et al., 2001, Biochem. 40:3943-4940; Wang and Guidotti, 1998, J. Biol. Chem. 273:11392-11399).

The modulation (e.g., decrease) in CD39 activity can be measured in any number of ways (e.g., according to measures described herein, including using controls, ratios, comparisons to baselines, and the like). For example, a CD39 activity modulator can decrease the catalytic activity of the ectonucleotidase or overall CD39 activity as compared to the level of such ectonucleotidase in the presence of a test agent. In one embodiment, CD39 activity is determined by analyzing the concentration of adenosine in a sample. The concentration can be assessed over time. In another embodiment, ATP is added in the sample tested and the concentration of remaining ATP, AMP or adenosine is determined or assessed. A modulation in this context, such as a decrease, can mean a decrease of 1%, 5%, 10% , 20%, 25%, 30%, 35%, 40%, 45%, 50%, 55%, 60%, 65%, 70%, 75%, 80%, 85%, 90%, 95%, 100%, 120%, 150%, 200%, 500%, 1000%, or more. In an embodiment, said increase is detected over time.

In certain embodiments, cells, such as eosinophils, exhibit expression of a gene (e.g., CD39) or other biomarker of interest (e.g., flow cytometry side scatter known as SSC) at a high level. In one embodiment, any method described herein to determine the level of expression of the gene or other biomarker may be used to determine the high level. For example, a representative, non-limiting method to define CD39^{high} eosinophils involves analysis of blood (or a blood derivative) or bone marrow, such as blood or bone marrow obtained from healthy or asthmatic hCD39KI mice (obtained from the Purinomia Animal Facility) or blood obtained from healthy human donors, under an antibody-based flow cytometry assay described herein in the Exemplary Materials and Methods using the detection antibodies as listed in Tables 1-3, such as using a Cytek® Aurora flow cytometer.

In certain embodiments, a CD39^{high} eosinophil can be identified as an eosinophil which, in addition to expressing markers characteristic of an eosinophil and/or having functional characteristics of an eosinophil, expresses CD39 at a high enough level such that by flow cytometry has CD39 detection antibody fluorescence intensity of at least 10³, e.g., such as detected using an antibody-based flow cytometry assay described herein in the Exemplary Materials and Methods using the detection antibodies as listed in Tables 1-3, such as using a Cytek® Aurora flow cytometer.

In some embodiments, a CD39^{high} population of cells, such as eosinophils, comprises a population of cells in which at least 60, 65, 70, 75, 80, 85, 90, 95, 96, 97, 98, 99, or greater, or any range in between, inclusive, such as 60-99%, 65-95%, 70-90% 70-80%, and the like, expresses high levels of CD39 (e.g., merely to illustrate, CD39 detection antibody fluorescence intensity is at least 10³ under this specific flow cytometry assay, such as at least 10⁴, 10⁵, 10⁶, 10⁷, or any range in between, inclusive, such as 10³-10⁷, 10⁴-10⁶, 10³-10⁶, and the like). In some embodiments, a CD39low population of cells, such as eosinophils, comprises a population of cells in which less than 41, 40, 39, 38, 37, 36, 35, 30, 25, 20, 15, 10, 5, or even lower, or any range in between, inclusive, such as 4-41%, 10-35%, 15-25%, and the like, expresses low levels of CD39 (e.g., merely to illustrate, CD39 detection antibody fluorescence intensity is less than 10³ under this specific flow cytometry assay, such as less than 10³, 10², 10¹, 10⁰, or any range in between, inclusive, such as less than 10⁰-10³, 10¹-10², 10¹-10³, and the like). In some embodiments, CD39^{high} cells, such as CD45⁺CD11b⁺CD39^{high}Siglec-8+eosinophil cells (which is the equivalent to CD45⁺CD11b⁺CD39^{high}Siglec-F+eosinophil cells in mice), account for 60-99% or any range in between, inclusive, such as 60-95%, 60-80%, 70-80%, 75-80%, etc. of a total population, such as a CD45⁺CD11b⁺Siglec-8+CD39+eosinophil population.

In certain embodiments, cells, such as eosinophils, are “inducible and/or activated” eosinophils. In one embodiment, any method described herein to determine the level of expression of the gene or other biomarker may be used to determine the cell subtypes. For example, a representative, non-limiting method to define “inducible and/or activated” eosinophils involves analysis of blood (or a blood derivative) or bone marrow, such as blood or bone marrow obtained from healthy or asthmatic hCD39KI mice (obtained from the Purinomia Animal Facility) or blood obtained from healthy human donors, under an antibody-based flow cytometry assay described herein in the Exemplary Materials and Methods using the detection antibodies as listed in Tables 1-3, such as using a Cytek® Aurora flow cytometer. In some embodiments, “inducible and/or activated” eosinophils are i) presented in pathological conditions such as asthma, vasculitis, dermatitis, or rhinosinusitis; and/or ii) located in a space selected from the group consisting of blood, bone marrow, lesions, and/or a combination thereof. In some embodiments, “inducible and/or activated” eosinophils are a CD39^{high} eosinophil population. In some embodiments, “inducible and/or activated” eosinophils are a CD45⁺CD11b⁺CD39^{high}Siglec-8+eosinophil population (which is the equivalent to CD45⁺CD11b⁺CD39^{high}Siglec-F+eosinophil cells in mice). In some embodiments, “inducible and/or activated” eosinophils are a CD45⁺CD11b⁺CD39^{high}Siglec-8^{high} eosinophil population.

A “CD39 Antibody” (alternatively an “anti-CD39 antibody”) refers to an antibody that selectively binds to one or more epitopes of the NTPDasel protein, and includes monoparatopic antibodies, as well as biparatopic and other multiparatopic format antibodies.

An “immune complex” (also known as an antigen-antibody complex or antigen-bound antibody) may refer, in some embodiments, to a composition formed through binding of an antigen (e.g. expressed on a medium such as a cell, or alone) to an antibody. A “stable immune complex” may be formed when loss of the interaction between the antigen and the antibody is less than 40% of the immune complex after 24 hours, less than 35%, less than 30%, less than 25%, less than 20%, less than 15% or less than 10% after 24 hours, or any range in between, inclusive, such as 40%-35%, 35%-30%, 30%-25%, 25%-20%, 20%-15%, 15%-10%, and the like (e.g., when incubating the antibody with cells expressing an antigen, such as HCC1739BL cells). In some embodiments, the immune complex formation is detected by fluorescence intensity using a fluorescently labeled secondary antibody (e.g., merely to illustrate, the stability of an immune complex formed with an anti-CD39 antibody can be determined by incubating anti-CD39 monoclonal antibodies (mAbs) (e.g., at 2 μg/mL or greater) with HCC1739BL cells for different times and then detecting the presence of immune complex by fluorescent conjugated secondary antibody).

a. Antibodies and other Polypeptides

The term “antibody” as used herein refers to an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term encompasses intact polyclonal antibodies, intact monoclonal antibodies, antibody fragments (such as Fab, Fab′, F(ab′)₂, and Fv fragments), single chain Fv (scFv) antibodies provided those fragments have been formatted to include an Fc or other FcγRIII binding domain, multispecific antibodies, bispecific antibodies, monospecific antibodies, monovalent antibodies, chimeric antibodies, humanized antibodies, human antibodies, fusion proteins comprising an antigen-binding site of an antibody (formatted to include an Fc or other FcγRIII binding domain), and any other modified immunoglobulin molecule comprising an antigen-binding site as long as the antibodies exhibit the desired biological activity.

The term “antigen-binding portion” or antigen-binding fragment” of an antibody, as used herein, refers to one or more fragments of an antibody that retain the ability to specifically bind to an antigen (e.g., human CD39). It has been shown that the antigen-binding function of an antibody can be performed by fragments of a full-length antibody. Examples of binding fragments encompassed within the term “antigen-binding portion” of an antibody, e.g., an anti-CD39 antibody described herein, include (i) a Fab fragment, a monovalent fragment consisting of the V_{L}, V_{H}, CL and CHI domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., 1989, Nature 341:544-546), which consists of a V_{H} domain; and (vi) an isolated complementarity determining region (CDR) or (vii) a combination of two or more isolated CDRs which may optionally be joined by a synthetic linker. Such single chain antibodies are also intended to be encompassed within the term “antigen-binding portion” of an antibody. These and other potential constructs are described at Chan and Carter (2010) Nat. Rev. Immunol. 10:301. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “variable region” of an antibody refers to the variable region of an antibody light chain, or the variable region of an antibody heavy chain, either alone or in combination. Generally, the variable region of heavy and light chains each consist of four framework regions (FR) and three complementarity determining regions (CDRs), also known as “hypervariable regions”. The CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.); and (2) an approach based on crystallographic studies of antigen-antibody complexes (A1 Lazikani et al., 1997, J. Mol. Biol. 273:927-948). In addition, combinations of these two approaches are sometimes used in the art to determine CDRs.

While the antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively, the preferred CD39 antibody is an IgG1 and IgG3 isotype in order to engage FcγRIII most effectively (i.e., with a Kd of 10-7 or smaller).

In certain embodiments, the antibody is “hypo-fucosylated” and may even be “afucosylated”. A “hypo-fucosylated” antibody preparation refers to an antibody preparation in which less than 50% of the oligosaccharide chains contain «-1,6-fucose. Typically, less than about 40%, less than about 30%, less than about 20%, less than about 10%, or less than 5% or less than 1% of the oligosaccharide chains contain «-1,6-fucose in a “hypo-fucosylated” antibody preparation. An “afucosylated” antibody lacks «-1,6-fucose in the carbohydrate attached to the CH2 domain of the IgG heavy chain.

The term “monoclonal antibody” as used herein refers to an antibody that displays a single binding specificity and affinity for a particular epitope or a composition of antibodies in which all antibodies display a single binding specificity and affinity for a particular epitope. Typically such monoclonal antibodies will be derived from a single cell or nucleic acid encoding the antibody, and will be propagated without intentionally introducing any sequence alterations. Accordingly, the term “human monoclonal antibody” refers to a monoclonal antibody that has variable and optional constant regions derived from human germline immunoglobulin sequences. In one embodiment, human monoclonal antibodies are produced by a hybridoma, for example, obtained by fusing a B cell obtained from a transgenic or transchromosomal non-human animal (e.g., a transgenic mouse having a genome comprising a human heavy chain transgene and a light chain transgene), to an immortalized cell.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability. In some instances, the Fv framework region residues of a human immunoglobulin are replaced with the corresponding residues in an antibody from a non-human species. The humanized antibody can be further modified by the substitution of additional residues either in the Fv framework region and/or within the replaced non-human residues to refine and optimize antibody specificity, affinity, and/or binding capability. The humanized antibody may comprise variable domains containing all or substantially all of the CDRs that correspond to the non-human immunoglobulin whereas all or substantially all of the framework regions are those of a human immunoglobulin sequence. In some embodiments, the variable domains comprise the framework regions of a human immunoglobulin sequence. In some embodiments, the variable domains comprise the framework regions of a human immunoglobulin consensus sequence. The humanized antibody can also comprise at least a portion of an immunoglobulin constant region or domain (Fc), typically that of a human immunoglobulin. A humanized antibody is usually considered distinct from a chimeric antibody.

The term “human antibody” as used herein refers to an antibody produced by a human or an antibody having an amino acid sequence corresponding to an antibody produced by a human made using any of the techniques known in the art.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human) to avoid eliciting an immune response in that species.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor.

An “FcγRIII binding moiety” is a peptide, protein, nucleic acid or other moiety which, when associated with an antigen binding site of an anti-CD39 antibody, is able to bind to FcγRIII (CD16) and, optionally, mediate antibody-dependent cellular cytotoxicity (ADCC) and/or antibody-dependent cellular phagocytosis (ADCP). The heavy chain Fc fragment containing the CH2 and CH3 domains of IgG1 and IgG3 isotypes are FcγRIII binding moiety. The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA).

In certain embodiments, an antibody that specifically binds to a target has a dissociation constant (Kd) of less than or equal to 1 uM, 100 nM, 10 nM, 1 nM, or even 0.1 nM. In certain embodiments, an antibody specifically binds to an epitope on a protein that is conserved among the protein from different species. In another embodiment, specific binding can include, but does not require exclusive binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides encompassed by the present invention may be based upon antibodies or other members of the immunoglobulin superfamily, in certain embodiments, the polypeptides can occur as single chains or as associated chains.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides encompassed by the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies encompassed by the present invention do not abrogate the binding of the polypeptide, soluble protein, or antibody containing the amino acid sequence, to the target binding site. Methods of identifying amino acid conservative substitutions which do not eliminate binding are well-known in the art.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The term “fusion protein” or “fusion polypeptide” as used herein refers to a hybrid protein expressed by a nucleic acid molecule comprising nucleotide sequences of at least two genes.

The term “linker” or “linker region” as used herein refers to a linker inserted between a first polypeptide (e.g., an anti-CD39 antibody) and a second polypeptide (e.g., an Fc or other FcγRIII binding moiety; an scFV, Vhh domain or the like the binds a different protein to create a bispecific antibody format maintaining the bivalency for CD39). In some embodiments, the linker is a peptide linker. Linkers should not adversely affect the expression, secretion, or bioactivity of the polypeptides. Preferably, linkers are not antigenic and do not elicit an immune response.

b. Treatments

The term “effective amount” as used herein refers to an amount to provide therapeutic or prophylactic benefit.

The term “treatment” as used herein refers to action by an individual to change the process of a clinical disease, which may be either preventive or an intervention to alter a course of clinical pathology. The term includes, but is not limited to, preventing the occurrence or recurrence of disease, alleviating symptoms, reducing the direct or indirect pathological consequences of any disease, preventing metastasis, slowing the rate of disease progression, ameliorating or remitting disease remission, and improving prognosis.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “agonist” and “agonistic” as used herein refer to or describe a therapeutic moiety that is capable of, directly or indirectly, substantially inducing, activating, promoting, increasing, or enhancing the biological activity of a target and/or a pathway. The term “agonist” is used herein to include any agent that partially or fully induces, activates, promotes, increases, or enhances the activity of a protein or other target of interest.

The terms “antagonist” and “antagonistic” as used herein refer to or describe a therapeutic moiety that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein or other target of interest.

The terms “modulation” and “modulate” as used herein refer to a change or an alteration in a biological activity. Modulation includes, but is not limited to, stimulating an activity or inhibiting an activity. Modulation may be an increase in activity or a decrease in activity, a change in binding characteristics, or any other change in the biological, functional, or immunological properties associated with the activity of a protein, a pathway, a system, or other biological targets of interest.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, etc.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one agent of the present disclosure, and which does not destroy the pharmacological activity thereof and is nontoxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The terms “effective amount” or “therapeutically effective amount” or “therapeutic effect” refer to an amount of an anti-CD39 antibody effective to “treat” a disease or disorder in a subject such as, a mammal.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented.

c. Miscellaneous

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used herein, reference to “about” or “approximately” a value or parameter includes (and describes) embodiments that are directed to that value or parameter. For example, description referring to “about X” includes description of “X”.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### III. Anti-CD39 Antibodies

a. Monoclonal Antibodies

The anti-CD39 antibodies useful in the methods and pharmaceutical preparations of the present invention may be monoclonal antibodies. Monoclonal antibodies may be prepared using hybridoma methods, such as those described by Kohler and Milstein, 1975, Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. In some embodiments, monoclonal antibodies, e.g., rabbit monoclonal antibodies, may be produced using single B cell cloning technology, such as those described in Rashidian and Lloyd, 2020, Methods Mol. Biol. 2070:423-441, the content of which is incorporated by reference herein in its entirety.

The immunizing agent will typically include the CD39 polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor et al., 1984, J. Immunol. 133:3001; Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the polypeptide. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, 1980, Anal. Biochem. 107:220.

After the desired hybridoma cells are identified, the clones may be subcloned by limiting dilution procedures and grown by standard methods (Goding, supra). Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies encompassed by the invention can be readily isolated and sequenced using conventional procedures (e.g., by using oligonucleotide probes that are capable of binding specifically to genes encoding the heavy and light chains of murine antibodies). The hybridoma cells encompassed by the present invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody encompassed by the present invention, or can be substituted for the variable domains of one antigen-combining site of an antibody encompassed by the present invention to create a chimeric bivalent antibody.

b. Human and Humanized Antibodies

The anti-CD39 antibodies encompassed by the present invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., 1986, Nature 321:522-525; Riechmann et al., 1988, Nature 332:323-329; and Presta, 1992, Curr. Op. Struct. Biol. 2:593-596).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., 1986, Nature 321:522-525; Riechmann et al., 1988, Nature 332:323-327; Verhoeyen et al., 1988, Science 239:1534-1536), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, 1991, J. Mol. Biol. 227:381; Marks et al., 1991, J. Mol. Biol. 222:581), and yeast display (Chao et al., 2006, Nat. Protoc. 1 (2): 755-68). The techniques of Cole et al. and Boerner et al. are also available for the preparation of human monoclonal antibodies (Cole et al., Monoclonal Antibodies and Cancer Therapy, Alan R. Liss, pp. 77 (1985) and Boerner et al., 1991, J. Immunol. 147 (1): 86-95). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016; and in the following scientific publications: Marks et al., 1992, Bio/Technology 10:779-783; Lonberg et al., 1994, Nature 368:856-859; Morrison, 1994, Nature 368:812-13; Fishwild et al., 1996, Nature Biotechnology 14:845-51; Neuberger, 1996, Nature Biotechnology 14:826; Lonberg and Huszar, 1995, Intern. Rev. Immunol. 13:65-93.

The antibodies may also be affinity matured using known selection and/or mutagenesis methods as described above. Preferred affinity matured antibodies have an affinity which is five times, more preferably 10 times, even more preferably 20 or 30 times greater than the starting antibody (generally murine, humanized or human) from which the matured antibody is prepared.

c. Bispecific Antibodies

Anti-CD39 antibodies described herein include bispecific molecules. An anti-CD39 antibody, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody described herein may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule described herein, an antibody described herein can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, provided herein are bispecific molecules comprising at least one first binding specificity for CD39 and a second binding specificity for a second target epitope. In an embodiment described herein in which the bispecific molecule is multi-specific, the molecule can further include a third binding specificity.

In certain embodiments, the present invention provides bispecific antibodies that bind to both CD39 and an eosinophil cell surface antigen selected from the group consisting of Siglec-8, IL-5Rα (CD125), IL-3Rα (CD123), IL-4R, IL-9R, IL-13R, IL-14R, ST2 (IL-33R), PIRA, PIRB, L-selectin, EMR-1, CCR3 (CD193), and CRTh2 (CD294), and preferably causes eosinophil depletion-such as by ADCC-mediated and/or ADCP-mediated killing or in the form of an antibody-drug conjugate that is preferentially taken up by and toxic to eosinophils.

In other embodiments, the bispecific is generated with binding domains against antigens that are upregulated on activated eosinophils (or eosinophils found in lesions), with the avidity of the bispecific for different antigens (CD39 and a second antigen) both expressed on eosinophils providing for the selectivity. Exemplary antigens for generation of bispecifics using the CD39 binders of the present invention include CD3, CD4, γδTCR, CD9, CD28, CD29, CD40, CD44, CD45, CD45RO, CD48, CD58, CD63 (lysosome-associated membrane protein 3), CD66b (CEACAM8), CD66e (CEACAM5), CD67, CD69, CD80, CD86, C5aR (CD88), CD101, CD122, CD137 (tumor necrosis factor receptor superfamily member 9, induced by lymphocyte activation, 4-1BB), CD274 (programmed death ligand 1), aub integrin (CD41), a2 integrin (CD49b), a4 integrin (CD49d), αL integrin (CD11a), αM integrin (CD11b), αX integrin (CD11c), αD integrin, β2 integrin (CD18), Aminopeptidase N (CD13), FcαRI (CD89), FcγRIII (CD16), FcγRII (CD32), Fc∈RII (CD23), Granulocyte monocyte-colony stimulating factorRα (CD116), HLA-DR, Intercellular adhesion molecule-1 (CD54), Interleukin (IL)-2Rα (CD25), IL-17RA, IL-17RB, Galectin-3, Neuropeptide S receptor, P-selectin glycoprotein ligand-1 (CD162), Semaphorin 7A (CD108), Thymic stromal lymphopoietin protein receptor (TSLPR), activated αM integrin, activated β1 integrin (CD29), activated β2 integrin, activated FcγRII, and activated CRTh2 (CD294). Such bispecifics bind to and preferably causes eosinophil depletion-such as by ADCC-mediated and/or ADCP-mediated killing or in the form of an antibody-drug conjugate that is preferentially taken up by and toxic to eosinophils.

In one embodiment, the bispecific molecules described herein comprise as a binding specificity at least one antibody, or an antibody fragment thereof, including, e.g., an Fab, Fab′, F (ab′)₂, Fv, or a single chain Fv. The antibody may also be a light chain or heavy chain dimer, or any minimal fragment thereof such as a Fv or a single chain (scFv) construct. Binding of the bispecific molecules to their specific targets can be confirmed using art-recognized methods, such as enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, 1983, Nature 305:537-539). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., 1991, EMBO J. 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., 1986, Methods in Enzymology 121:210.

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full-length antibodies or antibody fragments (e.g., F (ab′)₂ bispecific antibodies). Techniques for generating bispecific antibodies from antibody fragments have been described in the literature. For example, bispecific antibodies can be prepared can be prepared using chemical linkage. Brennan et al., 1985, Science 229:81 describe a procedure wherein intact antibodies are proteolytically cleaved to generate F (ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., 1992, J. Exp. Med. 175:217-225 describe the production of a fully humanized bispecific antibody F (ab′)₂ molecule. Each Fab′ fragment was separately secreted from E. coli and subjected to directed chemical coupling in vitro to form the bispecific antibody.

Various technique for making and isolating bispecific antibody fragments directly from recombinant cell culture have also been described. For example, bispecific antibodies have been produced using leucine zippers (Kostelny et al., 1992, J. Immunol. 148 (5): 1547-1553). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., 1993, Proc. Natl. Acad. Sci. USA 90:6444-6448 has provided an alternative mechanism for making bispecific antibody fragments. The fragments comprise a heavy-chain variable domain (V_{H}) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See, Gruber et al., 1994, J. Immunol. 152:5368.

Antibodies with more than two valencies are contemplated. As one nonlimiting example, trispecific antibodies can be prepared. See, e.g., Tutt et al., 1991, J. Immunol. 147:60.

d. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089).

It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

e. Effector Function Engineering

It may be desirable to modify the antibody encompassed by the present invention with respect to effector function, so as to further enhance, e.g., the effectiveness of the anti-CD39 antibody in depleting eosinophils. For example, cysteine residue(s) may be introduced into the Fc region, thereby allowing interchain disulfide bond formation in this region. The homodimeric antibody thus generated may have improved internalization capability, increased complement-mediated cell killing, improved antibody-dependent cellular cytotoxicity (ADCC), and/or improved antibody-dependent cellular phagocytosis (ADCP). See Caron et al., 1992, J. Exp Med. 176:1191-1195 and Shopes, 1992, J. Immunol., 148:2918-2922.

f. Representative anti-CD39 Antibody Sequences

In certain embodiments, the anti-CD39 antibody is a fully human antibody, such as generated from a human antibody library. An exemplary fully human anti-CD39 antibody is clone PEOWT22, the heavy and light variable domains (VH and VL) sequences provided as follows:

| | Nucleic Acid Sequence | Amino Acid Sequence |
|---|---|---|
| VH domain | SEQ ID No. 1 (VH) | SEQ ID No. 2 (VH) |
| VL domain | SEQ ID No. 3 (VL) | SEQ ID No. 4 (VL) |

For the PEOWT22 clone, the CDRs for each of the VH and VL domains are:

| | CDR1 | CDR2 | CDR3 |
|---|---|---|---|
| VH | SEQ ID No. 29 | SEQ ID No. 30 | SEQ ID No. 31 |
| VL | SEQ ID No. 32 | SEQ ID No. 33 | SEQ ID No. 34 |

The sequences for an exemplary full-length antibody and an exemplary single chain antibody (scFV) utilizing the VH and VL domains above are provided as follows:

| | Nucleic Acid Sequence | Amino Acid Sequence |
|---|---|---|
| Full Length Heavy | SEQ ID No. 35 | SEQ ID No. 36 |
| Chain | | |
| Full Length Light | SEQ ID No. 37 | SEQ ID No. 38 |
| Chain | | |
| scFV | SEQ ID No. 39 | SEQ ID No. 40 |

In some embodiments, the anti-CD39 antibody or antigen-binding fragment thereof comprises at least one heavy chain variable domain is at least 60% identical to SEQ ID No. 2 and even more preferably at least 65%, 70%, 75%, 80%, 85% or even 90% identical to SEQ ID No. 2, and able to specifically bind human CD39.

In some embodiments, the anti-CD39 antibody or antigen-binding fragment thereof comprises at least one light chain variable domain is at least 60% identical to SEQ ID No. 4, and even more preferably at least 65%, 70%, 75%, 80%, 85% or even 90% identical to SEQ ID No. 4 and able to specifically bind human CD39.

In certain embodiments, the anti-CD39 antibody is a humanized antibody comprising a VH domain having human framework sequences associated with CDRs of a VH domain shown in SEQ ID Nos. 29, 30 and 31, and the CDRs of the corresponding VL domain shown in SEQ ID Nos. 32, 33 and 34. The CDRs are preferably identical, but may vary by 1, 2 or 3 amino acids across each CDR so long as the resulting antibody specifically binds human CD39.

In certain embodiments, the heavy and light chains of the anti-CD39 antibody have variable domains that can be encoded by a nucleic acid which is identical to, or hybridizes under stringent conditions (such as the 6× sodium chloride/sodium citrate (SSC) at 45° C., and washing in 0.2×SSC/0.1% SDS at 50-65° C.) to the VH and VL domain (correspondingly) coding sequences shown in SEQ ID No. 1 (VH) and SEQ ID No. 3 (VL).

In some embodiments, anti-CD39 antibodies were generated in rabbits, and the variable domains of the heavy and light chains of these antibodies are rabbit sequence while the constant domains are human sequence. Exemplary sequences for the VH and VL domains of rabbit anti-CD39 antibodies are:

| | Nucleic Acid | | |
|---|---|---|---|
| Clone | Sequence | Amino Acid Sequence | CDR Sequences |
| PEO18 | SEQ ID No. 5 (VH) | SEQ ID No. 6 (VH) | SEQ ID No. 6 (VH) CDRs |
| | SEQ ID No. 7 (VL) | SEQ ID No. 8 (VL) | CDR1: GFSLSAYG |
| | | | CDR2: IYSSGRT |
| | | | CDR3: ARSRAGISSGDGFDS |
| | | | SEQ ID No. 8 (VL) CDRs |
| | | | CDR1: QNIYSN |
| | | | CDR2: RAS |
| | | | CDR3: QQGFDSSNIDNT |
| PEO19 | SEQ ID No. 9 (VH) | SEQ ID No. 10 (VH) | SEQ ID No. 10 (VH) CDRs |
| | SEQ ID No. 11 (VL) | SEQ ID No. 12 (VL) | CDR1: GFSLSKSI |
| | | | CDR2: IGSSGST |
| | | | CDR3: ARGLLYSGNKS |
| | | | SEQ ID No. 12 (VL) CDRs |
| | | | CDR1: QSVLLNNQ |
| | | | CDR2: DAS |
| | | | CDR3: LGGYSGNLYA |
| PEO20 | SEQ ID No. 13 (VH) | SEQ ID No. 14 (VH) | SEQ ID No. 14 (VH) CDRS |
| | SEQ ID No. 15 (VL) | SEQ ID No. 16 (VL) | CDR1: GFSLSSYA |
| | | | CDR2: INSYGTT |
| | | | CDR3: ARGDSYGSGVGLGL |
| | | | SEQ ID No. 16 (VL) CDRs |
| | | | CDR1: QNIYSN |
| | | | CDR2: RAS |
| | | | CDR3: QQGFSSNNVDNT |
| PEO21 | SEQ ID No. 17 (VH) | SEQ ID No. 18 (VH) | SEQ ID No. 18 (VH) CDRs |
| | SEQ ID No. 19 (VL) | SEQ ID No. 20 (VL) | CDR1: GFSLSSYA |
| | | | CDR2: ISSSGST |
| | | | CDR3: ARDRVIYSIGPYYFNL |
| | | | SEQ ID No. 20 (VL) CDRs |
| | | | CDR1: EIIYSN |
| | | | CDR2: GAS |
| | | | CDR3: QQSFSSNNVGNI |
| PEO23 | SEQ ID No. 21 (VH) | SEQ ID No. 22 (VH) | SEQ ID No. 22 (VH) CDRs |
| | SEQ ID No. 23 (VL) | SEQ ID No. 24 (VL) | CDR1: GFSLSTHA |
| | | | CDR2: TYASGRT |
| | | | CDR3: ARNGADETFYYFDL |
| | | | SEQ ID No. 24 (VL) CDRs |
| | | | CDR1: QNINTW |
| | | | CDR2: RAS |
| | | | CDR3: QQYDASINIDNA |
| PEO24 | SEQ ID No. 25 (VH) | SEQ ID No. 26 (VH) | SEQ ID No. 26 (VH) CDRS |
| | SEQ ID No. 27 (VL) | SEQ ID No. 28 (VL) | CDR1: GIDLSSNA |
| | | | CDR2: IRNNDIT |
| | | | CDR3: ARGGGSYSIVFWNL |
| | | | SEQ ID No. 28 (VL) CDRs |
| | | | CDR1: ERIYSN |
| | | | CDR2: YAS |
| | | | CDR3: QQGYSNNNVDNT |
| PEO25 | SEQ ID No. 41 (VH) | SEQ ID No. 42 (VH) | SEQ ID No. 42 (VH) CDRs |
| | SEQ ID No. 43 (VL) | SEQ ID No. 44 (VL) | CDR1: GIDLSNNA |
| | | | CDR2: IRSSGST |
| | | | CDR3: ARGGGSYSIVFWNL |
| | | | SEQ ID No. 44 (VL) CDRs |
| | | | CDR1: ERIYSN |
| | | | CDR2: YTS |
| | | | CDR3: QQGYSSSNVDNT |

In some embodiments, anti-CD39 antibodies were generated in rabbits, and then humanized by CDR grafting. Exemplary sequences for the VH and VL domains of rabbit anti-CD39 antibodies are:

| | Nucleic Acid | Amino Acid | |
|---|---|---|---|
| Clone | Sequence | Sequence | CDR Sequences |
| Humanized | SEQ ID No. 45 (VH) | SEQ ID No. 46 (VH) | SEQ ID No. 46 (VH) |
| PEO20 | SEQ ID No. 47 (VL) | SEQ ID No. 48 (VL) | CDRS |
| | | | CDR1: GFSLSSYA |
| | | | CDR2: INSYGTT |
| | | | CDR3: ARGDSYGSGVGLGL |
| | | | SEQ ID No. 48 (VL) |
| | | | CDRs |
| | | | CDR1: QNIYSN |
| | | | CDR2: RAS |
| | | | CDR3: QQGFSSNNVDNT |
| Humanized | SEQ ID No. 49 (VH) | SEQ ID No. 50 (VH) | SEQ ID No. 50 (VH) |
| PEO19 | SEQ ID No. 51 (VL) | SEQ ID No. 52 (VL) | CDRs |
| | | | CDR1: GFSLSKSI |
| | | | CDR2: IGSSGST |
| | | | CDR3: ARGLLYSGNKS |
| | | | SEQ ID No. 52 (VL) |
| | | | CDRs |
| | | | CDR1: QSVLLNNQ |
| | | | CDR2: DAS |
| | | | CDR3: LGGYSGNLYA |
| Humanized | SEQ ID No. 53 (VH) | SEQ ID No. 54 (VH) | SEQ ID No. 54 (VH) |
| PEO21 | SEQ ID No. 55 (VL) | SEQ ID No. 56 (VL) | CDRs |
| | | | CDR1: GFSLSSYA |
| | | | CDR2: ISSSGST |
| | | | CDR3: ARDRVIYSIGPYYFNL |
| | | | SEQ ID No. 56 (VL) |
| | | | CDRs |
| | | | CDR1: EIIYSN |
| | | | CDR2: GAS |
| | | | CDR3: QQSFSSNNVGNI |

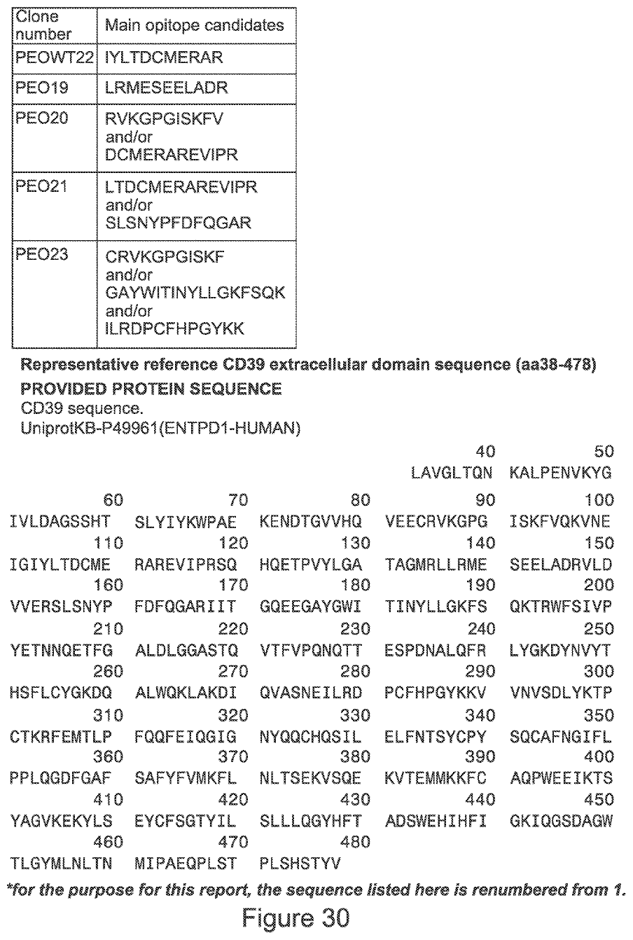

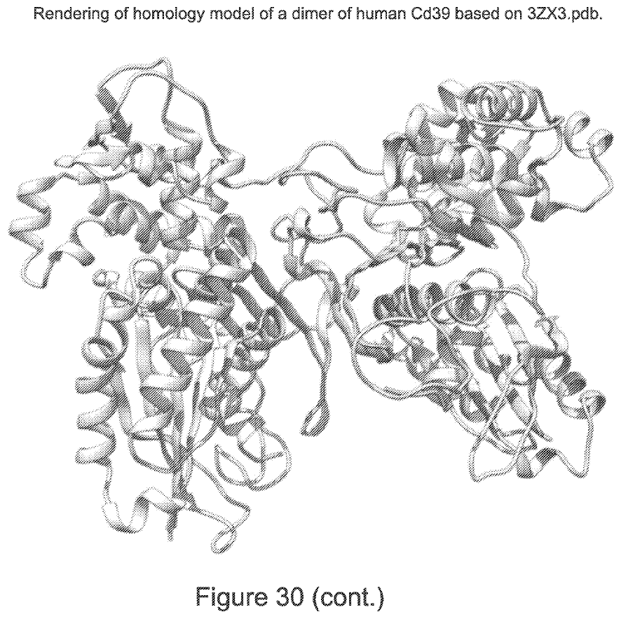

In some embodiments, anti-CD39 antibodies provided herein promote: (i) stable immune complex formation when incubated with HCC1739BL cells as characterized by loss of less than 30% of the immune complex after 24 hours, optionally wherein the immune complex formation is detected by fluorescence intensity using a fluorescently labeled secondary antibody; (ii) antibody-dependent cellular cytotoxicity (ADCC) and/or antibody-dependent cellular phagocytosis (ADCP) activity against CD39+ cells; (iii) depletion of CD39+eosinophils; (iv) binding to a CD39 epitope having a sequence selected from the group of CD39 amino acid epitope sequences listed in FIG. 30 (for example, binding one or more linear or conformational CD39 epitopes, such as selected from the group consisting of 1) IYLTDCMERAR, 2) LRMESEELADR, 3) RVKGPGISKFV, 4) DCMERAREVIPR, 5) LTDCMERAREVIPR, 6) SLSNYPFDFQGAR, 7 CRVKGPGISKF, 8) GAYGWITINYLLGKFSQK, 9) ILRDPCFHPGYKK, and any combination thereof, such as RVKGPGISKFV and DCMERAREVIPR, LTDCMERAREVIPR and SLSNYPFDFQGAR, or any combination of CRVKGPGISKF, GAYGWITINYLLGKFSQK, and ILRDPCFHPGYKK); and/or (v) binding to CD39 in a manner that is non-competitive or only partially competitive with monoclonal antibody Clone A1 binding to CD39.

Representative anti-CD39 antibody sequences described above according to sequence identification number correspond to the following:

SEQ ID No. 1 (Clone PEOWT22 vH nucleic acid sequence)

| | |
|---|---|
| gag gtg caa ctg gtg gag tct ggg gga ggt gtg gta agg cct ggg ggg | 48 |
| Glu Val Gln Leu Val Glu Ser Gly Gly Gly Val Val Arg Pro Gly Gly | |
| 1 5 10 15 | |
| tcc ctg aga ctc tcc tgt gca gcc tct gga ttc acc ttc agt agc tat | 96 |
| Ser Leu Arg Leu Ser Cys Ala Ala Ser Gly Phe Thr Phe Ser Ser Tyr | |
| 20 25 30 | |
| gct atg cac tgg gtc cgc cag gct cca ggc aag ggg ctg gag tgg gtg | 144 |
| Ala Met His Trp Val Arg Gln Ala Pro Gly Lys Gly Leu Glu Trp Val | |
| 35 40 45 | |
| gca gtt ata tca tat gat gta agc aat aaa tac tac gca gac tcc gtg | 192 |
| Ala Val Ile Ser Tyr Asp Val Ser Asn Lys Tyr Tyr Ala Asp Ser Val | |
| 50 55 60 | |
| aag ggc cga ttc acc atc tcc aga gac aat tcc aag aac acg ctg tat | 240 |
| Lys Gly Arg Phe Thr Ile Ser Arg Asp Asn Ser Lys Asn Thr Leu Tyr | |
| 65 70 75 80 | |
| ctg caa atg aac agc ctg aga gct gag gac acg gct gtg tat tac tgt | 288 |
| Leu Gln Met Asn Ser Leu Arg Ala Glu Asp Thr Ala Val Tyr Tyr Cys | |
| 85 90 95 | |
| gcg aga tct tac tac tac tac tac ggt atg gac gtc tgg ggc caa ggg | 336 |
| Ala Arg Ser Tyr Tyr Tyr Tyr Tyr Gly Met Asp Val Trp Gly Gln Gly | |
| 100 105 110 | |
| acc acg gtc acc gtc tcc tca | 357 |
| Thr Thr Val Thr Val Ser Ser | |
| 115 | |

```
SEQ ID No. 2 (Clone PEOWT22 vH amino acid sequence)
Glu Val Gln Leu Val Glu Ser Gly Gly Gly Val Val Arg Pro Gly Gly
1 5 10 15
Ser Leu Arg Leu Ser Cys Ala Ala Ser Gly Phe Thr Phe Ser Ser Tyr
                        20 25 30
Ala Met His Trp Val Arg Gln Ala Pro Gly Lys Gly Leu Glu Trp Val
                35 40 45
Ala Val Ile Ser Tyr Asp Val Ser Asn Lys Tyr Tyr Ala Asp Ser Val
        50 55 60
Lys Gly Arg Phe Thr Ile Ser Arg Asp Asn Ser Lys Asn Thr Leu Tyr
65 70 75 80
Leu Gln Met Asn Ser Leu Arg Ala Glu Asp Thr Ala Val Tyr Tyr Cys
                                85 90 95
Ala Arg Ser Tyr Tyr Tyr Tyr Tyr Gly Met Asp Val Trp Gly Gln Gly
                        100 105 110
Thr Thr Val Thr Val Ser Ser
                115
SEQ ID No. 3 (Clone PEOWT22 vL domain nucleic acid sequence)
```

| | |
|---|---|
| gat gtt gtg atg acc cag tct cca tcc tcc ctg tct gca tct gta gga | 48 |
| Asp Val Val Met Thr Gln Ser Pro Ser Ser Leu Ser Ala Ser Val Gly | |
| 1 5 10 15 | |
| gac aga gtc acc atc act tgc cgg gca agt cag agc att agc agg tac | 96 |
| Asp Arg Val Thr Ile Thr Cys Arg Ala Ser Gln Ser Ile Ser Arg Tyr | |
| 20 25 30 | |
| tta gcc tgg tac caa cag aaa cct ggc cag gct ccc agg ctc ctc atc | 144 |
| Leu Ala Trp Tyr Gln Gln Lys Pro Gly Gln Ala Pro Arg Leu Leu Ile | |
| 35 40 45 | |
| tat gat gca tec aac agg gcc act ggc atc cca gtc agg ttc agt ggc | 192 |
| Tyr Asp Ala Ser Asn Arg Ala Thr Gly Ile Pro Val Arg Phe Ser Gly | |
| 50 55 60 | |
| agt ggg tct ggg aca gac ttc act ctc acc atc agc aga ctg gag cca | 240 |
| Ser Gly Ser Gly Thr Asp Phe Thr Leu Thr Ile Ser Arg Leu Glu Pro | |
| 65 70 75 80 | |
| gaa gat ttt gca gtg tat tac tgt cag cag ttt ggt agg tca cct cgg | 288 |
| Glu Asp Phe Ala Val Tyr Tyr Cys Gln Gln Phe Gly Arg Ser Pro Arg | |
| 85 90 95 | |
| acg ttc ggc caa ggg aca cga ctg gag att aaa | 321 |
| Thr Phe Gly Gln Gly Thr Arg Leu Glu Ile Lys | |
| 100 105 | |

```
SEQ ID No. 4 (Clone PEOWT22 vL domain amino acid sequence)
Asp Val Val Met Thr Gln Ser Pro Ser Ser Leu Ser Ala Ser Val Gly
1 5 10 15
Asp Arg Val Thr Ile Thr Cys Arg Ala Ser Gln Ser Ile Ser Arg Tyr
                        20 25 30
Leu Ala Trp Tyr Gln Gln Lys Pro Gly Gln Ala Pro Arg Leu Leu Ile
                35 40 45
Tyr Asp Ala Ser Asn Arg Ala Thr Gly Ile Pro Val Arg Phe Ser Gly
        50 55 60
Ser Gly Ser Gly Thr Asp Phe Thr Leu Thr Ile Ser Arg Leu Glu Pro
65 70 75 80
Glu Asp Phe Ala Val Tyr Tyr Cys Gln Gln Phe Gly Arg Ser Pro Arg
                                85 90 95
Thr Phe Gly Gln Gly Thr Arg Leu Glu Ile Lys
                        100 105
SEQ ID No. 5 (Clone PEO18 vH domain nucleic acid sequence)
```

| | |
|---|---|
| cag tca gtg aag gag gcc ggg ggt cgc ctg gta acg cct gga gga tcc | 48 |
| Gln Ser Val Lys Glu Ala Gly Gly Arg Leu Val Thr Pro Gly Gly Ser | |
| 1 5 10 15 | |
| ctg aca ctc acc tgc aca gtc tct gga ttc tcc ctc agt gcg tat gga | 96 |
| Leu Thr Leu Thr Cys Thr Val Ser Gly Phe Ser Leu Ser Ala Tyr Gly | |
| 20 25 30 | |
| ata agt tgg gtc cgc cag gct cca ggg aag gga ctg gaa tgg atc gga | 144 |
| Ile Ser Trp Val Arg Gln Ala Pro Gly Lys Gly Leu Glu Trp Ile Gly | |
| 35 40 45 | |
| atc att tat agt agt ggt agg act tac tac gcg aac tgg gcg aaa ggc | 192 |
| Ile Ile Tyr Ser Ser Gly Arg Thr Tyr Tyr Ala Asn Trp Ala Lys Gly | |
| 50 55 60 | |
| cga ttc acc atc tcc aaa acc tcg tcg acc acg gtg gat ctg aaa atg | 240 |
| Arg Phe Thr Ile Ser Lys Thr Ser Ser Thr Thr Val Asp Leu Lys Met | |
| 65 70 75 80 | |
| acc agt ctg aca acc gag gac acg gcc gcc tat ttc tgt gcc aga tca | 288 |
| Thr Ser Leu Thr Thr Glu Asp Thr Ala Ala Tyr Phe Cys Ala Arg Ser | |
| 85 90 95 | |
| cgg gct ggt att agt agt ggt gat ggt ttt gat tcc tgg ggc cca ggc | 336 |
| Arg Ala Gly Ile Ser Ser Gly Asp Gly Phe Asp Ser Trp Gly Pro Gly | |
| 100 105 110 | |
| acc ctg gtc acc gtc tcc tca | 357 |
| Thr Leu Val Thr Val Ser Ser | |
| 115 | |

```
SEQ ID No. 6 (Clone PEO18 vH domain amino acid sequence)
Gln Ser Val Lys Glu Ala Gly Gly Arg Leu Val Thr Pro Gly Gly Ser
1 5 10 15
Leu Thr Leu Thr Cys Thr Val Ser Gly Phe Ser Leu Ser Ala Tyr Gly
                        20 25 30
Ile Ser Trp Val Arg Gln Ala Pro Gly Lys Gly Leu Glu Trp Ile Gly
                35 40 45
Ile Ile Tyr Ser Ser Gly Arg Thr Tyr Tyr Ala Asn Trp Ala Lys Gly
        50 55 60
Arg Phe Thr Ile Ser Lys Thr Ser Ser Thr Thr Val Asp Leu Lys Met
65 70 75 80
Thr Ser Leu Thr Thr Glu Asp Thr Ala Ala Tyr Phe Cys Ala Arg Ser
                                85 90 95
Arg Ala Gly Ile Ser Ser Gly Asp Gly Phe Asp Ser Trp Gly Pro Gly
                        100 105 110
Thr Leu Val Thr Val Ser Ser
                115
SEQ ID No. 7 (Clone PEO18 vL domain nucleic acid sequence)
```

| | |
|---|---|
| gcc ctt gtg atg acc cag act cca tcc tcc gtg tct gca gct gtg gga | 48 |
| Ala Leu Val Met Thr Gln Thr Pro Ser Ser Val Ser Ala Ala Val Gly | |
| 1 5 10 15 | |
| ggc aca gtc acc atc aat tgc cag gcc agt cag aac att tac agc aat | 96 |
| Gly Thr Val Thr Ile Asn Cys Gln Ala Ser Gln Asn Ile Tyr Ser Asn | |
| 20 25 30 | |
| tta gcc tgg tat cag cag aaa cca ggg cag cgt ccc cag ctc ctg atc | 144 |
| Leu Ala Trp Tyr Gln Gln Lys Pro Gly Gln Arg Pro Gln Leu Leu Ile | |
| 35 40 45 | |
| tac agg gca tcc act ctg gca tct ggg gtc cca tcg cgg ttc aaa ggc | 192 |
| Tyr Arg Ala Ser Thr Leu Ala Ser Gly Val Pro Ser Arg Phe Lys Gly | |
| 50 55 60 | |
| agt gca tct ggg aca gaa tac act ctc acc atc agc ggt gtg cag tgt | 240 |
| Ser Ala Ser Gly Thr Glu Tyr Thr Leu Thr Ile Ser Gly Val Gln Cys | |
| 65 70 75 80 | |
| gac gat gct gcc act tac tat tgt caa cag ggt ttt gat agt agt aac | 288 |
| Asp Asp Ala Ala Thr Tyr Tyr Cys Gln Gln Gly Phe Asp Ser Ser Asn | |
| 85 90 95 | |
| att gat aat act ttc ggc gga ggg acc gag gtg gtg gtc aca | 330 |
| Ile Asp Asn Thr Phe Gly Gly Gly Thr Glu Val Val Val Thr | |
| 100 105 110 | |

```
SEQ ID No. 8 (Clone PEO18 vL domain amino acid sequence)
Ala Leu Val Met Thr Gln Thr Pro Ser Ser Val Ser Ala Ala Val Gly
1 5 10 15
Gly Thr Val Thr Ile Asn Cys Gln Ala Ser Gln Asn Ile Tyr Ser Asn
                        20 25 30
Leu Ala Trp Tyr Gln Gln Lys Pro Gly Gln Arg Pro Gln Leu Leu Ile
                35 40 45
Tyr Arg Ala Ser Thr Leu Ala Ser Gly Val Pro Ser Arg Phe Lys Gly
        50 55 60
Ser Ala Ser Gly Thr Glu Tyr Thr Leu Thr Ile Ser Gly Val Gln Cys
65 70 75 80
Asp Asp Ala Ala Thr Tyr Tyr Cys Gln Gln Gly Phe Asp Ser Ser Asn
                                85 90 95
Ile Asp Asn Thr Phe Gly Gly Gly Thr Glu Val Val Val Thr
                        100 105 110
SEQ ID No. 9 (Clone PEO19 vH domain nucleic acid sequence)
```

| | |
|---|---|
| cag tcg gtg gag gag tcc ggg ggt cgc ctg gtc acg cct ggg aca cac | 48 |
| Gln Ser Val Glu Glu Ser Gly Gly Arg Leu Val Thr Pro Gly Thr His | |
| 1 5 10 15 | |
| ctg aca ctc acc tgc aca gtc tct gga ttc tcc ctc agt aag agt ata | 96 |
| Leu Thr Leu Thr Cys Thr Val Ser Gly Phe Ser Leu Ser Lys Ser Ile | |
| 20 25 30 | |
| ata agt tgg gtc cgc cag gct cca ggg aag ggg ctg gaa tac atc gga | 144 |
| Ile Ser Trp Val Arg Gln Ala Pro Gly Lys Gly Leu Glu Tyr Ile Gly | |
| 35 40 45 | |
| atc att ggt agt agt ggt agc aca tac tac gcg aac tgg gcg aaa ggc | 192 |
| Ile Ile Gly Ser Ser Gly Ser Thr Tyr Tyr Ala Asn Trp Ala Lys Gly | |
| 50 55 60 | |
| cga ttc acc atc tcc aaa acc tcg tcg acc acg gtg gat ctg aga atg | 240 |
| Arg Phe Thr Ile Ser Lys Thr Ser Ser Thr Thr Val Asp Leu Arg Met | |
| 65 70 75 80 | |
| acc agt ctg aca ccc gag gac acg gcc acc tat ttc tgt gcc aga gga | 288 |
| Thr Ser Leu Thr Pro Glu Asp Thr Ala Thr Tyr Phe Cys Ala Arg Gly | |
| 85 90 95 | |
| ctt ctt tat tct ggt aat aaa tcg tgg ggc ccg ggc acc ctg gtc acc | 336 |
| Leu Leu Tyr Ser Gly Asn Lys Ser Trp Gly Pro Gly Thr Leu Val Thr | |
| 100 105 110 | |
| gtc tcc tca | 345 |
| Val Ser Ser | |
| 115 | |

```
SEQ ID No. 10 (Clone PEO19 vH domain amino acid sequence)
Gln Ser Val Glu Glu Ser Gly Gly Arg Leu Val Thr Pro Gly Thr His
1 5 10 15
Leu Thr Leu Thr Cys Thr Val Ser Gly Phe Ser Leu Ser Lys Ser Ile
                        20 25 30
Ile Ser Trp Val Arg Gln Ala Pro Gly Lys Gly Leu Glu Tyr Ile Gly
                35 40 45
Ile Ile Gly Ser Ser Gly Ser Thr Tyr Tyr Ala Asn Trp Ala Lys Gly
        50 55 60
Arg Phe Thr Ile Ser Lys Thr Ser Ser Thr Thr Val Asp Leu Arg Met
65 70 75 80
Thr Ser Leu Thr Pro Glu Asp Thr Ala Thr Tyr Phe Cys Ala Arg Gly
                                85 90 95
Leu Leu Tyr Ser Gly Asn Lys Ser Trp Gly Pro Gly Thr Leu Val Thr
                        100 105 110
Val Ser Ser
                115
SEQ ID No. 11 (Clone PEO19 vL domain nucleic acid sequence)
```

| | |
|---|---|
| gcc att gat atg acc cag act cca tcc tcc gtg tct gca gct gtg gga | 48 |
| Ala Ile Asp Met Thr Gln Thr Pro Ser Ser Val Ser Ala Ala Val Gly | |
| 1 5 10 15 | |
| ggc aca gtc acc atc aac tgc cag tcc agt cag agt gtt tta ctg aac | 96 |
| Gly Thr Val Thr Ile Asn Cys Gln Ser Ser Gln Ser Val Leu Leu Asn | |
| 20 25 30 | |
| aac caa tta tcc tgg ttt cag cag aaa cca ggg cag cct ccc aag ctc | 144 |
| Asn Gln Leu Ser Trp Phe Gln Gln Lys Pro Gly Gln Pro Pro Lys Leu | |
| 35 40 45 | |
| ctg atc tat gat gca tcc act ctg gaa tct ggg gtc cca tct cgg ttc | 192 |
| Leu Ile Tyr Asp Ala Ser Thr Leu Glu Ser Gly Val Pro Ser Arg Phe | |
| 50 55 60 | |
| aca ggc agt gga tct ggg aca cag ttc act ctc acc atc agc gac ctg | 240 |
| Thr Gly Ser Gly Ser Gly Thr Gln Phe Thr Leu Thr Ile Ser Asp Leu | |
| 65 70 75 80 | |
| gag tgt gac gat gct gcc act tac tat tgt tta ggc ggt tat agt ggg | 288 |
| Glu Cys Asp Asp Ala Ala Thr Tyr Tyr Cys Leu Gly Gly Tyr Ser Gly | |
| 85 90 95 | |
| aac ctt tat gct ttc ggc gga ggg acc gag gtg cta gtc aaa | 330 |
| Asn Leu Tyr Ala Phe Gly Gly Gly Thr Glu Val Leu Val Lys | |
| 100 105 110 | |

```
SEQ ID No. 12 (Clone PEO19 vL domain amino acid sequence)
Ala Ile Asp Met Thr Gln Thr Pro Ser Ser Val Ser Ala Ala Val Gly
1 5 10 15
Gly Thr Val Thr Ile Asn Cys Gln Ser Ser Gln Ser Val Leu Leu Asn
                        20 25 30
Asn Gln Leu Ser Trp Phe Gln Gln Lys Pro Gly Gln Pro Pro Lys Leu
                35 40 45
Leu Ile Tyr Asp Ala Ser Thr Leu Glu Ser Gly Val Pro Ser Arg Phe
        50 55 60
Thr Gly Ser Gly Ser Gly Thr Gln Phe Thr Leu Thr Ile Ser Asp Leu
65 70 75 80
Glu Cys Asp Asp Ala Ala Thr Tyr Tyr Cys Leu Gly Gly Tyr Ser Gly
                                85 90 95
Asn Leu Tyr Ala Phe Gly Gly Gly Thr Glu Val Leu Val Lys
                        100 105 110
SEQ ID No. 13 (Clone PEO20 vH domain nucleic acid sequence)
```

| | |
|---|---|
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc aca gtc tct gga ttc tcc ctc agt agc tat gca | 96 |
| Leu Thr Leu Thr Cys Thr Val Ser Gly Phe Ser Leu Ser Ser Tyr Ala | |
| 20 25 30 | |
| ata agt tgg gtc cgc cag gct cca ggg aag ggg ctc gaa tat atc gcg | 144 |
| Ile Ser Trp Val Arg Gln Ala Pro Gly Lys Gly Leu Glu Tyr Ile Ala | |
| 35 40 45 | |
| atc att aat agt tat ggt acc aca tac tac gcg agc tgg gcg aaa ggc | 192 |
| Ile Ile Asn Ser Tyr Gly Thr Thr Tyr Tyr Ala Ser Trp Ala Lys Gly | |
| 50 55 60 | |
| cga gtc acc atc tcc aaa acc tcg agc acg gtg gat ctg aaa atc tcc | 240 |
| Arg Val Thr Ile Ser Lys Thr Ser Ser Thr Val Asp Leu Lys Ile Ser | |
| 65 70 75 80 | |
| agt ccg aca acc gag gac acg gcc acc tat ttc tgt gcc aga ggc gat | 288 |
| Ser Pro Thr Thr Glu Asp Thr Ala Thr Tyr Phe Cys Ala Arg Gly Asp | |
| 85 90 95 | |
| agt tat ggt agt ggt gtt ggt ttg ggc ttg tgg ggc cca ggc acc ctg | 336 |
| Ser Tyr Gly Ser Gly Val Gly Leu Gly Leu Trp Gly Pro Gly Thr Leu | |
| 100 105 110 | |
| gtc acc gtc tcc tca | 351 |
| Val Thr Val Ser Ser | |
| 115 | |

```
SEQ ID No. 14 (Clone PEO20 vH domain amino acid sequence)
Gln Ser Val Glu Glu Ser Gly Gly Arg Leu Val Thr Pro Gly Thr Pro
1 5 10 15
Leu Thr Leu Thr Cys Thr Val Ser Gly Phe Ser Leu Ser Ser Tyr Ala
                        20 25 30
Ile Ser Trp Val Arg Gln Ala Pro Gly Lys Gly Leu Glu Tyr Ile Ala
                35 40 45
Ile Ile Asn Ser Tyr Gly Thr Thr Tyr Tyr Ala Ser Trp Ala Lys Gly
        50 55 60
Arg Val Thr Ile Ser Lys Thr Ser Ser Thr Val Asp Leu Lys Ile Ser
65 70 75 80
Ser Pro Thr Thr Glu Asp Thr Ala Thr Tyr Phe Cys Ala Arg Gly Asp
                                85 90 95
Ser Tyr Gly Ser Gly Val Gly Leu Gly Leu Trp Gly Pro Gly Thr Leu
                        100 105 110
Val Thr Val Ser Ser
                115
SEQ ID No. 15 (Clone PEO20 vL domain nucleic acid sequence)
```

| | |
|---|---|
| gcc tat gat atg acc cag act cca gcc tct gtg gag gta gct gtg gga | 48 |
| Ala Tyr Asp Met Thr Gln Thr Pro Ala Ser Val Glu Val Ala Val Gly | |
| 1 5 10 15 | |
| ggc aca gtc acc atc aag tgc cag gcc agt cag aac att tac agc aat | 96 |
| Gly Thr Val Thr Ile Lys Cys Gln Ala Ser Gln Asn Ile Tyr Ser Asn | |
| 20 25 30 | |
| tta gcc tgg tat cag cag aaa cca ggg cag cgt ccc aag ctc ctg atc | 144 |
| Leu Ala Trp Tyr Gln Gln Lys Pro Gly Gln Arg Pro Lys Leu Leu Ile | |
| 35 40 45 | |
| tac agg gca tcc agt ctg gca tct ggg gtc ccg tcg cgg ttc agt ggc | 192 |
| Tyr Arg Ala Ser Ser Leu Ala Ser Gly Val Pro Ser Arg Phe Ser Gly | |
| 50 55 60 | |
| agt gga tct ggg aca gag ttc act ctc acc atc agc ggt gtg cag tgt | 240 |
| Ser Gly Ser Gly Thr Glu Phe Thr Leu Thr Ile Ser Gly Val Gln Cys | |
| 65 70 75 80 | |
| gac gat gct gcc act tac tac tgt caa cag ggt ttt agt agt aat aat | 288 |
| Asp Asp Ala Ala Thr Tyr Tyr Cys Gln Gln Gly Phe Ser Ser Asn Asn | |
| 85 90 95 | |
| gtt gat aat act ttc ggc gga ggg acc gag gtg gtg gtc aaa | 330 |
| Val Asp Asn Thr Phe Gly Gly Gly Thr Glu Val Val Val Lys | |
| 100 105 110 | |

```
SEQ ID No. 16 (Clone PEO20 vL domain amino acid sequence)
Ala Tyr Asp Met Thr Gln Thr Pro Ala Ser Val Glu Val Ala Val Gly
1 5 10 15
Gly Thr Val Thr Ile Lys Cys Gln Ala Ser Gln Asn Ile Tyr Ser Asn
                        20 25 30
Leu Ala Trp Tyr Gln Gln Lys Pro Gly Gln Arg Pro Lys Leu Leu Ile
                35 40 45
Tyr Arg Ala Ser Ser Leu Ala Ser Gly Val Pro Ser Arg Phe Ser Gly
        50 55 60
Ser Gly Ser Gly Thr Glu Phe Thr Leu Thr Ile Ser Gly Val Gln Cys
65 70 75 80
Asp Asp Ala Ala Thr Tyr Tyr Cys Gln Gln Gly Phe Ser Ser Asn Asn
                                85 90 95
Val Asp Asn Thr Phe Gly Gly Gly Thr Glu Val Val Val Lys
                        100 105 110
SEQ ID No. 17 (Clone PEO21 vH domain nucleic acid sequence)
```

| | |
|---|---|
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc acc gtc tcc gga ttc tcc ctc agt agc tat gca | 96 |
| Leu Thr Leu Thr Cys Thr Val Ser Gly Phe Ser Leu Ser Ser Tyr Ala | |
| 20 25 30 | |
| atg agc tgg gtc cgc cag gct cca ggg aag ggg ctg gaa tac atc gga | 144 |
| Met Ser Trp Val Arg Gln Ala Pro Gly Lys Gly Leu Glu5 Tyr Ile Gly | |
| 35 40 45 | |
| atc att agt agt agt ggt agc aca tac tac gcg agc tgg gcg aaa ggc | 192 |
| Ile Ile Ser Ser Ser Gly Ser Thr Tyr Tyr Ala Ser Trp Ala Lys Gly | |
| 50 55 60 | |
| cga ttc acc atc tcc aaa acc tcg acc acg gtg gat ctg aaa atc tcc | 240 |
| Arg Phe Thr Ile Ser Lys Thr Ser Thr Thr Val Asp Leu Lys Ile Ser | |
| 65 70 75 80 | |
| agt ccg aca acc gag gac acg gcc acc tat ttc tgt gcc aga gat cgt | 288 |
| Ser Pro Thr Thr Glu Asp Thr Ala Thr Tyr Phe Cys Ala Arg Asp Arg | |
| 85 90 95 | |
| gtt att tat agt att ggt ccg tat tat ttt aat ttg tgg ggc cca ggc | 336 |
| Val Ile Tyr Ser Ile Gly Pro Tyr Tyr Phe Asn Leu Trp Gly Pro Gly | |
| 100 105 110 | |
| acc ctg gtc acc gtc tcc tca | 357 |
| Thr Leu Val Thr Val Ser Ser | |
| 115 | |

```
SEQ ID No. 18 (Clone PEO21 vH domain amino acid sequence)
Gln Ser Val Glu Glu Ser Gly Gly Arg Leu Val Thr Pro Gly Thr Pro
1 5 10 15
Leu Thr Leu Thr Cys Thr Val Ser Gly Phe Ser Leu Ser Ser Tyr Ala
                        20 25 30
Met Ser Trp Val Arg Gln Ala Pro Gly Lys Gly Leu Glu Tyr Ile Gly
                35 40 45
Ile Ile Ser Ser Ser Gly Ser Thr Tyr Tyr Ala Ser Trp Ala Lys Gly
        50 55 60
Arg Phe Thr Ile Ser Lys Thr Ser Thr Thr Val Asp Leu Lys Ile Ser
65 70 75 80
Ser Pro Thr Thr Glu Asp Thr Ala Thr Tyr Phe Cys Ala Arg Asp Arg
                                85 90 95
Val Ile Tyr Ser Ile Gly Pro Tyr Tyr Phe Asn Leu Trp Gly Pro Gly
                        100 105 110
Thr Leu Val Thr Val Ser Ser
                115
SEQ ID No. 19 (Clone PEO21 vL domain nucleic acid sequence)
```

| | |
|---|---|
| gcc tat gat atg acc cag act cca tcc tcc gtg tct gca act gtg gga | 48 |
| Ala Tyr Asp Met Thr Gln Thr Pro Ser Ser Val Ser Ala Thr Val Gly | |
| 1 5 10 15 | |
| ggc aca gtc acc atc aat tgc cag gcc agt gag atc att tat agc aat | 96 |
| Gly Thr Val Thr Ile Asn Cys Gln Ala Ser Glu Ile Ile Tyr Ser Asn | |
| 20 25 30 | |
| tta gcc tgg tat cag cag aaa cca ggg cag cct ccc aag ctc ctg atc | 144 |
| Leu Ala Trp Tyr Gln Gln Lys Pro Gly Gln Pro Pro Lys Leu Leu Ile | |
| 35 40 45 | |
| tat ggc gca tcc act ctg gca tct ggg gtc cca tcg cgg ttc aaa ggc | 192 |
| Tyr Gly Ala Ser Thr Leu Ala Ser Gly Val Pro Ser Arg Phe Lys Gly | |
| 50 55 60 | |
| agt gga tct ggg aca gag tac act ctc acc atc agc gac ctg cag tgt | 240 |
| Ser Gly Ser Gly Thr Glu Tyr Thr Leu Thr Ile Ser Asp Leu Gln Cys | |
| 65 70 75 80 | |
| gac gat gct gcc act tac tac tgt caa cag agt ttt agt agt aat aat | 288 |
| Asp Asp Ala Ala Thr Tyr Tyr Cys Gln Gln Ser Phe Ser Ser Asn Asn | |
| 85 90 95 | |
| gtt ggg aat att ttc ggc gga ggg acc gag gtg gtg gtc aaa | 330 |
| Val Gly Asn Ile Phe Gly Gly Gly Thr Glu Val Val Val Lys | |
| 100 105 110 | |

```
SEQ ID No. 20 (Clone PEO21 vL domain amino acid sequence)
Ala Tyr Asp Met Thr Gln Thr Pro Ser Ser Val Ser Ala Thr Val Gly
1 5 10 15
Gly Thr Val Thr Ile Asn Cys Gln Ala Ser Glu Ile Ile Tyr Ser Asn
                        20 25 30
Leu Ala Trp Tyr Gln Gln Lys Pro Gly Gln Pro Pro Lys Leu Leu Ile
                35 40 45
Tyr Gly Ala Ser Thr Leu Ala Ser Gly Val Pro Ser Arg Phe Lys Gly
        50 55 60
Ser Gly Ser Gly Thr Glu Tyr Thr Leu Thr Ile Ser Asp Leu Gln Cys
65 70 75 80
Asp Asp Ala Ala Thr Tyr Tyr Cys Gln Gln Ser Phe Ser Ser Asn Asn
                                85 90 95
Val Gly Asn Ile Phe Gly Gly Gly Thr Glu Val Val Val Lys
                        100 105 110
SEQ ID No. 21 (Clone PEO23 vH domain nucleic acid sequence)
```

| | |
|---|---|
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc aca gcc tct gga ttc tcc ctc agt acc cat gca | 96 |
| Leu Thr Leu Thr Cys Thr Ala Ser Gly Phe Ser Leu Ser Thr His Ala | |
| 20 25 30 | |
| ata aac tgg gtc cgc cag gct cca ggg aag ggg ctg gaa tgg atc ggg | 144 |
| Ile Asn Trp Val Arg Gln Ala Pro Gly Lys Gly Leu Glu Trp Ile Gly | |
| 35 40 45 | |
| atc act tat gct agt ggt agg aca tat tac gcg agc tgg gcg aaa ggc | 192 |
| Ile Thr Tyr Ala Ser Gly Arg Thr Tyr Tyr Ala Ser Trp Ala Lys Gly | |
| 50 55 60 | |
| cga ttc acc atc tcc aaa acc tcg acc acg gtg gat ctg aaa atc acc | 240 |
| Arg Phe Thr Ile Ser Lys Thr Ser Thr Thr Val Asp Leu Lys Ile Thr | |
| 65 70 75 80 | |
| agt ccg aca acc gag gac acg gcc acc tat ttc tgt gcc aga aat ggg | 288 |
| Ser Pro Thr Thr Glu Asp Thr Ala Thr Tyr Phe Cys Ala Arg Asn Gly | |
| 85 90 95 | |
| gct gat gaa aca ttt tac tac ttt gac ttg tgg ggc cca ggc acc ctg | 336 |
| Ala Asp Glu Thr Phe Tyr Tyr Phe Asp Leu Trp Gly Pro Gly Thr Leu | |
| 100 105 110 | |
| gtc acc gtc tcc tca | 351 |
| Val Thr Val Ser Ser | |
| 115 | |

```
SEQ ID No. 22 (Clone PEO23 vH domain amino acid sequence)
Gln Ser Val Glu Glu Ser Gly Gly Arg Leu Val Thr Pro Gly Thr Pro
1 5 10 15
Leu Thr Leu Thr Cys Thr Ala Ser Gly Phe Ser Leu Ser Thr His Ala
                        20 25 30
Ile Asn Trp Val Arg Gln Ala Pro Gly Lys Gly Leu Glu Trp Ile Gly
                35 40 45
Ile Thr Tyr Ala Ser Gly Arg Thr Tyr Tyr Ala Ser Trp Ala Lys Gly
        50 55 60
Arg Phe Thr Ile Ser Lys Thr Ser Thr Thr Val Asp Leu Lys Ile Thr
65 70 75 80
Ser Pro Thr Thr Glu Asp Thr Ala Thr Tyr Phe Cys Ala Arg Asn Gly
                                85 90 95
Ala Asp Glu Thr Phe Tyr Tyr Phe Asp Leu Trp Gly Pro Gly Thr Leu
                        100 105 110
Val Thr Val Ser Ser
                115
SEQ ID No. 23 (Clone PEO23 vL domain nucleic acid sequence)
```

| | |
|---|---|
| gcc tat gat atg acc cag act cca gcc tcc gtg gag gca gct gtg gga | 48 |
| Ala Tyr Asp Met Thr Gln Thr Pro Ala Ser Val Glu Ala Ala Val Gly | |
| 1 5 10 15 | |
| ggc aca gtc acc atc aag tgc cag gcc agt cag aat att aat act tgg | 96 |
| Gly Thr Val Thr Ile Lys Cys Gln Ala Ser Gln Asn Ile Asn Thr Trp | |
| 20 25 30 | |
| tta tcc tgg tat cag cag aag gca ggg cag cct ccc aag ctc ctg atc | 144 |
| Leu Ser Trp Tyr Gln Gln Lys Ala Gly Gln Pro Pro Lys Leu Leu Ile | |
| 35 40 45 | |
| tac agg gca tcc act ctg gca tct ggg gtc tca tcg cgg ttc aaa ggc | 192 |
| Tyr Arg Ala Ser Thr Leu Ala Ser Gly Val Ser Ser Arg Phe Lys Gly | |
| 50 55 60 | |
| agt gga tct ggg aca cag ttc act ctc acc atc agc ggc gtg gag tgt | 240 |
| Ser Gly Ser Gly Thr Gln Phe Thr Leu Thr Ile Ser Gly Val Glu Cys | |
| 65 70 75 80 | |
| gcc gat gct gcc act tac tac tgt caa caa tat gat gct agt att aat | 288 |
| Ala Asp Ala Ala Thr Tyr Tyr Cys Gln Gln Tyr Asp Ala Ser Ile Asn | |
| 85 90 95 | |
| | 330 |
| att gat aat gct ttc ggc gga ggg acc gag gtg gtg gtc aaa | |
| Ile Asp Asn Ala Phe Gly Gly Gly Thr Glu Val Val Val Lys | |
| 100 105 110 | |

```
SEQ ID No. 24 (Clone PEO23 vL domain amino acid sequence)
Ala Tyr Asp Met Thr Gln Thr Pro Ala Ser Val Glu Ala Ala Val Gly
1 5 10 15
Gly Thr Val Thr Ile Lys Cys Gln Ala Ser Gln Asn Ile Asn Thr Trp
                        20 25 30
Leu Ser Trp Tyr Gln Gln Lys Ala Gly Gln Pro Pro Lys Leu Leu Ile
                35 40 45
Tyr Arg Ala Ser Thr Leu Ala Ser Gly Val Ser Ser Arg Phe Lys Gly
        50 55 60
Ser Gly Ser Gly Thr Gln Phe Thr Leu Thr Ile Ser Gly Val Glu Cys
65 70 75 80
Ala Asp Ala Ala Thr Tyr Tyr Cys Gln Gln Tyr Asp Ala Ser Ile Asn
                                85 90 95
Ile Asp Asn Ala Phe Gly Gly Gly Thr Glu Val Val Val Lys
                        100 105 110
SEQ ID No. 25 (Clone PEO24 vH domain nucleic acid sequence)
```

| | |
|---|---|
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc aca gtc tct gga atc gac ctc agt agc aat gca | 96 |
| Leu Thr Leu Thr Cys Thr Val Ser Gly Ile Asp Leu Ser Ser Asn Ala | |
| 20 25 30 | |
| atg agc tgg gtc cgc cag gct cca ggg aag ggg ctg gaa tat atc gga | 144 |
| Met Ser Trp Val Arg Gln Ala Pro Gly Lys Gly Leu Glu Tyr Ile Gly | |
| 35 40 45 | |
| att att agg aat aat gat atc aca tac tac gcg agc tgg gcg aaa ggc | 192 |
| Ile Ile Arg Asn Asn Asp Ile Thr Tyr Tyr Ala Ser Trp Ala Lys Gly | |
| 50 55 60 | |
| cga ttc acc atc tcc aaa acc tcg acc acg gtg gat ctg ata atc acc | 240 |
| Arg Phe Thr Ile Ser Lys Thr Ser Thr Thr Val Asp Leu Ile Ile Thr | |
| 65 70 75 80 | |
| agt ccg aca acc gag gac acg gcc acc tat ttc tgt gcc aga ggg ggt | 288 |
| Ser Pro Thr Thr Glu Asp Thr Ala Thr Tyr Phe Cys Ala Arg Gly Gly | |
| 85 90 95 | |
| ggt tct tac agt att gtc ttc tcg aac tta tgg ggc cca ggc acc ctg | 336 |
| Gly Ser Tyr Ser Ile Val Phe Trp Asn Leu Trp Gly Pro Gly Thr Leu | |
| 100 105 110 | |
| gtc acc gtc tcc tca | 351 |
| Val Thr Val Ser Ser | |
| 115 | |

```
SEQ ID No. 26 (Clone PEO24 vH domain amino acid sequence)
Gln Ser Val Glu Glu Ser Gly Gly Arg Leu Val Thr Pro Gly Thr Pro
1 5 10 15
Leu Thr Leu Thr Cys Thr Val Ser Gly Ile Asp Leu Ser Ser Asn Ala
                        20 25 30
Met Ser Trp Val Arg Gln Ala Pro Gly Lys Gly Leu Glu Tyr Ile Gly
                35 40 45
Ile Ile Arg Asn Asn Asp Ile Thr Tyr Tyr Ala Ser Trp Ala Lys Gly
        50 55 60
Arg Phe Thr Ile Ser Lys Thr Ser Thr Thr Val Asp Leu Ile Ile Thr
65 70 75 80
Ser Pro Thr Thr Glu Asp Thr Ala Thr Tyr Phe Cys Ala Arg Gly Gly
                                85 90 95
Gly Ser Tyr Ser Ile Val Phe Trp Asn Leu Trp Gly Pro Gly Thr Leu
                        100 105 110
Val Thr Val Ser Ser
                115
SEQ ID No. 27 (Clone PEO24 vL domain nucleic acid sequence)
```

| | |
|---|---|
| gcc tat gat atg acc cag act cca gcc tct gtg gag gta gct gtg gga | 48 |
| Ala Tyr Asp Met Thr Gln Thr Pro Ala Ser Val Glu Val Ala Val Gly | |
| 1 5 10 15 | |
| ggc aca gtc acc atc aat tgc cag gcc agt gag agg att tat agc aat | 96 |
| Gly Thr Val Thr Ile Asn Cys Gln Ala Ser Glu Arg Ile Tyr Ser Asn | |
| 20 25 30 | |
| tta gcc tgg tat cag cag aaa cca ggg cag cgt ccc aaa ctc ctg atc | 144 |
| Leu Ala Trp Tyr Gln Gln Lys Pro Gly Gln Arg Pro Lys Leu Leu Ile | |
| 35 40 45 | |
| tat tat gca tcc act ctg gca tct ggg gtc tca tcg cgg ttc aaa ggc | 192 |
| Tyr Tyr Ala Ser Thr Leu Ala Ser Gly Val Ser Ser Arg Phe Lys Gly | |
| 50 55 60 | |
| agt gga tct ggg aca cag ttc act ctc acc atc agc ggc gtg cag tgt | 240 |
| Ser Gly Ser Gly Thr Gln Phe Thr Leu Thr Ile Ser Gly Val Gln Cys | |
| 65 70 75 80 | |
| gcc gat gct gcc act tac tac tgt cag cag ggt tat agt aat aat aat | 288 |
| Ala Asp Ala Ala Thr Tyr Tyr Cys Gln Gln Gly Tyr Ser Asn Asn Asn | |
| 85 90 95 | |
| gtt gac aat act ttc ggc gga ggg acc gag gtg gtg gtc aga | 330 |
| Val Asp Asn Thr Phe Gly Gly Gly Thr Glu Val Val Val Arg | |
| 100 105 110 | |

```
SEQ ID No. 28 (Clone PEO24 vL domain amino acid sequence)
Ala Tyr Asp Met Thr Gln Thr Pro Ala Ser Val Glu Val Ala Val Gly
1 5 10 15
Gly Thr Val Thr Ile Asn Cys Gln Ala Ser Glu Arg Ile Tyr Ser Asn
                        20 25 30
Leu Ala Trp Tyr Gln Gln Lys Pro Gly Gln Arg Pro Lys Leu Leu Ile
                35 40 45
Tyr Tyr Ala Ser Thr Leu Ala Ser Gly Val Ser Ser Arg Phe Lys Gly
        50 55 60
Ser Gly Ser Gly Thr Gln Phe Thr Leu Thr Ile Ser Gly Val Gln Cys
65 70 75 80
Ala Asp Ala Ala Thr Tyr Tyr Cys Gln Gln Gly Tyr Ser Asn Asn Asn
                                85 90 95
Val Asp Asn Thr Phe Gly Gly Gly Thr Glu Val Val Val Arg
                        100 105 110
SEQ ID No. 29 (Clone PEOWT22 vH domain CDR1 amino acid sequence)
Gly Phe Thr Phe Ser Ser Tyr Ala
1 5
SEQ ID No. 30 (Clone PEOWT22 vH domain CDR2 amino acid sequence)
Ile Ser Tyr Asp Val Ser Asn Lys
1 5
SEQ ID No. 31 (Clone PEOWT22 vH domain CDR3 amino acid sequence)
Ala Arg Ser Tyr Tyr Tyr Tyr Tyr Gly Met Asp Val
1 5 10
SEQ ID No. 32 (Clone PEOWT22 vL domain CDR1 amino acid sequence)
Gln Ser Ile Ser Arg Tyr
1 5
SEQ ID No. 33 (Clone PEOWT22 vL domain CDR2 amino acid sequence)
Asp Ala Ser
1
SEQ ID No. 34 (Clone PEOWT22 vL domain CDR3 amino acid sequence)
Gln Gln Phe Gly Arg Ser Pro Arg Thr
1 5
SEQ ID No. 35 (Clone PEOWT22 full-length vH chain nucleic acid sequence)
```

| | |
|---|---|
| gag gtg caa ctg gtg gag tct ggg gga ggt gtg gta agg cct ggg ggg | 48 |
| Glu Val Gln Leu Val Glu Ser Gly Gly Gly Val Val Arg Pro Gly Gly | |
| 1 5 10 15 | |
| tcc ctg aga ctc tcc tgt gca gcc tct gga ttc acc ttc agt agc tat | 96 |
| Ser Leu Arg Leu Ser Cys Ala Ala Ser Gly Phe Thr Phe Ser Ser Tyr | |
| 20 25 30 | |
| gct atg cac tgg gtc cgc cag gct cca ggc aag ggg ctg gag tgg gtg | 144 |
| Ala Met His Trp Val Arg Gln Ala Pro Gly Lys Gly Leu Glu Trp Val | |
| 35 40 45 | |
| gca gtt ata tca tat gat gta agc aat aaa tac tac gca gac tcc gtg | 192 |
| Ala Val Ile Ser Tyr Asp Val Ser Asn Lys Tyr Tyr Ala Asp Ser Val | |
| 50 55 60 | |
| aag ggc cga ttc acc atc tcc aga gac aat tcc aag aac acg ctg tat | 240 |
| Lys Gly Arg Phe Thr Ile Ser Arg Asp Asn Ser Lys Asn Thr Leu Tyr | |
| 65 70 75 80 | |
| ctg caa atg aac agc ctg aga gct gag gac acg gct gtg tat tac tgt | 288 |
| Leu Gln Met Asn Ser Leu Arg Ala Glu Asp Thr Ala Val Tyr Tyr Cys | |
| 85 90 95 | |
| gcg aga tct tac tac tac tac tac ggt atg gac gtc tgg ggc caa ggg | 336 |
| Ala Arg Ser Tyr Tyr Tyr Tyr Tyr Gly Met Asp Val Trp Gly Gln Gly | |
| 100 105 110 | |
| acc acg gtc acc gtc tcc tca gcc tcc act aag ggc cca tcc gtg ttc | 384 |
| Thr Thr Val Thr Val Ser Ser Ala Ser Thr Lys Gly Pro Ser Val Phe | |
| 115 120 125 | |
| cca ctg gca ccc tct agt aag agc aca tct ggg ggt act gcc gct ctg | 432 |
| Pro Leu Ala Pro Ser Ser Lys Ser Thr Ser Gly Gly Thr Ala Ala Leu | |
| 130 135 140 | |
| gga tgt ctg gtg aag gat tac ttc cca gag cca gtc acc gtg tcc tgg | 480 |
| Gly Cys Leu Val Lys Asp Tyr Phe Pro Glu Pro Val Thr Val Ser Trp | |
| 145 150 155 160 | |
| aac agc ggg gcc ctg act tcc ggt gtc cat acc ttt cca gct gtg ctg | 528 |
| Asn Ser Gly Ala Leu Thr Ser Gly Val His Thr Phe Pro Ala Val Leu | |
| 165 170 175 | |
| cag tca tcc ggc ctg tac agc ctg agc tct gtg gtc acc gtc ccc agt | 576 |
| Gln Ser Ser Gly Leu Tyr Ser Leu Ser Ser Val Val Thr Val Pro Ser | |
| 180 185 190 | |
| tca tcc ctg gga aca cag act tat atc tgc aac gtg aat cac aag cca | 624 |
| Ser Ser Leu Gly Thr Gln Thr Tyr Ile Cys Asn Val Asn His Lys Pro | |
| 195 200 205 | |
| tcc aat aca aaa gtc gac aag aaa gtg gaa ccc aag agc tgt gat aaa | 672 |
| Ser Asn Thr Lys Val Asp Lys Lys Val Glu Pro Lys Ser Cys Asp Lys | |
| 210 215 220 | |
| acc cat aca tgc ccc cct tgt cct gct cca gag ctg ctg gga gga cca | 720 |
| Thr His Thr Cys Pro Pro Cys Pro Ala Pro Glu Leu Leu Gly Gly Pro | |
| 225 230 235 240 | |
| tcc gtg ttc ctg ttt cca ccc aag cct aaa gac act ctg atg att tct | 768 |
| Ser Val Phe Leu Phe Pro Pro Lys Pro Lys Asp Thr Leu Met Ile Ser | |
| 245 250 255 | |
| cga acc ccc gaa gtc aca tgc gtg gtc gtg gac gtg tcc cac gag gat | 816 |
| Arg Thr Pro Glu Val Thr Cys Val Val Val Asp Val Ser His Glu Asp | |
| 260 265 270 | |
| cct gaa gtc aag ttc aac tgg tac gtg gat ggc gtc gag gtg cat aat | 864 |
| Pro Glu Val Lys Phe Asn Trp Tyr Val Asp Gly Val Glu Val His Asn | |
| 275 280 285 | |
| gcc aag aca aaa cca cga gag gaa cag tac aac agt acc tat cgt gtc | 912 |
| Ala Lys Thr Lys Pro Arg Glu Glu Gln Tyr Asn Ser Thr Tyr Arg Val | |
| 290 295 300 | |
| gtg tca gtc ctg aca gtg ctg cac cag gac tgg ctg aac ggg aag gaa | 960 |
| Val Ser Val Leu Thr Val Leu His Gln Asp Trp Leu Asn Gly Lys Glu | |
| 305 310 315 320 | |
| tat aag tgc aaa gtg agc aat aag gca ctg ccc gcc cct atc gag aaa | 1008 |
| Tyr Lys Cys Lys Val Ser Asn Lys Ala Leu Pro Ala Pro Ile Glu Lys | |
| 325 330 335 | |
| aca att tct aag gct aaa gga cag cct agg gaa cca cag gtg tac act | 1056 |
| Thr Ile Ser Lys Ala Lys Gly Gln Pro Arg Glu Pro Gln Val Tyr Thr | |
| 340 345 350 | |
| ctg cct cca tca cgg gac gag ctg aca aag aac cag gtc agt ctg act | 1104 |
| Leu Pro Pro Ser Arg Asp Glu Leu Thr Lys Asn Gln Val Ser Leu Thr | |
| 355 360 365 | |
| tgt ctg gtg aaa ggg ttc tat cct tct gat atc gcc gtg gag tgg gaa | 1152 |
| Cys Leu Val Lys Gly Phe Tyr Pro Ser Asp Ile Ala Val Glu Trp Glu | |
| 370 375 380 | |
| agt aat ggt cag cca gag aac aat tac aag acc aca ccc cct gtc ctg | 1200 |
| Ser Asn Gly Gln Pro Glu Asn Asn Tyr Lys Thr Thr Pro Pro Val Leu | |
| 385 390 395 400 | |
| gac tct gat ggg agt ttc ttt ctg tat tcc aag ctg acc gtg gat aaa | 1248 |
| Asp Ser Asp Gly Ser Phe Phe Leu Tyr Ser Lys Leu Thr Val Asp Lys | |
| 405 410 415 | |
| agc cgg tgg cag cag ggt aat gtc ttt agt tgt tca gtg atg cac gag | 1296 |
| Ser Arg Trp Gln Gln Gly Asn Val Phe Ser Cys Ser Val Met His Glu | |
| 420 425 430 | |
| gca ctg cac aat cac tac acc cag aaa tea ctg tca ctg tca cca ggt | 1344 |
| Ala Leu His Asn His Tyr Thr Gln Lys Ser Leu Ser Leu Ser Pro Gly | |
| 435 440 445 | |
| aaa tga | 1350 |
| Lys | |

```
SEQ ID No. 36 (Clone PEOWT22 full-length vH chain amino acid sequence)
Glu Val Gln Leu Val Glu Ser Gly Gly Gly Val Val Arg Pro Gly Gly
1 5 10 15
Ser Leu Arg Leu Ser Cys Ala Ala Ser Gly Phe Thr Phe Ser Ser Tyr
                        20 25 30
Ala Met His Trp Val Arg Gln Ala Pro Gly Lys Gly Leu Glu Trp Val
                35 40 45
Ala Val Ile Ser Tyr Asp Val Ser Asn Lys Tyr Tyr Ala Asp Ser Val
        50 55 60
Lys Gly Arg Phe Thr Ile Ser Arg Asp Asn Ser Lys Asn Thr Leu Tyr
65 70 75 80
Leu Gln Met Asn Ser Leu Arg Ala Glu Asp Thr Ala Val Tyr Tyr Cys
                                85 90 95
Ala Arg Ser Tyr Tyr Tyr Tyr Tyr Gly Met Asp Val Trp Gly Gln Gly
                        100 105 110
Thr Thr Val Thr Val Ser Ser Ala Ser Thr Lys Gly Pro Ser Val Phe
                115 120 125
Pro Leu Ala Pro Ser Ser Lys Ser Thr Ser Gly Gly Thr Ala Ala Leu
        130 135 140
Gly Cys Leu Val Lys Asp Tyr Phe Pro Glu Pro Val Thr Val Ser Trp
145 150 155 160
Asn Ser Gly Ala Leu Thr Ser Gly Val His Thr Phe Pro Ala Val Leu
                                165 170 175
Gln Ser Ser Gly Leu Tyr Ser Leu Ser Ser Val Val Thr Val Pro Ser
                        180 185 190
Ser Ser Leu Gly Thr Gln Thr Tyr Ile Cys Asn Val Asn His Lys Pro
                195 200 205
Ser Asn Thr Lys Val Asp Lys Lys Val Glu Pro Lys Ser Cys Asp Lys
        210 215 220
Thr His Thr Cys Pro Pro Cys Pro Ala Pro Glu Leu Leu Gly Gly Pro
225 230 235 240
Ser Val Phe Leu Phe Pro Pro Lys Pro Lys Asp Thr Leu Met Ile Ser
                                245 250 255
Arg Thr Pro Glu Val Thr Cys Val Val Val Asp Val Ser His Glu Asp
                        260 265 270
Pro Glu Val Lys Phe Asn Trp Tyr Val Asp Gly Val Glu Val His Asn
                275 280 285
Ala Lys Thr Lys Pro Arg Glu Glu Gln Tyr Asn Ser Thr Tyr Arg Val
        290 295 300
Val Ser Val Leu Thr Val Leu His Gln Asp Trp Leu Asn Gly Lys Glu
305 310 315 320
Tyr Lys Cys Lys Val Ser Asn Lys Ala Leu Pro Ala Pro Ile Glu Lys
                                325 330 335
Thr Ile Ser Lys Ala Lys Gly Gln Pro Arg Glu Pro Gln Val Tyr Thr
                        340 345 350
Leu Pro Pro Ser Arg Asp Glu Leu Thr Lys Asn Gln Val Ser Leu Thr
                355 360 365
Cys Leu Val Lys Gly Phe Tyr Pro Ser Asp Ile Ala Val Glu Trp Glu
        370 375 380
Ser Asn Gly Gln Pro Glu Asn Asn Tyr Lys Thr Thr Pro Pro Val Leu
385 390 395 400
Asp Ser Asp Gly Ser Phe Phe Leu Tyr Ser Lys Leu Thr Val Asp Lys
                                405 410 415
Ser Arg Trp Gln Gln Gly Asn Val Phe Ser Cys Ser Val Met His Glu
                        420 425 430
Ala Leu His Asn His Tyr Thr Gln Lys Ser Leu Ser Leu Ser Pro Gly
                435 440 445
Lys
SEQ ID No. 37 (Clone PEOWT22 full-length vL chain nucleic acid sequence)
```

| | |
|---|---|
| gat gtt gtg atg acc cag tct cca tcc tcc ctg tct gca tct gta gga | 48 |
| Asp Val Val Met Thr Gln Ser Pro Ser Ser Leu Ser Ala Ser Val Gly | |
| 1 5 10 15 | |
| gac aga gtc acc atc act tgc cgg gca agt cag agc att agc agg tac | 96 |
| Asp Arg Val Thr Ile Thr Cys Arg Ala Ser Gln Ser Ile Ser Arg Tyr | |
| 20 25 30 | |
| tta gcc tgg tac caa cag aaa cct ggc cag gct ccc agg ctc ctc atc | 144 |
| Leu Ala Trp Tyr Gln Gln Lys Pro Gly Gln Ala Pro Arg Leu Leu Ile | |
| 35 40 45 | |
| tat gat gca tcc aac agg gcc act ggc atc cca gtc agg ttc agt ggc | 192 |
| Tyr Asp Ala Ser Asn Arg Ala Thr Gly Ile Pro Val Arg Phe Ser Gly | |
| 50 55 60 | |
| agt ggg tct ggg aca gac ttc act ctc acc atc agc aga ctg gag cca | 240 |
| Ser Gly Ser Gly Thr Asp Phe Thr Leu Thr Ile Ser Arg Leu Glu Pro | |
| 65 70 75 80 | |
| gaa gat ttt gca gtg tat tac tgt cag cag ttt ggt agg tca cct cgg | 288 |
| Glu Asp Phe Ala Val Tyr Tyr Cys Gln Gln Phe Gly Arg Ser Pro Arg | |
| 85 90 95 | |
| acg ttc ggc caa ggg aca cga ctg gag att aaa cga act gtg gct gca | 336 |
| Thr Phe Gly Gln Gly Thr Arg Leu Glu Ile Lys Arg Thr Val Ala Ala | |
| 100 105 110 | |
| cca tct gtc ttc atc ttc ccg cca tct gat gag cag ttg aaa tct gga | 384 |
| Pro Ser Val Phe Ile Phe Pro Pro Ser Asp Glu Gln Leu Lys Ser Gly | |
| 115 120 125 | |
| act gcc tct gtt gtg tgc ctg ctg aat aac ttc tat ccc aga gag gcc | 432 |
| Thr Ala Ser Val Val Cys Leu Leu Asn Asn Phe Tyr Pro Arg Glu Ala | |
| 130 135 140 | |
| aaa gta cag tgg aag gtg gat aac gcc ctc caa tcg ggt aac tcc cag | 480 |
| Lys Val Gln Trp Lys Val Asp Asn Ala Leu Gln Ser Gly Asn Ser Gln | |
| 145 150 155 160 | |
| gag agt gtc aca gag cag gac agc aag gac agc acc tac agc ctc agc | 528 |
| Glu Ser Val Thr Glu Gln Asp Ser Lys Asp Ser Thr Tyr Ser Leu Ser | |
| 165 170 175 | |
| agc acc ctg acg ctg agc aaa gca gac tac gag aaa cac aaa gtc tac | 576 |
| Ser Thr Leu Thr Leu Ser Lys Ala Asp Tyr Glu Lys His Lys Val Tyr | |
| 180 185 190 | |
| gcc tgc gaa gtc acc cat cag ggc ctg agc tcg ccc gtc aca aag agc | 624 |
| Ala Cys Glu Val Thr His Gln Gly Leu Ser Ser Pro Val Thr Lys Ser | |
| 195 200 205 | |
| ttc aac agg gga gag tgt tag | 645 |
| Phe Asn Arg Gly Glu Cys | |
| 210 | |

```
SEQ ID No. 38 (Clone PEOWT22 full-length vL chain amino acid sequence)
Asp Val Val Met Thr Gln Ser Pro Ser Ser Leu Ser Ala Ser Val Gly
1 5 10 15
Asp Arg Val Thr Ile Thr Cys Arg Ala Ser Gln Ser Ile Ser Arg Tyr
                        20 25 30
Leu Ala Trp Tyr Gln Gln Lys Pro Gly Gln Ala Pro Arg Leu Leu Ile
                35 40 45
Tyr Asp Ala Ser Asn Arg Ala Thr Gly Ile Pro Val Arg Phe Ser Gly
        50 55 60
Ser Gly Ser Gly Thr Asp Phe Thr Leu Thr Ile Ser Arg Leu Glu Pro
65 70 75 80
Glu Asp Phe Ala Val Tyr Tyr Cys Gln Gln Phe Gly Arg Ser Pro Arg
                                85 90 95
Thr Phe Gly Gln Gly Thr Arg Leu Glu Ile Lys Arg Thr Val Ala Ala
                        100 105 110
Pro Ser Val Phe Ile Phe Pro Pro Ser Asp Glu Gln Leu Lys Ser Gly
                115 120 125
Thr Ala Ser Val Val Cys Leu Leu Asn Asn Phe Tyr Pro Arg Glu Ala
        130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
                                165 170 175
Ser Thr Leu Thr Leu Ser Lys Ala Asp Tyr Glu Lys His Lys Val Tyr
                        180 185 190
Ala Cys Glu Val Thr His Gln Gly Leu Ser Ser Pro Val Thr Lys Ser
                195 200 205
Phe Asn Arg Gly Glu Cys
        210
SEQ ID No. 39 (Clone PEOWT22 scFv nucleic acid sequence)
```

| | |
|---|---|
| gat gtt gtg atg acc cag tct cca tcc tcc ctg tct gca tct gta gga | 48 |
| Asp Val Val Met Thr Gln Ser Pro Ser Ser Leu Ser Ala Ser Val Gly | |
| 1 5 10 15 | |
| gac aga gtc acc atc act tgc cgg gca agt cag agc att agc agg tac | 96 |
| Asp Arg Val Thr Ile Thr Cys Arg Ala Ser Gln Ser Ile Ser Arg Tyr | |
| 20 25 30 | |
| tta gcc tgg tac caa cag aaa cct ggc cag gct ccc agg ctc ctc atc | 144 |
| Leu Ala Trp Tyr Gln Gln Lys Pro Gly Gln Ala Pro Arg Leu Leu Ile | |
| 35 40 45 | |
| tat gat gca tcc aac agg gcc act ggc atc cca gtc agg ttc agt ggc | 192 |
| Tyr Asp Ala Ser Asn Arg Ala Thr Gly Ile Pro Val Arg Phe Ser Gly | |
| 50 55 60 | |
| agt ggg tct ggg aca gac ttc act ctc acc atc agc aga ctg gag cca | 240 |
| Ser Gly Ser Gly Thr Asp Phe Thr Leu Thr Ile Ser Arg Leu Glu Pro | |
| 65 70 75 80 | |
| gaa gat ttt gca gtg tat tac tgt cag cag ttt ggt agg tca cct cgg | 288 |
| Glu Asp Phe Ala Val Tyr Tyr Cys Gln Gln Phe Gly Arg Ser Pro Arg | |
| 85 90 95 | |
| acg ttc ggc caa ggg aca cga ctg gag att aaa ggc gga tcc tct agg | 336 |
| Thr Phe Gly Gln Gly Thr Arg Leu Glu Ile Lys Gly Gly Ser Ser Arg | |
| 100 105 110 | |
| tca agt tcc agc ggc ggc ggt ggc agc gga ggc ggc ggt gag gtg caa | 384 |
| Ser Ser Ser Ser Gly Gly Gly Gly Ser Gly Gly Gly Gly Glu Val Gln | |
| 115 120 125 | |
| ctg gtg gag tct ggg gga ggt gtg gta agg cct ggg ggg tcc ctg aga | 432 |
| Leu Val Glu Ser Gly Gly Gly Val Val Arg Pro Gly Gly Ser Leu Arg | |
| 130 135 140 | |
| ctc tcc tgt gca gcc tct gga ttc acc ttc agt agc tat gct atg cac | 480 |
| Leu Ser Cys Ala Ala Ser Gly Phe Thr Phe Ser Ser Tyr Ala Met His | |
| 145 150 155 160 | |
| tgg gtc cgc cag gct cca ggc aag ggg ctg gag tgg gtg gca gtt ata | 528 |
| Trp Val Arg Gln Ala Pro Gly Lys Gly Leu Glu Trp Val Ala Val Ile | |
| 165 170 175 | |
| tca tat gat gta agc aat aaa tac tac gca gac tcc gtg aag ggc cga | 576 |
| Ser Tyr Asp Val Ser Asn Lys Tyr Tyr Ala Asp Ser Val Lys Gly Arg | |
| 180 185 190 | |
| ttc acc atc tcc aga gac aat tcc aag aac acg ctg tat ctg caa atg | 624 |
| Phe Thr Ile Ser Arg Asp Asn Ser Lys Asn Thr Leu Tyr Leu Gln Met | |
| 195 200 205 | |
| aac agc ctg aga gct gag gac acg gct gtg tat tac tgt gcg aga tct | 672 |
| Asn Ser Leu Arg Ala Glu Asp Thr Ala Val Tyr Tyr Cys Ala Arg Ser | |
| 210 215 220 | |
| tac tac tac tac tac ggt atg gac gtc tgg ggc caa ggg acc acg gtc | 720 |
| Tyr Tyr Tyr Tyr Tyr Gly Met Asp Val Trp Gly Gln Gly Thr Thr Val | |
| 225 230 235 240 | |
| acc gtc tcc tca | 732 |
| Thr Val Ser Ser | |

```
SEQ ID No. 40 (Clone PEOWT22 scFv amino acid sequence)
Asp Val Val Met Thr Gln Ser Pro Ser Ser Leu Ser Ala Ser Val Gly
1 5 10 15
Asp Arg Val Thr Ile Thr Cys Arg Ala Ser Gln Ser Ile Ser Arg Tyr
                        20 25 30
Leu Ala Trp Tyr Gln Gln Lys Pro Gly Gln Ala Pro Arg Leu Leu Ile
                35 40 45
Tyr Asp Ala Ser Asn Arg Ala Thr Gly Ile Pro Val Arg Phe Ser Gly
        50 55 60
Ser Gly Ser Gly Thr Asp Phe Thr Leu Thr Ile Ser Arg Leu Glu Pro
65 70 75 80
Glu Asp Phe Ala Val Tyr Tyr Cys Gln Gln Phe Gly Arg Ser Pro Arg
                                85 90 95
Thr Phe Gly Gln Gly Thr Arg Leu Glu Ile Lys Gly Gly Ser Ser Arg
                        100 105 110
Ser Ser Ser Ser Gly Gly Gly Gly Ser Gly Gly Gly Gly Glu Val Gln
                115 120 125
Leu Val Glu Ser Gly Gly Gly Val Val Arg Pro Gly Gly Ser Leu Arg
        130 135 140
Leu Ser Cys Ala Ala Ser Gly Phe Thr Phe Ser Ser Tyr Ala Met His
145 150 155 160
Trp Val Arg Gln Ala Pro Gly Lys Gly Leu Glu Trp Val Ala Val Ile
                                165 170 175
Ser Tyr Asp Val Ser Asn Lys Tyr Tyr Ala Asp Ser Val Lys Gly Arg
                        180 185 190
Phe Thr Ile Ser Arg Asp Asn Ser Lys Asn Thr Leu Tyr Leu Gln Met
                195 200 205
Asn Ser Leu Arg Ala Glu Asp Thr Ala Val Tyr Tyr Cys Ala Arg Ser
        210 215 220
Tyr Tyr Tyr Tyr Tyr Gly Met Asp Val Trp Gly Gln Gly Thr Thr Val
225 230 235 240
Thr Val Ser Ser
SEQ ID No. 41 (Clone PEO25 vH domain nucleic acid sequence)
```

| | |
|---|---|
| cag tcg gtg gag gag tcc ggg ggt cgc ctg gtc acg cct ggg aca ccc | 48 |
| Gln Ser Val Glu Glu Ser Gly Gly Arg Leu Val Thr Pro Gly Thr Pro | |
| 1 5 10 15 | |
| ctg aca ctc acc tgc aca gtc tct gga atc gac ctc agt aac aat gca | 96 |
| Leu Thr Leu Thr Cys Thr Val Ser Gly Ile Asp Leu Ser Asn Asn Ala | |
| 20 25 30 | |
| atg agc tgg gtc cgc cag gct cca ggg aag ggg ctg gaa tat atc gga | 144 |
| Met Ser Trp Val Arg Gln Ala Pro Gly Lys Gly Leu Glu Tyr Ile Gly | |
| 35 40 45 | |
| atc att agg agt agt ggt agt aca tat tac gcg aac tgg gca aaa ggc | 192 |
| Ile Ile Arg Ser Ser Gly Ser Thr Tyr Tyr Ala Asn Trp Ala Lys Gly | |
| 50 55 60 | |
| cgg ttc acc atc tcc aaa acc tcg acc acg gtg gat ctg ata atc acc | 240 |
| Arg Phe Thr Ile Ser Lys Thr Ser Thr Thr Val Asp Leu Ile Ile Thr | |
| 65 70 75 80 | |
| agt ccg aca acc gag gac acg gcc acc tat ttc tgt gcc aga ggg ggt | 288 |
| Ser Pro Thr Thr Glu Asp Thr Ala Thr Tyr Phe Cys Ala Arg Gly Gly | |
| 85 90 95 | |
| ggt tct tac agt att gtc ttc tgg aac ttg tgg ggc cca ggc acc ctg | 336 |
| Gly Ser Tyr Ser Ile Val Phe Trp Asn Leu Trp Gly Pro Gly Thr Leu | |
| 100 105 110 | |
| gtc acc gtc tcc tca | 351 |
| Val Thr Val Ser Ser | |
| 115 | |

```
SEQ ID No. 42 (Clone PEO25 vH domain amino acid sequence)
Gln Ser Val Glu Glu Ser Gly Gly Arg Leu Val Thr Pro Gly Thr Pro
1 5 10 15
Leu Thr Leu Thr Cys Thr Val Ser Gly Ile Asp Leu Ser Asn Asn Ala
                        20 25 30
Met Ser Trp Val Arg Gln Ala Pro Gly Lys Gly Leu Glu Tyr Ile Gly
                35 40 45
Ile Ile Arg Ser Ser Gly Ser Thr Tyr Tyr Ala Asn Trp Ala Lys Gly
        50 55 60
Arg Phe Thr Ile Ser Lys Thr Ser Thr Thr Val Asp Leu Ile Ile Thr
65 70 75 80
Ser Pro Thr Thr Glu Asp Thr Ala Thr Tyr Phe Cys Ala Arg Gly Gly
                                85 90 95
Gly Ser Tyr Ser Ile Val Phe Trp Asn Leu Trp Gly Pro Gly Thr Leu
                        100 105 110
Val Thr Val Ser Ser
                115
SEQ ID No. 43 (Clone PEO25 vL domain nucleic acid sequence)
```

| | |
|---|---|
| gcc tat gat atg acc cag act cca gcc tct gtg gag gta gct gtg gga | 48 |
| Ala Tyr Asp Met Thr Gln Thr Pro Ala Ser Val Glu Val Ala Val Gly | |
| 1 5 10 15 | |
| ggc aca gtc acc atc aat tgc cag gcc agt gag agg att tat agc aat | 96 |
| Gly Thr Val Thr Ile Asn Cys Gln Ala Ser Glu Arg Ile Tyr Ser Asn | |
| 20 25 30 | |
| tta gcc tgg tat cag cag aaa cca ggg cag cgt ccc aag ctc ctg atc | 144 |
| Leu Ala Trp Tyr Gln Gln Lys Pro Gly Gln Arg Pro Lys Leu Leu Ile | |
| 35 40 45 | |
| tat tat aca tcc act ctg gca tct ggg gtc tca tcg cgg ttc aaa ggc | 192 |
| Tyr Tyr Thr Ser Thr Leu Ala Ser Gly Val Ser Ser Arg Phe Lys Gly | |
| 50 55 60 | |
| agt gga tct ggg aca cag ttc act ctc acc atc agc ggc gtg gag tgt | 240 |
| Ser Gly Ser Gly Thr Gln Phe Thr Leu Thr Ile Ser Gly Val Glu Cys | |
| 65 70 75 80 | |
| gcc gat gct gcc act tac tac tgt caa cag ggt tat agt agt agt aat | 288 |
| Ala Asp Ala Ala Thr Tyr Tyr Cys Gln Gln Gly Tyr Ser Ser Ser Asn | |
| 85 90 95 | |
| gtt gac aat act ttc ggc gga ggg acc gag gtg gtg gtc aaa ggt | 333 |
| Val Asp Asn Thr Phe Gly Gly Gly Thr Glu Val Val Val Lys Gly | |
| 100 105 110 | |

```
SEQ ID No. 44 (Clone PEO25 vL domain amino acid sequence)
Ala Tyr Asp Met Thr Gln Thr Pro Ala Ser Val Glu Val Ala Val Gly
1 5 10 15
Gly Thr Val Thr Ile Asn Cys Gln Ala Ser Glu Arg Ile Tyr Ser Asn
                        20 25 30
Leu Ala Trp Tyr Gln Gln Lys Pro Gly Gln Arg Pro Lys Leu Leu Ile
                35 40 45
Tyr Tyr Thr Ser Thr Leu Ala Ser Gly Val Ser Ser Arg Phe Lys Gly
        50 55 60
Ser Gly Ser Gly Thr Gln Phe Thr Leu Thr Ile Ser Gly Val Glu Cys
65 70 75 80
Ala Asp Ala Ala Thr Tyr Tyr Cys Gln Gln Gly Tyr Ser Ser Ser Asn
                                85 90 95
Val Asp Asn Thr Phe Gly Gly Gly Thr Glu Val Val Val Lys Gly
                        100 105 110
SEQ ID No. 45 (Humanized PEO20 vH domain nucleic acid sequence)
```

| | |
|---|---|
| ggc gag cag cag ctg gtg gag agc ggc gga ggc ctg gtg cag cct gga | 48 |
| Gly Glu Gln Gln Leu Val Glu Ser Gly Gly Gly Leu Val Gln Pro Gly | |
| 1 5 10 15 | |
| gga agc ctg agg ctg agc tgc gcc gtg tcc ggc ttc agc ctg agc agc | 96 |
| Gly Ser Leu Arg Leu Ser Cys Ala Val Ser Gly Phe Ser Leu Ser Ser | |
| 20 25 30 | |
| tac gcc atc agc tgg gtg agg cag gcc ccc gga aag ggc ctg gag tac | 144 |
| Tyr Ala Ile Ser Trp Val Arg Gln Ala Pro Gly Lys Gly Leu Glu Tyr | |
| 35 40 45 | |
| atc gcc atc atc aac agc tac ggc acc acc tac tac gcc agc tgg gcc | 192 |
| Ile Ala Ile Ile Asn Ser Tyr Gly Thr Thr Tyr Tyr Ala Ser Trp Ala | |
| 50 55 60 | |
| aag ggc aga gtg acc atc tcc aag gat tcc tcc aag aac acc gtg tac | 240 |
| Lys Gly Arg Val Thr Ile Ser Lys Asp Ser Ser Lys Asn Thr Val Tyr | |
| 65 70 75 80 | |
| ctg cag atg ggc tcc ctg aga gcc gag gat atg gcc gtg tac ttt tgc | 288 |
| Leu Gln Met Gly Ser Leu Arg Ala Glu Asp Met Ala Val Tyr Phe Cys | |
| 85 90 95 | |
| gcc aga ggc gat tcc tac ggc tcc ggc gtg ggc ctg ggc ctg tgg gga | 336 |
| Ala Arg Gly Asp Ser Tyr Gly Ser Gly Val Gly Leu Gly Leu Trp Gly | |
| 100 105 110 | |
| cct gga acc ctg gtg aca gtg tcc tcc | 363 |
| Pro Gly Thr Leu Val Thr Val Ser Ser | |
| 115 120 | |

```
SEQ ID No. 46 (Humanized PEO20 vH domain amino acid sequence)
Gly Glu Gln Gln Leu Val Glu Ser Gly Gly Gly Leu Val Gln Pro Gly
1 5 10 15
Gly Ser Leu Arg Leu Ser Cys Ala Val Ser Gly Phe Ser Leu Ser Ser
                        20 25 30
Tyr Ala Ile Ser Trp Val Arg Gln Ala Pro Gly Lys Gly Leu Glu Tyr
                35 40 45
Ile Ala Ile Ile Asn Ser Tyr Gly Thr Thr Tyr Tyr Ala Ser Trp Ala
        50 55 60
Lys Gly Arg Val Thr Ile Ser Lys Asp Ser Ser Lys Asn Thr Val Tyr
65 70 75 80
Leu Gln Met Gly Ser Leu Arg Ala Glu Asp Met Ala Val Tyr Phe Cys
                                85 90 95
Ala Arg Gly Asp Ser Tyr Gly Ser Gly Val Gly Leu Gly Leu Trp Gly
                        100 105 110
Pro Gly Thr Leu Val Thr Val Ser Ser
                115 120
SEQ ID No. 47 (Humanized PEO20 vL domain nucleic acid sequence)
```

| | |
|---|---|
| gga gac tac cag atg aca cag tcc cct agc acc ctg tcc gcc tcc gtg | 48 |
| Gly Asp Tyr Gln Met Thr Gln Ser Pro Ser Thr Leu Ser Ala Ser Val | |
| 1 5 10 15 | |
| ggc gac aga gtg aca atc acc tgt cag gcc tcc cag aat atc tac agc | 96 |
| Gly Asp Arg Val Thr Ile Thr Cys Gln Ala Ser Gln Asn Ile Tyr Ser | |
| 20 25 30 | |
| aat ctg gcc tgg tac cag cag aag cct ggc aag agg ccc aag ctg ctg | 144 |
| Asn Leu Ala Trp Tyr Gln Gln Lys Pro Gly Lys Arg Pro Lys Leu Leu | |
| 35 40 45 | |
| atc tac aga gcc agc tcc ctg gcc tcc ggc gtg cca tct aga ttt tcc | 192 |
| Ile Tyr Arg Ala Ser Ser Leu Ala Ser Gly Val Pro Ser Arg Phe Ser | |
| 50 55 60 | |
| ggc tcc ggc agc ggc aca gag ttt acc ctg aca atc agc agc ctg cag | 240 |
| Gly Ser Gly Ser Gly Thr Glu Phe Thr Leu Thr Ile Ser Ser Leu Gln | |
| 65 70 75 80 | |
| ccc gat gat ttc gcc acc tac tac tgt cag cag ggc ttc agc agc aat | 288 |
| Pro Asp Asp Phe Ala Thr Tyr Tyr Cys Gln Gln Gly Phe Ser Ser Asn | |
| 85 90 95 | |
| aat gtg gac aat aca ttt ggc ggc ggc aca aag gtg gag atc aag | 333 |
| Asn Val Asp Asn Thr Phe Gly Gly Gly Thr Lys Val Glu Ile Lys | |
| 100 105 110 | |

```
SEQ ID No. 48 (Humanized PEO20 vL domain amino acid sequence)
Gly Asp Tyr Gln Met Thr Gln Ser Pro Ser Thr Leu Ser Ala Ser Val
1 5 10 15
Gly Asp Arg Val Thr Ile Thr Cys Gln Ala Ser Gln Asn Ile Tyr Ser
                        20 25 30
Asn Leu Ala Trp Tyr Gln Gln Lys Pro Gly Lys Arg Pro Lys Leu Leu
                35 40 45
Ile Tyr Arg Ala Ser Ser Leu Ala Ser Gly Val Pro Ser Arg Phe Ser
        50 55 60
Gly Ser Gly Ser Gly Thr Glu Phe Thr Leu Thr Ile Ser Ser Leu Gln
65 70 75 80
Pro Asp Asp Phe Ala Thr Tyr Tyr Cys Gln Gln Gly Phe Ser Ser Asn
                                85 90 95
Asn Val Asp Asn Thr Phe Gly Gly Gly Thr Lys Val Glu Ile Lys
                        100 105 110
SEQ ID No. 49 (Humanized PEO19 vH domain nucleic acid sequence)
```

| | |
|---|---|
| ggc gag cag cag ctg gtg gag agc ggc gga ggc ctg gtg cag cct gga | 48 |
| Gly Glu Gln Gln Leu Val Glu Ser Gly Gly Gly Leu Val Gln Pro Gly | |
| 1 5 10 15 | |
| gga agc ctg agg ctg agc tgc gcc gtg tcc ggc ttt tcc ctg agc aag | 96 |
| Gly Ser Leu Arg Leu Ser Cys Ala Val Ser Gly Phe Ser Leu Ser Lys | |
| 20 25 30 | |
| agc atc atc agc tgg gtg agg cag gcc cct ggc aag ggc ctg gag tac | 144 |
| Ser Ile Ile Ser Trp Val Arg Gln Ala Pro Gly Lys Gly Leu Glu Tyr | |
| 35 40 45 | |
| atc ggc atc atc ggc agc agc ggc tcc acc tac tac gcc aac tgg gcc | 192 |
| Ile Gly Ile Ile Gly Ser Ser Gly Ser Thr Tyr Tyr Ala Asn Trp Ala | |
| 50 55 60 | |
| aag ggc aga ttc aca atc tcc aag gac tcc tcc aag aat acc gtg tac | 240 |
| Lys Gly Arg Phe Thr Ile Ser Lys Asp Ser Ser Lys Asn Thr Val Tyr | |
| 65 70 75 80 | |
| ctg cag atg ggc tcc ctg agg gcc gag gat atg gcc gtg tac ttt tgt | 288 |
| Leu Gln Met Gly Ser Leu Arg Ala Glu Asp Met Ala Val Tyr Phe Cys | |
| 85 90 95 | |
| gcc aga ggc ctg ctg tac tcc ggc aat aag tcc tgg ggc ccc ggc aca | 336 |
| Ala Arg Gly Leu Leu Tyr Ser Gly Asn Lys Ser Trp Gly Pro Gly Thr | |
| 100 105 110 | |
| ctg gtg acc gtg agc tcc | 354 |
| Leu Val Thr Val Ser Ser | |
| 115 | |

```
SEQ ID No. 50 (Humanized PEO19 vH domain amino acid sequence)
Gly Glu Gln Gln Leu Val Glu Ser Gly Gly Gly Leu Val Gln Pro Gly
1 5 10 15
Gly Ser Leu Arg Leu Ser Cys Ala Val Ser Gly Phe Ser Leu Ser Lys
                        20 25 30
Ser Ile Ile Ser Trp Val Arg Gln Ala Pro Gly Lys Gly Leu Glu Tyr
                35 40 45
Ile Gly Ile Ile Gly Ser Ser Gly Ser Thr Tyr Tyr Ala Asn Trp Ala
        50 55 60
Lys Gly Arg Phe Thr Ile Ser Lys Asp Ser Ser Lys Asn Thr Val Tyr
65 70 75 80
Leu Gln Met Gly Ser Leu Arg Ala Glu Asp Met Ala Val Tyr Phe Cys
                                85 90 95
Ala Arg Gly Leu Leu Tyr Ser Gly Asn Lys Ser Trp Gly Pro Gly Thr
                        100 105 110
Leu Val Thr Val Ser Ser
                115
SEQ ID No. 51 (Humanized PEO19 vL domain nucleic acid sequence)
```

| | |
|---|---|
| ggc gac atc gtg atg acc cag tcc ccc gat tcc ctg gcc gtg tcc ctg | 48 |
| Gly Asp Ile Val Met Thr Gln Ser Pro Asp Ser Leu Ala Val Ser Leu | |
| 1 5 10 15 | |
| ggc gag aga gcc aca atc aat tgt cag toc toc cag agc gtg ctg ctg | 96 |
| Gly Glu Arg Ala Thr Ile Asn Cys Gln Ser Ser Gln Ser Val Leu Leu | |
| 20 25 30 | |
| aac aat cag ctg tcc tgg ttc cag cag aag cct ggc cag cct ccc aag | 144 |
| Asn Asn Gln Leu Ser Trp Phe Gln Gln Lys Pro Gly Gln Pro Pro Lys | |
| 35 40 45 | |
| ctg ctg atc tac gac gcc tcc aca ctg gag tcc ggc gtg ccc gat agg | 192 |
| Leu Leu Ile Tyr Asp Ala Ser Thr Leu Glu Ser Gly Val Pro Asp Arg | |
| 50 55 60 | |
| ttc agc ggc tcc ggc agc ggc acc gac ttt acc ctg acc atc tec agc | 240 |
| Phe Ser Gly Ser Gly Ser Gly Thr Asp Phe Thr Leu Thr Ile Ser Ser | |
| 65 70 75 80 | |
| ctg cag gcc gag gat gtg gcc gtg tac tac tgc ctg ggc ggc tac agc | 288 |
| Leu Gln Ala Glu Asp Val Ala Val Tyr Tyr Cys Leu Gly Gly Tyr Ser | |
| 85 90 95 | |
| ggc aac ctg tac gcc ttt ggc ggc ggc acc aag gtg gag atc aag | 333 |
| Gly Asn Leu Tyr Ala Phe Gly Gly Gly Thr Lys Val Glu Ile Lys | |
| 100 105 110 | |

```
SEQ ID No. 52 (Humanized PEO19 vL domain amino acid sequence)
Gly Asp Ile Val Met Thr Gln Ser Pro Asp Ser Leu Ala Val Ser Leu
1 5 10 15
Gly Glu Arg Ala Thr Ile Asn Cys Gln Ser Ser Gln Ser Val Leu Leu
                        20 25 30
Asn Asn Gln Leu Ser Trp Phe Gln Gln Lys Pro Gly Gln Pro Pro Lys
                35 40 45
Leu Leu Ile Tyr Asp Ala Ser Thr Leu Glu Ser Gly Val Pro Asp Arg
        50 55 60
Phe Ser Gly Ser Gly Ser Gly Thr Asp Phe Thr Leu Thr Ile Ser Ser
65 70 75 80
Leu Gln Ala Glu Asp Val Ala Val Tyr Tyr Cys Leu Gly Gly Tyr Ser
                                85 90 95
Gly Asn Leu Tyr Ala Phe Gly Gly Gly Thr Lys Val Glu Ile Lys
                        100 105 110
SEQ ID No. 53 (Humanized PEO21 vH domain nucleic acid sequence)
```

| | |
|---|---|
| ggc gag cag cag ctg gtg gag tcc ggc gga ggc ctg gtg cag cca gga | 48 |
| Gly Glu Gln Gln Leu Val Glu Ser Gly Gly Gly Leu Val Gln Pro Gly | |
| 1 5 10 15 | |
| gga agc ctg agg ctg tcc tgt gcc gtg agc ggc ttc tcc ctg agc tcc | 96 |
| Gly Ser Leu Arg Leu Ser Cys Ala Val Ser Gly Phe Ser Leu Ser Ser | |
| 20 25 30 | |
| tac gcc atg agc tgg gtg agg cag gcc ccc gga aag ggc ctg gag tac | 144 |
| Tyr Ala Met Ser Trp Val Arg Gln Ala Pro Gly Lys Gly Leu Glu Tyr | |
| 35 40 45 | |
| atc ggc atc atc agc agc agc ggc agc aca tac tac gcc agc tgg gcc | 192 |
| Ile Gly Ile Ile Ser Ser Ser Gly Ser Thr Tyr Tyr Ala Ser Trp Ala | |
| 50 55 60 | |
| aag ggc agg ttc aca atc agc aag gat tcc tcc aag aat aca gtg tac | 240 |
| Lys Gly Arg Phe Thr Ile Ser Lys Asp Ser Ser Lys Asn Thr Val Tyr | |
| 65 70 75 80 | |
| ctg cag atg ggc tcc ctg agg gcc gag gac atg gcc gtg tac ttc tgt | 288 |
| Leu Gln Met Gly Ser Leu Arg Ala Glu Asp Met Ala Val Tyr Phe Cys | |
| 85 90 95 | |
| gcc aga gac agg gtc atc tat tcc atc ggc cct tac tac ttc aac ctg | 336 |
| Ala Arg Asp Arg Val Ile Tyr Ser Ile Gly Pro Tyr Tyr Phe Asn Leu | |
| 100 105 110 | |
| tgg ggc ccc ggc aca ctg gtg aca gtg tcc agc | 369 |
| Trp Gly Pro Gly Thr Leu Val Thr Val Ser Ser | |
| 115 120 | |

```
SEQ ID No. 54 (Humanized PEO21 vH domain amino acid sequence)
Gly Glu Gln Gln Leu Val Glu Ser Gly Gly Gly Leu Val Gln Pro Gly
1 5 10 15
Gly Ser Leu Arg Leu Ser Cys Ala Val Ser Gly Phe Ser Leu Ser Ser
                        20 25 30
Tyr Ala Met Ser Trp Val Arg Gln Ala Pro Gly Lys Gly Leu Glu Tyr
                35 40 45
Ile Gly Ile Ile Ser Ser Ser Gly Ser Thr Tyr Tyr Ala Ser Trp Ala
        50 55 60
Lys Gly Arg Phe Thr Ile Ser Lys Asp Ser Ser Lys Asn Thr Val Tyr
65 70 75 80
Leu Gln Met Gly Ser Leu Arg Ala Glu Asp Met Ala Val Tyr Phe Cys
                                85 90 95
Ala Arg Asp Arg Val Ile Tyr Ser Ile Gly Pro Tyr Tyr Phe Asn Leu
                        100 105 110
Trp Gly Pro Gly Thr Leu Val Thr Val Ser Ser
                115 120
SEQ ID No. 55 (Humanized PEO21 vL domain nucleic acid sequence)
```

| | |
|---|---|
| ggc gat tac cag atg aca cag tcc ccc tcc tcc ctg agc gcc tcc gtg | 48 |
| Gly Asp Tyr Gln Met Thr Gln Ser Pro Ser Ser Leu Ser Ala Ser Val | |
| 1 5 10 15 | |
| gga gat agg gtg acc atc aca tgc cag gcc agc gag atc atc tac agc | 96 |
| Gly Asp Arg Val Thr Ile Thr Cys Gln Ala Ser Glu Ile Ile Tyr Ser | |
| 20 25 30 | |
| aat ctg gcc tgg tac cag cag aag ccc ggc aag ccc ccc aag ctg ctg | 144 |
| Asn Leu Ala Trp Tyr Gln Gln Lys Pro Gly Lys Pro Pro Lys Leu Leu | |
| 35 40 45 | |
| atc tac ggc gcc tcc aca ctg gcc agc ggc gtg cct agc aga ttc agc | 192 |
| Ile Tyr Gly Ala Ser Thr Leu Ala Ser Gly Val Pro Ser Arg Phe Ser | |
| 50 55 60 | |
| ggc agc ggc tcc ggc acc gat tac acc ctg aca atc tcc agc ctg cag | 240 |
| Gly Ser Gly Ser Gly Thr Asp Tyr Thr Leu Thr Ile Ser Ser Leu Gln | |
| 65 70 75 80 | |
| cct gag gat ttt gcc aca tac tac tgt cag cag tcc ttc agc toc aat | 288 |
| Pro Glu Asp Phe Ala Thr Tyr Tyr Cys Gln Gln Ser Phe Ser Ser Asn | |
| 85 90 95 | |
| aac gtg ggc aac atc ttc ggc ggc ggc aca aag gtg gag atc aag | 333 |
| Asn Val Gly Asn Ile Phe Gly Gly Gly Thr Lys Val Glu Ile Lys | |
| 100 105 110 | |

```
SEQ ID No. 56 (Humanized PEO21 vL domain amino acid sequence)
Gly Asp Tyr Gln Met Thr Gln Ser Pro Ser Ser Leu Ser Ala Ser Val
1 5 10 15
Gly Asp Arg Val Thr Ile Thr Cys Gln Ala Ser Glu Ile Ile Tyr Ser
                        20 25 30
Asn Leu Ala Trp Tyr Gln Gln Lys Pro Gly Lys Pro Pro Lys Leu Leu
                35 40 45
Ile Tyr Gly Ala Ser Thr Leu Ala Ser Gly Val Pro Ser Arg Phe Ser
        50 55 60
Gly Ser Gly Ser Gly Thr Asp Tyr Thr Leu Thr Ile Ser Ser Leu Gln
65 70 75 80
Pro Glu Asp Phe Ala Thr Tyr Tyr Cys Gln Gln Ser Phe Ser Ser Asn
                                85 90 95
Val Gly Asn Ile Phe Gly Gly Gly Thr Lys Val Glu Ile Lys
                        100 105 110
```

For use in human patients, it will be desirable to humanize these antibodies, replacing both the constant regions of the heavy and light chains with human constant regions, as well as replacing the framework regions of the variable regions with human antibody framework regions. In some embodiments, the anti-CD39 antibody or antigen-binding fragment thereof, is a humanized version of a rabbit antibody.

In some embodiments, the anti-CD39 antibody or antigen-binding fragment thereof comprises at least one heavy chain variable is at least 60% identical to SEQ ID No. 2, 6, 10, 14, 18, 22, 26, 42, 46, 50, or 54, and even more preferably at least 65%, 70%, 75%, 80%, 85% or even 90% identical to SEQ ID No. 2, 6, 10, 14, 18, 22, 26, 42, 46, 50, or 54, and able to specifically bind human CD39.

In some embodiments, the anti-CD39 antibody or antigen-binding fragment thereof comprises at least one light chain variable is at least 60% identical to SEQ ID No. 4, 8, 12, 16, 20, 24, 28, 44, 48, 52, or 56, and even more preferably at least 65%, 70%, 75%, 80%, 85% or even 90% identical to SEQ ID No. 4, 8, 12, 16, 20, 24, 28, 44, 48, 52, or 56, and able to specifically bind human CD39.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

In certain embodiments, the anti-CD39 antibody is a humanized antibody comprising a VH domain having human framework sequences associated with CDRs of a VH domain selected from SEQ ID No. 2, 6, 10, 14, 18, 22, 26, 42, 46, 50, or 54, and the CDRs of the corresponding VL domain selected from SEQ ID No. 4, 8, 12, 16, 20, 24, 28, 44, 48, 52, or 56. The CDRs are preferably identical, but may vary by 1, 2 or 3 amino acids across each CDR so long as the resulting antibody specifically binds human CD39.

Humanized antibodies and methods of making them are reviewed, e.g., in Almagro and Fransson, 2008, Front. Biosci. 13:1619-1633, and are further described, e.g., in Riechmann et al., 1988, Nature 332:323-329; Queen et al., 1989, Proc. Natl Acad. Sci. USA 86:10029-10033; U.S. Pat. Nos. 5,821,337; 7,527,791; 6,982,321; and U.S. Pat. No. 7,087,409; Kashmiri et al., 2005, Methods 36:25-34 (describing specificity determining region (SDR) grafting); Padlan, 1991, Mol. Immunol. 28:489-498 (describing “resurfacing”); Dall'Acqua et al., 2005, Methods 36:43-60 (describing “FR shuffling”); and Osbourn et al., 2005, Methods 36:61-68 and Klimka et al., 2000, Br. J. Cancer 83:252-260 (describing the “guided selection” approach to FR shuffling).

Human framework regions that may be used for humanization include but are not limited to: framework regions selected using the “best-fit” method (see, e.g., Sims et al. 1993, J. Immunol. 151:2296); framework regions derived from the consensus sequence of human antibodies of a particular subgroup of light or heavy chain variable regions (see, e.g., Carter et al. 1992, Proc. Natl. Acad. Sci. USA 89:4285; and Presta et al. 1993, J. Immunol. 151:2623); human mature (somatically mutated) framework regions or human germline framework regions (see, e.g., Almagro and Fransson, 2008, Front. Biosci. 13:1619-1633); and framework regions derived from screening FR libraries (see, e.g., Baca et al., 1997, J. Biol. Chem. 272:10678-10684 and Rosok et al., 1996, J Biol. Chem. 271:22611-22618).

In certain embodiments, an anti-CD39 antibody provided herein is a human antibody. Human antibodies can be produced using various techniques known in the art. Human antibodies are described generally in van Dijk and van de Winkel, 2001, Curr. Opin. Pharmacol. 5:368-74 and Lonberg, 2008, Curr. Opin. Immunol. 20:450-459.

For instance, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, 2005, Nat. Biotech. 23:1117-1125. (See also, e.g., U.S. Pat. Nos. 6,075,181 and 6,150,584 describing XENOMOUSE technology; U.S. Pat. No. 5,770,429 describing HuMAB technology; U.S. Pat. No. 7,041,870 describing K-M MOUSE technology; and U.S. Patent Application Publication No. US 2007/0061900 describing VELOCIMOUSE technology.) Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor, 1984, J. Immunol. 133:3001; Brodeur et al., Monoclonal Antibody Production Techniques and Applications, pp. 51-63 (Marcel Dekker, Inc., New York, 1987); and Boerner et al., 1991, J. Immunol. 147:86.) Human antibodies generated via human B-cell hybridoma technology are also described in Li et al., 2006, Proc. Natl. Acad. Sci USA 103:3557-3562. Additional methods include those described, for example, in U.S. Pat. No. 7,189,826 (describing production of monoclonal human IgM antibodies from hybridoma cell lines) and Ni, 2006, Xiandai Mianyixue, 26 (4): 265-268 (describing human-human hybridomas). Human hybridoma technology (Trioma technology) is also described in Vollmers and Brandlein, 2005, Histology and Histopathology 20 (3): 927-937 and Vollmers and Brandlein, 2005, Methods and Findings in Experimental and Clinical Pharmacology 27 (3): 185-91.

Human antibodies may also be generated by isolating Fv clone variable domain sequences selected from human-derived phage, yeast or bacterial display libraries. Such variable domain sequences may then be combined with a desired human constant domain. Techniques for selecting human antibodies from antibody libraries are described below.

To illustrate, anti-CD39 antibodies encompassed by the present invention may be isolated by screening combinatorial libraries for antibodies with the desired activity or activities. For example, a variety of methods are known in the art for generating phage or yeast display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in McCafferty et al., 1990, Nature 348:552-554; Clackson et al., 1991, Nature 352:624-628; Marks et al., 1992, J. Mol. Biol. 222:581-597; Marks and Bradbury, in Methods in Molecular Biology 248:161-175 (Lo, ed., Human Press, Totowa, N.J., 2003); Sidhu et al., 2004, J. Mol. Biol. 338 (2): 299-310; Lee et al., 2004, J. Mol. Biol. 340 (5): 1073-1093; Fellouse, 2004, Proc. Natl. Acad. Sci. USA 101 (34): 12467-12472; and Lee et al., 2004, J. Immunol. Methods 284 (1-2): 119-132.

As an example of phage display methods, repertoires of VH and VL genes are separately cloned by polymerase chain reaction (PCR) and recombined randomly in phage libraries, which can then be screened for antigen-binding phage as described in Winter et al., 1994, Ann. Rev. Immunol., 12:433-455. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., 1993, EMBO J. 12:725-734. Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, 1992, J. Mol. Biol. 227:381-388. Patent publications describing human antibody phage libraries include, for example: U.S. Pat. No. 5,750,373; and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

FcγRIII binding can also be increased by methods according to the state of the art, e.g., by modifying the amino acid sequence of the Fc part or the glycosylation of the Fc part of the antibody (see e.g., EP2235061). In certain embodiments, the subject antibodies are produced by cells in which, when glycosylated, less than 50% of the oligosaccharide chains on the antibody contain «-1,6-fucose. Typically, less than about 40%, less than about 30%, less than about 20%, less than about 10%, or less than 5% or less than 1% of the oligosaccharide chains contain «-1,6-fucose in a “hypo-fucosylated” antibody preparation. An “afucosylated” antibody lacks «-1,6-fucose in the carbohydrate attached to the CH2 domain of the IgG heavy chain. Mori et al., 2007, Cytotechnology 55 (2-3): 109-114 and Satoh et al., 2006, Expert Opin Biol Ther. 6:1161-1173 relate to a FUT8 (a-1,6-fucosyltransferase) gene knockout CHO line for the generation of afucosylated antibodies.

### IV. Expression Vectors

In certain embodiments, a recombinant expression vector is used to amplify and express DNA encoding the anti-CD39 antibody described herein. For example, a recombinant expression vector can be a replicable DNA construct which has synthetic or cDNA-derived DNA fragments encoding the polypeptide chains of the anti-CD39 antibody operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host cell. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of the polypeptide chains of the anti-CD39 antibody (or a protein to use as a target) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are well known by those skilled in the art.

Various mammalian cell culture systems are used to express recombinant polypeptides. Expression of recombinant proteins in mammalian cells can be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), and HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

In certain embodiments, the polynucleotide comprises a polynucleotide encoding an antibody light chain comprising a variable region at least 60% identical to SEQ ID No. 1, and even more preferably at least 65%, 70%, 75%, 80%, 85% or even 90% identical to SEQ ID No. 1, and able to specifically bind human CD39.

In certain embodiments, the polynucleotide comprises a polynucleotide encoding an antibody heavy chain comprising a variable region at least 60% identical to SEQ ID No. 2, and even more preferably at least 65%, 70%, 75%, 80%, 85% or even 90% identical to SEQ ID No. 2, and able to specifically bind human CD39.

V. Encoded anti-CD39 Antibodies for In Vivo Delivery

Therapeutic vectors for delivering the coding sequence for an anti-CD39 antibody to be expressed in the patient can be viral, non-viral, or physical. See, for example, Rosenberg et al., 1988, Science 242:1575-1578 and Wolff et al., 1989, Proc. Natl. Acad. Sci. USA 86:9011-9014. Discussion of methods and compositions for use in gene therapy include Eck et al., 1996, in Goodman & Gilman's The Pharmacological Basis of Therapeutics, Ninth Edition, Hardman et al., eds., McGraw-Hill, New York, Chapter 5, pp. 77-101; Wilson et al., 1997, Clin. Exp. Immunol. 107 (Suppl. 1): 31-32; Wivel et al., 1998, Hematology/Oncology Clinics of North America, Gene Therapy, S. L. Eck, ed., 12 (3): 483-501; Romano et al., 2000, Stem Cells 18:19-39; and the references cited therein. U.S. Pat. No. 6,080,728 also provides a discussion of a wide variety of gene delivery methods and compositions. The routes of delivery include, for example, systemic administration and administration in situ. Well-known viral delivery techniques include the use of adenovirus, retrovirus, lentivirus, foamy virus, herpes simplex virus, vaccinia virus and adeno-associated virus vectors.

a. Viral Vectors

Preferred viral vectors are based on non-cytopathic eukaryotic viruses in which non-essential genes have been replaced with the nucleic acid construct carrying the nucleic acid sequences encoding the epitopes and targeting sequences of interest. Preferred viruses for certain embodiments encompassed by the present invention are the adenoviruses and adeno-associated (AAV) viruses, which are double-stranded DNA viruses that have already been approved for human use in gene therapy. In addition, preferred vectors for tolerizing do not include immune-stimulating sequences.

### Adenovirus Vectors

One illustrative method for in vivo delivery of one or more nucleic acid sequences involves the use of an adenovirus expression vector. “Adenovirus expression vector” is meant to include those constructs containing adenovirus sequences sufficient to (a) support packaging of the construct and (b) to express a polynucleotide that has been cloned therein in a sense or antisense orientation. Of course, in the context of an antisense construct, expression does not require that the gene product be synthesized. In a specific embodiment, the delivery vector pertains to commercially available ORF of cytochrome b5 reductase 3 (CYB5R3), transcript variant 1 in adenoviral vector pAd, with C terminal Flag and His tag (Vigene Biosciences Product code AH889428). WIPO Patent Application WO/2015/050364 also teaches vectors with expression constructs including a Cyb5r3 gene.

Adenoviral vectors are highly immunogenic and therefore are less preferred for administration to induce tolerance by presenting antigens, or in the case of autoimmune diseases. These vectors can be used, however to induce immunity, for example in treatment of infectious diseases and the like, include, for example, influenza, HBV, HCV and HIV.

### Adeno-Associated Virus Vectors (AAV)

AAV is a good choice of delivery vehicles due to its safety, i.e., genetically engineered (recombinant) does not integrate into the host genome. Likewise, AAV is not pathogenic and not associated with any disease. The removal of viral coding sequences minimizes immune reactions to viral gene expression, and therefore, rAAV does not evoke an inflammatory response. According to a specific embodiment, an AAV vector containing an epitope sequence containing nucleic acid construct described herein is useful for transducing APCs.

Typically, viral vectors containing an epitope containing nucleic acid construct are assembled from polynucleotides encoding the desired epitopes, suitable regulatory elements and elements necessary for epitope expression which mediate cell transduction. In one embodiment, adeno-associated viral (AAV) vectors are employed. In a more specific embodiment, the AAV vector is an AAV1, AAV6, or AAV8.

The AAV expression vector which harbors the DNA molecule of interest bounded by AAV ITRs, can be constructed by directly inserting the selected sequence(s) into an AAV genome which has had the major AAV open reading frames (“ORFs”) excised therefrom. Examples of constitutive promoters which may be included in the AAV of this invention include, without limitation, the exemplified CMV immediate early enhancer/chicken β-actin (CBA) promoter.

For eukaryotic cells, expression control sequences typically include a promoter, an enhancer, such as one derived from an immunoglobulin gene, SV40, cytomegalovirus, etc., and a polyadenylation sequence which may include splice donor and acceptor sites. The polyadenylation sequence generally is inserted following the transgene sequences and before the 3′ ITR sequence. In one embodiment, the bovine growth hormone polyA may be used.

Selection of these and other common vector and regulatory elements are conventional, and many such sequences are available. See, e.g., Sambrook et al., and references cited therein at, for example, pages 3.18-3.26 and 16.17-16.27 and Ausubel et al., 1989, Current Protocols in Molecular Biology, John Wiley & Sons, New York). Of course, not all vectors and expression control sequences will function equally well to express all of the transgenes of this invention. However, one of skill in the art may make a selection among these expression control sequences without departing from the scope of this invention. Suitable promoter/enhancer sequences may be selected by one of skill in the art using the guidance provided by this application. Such selection is a routine matter and is not a limitation of the molecule or construct.

### Retrovirus Vectors

In certain embodiments, the viral vector may be a retroviral vector. “Retroviruses” are viruses having an RNA genome. In particular embodiments, a retroviral vector contains all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail regarding retroviral vectors can be found in Boesen et al., 1994, Biotherapy 6:291-302; Clowes et al., 1994, J. Clin. Invest. 93:644-651; Kiem et al., 1994, Blood 83:1467-1473; Salmons and Gunzberg, 1993, Human Gene Therapy 4:129-141; Miller et al., 1993, Meth. Enzymol. 217:581-599; and Grossman and Wilson, 1993, Curr. Opin. in Genetics and Devel. 3:110-114.

“Gammaretroviruses” refers to a genus of the retroviridae family. Exemplary gammaretroviruses include mouse stem cell virus, murine leukemia virus, feline leukemia virus, feline sarcoma virus, and avian reticuloendotheliosis viruses.

Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., 1992, J. Virol. 66:2731-2739; Johann et al., 1992, J. Virol. 66:1635-1640; Sommerfelt et al., 1990, Virol. 176:58-59; Wilson et al., 1989, J. Virol. 63:2374-2378; Miller et al., 1991, J. Virol. 65:2220-2224; and PCT/US94/05700).

Lentiviral vectors refer to a genus of retroviruses that are capable of infecting dividing and non-dividing cells and typically produce high viral titers. Several examples of lentiviruses include HIV (human immunodeficiency virus: including HIV type 1 and HIV type 2); equine infectious anemia virus; feline immunodeficiency virus (FIV); bovine immune deficiency virus (BIV); and simian immunodeficiency virus (SIV).

In particular embodiments, other retroviral vectors can be used. These include, e.g., vectors based on human foamy virus (HFV) or other viruses in the Spumavirus genera. Foamy viruses (FVes) are the largest retroviruses known today and are widespread among different mammals, including all non-human primate species, however are absent in humans. This complete apathogenicity qualifies FV vectors as ideal gene transfer vehicles for genetic therapies in humans and clearly distinguishes FV vectors as gene delivery system from HIV-derived and also gammaretrovirus-derived vectors.

Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are known to those of skill in the art.

The retroviral genome contains three genes, gag, pol, and env that code for capsid proteins, polymerase enzyme, and envelope components, respectively. A sequence found upstream from the gag gene contains a signal for packaging of the genome into virions. Retroviral vectors are gene transfer plasmids wherein the heterologous nucleic acid resides between two retroviral LTRs. Retroviral vectors typically contain appropriate packaging signals that enable the retroviral vector, or RNA transcribed using the retroviral vector as a template, to be packaged into a viral virion in an appropriate packaging cell line (see, e.g., U.S. Pat. No. 4,650,764). These two long terminal repeat (LTR) sequences are present at the 5′ and 3′ ends of the viral genome. These contain strong promoter and enhancer sequences and are also required for integration in the host cell genome (Coffin, 1990). In order to construct a retroviral vector, a nucleic acid encoding one or more oligonucleotide or polynucleotide sequences of interest is inserted into the viral genome in the place of certain viral sequences to produce a virus that is replication-defective. Also included are episomal or non-integrating forms of retroviral vectors based on lentiviruses (e.g., a type of retrovirus).

Lentiviral vectors are useful when stable expression is needed, but lentiviral vectors can be immunogenic, and possibly have other undesirable effects. Therefore, although lentiviral vectors are convenient for research, care should be taken when using them for human administration, particularly where it is desired to induce tolerance rather than immunity. Lentiviruses are suitable for engineering T cells or dendritic cells or other antigen presenting cells ex vivo for cancer therapy, although mRNA electroporation is more safe. However, two recent advances have made the use of lentiviruses safer and more clinically translatable. First, the coexpression of a suicide gene along with the antigens whose products become functional when a drug is administered. A typical example is Herpes simplex virus thymidine kinase (HSV-Tk). Cells that express these genes can metabolize the drug ganciclovir into a cytotoxic product that induces cell death. Thus, in case some transduced cells become malignant, they can be eradicated. About a dozen such systems exist (Duarte et al., 2012, Cancer Letters 324:160-170). Second, there are now non-integrating lentiviral vectors being developed that are therefore non-oncogenic (Nightingale et al., 2006, Mol. Ther. 13:1121-1132). These methods can be used with the invention according to the judgement of the person of skill in the art.

Suitable retroviral vectors for use herein are described, for example, in U.S. Pat. Nos. 5,399,346 and 5,252,479; and in WIPO publications WO 92/07573, WO 90/06997, WO 89/05345, WO 92/05266, and WO 92/14829, which provide a description of methods for efficiently introducing nucleic acids into human cells using such retroviral vectors. Other retroviral vectors include, for example, mouse mammary tumor virus vectors (e.g., Shackleford et al., 1998, Proc. Natl. Acad. Sci. USA 85:9655-9659), lentiviruses, and the like. An exemplary viral vector is plentilox-IRES-GFP.

Additional retroviral viral delivery systems that can be readily adapted for delivery of a transgene encoding an Anti-CD39 antibody Agent include, merely to illustrate Published PCT Applications WO/2010/045002, WO/2010/148203, WO/2011/126864, WO/2012/058673, WO/2014/066700, WO/2015/021077, WO/2015/148683, WO/2017/040815—the specifications and figures of each of which are incorporated by reference herein.

In certain embodiments, the retrovirus is a recombinant replication competent retrovirus comprising: a nucleic acid sequence encoding a retroviral GAG protein; a nucleic acid sequence encoding a retroviral POL protein; a nucleic acid sequence encoding a retroviral envelope; an oncoretroviral polynucleotide sequence comprising Long-Terminal Repeat (LTR) sequences at the 5′ and 3′ end of the oncoretroviral polynucleotide sequence; a cassette comprising an internal ribosome entry site (IRES) operably linked to a coding sequence for an Anti-CD39 antibody Agent, wherein the cassette is positioned 5′ to the U3 region of the 3′ LTR and 3′ to the sequence encoding the retroviral envelope; and cis-acting sequences for reverse transcription, packaging and integration in a target cell.

In certain embodiments, the retrovirus is a recombinant replication competent retrovirus comprising: a retroviral GAG protein; a retroviral POL protein; a retroviral envelope; a retroviral polynucleotide comprising Long-Terminal Repeat (LTR) sequences at the 3′ end of the retroviral polynucleotide sequence, a promoter sequence at the 5′ end of the retroviral polynucleotide, the promoter being suitable for expression in a mammalian cell, a gag nucleic acid domain, a pol nucleic acid domain and an env nucleic acid domain; a cassette comprising an Anti-CD39 antibody Agent coding sequence operably linked to a heterologous polynucleotide, wherein the cassette is positioned 5′ to the 3′ LTR and is operably linked and 3′ to the env nucleic acid domain encoding the retroviral envelope; and cis-acting sequences necessary for reverse transcription, packaging and integration in a target cell.

In certain preferred embodiments of the recombinant replication competent retrovirus, the envelope is chosen from one of amphotropic, polytropic, xenotropic, 10A1, GALV, Baboon endogenous virus, RD114, rhabdovirus, alphavirus, measles or influenza virus envelopes.

In certain preferred embodiments of the recombinant replication competent retrovirus, the retroviral polynucleotide sequence is engineered from a virus selected from the group consisting of murine leukemia virus (MLV), Moloney murine leukemia virus (MoMLV), Feline leukemia virus (FeLV), Baboon endogenous retrovirus (BEV), porcine endogenous virus (PERV), the cat derived retrovirus RD114, squirrel monkey retrovirus, Xenotropic murine leukemia virus-related virus (XMRV), avian reticuloendotheliosis virus (REV), or Gibbon ape leukemia virus (GALV).

In certain preferred embodiments of the recombinant replication competent retrovirus, retrovirus is a gammaretrovirus.

In certain preferred embodiments of the recombinant replication competent retrovirus, there is a second cassette comprising a coding sequence for a second therapeutic protein, such as another checkpoint inhibitor polypeptide, a co-stimulatory polypeptide and/or a immunostimulatory cytokine (merely as examples), e.g., downstream of the cassette. In certain instances, the second cassette can include an internal ribosome entry site (IRES) or a minipromoter or a polIII promoter operably linked to the coding sequence for the second therapeutic protein.

In certain preferred embodiments of the recombinant replication competent retrovirus, it is a nonlytic, amphotropic retroviral replicating vector which, preferably, selectively infects and replicates in the cells of the inflammatory tissue microenvironment.

### Other Viral Vectors as Expression Constructs

Other viral vectors may be employed as expression constructs in the present invention for the delivery of oligonucleotide or polynucleotide sequences to a host cell. Vectors derived from viruses such as vaccinia virus, polioviruses and herpes viruses may be employed. They offer several attractive features for various mammalian cells. Also included are hepatitis B viruses.

b. Non-Viral Vectors

### Plasmid Vectors

Other vectors include plasmid vectors. Plasmid vectors have been extensively described in the art and are well known to those of skill in the art. See e.g. Sambrook et al., 1989, cited above. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide epitope encoded by nucleic acid within the plasmid. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Thus, in one aspect, a plasmid is provided for expression of the epitope containing nucleic acid construct which includes an expression cassette; also referred to as a transcription unit. When a plasmid is placed in an environment suitable for epitope expression, the transcriptional unit will express the polynucleotide including a sequence encoding the epitopes, ETS and MHCII activator sequence, or sequence encoding the epitopes and secretion signal sequence, and anything else encoded in the construct. The transcription unit includes a transcriptional control sequence, which is transcriptionally linked with a cellular immune response element coding sequence. Transcriptional control sequence may include promoter/enhancer sequences such as cytomegalovirus (CMV) promoter/enhancer sequences. However, those skilled in the art will recognize that a variety of other promoter sequences suitable for expression in eukaryotic cells are known and can similarly be used in the constructs disclosed herein. The level of expression of the nucleic acid product will depend on the associated promoter and the presence and activation of an associated enhancer element.

In certain embodiments, a sequence encoding the desired epitopes and targeting sequence can be cloned into an expression plasmid which contains the regulatory elements for transcription, translation, RNA stability and replication (i.e., including a transcriptional control sequence). Such expression plasmids are well known in the art and one of ordinary skill would be capable of designing an appropriate expression construct with a polynucleotide including a sequence encoding a cellular immune response element or fragment thereof in such a manner that the cellular immune response element is expressible. There are numerous examples of suitable expression plasmids into which a polynucleotide including a sequence could be cloned such as pCI-neo, pUMVC or pcDNA3.

Large quantities of a bacterial host harboring a plasmid for expression of cellular immune response element or fragment thereof may be fermented and the plasmid can be purified for subsequent use. Current human clinical trials using plasmids utilize this approach (Recombinant DNA Advisory Committee Data Management Report, 1994, Human Gene Therapy 6:535-548). Current DNA isolation methods known in the art include removal of lipopolysaccharides (endotoxins) that are contaminants from the bacteria used to propagate the plasmids. This step is most preferably taken for use of tolerogenic DNA vaccines as endotoxins act as strong adjuvants and can produce undesired immune stimulation.

The purpose of the plasmid is the efficient delivery of nucleic acid sequences to and expression of therapeutic epitopes in a cell or tissue. In particular, the purpose of the plasmid may be to achieve high copy number, avoid potential causes of plasmid instability and provide a means for plasmid selection. As for expression, the nucleic acid cassette contains the necessary elements for expression of the nucleic acid within the cassette. Expression includes the efficient transcription of an inserted gene, nucleic acid sequence, or nucleic acid cassette with the plasmid. Expression products may be proteins, polypeptides or RNA. The nucleic acid sequence can be contained in a nucleic acid cassette. Expression of the nucleic acid can be continuous or regulated.

### Minicircle

Embodiments of nucleic acid constructs described herein may be processed in the form of minicircle DNA. Minicircle DNA pertains to small (2-4 kb) circular plasmid derivatives that have been freed from all prokaryotic vector parts. Since minicircle DNA vectors contain no bacterial DNA sequences, they are less likely to be perceived as foreign and destroyed. (Typical transgene delivery methods involve plasmids, which contain foreign DNA.) As a result, these vectors can be expressed for longer periods of time (in order of weeks or months) compared to conventional plasmids (days to weeks). The smaller size of minicircles also extends their cloning capacity and facilitates their delivery into cells. Kits for producing minicircle DNA are known in the art and are commercially available (System Biosciences, Inc., Palo Alto, Calif.). Information on minicircle DNA is provided in Dietz et al., 2013, Vector Engineering and Delivery Molecular Therapy 21 (8): 1526-1535 and Hou et al., 2015, Molecular Therapy-Methods & Clinical Development, Article number: 14062 doi: 10.1038/mtm.2014.62. More information on Minicircles is provided in Chen et al., 2003 September, Mol. Ther. 8 (3): 495-500 and Minicircle DNA vectors achieve sustained expression reflected by active chromatin and transcriptional level (Gracey Maniar et al., 2013 January, Mol. Ther. 21 (1): 131-8).

As an initial step in the process of ultimately obtaining expression of a product encoded by a nucleic acid, is to effect the uptake of the nucleic acid by cells. Uptake of nucleic acid by cells is dependent on a number of factors, one of which is the length of time during which a nucleic acid is in proximity to a cellular surface. For instance, after intramuscular (i.m.) administration of plasmid DNA in buffer, a marked reduction in gene expression was observed if the muscle is massaged, presumably due to DNA leakage out of the muscle either directly or via lymphatic vessels (Human Gene Therapy 4:151-159 (1993)). Accordingly, it may be desirable to formulate nucleic acids with compounds which would retard the rate at which nucleic acids diffuse or are carried away from a site at which cellular uptake of the nucleic acid is desired. Further, these compounds could be suitable for administration to an organism by means such as injection while maintaining or regaining the physical characteristics necessary to increase cellular uptake of nucleic acids.

In order to effect expression of oligonucleotide or polynucleotide sequences, the expression construct must be delivered into a cell. In certain embodiments encompassed by the present invention, an expression construct comprising one or more oligonucleotide or polynucleotide sequences may simply consist of naked recombinant DNA or plasmids.

To prime immunity, DNA vaccine vectors of any type preferably are engineered to be CpG-rich (to stimulate TLR9 on immune cells) or conversely are engineered to remove CpG, and when possible, replace CpG motifs with GpG motifs (Ho et al., 2003, J. Immunol. 71 (9): 4920-6; Ho et al., 2005, J. Immunol. 175 (9): 6226-34). DNA vaccines can be engineered to contain the antigen(s)/epitope(s), and also can contain additional genes for co-expression with the antigens to act as adjuvants or immunomodulators (multiple promoter vectors. These DNA vaccines have been found to be safe clinically, for example in TID patients (Roep et al., 2013, Sci. Transl. Med. 5 (191): 191ra82).

### Mechanical Delivery Systems

Additional non-viral delivery methods include but are not limited to mechanical delivery systems that can be used in vitro such as the approach described in Woffendin et al., 1994, Proc. Natl. Acad. Sci. USA 91 (24): 11581; deposition of photopolymerized hydrogel materials or use of ionizing radiation (see, e.g., U.S. Pat. No. 5,206,152 and WO 92/11033); the use of a hand-held gene transfer particle gun (see, e.g., U.S. Pat. No. 5,149,655); and the use of ionizing radiation for activating transferred gene (see, e.g., U.S. Pat. No. 5,206,152 and WO 92/11033). Delivery devices can also be biocompatible, and may also be biodegradable. The formulation preferably provides a relatively constant level of active component release. On the other hand, a more rapid rate of release immediately upon administration may be desired. The formulation of such compositions is well within the level of ordinary skill in the art using known techniques.

Physical methods to enhance delivery include electroporation (where short pulses of high voltage carries the nucleic acid across the membrane), a gene gun (where DNA is loaded onto gold particles and forced to achieve penetration of the DNA into the cells), sonoporation, magnetofection, hydrodynamic delivery and the like, all of which are known to those of skill in the art. DNA also can be encapsulated in liposomes, preferably cationic liposomes, or polymersomes (synthetic liposomes) which can interact with the cell membrane and fuse or undergo endocytosis to effect DNA transfer into the cell. The DNA also can be formed into complexes with polymers (polyplexes) or with dendrimers which can directly release their load into the cytoplasm of a cell.

Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active agent contained within a sustained release formulation depends upon the site of implantation, the rate and expected duration of release and the nature of the condition to be treated or prevented.

Biodegradable microspheres (e.g., polylactate polyglycolate) may be employed as carriers for compositions. Suitable biodegradable microspheres are disclosed, for example, in U.S. Pat. Nos. 4,897,268; 5,075,109; 5,928,647; 5,811,128; 5,820,883; 5,853,763; 5,814,344; 5,407,609; and 5,942,252. Modified hepatitis B core protein carrier systems such as described in WO/99 40934, and references cited therein, will also be useful for many applications. Another illustrative carrier/delivery system employs a carrier comprising particulate-protein complexes, such as those described in U.S. Pat. No. 5,928,647.

Biodegradable polymeric nanoparticles facilitate nonviral nucleic acid transfer to cells. Small (approximately 200 nm), positively charged (approximately 10 mV) particles are formed by the self-assembly of cationic, hydrolytically degradable poly(beta-amino esters) and plasmid DNA.

Polynucleotides may also be administered to cells by direct microinjection, temporary cell permeabilizations (e.g., co-administration of repressor and/or activator with a cell permeabilizing agent), fusion to membrane translocating peptides, and the like.

In certain particular embodiments of the present disclosure, the gene construct is introduced into target cells via electroporation. Electroporation involves the exposure of cells (or tissues) and DNA (or a DNA complex) to a high-voltage electric discharge. In vivo electroporation is a gene delivery technique that has been used successfully for efficient delivery of plasmid DNA to many different tissues. Systemic and local expression of a gene or cDNA encoded by a plasmid can be obtained with administration of in vivo electroporation. Use of in vivo electroporation enhances plasmid DNA uptake in the target inflammatory tissue, resulting in expression within the inflamed tissue, and delivers plasmids to muscle tissue, resulting in systemic expression of the anti-CD39 antibody (see, e.g., U.S. Pat. No. 8,026,223). Exemplary techniques, vectors and devices for electroporating anti-CD39 antibody transgenes into cells in vivo include PCT Publications WO/2017/106795, WO/2016/161201, WO/2016/154473, WO/2016/112359, and WO/2014/066655.

U.S. Pat. No. 7,245,963 describes modular electrode systems and their use for facilitating the introduction of a biomolecule into cells of a selected tissue in a body or plant. The modular electrode systems comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the ceil between the plurality of electrodes. The entire content of U.S. Pat. No. 7,245,963 is hereby incorporated by reference.

U.S. Patent Pub. 2005/0052630 describes an electroporation device which may be used to effectively facilitate the introduction of a biomolecule into ceils of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk (see, e.g., U.S. Patent Pub. 2005/0052630) is hereby incorporated by reference.

The electrode arrays and methods described in U.S. Pat. No. 7,245,963 and U.S. Patent Pub. 2005/0052630 are adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes.

Typically, the electric fields needed for in vivo cell electroporation are generally similar in magnitude to the fields required for cells in vitro. In one embodiment, the magnitude of the electric field range from approximately, 10 V/cm to about 1500 V/cm, preferably from about 300 V/cm to 1500 V/cm and preferably from about 1000 V/cm to 1500 V/cm. Alternatively, lower field strengths (from about 10 V/cm to 100 V/cm, and more preferably from about 25 V/cm to 75 V/cm) the pulse length is long. For example, when the nominal electric field is about 25-75 V/cm, if is preferred that the pulse length is about 10 msec.

The pulse length can be about 10 s to about 100 ms. There can be any desired number of pulses, typically one to 100 pulses per second. The delay between pulses sets can be any desired time, such as one second. The waveform, electric field strength and pulse duration may also depend upon the type of cells and the type of molecules that are to enter the cells via electroporation.

Also encompassed are electroporation devices incorporating electrochemical impedance spectroscopy (“EIS”). Such devices provide real-time information on in vivo, in particular, inflammatory tissue electroporation efficiency, allowing for the the optimization of conditions. Examples of electroporation devices incorporating EIS can be found, e.g., in WO2016/161201, which is hereby incorporated by reference.

Uptake of the non-viral delivery vectors encompassed by the present invention may also be enhanced by plasma electroporation also termed avalanche transfection. Briefly, microsecond discharges create cavitation microbubbles at electrode surface. The mechanical force created by the collapsing microbubbles combined with the magnetic field serve to increase transport efficiency across the cell membrane as compared with the diffusion mediated transport associated with conventional electroporation. The technique of plasma electroporation is described in U.S. Pat. Nos. 7,923,251 and 8,283,171. This technique may also be employed in vivo for the transformation of cells (Chaiberg et al., 2006, Investigative Ophthalmology & Visual Science 47:4083-4090; Chaiberg et al., United States Patent No 8, 101 169 Issued Jan. 24, 2012).

Other alternative electroporation technologies are also contemplated. In vivo plasmid delivery can also be performed using cold plasma. Plasma is one of the four fundamental states of matter, the others being solid, liquid, and gas. Plasma is an electrically neutral medium of unbound positive and negative particles (i.e. the overall charge of a plasma is roughly zero). A plasma can be created by heating a gas or subjecting it to a strong electromagnetic field, applied with a laser or microwave generator. This decreases or increases the number of electrons, creating positive or negative charged particles called ions (Luo et al., 1998, Phys. Plasma 5:2868-2870) and is accompanied by the dissociation of molecular bonds, if present.

Cold plasmas (i.e., non-thermal plasmas) are produced by the delivery of pulsed high voltage signals to a suitable electrode. Cold plasma devices may take the form of a gas jet device or a dielectric barrier discharge (DBD) device. Cold temperature plasmas have attracted a great deal of enthusiasm and interest by virtue of their provision of plasmas at relatively low gas temperatures. The provision of plasmas at such a temperature is of interest to a variety of applications, including wound healing, anti-bacterial processes, various other medical therapies and sterilization. As noted earlier, cold plasmas (i.e., non-thermal plasmas) are produced by the delivery of pulsed high voltage signals to a suitable electrode. Cold plasma devices may take the form of a gas jet device, a dielectric barrier discharge (DBD) device or multi-frequency harmonic-rich power supply.

Dielectric barrier discharge device relies on a different process to generate the cold plasma. A dielectric barrier discharge (DBD) device contains at least one conductive electrode covered by a dielectric layer. The electrical return path is formed by the ground that can be provided by the target substrate undergoing the cold plasma treatment or by providing an in-built ground for the electrode. Energy for the dielectric barrier discharge device can be provided by a high voltage power supply, such as that mentioned above. More generally, energy is input to the dielectric barrier discharge device in the form of pulsed DC electrical voltage to form the plasma discharge. By virtue of the dielectric layer, the discharge is separated from the conductive electrode and electrode etching and gas heating is reduced. The pulsed DC electrical voltage can be varied in amplitude and frequency to achieve varying regimes of operation. Any device incorporating such a principle of cold plasma generation (e.g., a DBD electrode device) falls within the scope of various embodiments encompassed by the present invention.

In certain illustrative embodiments, the transgene construct encoding the anti-CD39 antibody agent encompassed by the present invention is delivered using an electroporation device comprising: an applicator; a plurality of electrodes extending from the applicator, the electrodes being associated with a cover area; a power supply in electrical communication with the electrodes, the power supply configured to generate one or more electroporating signals to cells within the cover area; and a guide member coupled to the electrodes, wherein the guide member is configured to adjust the cover area of the electrodes. At least a portion of the electrodes can be positioned within the applicator in a conical arrangement. The one or more electroporating signals may be each associated with an electric field. The device may further comprise a potentiometer coupled to the power supply and electrodes. The potentiometer may be configured to maintain the electric field substantially within a predetermined range.

The one or more electroporating signals may be each associated with an electric field. The device may further comprise a potentiometer coupled to the power supply and the electrodes. The potentiometer may be configured to maintain the electric field within a predetermined range so as to substantially prevent permanent damage in the cells within the cover area and/or substantially minimize pain. For instance, potentiometer may be configured to maintain the electric field to about 1300 V/cm.

The power supply may provide a first electrical signal to a first electrode and a second electrical signal to a second electrode. The first and second electrical signals may combine to produce a wave having a beat frequency. The first and second electrical signals may each have at least one of a unipolar waveform and a bipolar waveform. The first electrical signal may have a first frequency and a first amplitude. The second electrical signal may have a second frequency and a second amplitude. The first frequency may be different from or the same as the second frequency. The first amplitude may be different from or the same as the second amplitude.

In certain embodiments, the present invention provides a method for treating a subject having an inflammatory condition or suffering from eosinophilia, the method comprising: injecting the inflamed tissue (or tissue proximate to it) with an effective dose of plasmid coding for an anti-CD39 antibody; and administering electroporation therapy to the target tissue. In certain embodiments, the electroporation therapy further comprises the administration of at least one voltage pulse of about 200 V/cm to about 1500 V/cm over a pulse width of about 100 microseconds to about 20 milliseconds.

In certain embodiments, the plasmid (or a second electroporated plasmid) further encodes at least one or more additional immunosuppressive biologic(s), such as adalimumab, certolizumab, etanercept, golimumab, infliximab, risankizumab, and ustekinumab.

Lipids and Polycationic Molecules for Delivering Anti-CD39 antibody Encoding Nucleic Constructs

Lipid-mediated nucleic acid delivery and expression of foreign nucleic acids, including mRNA, in vitro and in vivo has been very successful. Lipid based non-viral formulations provide an alternative to adenoviral gene therapies. Current in vivo lipid delivery methods use subcutaneous, intradermal, pulmonary, gastrointestinal, submucosal, intrasynovial, intrathecal or intracranial injection. Advances in lipid formulations have improved the efficiency of gene transfer in vivo (see PCT Application WO 98/07408). For instance, a lipid formulation composed of an equimolar ratio of 1,2-bis(oleoyloxy)-3-(trimethyl ammonio) propane (DOTAP) and cholesterol can significantly enhances systemic in vivo gene transfer. The DOTAP: cholesterol lipid formulation forms unique structure termed a “sandwich liposome”. This formulation is reported to “sandwich” DNA between an invaginated bi-layer or “vase” structure. Beneficial characteristics of these lipid structures include a positive p, colloidal stabilization by cholesterol, two dimensional nucleic acid packing and increased serum stability.

Cationic liposome technology is based on the ability of amphipathic lipids, possessing a positively charged head group and a hydrophobic lipid tail, to bind to negatively charged DNA or RNA and form particles that generally enter cells by endocytosis. Some cationic liposomes also contain a neutral co-lipid, thought to enhance liposome uptake by mammalian cells. Similarly, other polycations, such as poly−1-lysine and polyethylene-imine, complex with nucleic acids via charge interaction and aid in the condensation of DNA or RNA into nanoparticles, which are then substrates for endosome-mediated uptake. Several of these cationic-nucleic acid complex technologies have been developed as potential clinical products, including complexes with plasmid DNA (pDNA), oligodeoxynucleotides, and various forms of synthetic RNA.

The nucleic acid constructs disclosed herein may be associated with polycationic molecules that serve to enhance uptake into cells. Complexing the nucleic acid construct with polycationic molecules also helps in packaging the construct such their size is reduced, which is believed to assist with cellular uptake. Once in the endosome, the complex dissociates due to the lower pH, and the polycationic molecules can disrupt the endosome's membrane to facilitate DNA escape into the cytoplasm before it can be degraded. Preliminary data shows that the nucleic acid construct embodiments had enhanced uptake into SCs over DCs when complexed with the polycationic molecules polylysine or polyethyleneimine.

One example of polycationic molecules useful for complexing with nucleic acid constructs includes cell penetrating peptides (CPP), examples include polylysine (described above), polyarginine and Tat peptides. Cell penetrating peptides (CPP) are small peptides which can bind to DNA and, once released, penetrate cell membranes to facilitate escape of the DNA from the endosome to the cytoplasm. Another example of a CPP pertains to a 27-residue chimeric peptide, termed MPG, was shown some time ago to bind ss- and ds-oligonucleotides in a stable manner, resulting in a non-covalent complex that protected the nucleic acids from degradation by DNase and effectively delivered oligonucleotides to cells in vitro (Mahapatro et al., 2011, J Nanobiotechnol 9:55). The complex formed small particles of approximately 150 nm to 1 μm when different peptide: DNA ratios were examined, and the 10:1 and 5:1 ratios (150 nm and 1 μm respectively). Another CPP pertains to a modified tetrapeptide (tetralysine containing guanidinocarbonylpyrrole (GCP) groups (TL-GCP)), which was reported to bind with high affinity to a 6.2 kb plasmid DNA resulting in a positive charged aggregate of 700-900 nm (Li et al., 2015, Agnew Chem Int Ed Enl, 54 (10): 2941-4). RNA can also be complexed by such polycationic molecules for in vivo delivery.

Other examples of polycationic molecules that may be complexed with the nucleic acid constructs described herein include polycationic polymers commercially available as JETPRIME® and In Vivo JET (Polypus-transfection, S.A., Illkirch, France).

### VI. Methods of Use and Pharmaceutical Compositions

The anti-CD39 antibodies encompassed by the present invention are useful in a variety of applications including, but not limited to, therapeutic treatment methods, such as therapy for inflammatory diseases and disorders as well as conditions marked by eosinophilia generally. In certain embodiments, an anti-CD39 antibody described herein is useful for inactivating or otherwise decreasing eosinophil mediated aspects of inflammatory and immune responses as well as systemic or localized eosinophilia or drug-induced eosinophilia.

In general, “eosinophils” encompass a class of hematopoietic cells. In certain embodiments, eosinophils encompassed by the present invention (i) are CD39+eosinophils; (ii) co-express one or more of the cell surface markers selected from the group consisting of CD45, CD11b, Siglec-8, the a subunit of IL-5 receptor (IL-5Rα or CD125), the a subunit of IL-3 receptor (IL-3Rα or CD123), IL-4R, IL-9R, IL-13R, IL-14R, ST2 (IL-33R), PIRA, PIRB, L-selectin, EMR1, CCR3 (CD193), and CRTh2 (CD294); (iii) are CD45⁺CD11b⁺eosinophil cells; and/or (iv) are CD45⁺CD11b⁺Siglec-8+eosinophil cells.

In certain embodiments, the subject CD39-targeted eosinophil-depleting agents can be used to treat gastrointestinal inflammatory disorders. The present disclosure provides methods of administering pharmaceutical compositions comprising a CD39-targeted eosinophil-depleting agents to treat, prevent, ameliorate, or delay the symptoms and/or inflammation associated with a gastrointestinal inflammatory disorder. In some embodiments, the gastrointestinal inflammatory disorder is in the esophagus. In some embodiments, the gastrointestinal inflammatory disorder is eosinophilic esophagitis. In some embodiments, the patient exhibits substantial improvement in esophageal function and morphology, including lessening of esophageal furrows, lessening of esophageal focal narrowing, increased esophageal diameter, increased esophageal compliance, increased esophageal body distensibility, increased ease swallowing, reduced edema, improved vascularity, reduction of rings, decrease or absence of exudate, and/or absence of stricture.

To illustrate, the CD39-targeted eosinophil-depleting agents can be used as part of a treatment for eosinophilic esophagitis (EoE), an allergic/immune condition where the subject suffers from inflammation and/or swelling of the esophagus, affect a patient's ability to swallow food and can consequently cause malnutrition and failure to thrive. Typically, eosinophils are not found in the esophagus, but in EoE these cells accumulate and produce swelling that reduces the interior diameter of the esophagus making swallowing and eating very difficult. Often patients experience episodes of food impaction where food becomes lodged in the patient's esophagus, which can require emergency treatment. Because of the difficulty swallowing, and fear of food impaction, many patients with EoE limit themselves to eating soft foods such as yogurt, soups, and smoothies. In severe cases of EoE patients receive parenteral nutrition (e.g., intravenous feeding), which can provide required sustenance but limits the patient's activities and can lead to increased infection at the site of the catheter.

In certain embodiments, the present invention provides methods of administering pharmaceutical compositions comprising a CD39-targeted eosinophil-depleting agent to treat the symptoms and/or inflammation associated with an eosinophilic esophagitis. Pharmaceutical compositions can be delivered, merely to illustrate, topically to the esophagus, including by submucosal injection, as well as systemically. In the case of nucleic acid encoded CD39-targeted eosinophil-depleting agents, the nucleic acid can be transfected into esophageal tissue including the eosinophil infiltrated portions, or tissue adjacent thereto.

In certain embodiments, the CD39-targeted eosinophil-depleting agent can be part of a treatment including one or more glucocorticosteroids, leukotriene antagonists, mast cell stabilizers, immunomodulators, Proton Pump Inhibitors (PPIs).

In one embodiment the antibody or antigen-binding fragment thereof or formulation, according to the present disclosure is employed for the treatment of a chronic inflammatory condition wherein the condition associated with inappropriate inflammation. Such conditions include, but are not limited to, rheumatoid arthritis (RA), autoimmune conditions, inflammatory bowel diseases, non-healing wounds, multiple sclerosis, cancer, atherosclerosis, vasculitis, Sjogren's disease, diabetes, lupus erythematosus (including systemic lupus erythematosus), asthma, fibrotic diseases (including liver cirrhosis), pulmonary fibrosis, and UV damage and psoriasis.

In certain embodiments, the CD39-targeted eosinophil-depleting agent can be part of treatment for a chronic inflammatory disease or disorder. Chronic inflammation is a debilitating and serious condition associated with many of the above diseases and is characterised by persistent inflammation at a site of infection or injury, or persistent inflammation of an unknown origin, or in relation to altered immune responses such as in autoimmune disease.

Thus, in one embodiment, the CD39-targeted eosinophil-depleting agent is employed in the treatment of a chronic inflammatory condition wherein the condition is associated with any condition associated with inappropriate inflammation. Such conditions include, but are not limited to, rheumatoid arthritis (RA), autoimmune conditions, inflammatory bowel diseases, non-healing wounds, multiple sclerosis, cancer, atherosclerosis, vasculitis, Sjogren's disease, diabetes, lupus erythematosus (including systemic lupus erythematosus), asthma, fibrotic diseases (including liver cirrhosis), pulmonary fibrosis, UV damage and psoriasis.

In certain embodiments, the CD39-targeted eosinophil-depleting agent is employed in the treatment of a condition selected from axial spondyloarthropathy, primary biliary cholangitis, and allergy, for example a food allergy such as a peanut allergy, or a pollen allergy.

CD39-targeted eosinophil-depleting agents according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics.

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example anti-inflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterised by asthma attack, e.g. between the hours of about 4 to 6 am, i.e. at a time normally substantially distant from any previously administered symptomatic asthma therapy.

CD39-targeted eosinophil-depleting agents of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activity, CD39-targeted eosinophil-depleting agent of the invention are also useful in the treatment of eosinophil related disorders, e.g. eosinophilia, in particular eosinophil related disorders of the airways (e.g., involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

CD39-targeted eosinophil-depleting agents of the invention are also useful in the treatment of inflammatory or allergic conditions of the skin, for example psoriasis, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

CD39-targeted eosinophil-depleting agents of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g., hemolytic anemia, aplastic anemia, pure red cell anemia and idiopathic thrombocytopenia), systemic lupus erythematosus, rheumatoid arthritis, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine ophthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minimal change nephropathy), chronic granulomatous disease, endometriosis, leptospirosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods of this invention is a disease of the skin. In some embodiments, the inflammatory disease of the skin is selected from contact dermatitis, atopic dermatitis, alopecia areata, erythema multiforme, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, and other inflammatory or allergic conditions of the skin.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), and osteoarthritis.

In certain embodiments, CD39-targeted eosinophil-depleting agents of the invention may also be used for the treatment of drug-induced eosinophilia, such as eosinophilic asthma and hypereosinophilic disorders that are secondary to ICI therapies and/or other drugs including but not limited to antimalarials (e.g., pyrimethamine and dapson), penicillins, glycopeptides, cephalosporins, sulphonamides, tetracyclines (especially minocycline), nitrofurantoin, anti-tuberculous therapies, ACE inhibitors, tryptophan, anticonvulsants (e.g., phenytoin, carbamazepine, and phenobarbitone), NSAIDs, gold, H2-receptor antagonists, proton pump inhibitors, aminosalicylates, and chlorpropamide.

The present invention provides compositions comprising a CD39-targeted eosinophil-depleting agent, such as an anti-CD39 antibody described herein. The present invention also provides pharmaceutical compositions comprising an anti-CD39 antibody described herein and a pharmaceutically acceptable vehicle. In some embodiments, the pharmaceutical compositions find use in immunotherapy. In some embodiments, the pharmaceutical compositions find use in inflammatory diseases and/or autoimmune diseases. In some embodiments, the pharmaceutical compositions find use in treating a human patient.

Formulations are prepared for storage and use by combining a purified agent encompassed by the present invention with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

In some embodiments, the anti-CD39 antibody is lyophilized and/or stored in a lyophilized form. In some embodiments, a formulation comprising an anti-CD39 antibody described herein is lyophilized.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22.sup.nd Edition, 2012, Pharmaceutical Press, London.).

The pharmaceutical compositions encompassed by the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intrastital, subcutaneous, intraperitoneal, intramuscular (e.g., injection or infusion), or intracranial (e.g., intrathecal or intraventricular) and intrasynovial.

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound encompassed by the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The anti-CD39 antibody can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22.sup.nd Edition, 2012, Pharmaceutical Press, London.

In certain embodiments, pharmaceutical formulations include an anti-CD39 antibody complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the anti-CD39 antibody can be produced. Suitable examples of sustained-release preparations include semi-permeable matrices of solid hydrophobic polymers containing an anti-CD39 antibody, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), sucrose acetate isobutyrate, and poly-D-(−)-3-hydroxybutyric acid.

In certain embodiments, in addition to administering an anti-CD39 antibody, the method or treatment further comprises administering at least one additional immune response stimulating agent. In some embodiments, the additional immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), a checkpoint inhibitor, an antibody that blocks immunosuppressive functions (e.g., an anti-CTLA-4 antibody, anti-CD28 antibody, anti-CD3 antibody), a toll-like receptor (e.g., TLR4, TLR7, TLR9), or a member of the B7 family (e.g., CD80, CD86). An additional immune response stimulating agent can be administered prior to, concurrently with, and/or subsequently to, administration of the anti-CD39 antibody. Pharmaceutical compositions comprising an anti-CD39 antibody and the immune response stimulating agent(s) are also provided. In some embodiments, the immune response stimulating agent comprises 1, 2, 3, or more immune response stimulating agents.

In certain embodiments, in addition to administering an anti-CD39 antibody, the method or treatment further comprises administering at least one additional therapeutic agent. An additional therapeutic agent can be administered prior to, concurrently with, and/or subsequently to, administration of the anti-CD39 antibody. Pharmaceutical compositions comprising an anti-CD39 antibody and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects.

Combination therapy may allow for a lower dose of each agent than is used in monotherapy, thereby reducing toxic side effects and/or increasing the therapeutic index of the anti-CD39 antibody. In some embodiments, combination therapy comprises a therapeutic agent that inhibits immune activation, or inhibits immunostimulatory signals, or may be an agent that promotes normal tissue regeneration at the site of the inflammatory damage.

In certain embodiments, the present invention provides a method of treating an inflammatory disease, disorder or condition by administering to a patient in need thereof with a CD39-targeted eosinophil-depleting agent and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), “anti-Jak” inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bidt, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a CD39-targeted eosinophil-depleting agent and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a CD39-targeted eosinophil-depleting agent and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a CD39-targeted eosinophil-depleting agent and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a CD39-targeted eosinophil-depleting agent and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a CD39-targeted eosinophil-depleting agent and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a CD39-targeted eosinophil-depleting agent and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bidt, Uniphyl®, Theo-24®) and aminophylline, and IgE antibodies such as omalizumab (Xolair®).

In some embodiments, the present invention provides a method of treating COPD comprising administering to a patient in need thereof a CD39-targeted eosinophil-depleting agent and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bidt, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®

For the treatment of a disease, the appropriate dosage of an anti-CD39 antibody depends on the type of disease to be treated, the severity and course of the disease, the responsiveness of the disease, whether the anti-CD39 antibody is administered for therapeutic or preventative purposes, previous therapy, the patient's clinical history, and so on, all at the discretion of the treating physician. The anti-CD39 antibody can be administered one time or over a series of treatments lasting from several days to several months, or until a cure is effected or a diminution of the disease state is achieved. Optimal dosing schedules can be calculated from measurements of drug accumulation in the body of the patient and will vary depending on the relative potency of an individual agent. The administering physician can determine optimum dosages, dosing methodologies, and repetition rates. In certain embodiments, dosage is from 0.01 μg to 100 mg/kg of body weight, from 0.01 μg to 10 mg/kg of body weight, from 0.1 μg to 100 mg/kg of body weight, from 0.1 μg to 10 mg/kg of body weight, from 1 μg to 100 mg/kg of body weight, from 1 μg to 10 mg/kg of body weight, from 0.01 mg to 100 mg/kg of body weight, from 0.01 mg to 50 mg/kg of body weight, from 0.01 mg to 25 mg/kg of body weight, from 0.01 mg to 10 mg/kg of body weight, from 0.01 mg to 5 mg/kg of body weight, from 0.1 mg to 100 mg/kg of body weight, from 0.1 mg to 50 mg/kg of body weight, from 0.1 mg to 25 mg/kg of body weight, from 0.1 mg to 10 mg/kg of body weight, from 0.1 mg to 5 mg/kg of body weight, from 1 mg to 100 mg/kg of body weight, 1 mg to 50 mg/kg of body weight, from 1 mg to 25 mg/kg of body weight, from 1 mg to 10 mg/kg of body weight, or from 1 mg to 5 mg/kg of body weight. In certain embodiments, the dosage of the anti-CD39 antibody is from about 0.01 mg to about 10 mg/kg of body weight. In some embodiments, the dosage of the anti-CD39 antibody is about 0.01 mg/kg of body weight.

In some embodiments, the dosage of the anti-CD39 antibody is about 0.025 mg/kg of body weight. In some embodiments, the dosage of the anti-CD39 antibody is about 0.05 mg/kg of body weight. In some embodiments, the dosage of the anti-CD39 antibody is about 0.1 mg/kg of body weight. In some embodiments, the dosage of the anti-CD39 antibody is about 0.25 mg/kg of body weight. In some embodiments, the dosage of the anti-CD39 antibody is about 0.5 mg/kg of body weight. In some embodiments, the dosage of the anti-CD39 antibody is about 1 mg/kg of body weight. In some embodiments, the dosage of the anti-CD39 antibody is about 1.5 mg/kg of body weight. In some embodiments, the dosage of the anti-CD39 antibody is about 2 mg/kg of body weight. In some embodiments, the dosage of the anti-CD39 antibody is about 2.5 mg/kg of body weight. In some embodiments, the dosage of the anti-CD39 antibody is about 5 mg/kg of body weight. In some embodiments, the dosage of the anti-CD39 antibody is about 7.5 mg/kg of body weight. In some embodiments, the dosage of the anti-CD39 antibody is about 10 mg/kg of body weight. In some embodiments, the dosage of the anti-CD39 antibody is about 25 mg/kg of body weight. In some embodiments, the dosage is a range bounded by dosages described herein, such as 0.025 mg/kg to 25 mg/kg of body weight, or any range in between, such as 2-25 mg/kg body weight, 5-10 mg/kg, and the like. In certain embodiments, the dosage can be given once or more daily, trice a week, twice a week, weekly, monthly, or yearly. In certain embodiments, the anti-CD39 antibody is given once every week, once every two weeks, once every three weeks, or once every four weeks.

In some embodiments, an anti-CD39 antibody may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. In some embodiments, a dosing regimen may comprise administering an initial loading dose, followed by maintenance doses of, for example one-half of the initial dose every other week. In some embodiments, a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the anti-CD39 antibody is administered for 2, 3, 4, 5, 6, 7, 8, or more cycles. For example, the anti-CD39 antibody is administered every 2 weeks for 6 cycles, the anti-CD39 antibody is administered every 3 weeks for 6 cycles, the anti-CD39 antibody is administered every 2 weeks for 4 cycles, the anti-CD39 antibody is administered every 3 weeks for 4 cycles, etc. Dosing schedules can be decided upon and subsequently modified by those skilled in the art.

Thus, the present invention provides methods of administering to a subject the anti-CD39 antibody described herein comprising using an intermittent dosing strategy for administering one or more agents, which may reduce side effects and/or toxicities associated with administration of an anti-CD39 antibody, anti-inflammatory agent, etc. In some embodiments, a method for treating a disease or condition associated with unwanted eosinophil activity in a human subject comprises administering to the subject a therapeutically effective dose of an anti-CD39 antibody in combination with a therapeutically effective dose of an anti-inflammatory agent, wherein one or both of the agents are administered according to an intermittent dosing strategy. In some embodiments, the intermittent dosing strategy comprises administering an initial dose of an anti-CD39 antibody to the subject, and administering subsequent doses of the anti-CD39 antibody about once every 2 weeks. In some embodiments, the intermittent dosing strategy comprises administering an initial dose of an anti-CD39 antibody to the subject, and administering subsequent doses of the anti-CD39 antibody about once every 3 weeks. In some embodiments, the intermittent dosing strategy comprises administering an initial dose of an anti-CD39 antibody to the subject, and administering subsequent doses of the anti-CD39 antibody about once every 4 weeks. In some embodiments, the anti-CD39 antibody is administered using an intermittent dosing strategy and the anti-inflammatory agent is administered weekly.

### VII. Anti-CD39 Antibody Conjugates

The anti-CD39 antibodies disclosed herein may also be conjugated to a cytotoxic moiety. In some embodiments, bispecific anti-CD39 antibodies disclosed herein are conjugated to a cytotoxic moiety to further improve the specificity.

In certain embodiments, the anti-CD39 antibody (e.g., the bispecific anti-CD39 antibody described herein) is capable of inducing cytotoxicity in a CD39-expressing cell (e.g., eosinophils) by internalization of the antibody conjugated to or associated with a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin-inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, Momordica charantia inhibitor, curcin, crotin, Sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In one embodiment, the anti-CD39 antibody (e.g., the bispecific anti-CD39 antibody described herein) is conjugated to an auristatin or a peptide analog, derivative or prodrug thereof. Auristatins have been shown to interfere with microtubule dynamics, GTP hydrolysis and nuclear and cellular division (Woyke et al., 2001, Antimicrob. Agents and Chemother. 45 (12): 3580-3584) and have anti-cancer (U.S. Pat. No. 5,663,149) and anti-fungal activity (Pettit et al., 1998, Antimicrob. Agents and Chemother. 42:2961-2965). For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968, and WO205082023.

In another embodiment, the anti-CD39 antibody (e.g., the bispecific anti-CD39 antibody described herein) is conjugated to pyrrolo[2,1-c][1,4]-benzodiazepine (PDB) or an analog, derivative or prodrug thereof. Suitable PDBs and PDB derivatives, and related technologies are described in, e.g., Sagnou et al., 2000, Bioorg Med Chem Lett 10 (18): 2083-2086; Antonow et al., 2008, Cancer J 14 (3): 154-169; Howard et al., 2009, Bioorg Med Chem Lett 19:6463-6466; and Hartley et al., 2010, Cancer Res 70 (17): 6849-6858.

In another embodiment, the anti-CD39 antibody (e.g., the bispecific anti-CD39 antibody described herein) is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In a particular embodiment, the anti-CD39 antibody (e.g., the bispecific anti-CD39 antibody described herein) is conjugated to an anthracycline or an analog, derivative or prodrug thereof. In another particular embodiment, the anti-CD39 antibody (e.g., the bispecific anti-CD39 antibody described herein) is conjugated to maytansine or an analog, derivative or prodrug thereof. In another particular embodiment, the anti-CD39 antibody (e.g., the bispecific anti-CD39 antibody described herein) is conjugated to calicheamicin or an analog, derivative or prodrug thereof. In another particular embodiment, the anti-CD39 antibody (e.g., the bispecific anti-CD39 antibody described herein) is conjugated to duocarmycin or an analog, derivative or prodrug thereof. In another particular embodiment, the anti-CD39 antibody (e.g., the bispecific anti-CD39 antibody described herein) is conjugated to rachelmycin (CC-1065) or an analog, derivative or prodrug thereof. In another particular embodiment, the anti-CD39 antibody (e.g., the bispecific anti-CD39 antibody described herein) is conjugated to dolastatin 10 or an analog, derivative or prodrug thereof. In another particular embodiment, the anti-CD39 antibody (e.g., the bispecific anti-CD39 antibody described herein) is conjugated to dolastatin 15 or an analog, derivative or prodrug thereof. In another particular embodiment, the anti-CD39 antibody (e.g., the bispecific anti-CD39 antibody described herein) is conjugated to monomethyl auristatin E or an analog, derivative or prodrug thereof. In another particular embodiment, the anti-CD39 antibody (e.g., the bispecific anti-CD39 antibody described herein) is conjugated to monomethyl auristatin For an analog, derivative or prodrug thereof. In another particular embodiment, the anti-CD39 antibody (e.g., the bispecific anti-CD39 antibody described herein) is conjugated to pyrrolo[2,1-c][1,4]-benzodiazepine or an analog, derivative or prodrug thereof. In another particular embodiment, the anti-CD39 antibody (e.g., the bispecific anti-CD39 antibody described herein) is conjugated to irinotecan or an analog, derivative or prodrug thereof.

### VIII. Pharmaceutical Compositions

Anti-CD39 antibodies, antibody fragments, nucleic acids, or vectors encompassed by the present invention can be formulated in compositions, especially pharmaceutical compositions. Such compositions comprise a therapeutically or prophylactically effective amount of an anti-CD39 antibody, antibody fragment, nucleic acid, or vector encompassed by the present invention in admixture with a suitable carrier, e.g., a pharmaceutically acceptable agent. Typically, anti-CD39 antibodies, antibody fragments, nucleic acids, or vectors encompassed by the present invention are sufficiently purified for administration to an animal before formulation in a pharmaceutical composition.

Pharmaceutically acceptable agents for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin are exemplary appropriate carriers. The pharmaceutical compositions can include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also can be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers can be conventional buffers such as acetate, borate, citrate, phosphate, bicarbonate, or Tris-HCl. Acetate buffer may be about pH 4-5.5, and Tris buffer can be about pH 7-8.5. Additional pharmaceutical agents are set forth in Remington's Pharmaceutical Sciences, 18th Edition, A. R. Gennaro, ed., Mack Publishing Company, 1990.

The composition can be in liquid form or in a lyophilized or freeze-dried form and may include one or more lyoprotectants, excipients, surfactants, high molecular weight structural additives and/or bulking agents (see for example U.S. Pat. Nos. 6,685,940; 6,566,329; and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g. polysorbate 20, polysorbate 80); poloxamers (e.g. poloxamer 188); poly(ethylene glycol)phenyl ethers (e.g. Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, Hnoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl ofeyl-taurate; and the MONAQUAT™. series (Mona Industries, Inc., Paterson, NJ.), polyethyl glycol, polypropyl glycol, and copolymers of ethylene and propylene glycol (e.g. Pluronics, PF68 etc). Exemplary amounts of surfactant that may be present in the pre-lyophilized formulation are from about 0.001-0.5%. High molecular weight structural additives (e.g. fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions can be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replem′shers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980, which is incorporated herein by reference.

Pharmaceutical compositions described herein can be formulated for controlled or sustained delivery in a manner that provides local concentration of the product (e.g., bolus, depot effect) and/or increased stability or half-life in a particular local environment. The compositions can include the formulation of anti-CD39 antibodies, antibody fragments, nucleic acids, or vectors encompassed by the present invention with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then can be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents (e.g., antibodies). See, for example, the description of controlled release porous polymeric microparticles for the delivery of pharmaceutical compositions in PCT Application Publication WO 93/15722.

Suitable materials for this purpose include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al., 1983, Biopolymers 22:547-556), poly(2-hydroxyethyl-methacrylate) (Langer et al., 1981, J. Biomed. Mater. Res. 15:167-277 and Langer et al., 1982, Chem. Tech. 12:98-105), ethylene vinyl acetate, or poly-D (˜)˜3-hydroxybutyric acid. Other biodegradable polymers include poly(lactones), poly(acetals), poly(orthoesters), and poly(orthocarbonates). Sustained-release compositions also may include liposomes, which can be prepared by any of several methods known in the art (see, e.g., Eppstein et al., 1985, Proc. Natl. Acad. Sci. USA 82:3688-92). The carrier itself, or its degradation products, should be nontoxic in the target tissue and should not further aggravate the condition. This can be determined by routine screening in animal models of the target disorder or, if such models are unavailable, in normal animals. Microencapsulation of recombinant proteins for sustained release has been performed successfully with human growth hormone (rhGH), interferon-(rhIFN--), interleukin-2, and MNrgp120 (Johnson et al., 1996, Nat. Med. 2:795-799; Yasuda et al., 1993, Biomed. Ther. 27:1221-1223; Hora et al., 1990, Bio/Technology. 8:755-758; Cleland et al., “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010). The sustained-release formulations of these proteins were developed using poly-lactic-coglycolic acid (PLGA) polymer due to its biocompatibility and wide range of biodegradable properties. The degradation products of PLGA, lactic and glycolic acids can be cleared quickly within the human body. Moreover, the degradability of this polymer can be depending on its molecular weight and composition. Lewis et al., “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41. Additional examples of sustained release compositions include, for example, EP 58,48 IA, U.S. Pat. No. 3,887,699, EP 158,277A, Canadian Patent No. 1176565; Sidman et al., 1983, Biopolymers 22:547; Langer et al., 1982, Chem. Tech. 12:98; Sinha et al., 2003, J. Control. Release 90:261; Zhu et al., 2000, Nat. Biotechnol. 18:24; and Dai et al., 2005, Colloids Surf B Biointerfaces 41:117.

Bioadhesive polymers are also contemplated for use in or with compositions encompassed by the present invention. Bioadhesives are synthetic and naturally occurring materials able to adhere to biological substrates for extended time periods. For example, Carbopol and polycarbophil are both synthetic cross-linked derivatives of poly(acrylic acid). Bioadhesive delivery systems based on naturally occurring substances include for example hyaluronic acid, also known as hyaluronan. Hyaluronic acid is a naturally occurring mucopolysaccharide consisting of residues of D-glucuronic and N-acetyl-D-glucosamine.

Hyaluronic acid is found in the extracellular tissue matrix of vertebrates, including in connective tissues, as well as in synovial fluid and in the vitreous and aqueous humour of the eye. Esterified derivatives of hyaluronic acid have been used to produce microspheres for use in delivery that are biocompatible and biodegrable (see for example, Cortivo et al., 1991, Biomaterials 12:727-730; European Publication No. 517,565; International Publication No. WO 96/29998; Ilium et al., 1994, J. Controlled Rel. 29:133-141). Exemplary hyaluronic acid containing compositions encompassed by the present invention comprise a hyaluronic acid ester polymer in an amount of approximately 0.1% to about 40% (w/w) of an IL-1/3 binding antibody or fragment to hyaluronic acid polymer. Both biodegradable and non-biodegradable polymeric matrices can be used to deliver compositions encompassed by the present invention, and such polymeric matrices may comprise natural or synthetic polymers. Biodegradable matrices are preferred. The period of time over which release occurs is based on selection of the polymer. Typically, release over a period ranging from between a few hours and three to twelve months is most desirable. Exemplary synthetic polymers which can be used to form the biodegradable delivery system include: polymers of lactic acid and glycolic acid, polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyanhydrides, polyurethanes and co-polymers thereof, poly(butic acid), poly(valeric acid), alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose methacrylate), poly(ethyl methacrylate), sulphate sodium salt, poly(methyl poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), polyethylene oxide), polyethylene terephthalate), poly(vinyl alcohols), polyvinyl acetate, poly vinyl chloride, polystyrene and polyvinylpyrrolidone. Exemplary natural polymers include alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The polymer optionally is in the form of a hydrogel (see for example WO 04/009664; WO 05/087201; Sawhney et al., 1993, Macromolecules 26:581-587) that can absorb up to about 90% of its weight in water and further, optionally is cross-linked with multi-valent ions or other polymers.

Delivery systems also include non-polymer systems that are lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the product is contained in a form within a matrix such as those described in U.S. Pat. Nos. 4,452,775; 4,675,189; and 5,736,152; and (b) diffusional systems in which a product permeates at a controlled rate from a polymer such as described in U.S. Pat. Nos. 3,854,480; 5,133,974; and 5,407,686. Liposomes containing the product may be prepared by methods known methods, such as for example (DE 3,218,121; Epstein et al., 1985, Proc. Natl. Acad. Sci. USA 82:3688-3692; Hwang et al., 1980, Proc. Natl. Acad. Sci. USA 77:4030-4034; EP 52,322; EP 36,676; EP 88,046; EP 143,949; EP 142,641; Japanese patent application 83-118008; U.S. Pat. Nos. 4,485,045 and 4,544,545; and EP 102,324).

Alternatively or additionally, the compositions can be administered locally via implantation into the affected area of a membrane, sponge, or other appropriate material on to which an anti-CD39 antibody, antibody fragment, nucleic acid, or vector encompassed by the present invention has been absorbed or encapsulated. Where an implantation device is used, the device can be implanted into any suitable tissue or organ, and delivery of an anti-CD39 antibody, antibody fragment, nucleic acid, or vector encompassed by the present invention can be directly through the device via bolus, or via continuous administration, or via catheter using continuous infusion.

A pharmaceutical composition comprising an anti-CD39 antibody, antibody fragment, nucleic acid, or vector encompassed by the present invention can be formulated for inhalation, such as for example, as a dry powder. Inhalation solutions also can be formulated in a liquefied propellant for aerosol delivery. In yet another formulation, solutions may be nebulized. Additional pharmaceutical composition for pulmonary administration includes, those described, for example, in PCT Application Publication WO 94/20069, which discloses pulmonary delivery of chemically modified proteins. For pulmonary delivery, the particle size should be suitable for delivery to the distal lung. For example, the particle size can be from 1 μm to 5 μm; however, larger particles may be used, for example, if each particle is fairly porous.

Certain formulations containing anti-CD39 antibody, antibody fragments, nucleic acids, or vectors encompassed by the present invention can be administered orally. Formulations administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of a selective binding agent. Diluents, flavorings, low melting point waxes, vegetable oils, lubricants, suspending agents, tablet disintegrating agents, and binders also can be employed.

Another preparation can involve an effective quantity of an anti-CD39 antibody, antibody fragment, nucleic acid, or vector encompassed by the present invention in a mixture with non-toxic excipients which are suitable for the manufacture of tablets. By dissolving the tablets in sterile water, or another appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

### IX. Exemplary Materials and Methods

### Reagents

All chemical reagents were purchased from Sigma-Aldrich (St. Louis, MO), cell culture media from Life Technologies (Carlsbad, CA), cell culture consumables from CELLTREAT® Scientific Products (Shirley, MA), and commercial antibodies from Biolegend (San Diego, CA), unless otherwise stated. Secondary antibody Alexa Fluor® 488-conjugated AffiniPure Donkey anti-human IgG (Fc specific) (#709-545-098) was obtained from Jackson ImmunoResearch (West Grove, PA), Bio-Glo™ (#G7941) from Promega (Madison, WI), 2-Deoxy-2-fluoro-L-fucose (#MD06089) from BIOSYNTH Carbosynth (Gardner, MA), and Human Recombinant IL-2 (#78036.1) and Human Recombinant IL-15 (#78218) from STEMCELL Technologies (Cambridge, MA).

The anti-human CD39 reference antibody (hCD39 Ref) was produced by transient transfection using the ExpiCHO™ Expression System Kit (#A29133; Thermo Fisher Scientific, Waltham, MA) and antibody sequences were obtained as published (Perrot et al., Cell Reports 27:2411-2425 (2019)). Both hCD39 Ref antibody and our fully human anti-CD39 monoclonal antibody (PEOWT22) contain the same ADCC-competent human IgG1 Fc fraction. The hCD39 Ref antibody shares the antigen binding sites with an antibody in the art. However, that prior art antibody, unlike the Ref antibody used in the current examples, was generated with an Fc portion specifically designed to have an abrogated ADCC function (i.e., was taught to have been generated specifically to bind CD39 and inhibit NTPase activity without invoking CD39-dependent ADCC cell killing).

### Cell Culture

Epstein-Barr virus (EBV)-transformed human B lymphoblastoid HCC1739BL cells (ATCC #CRL-2334), Raji cells (Raji-hCD39neg), and human CD39 stably transfected Raji cells (Raji-hCD39hi expressing high level of hCD39 and Raji-hCD39lo expressing low level of hCD39) were cultured in RPMI-1640 supplemented with 10% FBS, 1% penicillin-streptomycin. Human melanoma cells (SK-MEL-28, ATCC #HTB-72) were grown in EMEM plus 10% FBS, 1% penicillin-streptomycin. Human natural killer cells (NK-92-CD16 V/V) (ATCC #PTA-6967) were cultured in MEM-Alpha medium with IL-2 (10 ng/ml). Human umbilical vein endothelial cells (HUVEC), Single Donor, EGM™-2 (Lonza #C2517A, Basel, Switzerland) were grown in EGM™ Endothelial Cell Growth Medium BulletKit (Lonza #CC3124). All cell lines were maintained in culture flasks at 37° C. in a 5% CO₂ atmosphere at 100% humidity, except for Jurkat cells/NFAT-luc+FcγRIIIA (Promega #G7011), which were thawed in water bath at 37° C. prior to use for experiments.

### Production, Afucosylation, and Optimization of Our Fully Human Anti-CD39 Antibodies

Fully human anti-CD39 antibodies were produced by transient transfection using FreeStyle™ 293-F cells (Thermo Fisher Scientific #R79007) either in the absence (PEOWT22, the parent fully fucosylated version of anti-CD39 antibody) or presence of fucosylation inhibitor 2-Deoxy-2-fluoro-L-fucose (PEOAF22, the afucosylated version of anti-CD39 antibody). Antibody ADCC activity ranking is: PEOAF22>PEOWT22.

Optimized counterparts PEONP22 and PEO22 (afucosylated versions of anti-CD39 antibody) were produced by CHO cells stably transfected with the parental PEOWT22 plasmid using chemically modified cell culture media for ADCC activity enhancement. Antibody ADCC activity ranking is: PEO22>PEONP22>PEOWT22.

No genetic modifications were employed during either afucosylation process.

### NK Cell-Mediated Antibody-Dependent Cellular Cytotoxicity (ADCC: Nk Cytotoxicity Assay)

HCC1739BL target cells were pre-labeled with CFSE (0.025 uM) for 5 minutes at 37° C. in water bath. After two washes with 1×DPBS, cells were incubated in MEM-Alpha medium (Thermo Fisher Scientific #32561037) containing 4% ultra-low IgG FBS (Thermo Fisher Scientific #A3381901) with or without monoclonal antibodies for 30 minutes at 37° C. in 5% CO₂. Target cells were then co-cultured with NK-92-CD16 V/V effector cells at E:T=1:8 ratio for 6 hours at 37° C. in 5% CO₂. After incubation, cells were stained with Propidium Iodide (P/I) (200 ng/mL) for 10 minutes at room temperature and target cell death was analyzed by a Cytek™ Aurora flow cytometer (Cytek Biosciences). Results were expressed as % of cytotoxicity which represents the % of CFSE+P/I+ cells.

For NK cell killing assay on human eosinophils (EO), NK cells were first isolated from healthy human blood by using the EasySep™ Direct Human NK Cell Isolation Kit (STEMCELL Technologies #19665) according to the manufacturer's protocol. NK cells were then washed, resuspended in growth media (RPMI-1640+10% FBS+10% horse serum+10 ng/ml IL-2+10 ng/ml IL-15), and incubated for 24 hours at 37° C. in 5% CO₂. The following day, EO cells were isolated from healthy human blood by using the EasySep™ Direct Human Eosinophil Isolation Kit (STEMCELL Technologies #19656) according to the manufacturer's instructions. Isolated EO cells were then washed, resuspended in ADCC media (RPMI-1640+4% ultra-low IgG FBS), and incubated with serially diluted monoclonal antibodies for 30 minutes at 37° C. in 5% CO₂. Target EO cells and effector NK cells were then co-cultured at E:T=3:1 ratio for 6 hours at 37° C. in 5% CO₂. After incubation, cells were stained with 7-AAD (1:20) for 5 minutes at room temperature and target cell death was analyzed by a Cytek™ Aurora flow cytometer (Cytek Biosciences). Results were expressed as % of cytotoxicity which represents the % of 7-AAD⁺EO cells.

### Antibody-Dependent Cellular Phagocytosis (ADCP) Assay

Human monocytes were first isolated from healthy human blood by using the EasySep™ Direct Human Monocyte Isolation Kit (STEMCELL Technologies #19669) according to the manufacturer's protocol. Monocytes were then washed, resuspended in growth/differentiation media (RPMI-1640+10% FBS+10% Horse serum+5% Human serum +50 ng/ml M-CSF), and incubated for 7 days at 37° C. in 5% CO₂ to promote macrophage differentiation. After 7 days, EO cells freshly isolated from healthy human blood were washed, labelled with Tag-it Violet (5 mM) (BioLegend #425101), resuspended in ADCP media (RPMI-1640+4% ultra-low IgG FBS+50 ng/ml M-CSF), and incubated with Isotype Control (10 μg/mL) or PEO22 (1 or 10 μg/mL) for 30 minutes at 37° C. in 5% CO₂. Violet labelled EO target cells were washed twice and co-cultured with macrophages effector cells at E:T=1:5 ratio for 6 hours at 37° C. in 5% CO₂. After incubation, attached macrophages were removed, washed, and analyzed by flow cytometry. Macrophages positive for violet fluorochrome (Pacific Blue channel) represent the ADCP induction-EO-engulfment. Macrophages incubated with ADCP media alone were used as the negative control.

### NFAT Luciferase Reporter Jurkat System (Luc-Reporter ADCC Assay)

Attached target cells (SK-MEL-28 human melanoma cells endogenously expressing CD39 at an intermediate level or HUVEC cells endogenously expressing low level of CD39) were seeded in a 96-well plate (8×10³ cells/100 μL/well) (BRANDplates #781965) and grown for 24 hours, while suspension target cells (HCC1739BL) were seeded right before the experiment (5×10⁵ cells/mL). Cells were then washed twice with ADCC assay buffer (DMEM or RPMI-1640 medium supplemented with 4% ultra-low IgG FBS) and incubated with serially diluted monoclonal antibodies for 30 minutes at 37° C. Effector cells (Jurkat cells/NFAT-luc+FcγRIIIA; 3×10⁶ cells/mL) were then added to the wells and the mixture (E:T=6:1) was incubated for 6 hours at 37° C. Bio-Glo™ was finally added into wells and luminescence values were read at 5, 15, and 30 minutes using a Synergy™ Neo2 Multi-Mode Reader (BioTeK Instruments Inc.). ADCC activity was indicated by an increase of luciferase activity over background.

### Epitope Competition Assay

Unconjugated anti-human CD39 monoclonal antibodies (Human/Rabbit chimeric clones; 2 μg/mL) were incubated with HCC1739BL cells (1×10⁵ cells) for 30 minutes at 4° C. Cells were then washed twice with cell staining buffer and stained with PE-conjugated mouse anti-human CD39 monoclonal antibody Clone A1 (0.25 μg/mL, Biolegend #328208) for 30 minutes at 4° C. Cells were then washed twice and analyzed by a Cytek™ Aurora flow cytometer. PE median fluorescence intensity (MFI) was detected, and data was analyzed by FCS 30 Express 7 software (De Novo Software). Cells incubated with media instead of the chimeric antibody were used as control.

### Stable Immune Complex Assay

HCC1739BL cells (5×10⁵ cells/mL) were incubated with anti-human CD39 monoclonal antibodies (2 μg/mL) or left untreated for 24 hours at 37° C. in 5% CO₂. The following day, untreated cells were exposed to the same panel of monoclonal antibodies (2 μg/mL) but for 20 minutes at 4° C. to obtain the basal level of CD39 expression. Cells were then washed twice with cell staining buffer and stained with anti-human IgG (Fc specific) Alexa Fluor® 488 (1:2000) for 30 minutes at 4° C., followed by two additional washes and fixation with paraformaldehyde (PFA, 2%) for 10 minutes at room temperature. Lastly, cells were washed twice and analyzed by a Cytek™ Aurora flow cytometer (Cytek Biosciences). Alexa Fluor® 488 (AF488) MFI was detected, and data was analyzed by FCS Express 7 software (De Novo Software). The percentage of human CD39 loss on cell membrane at 24 hours was calculated as: [(20 min MFI-24 h MFI/20 min MFI)]X100.

### Conformational Epitope Mapping

This was done using a proprietary CLIPS technology by Pepscan (Lelystad, The Netherlands).

### Animals

C57BL6 human CD39 knock-in (hCD39KI) mice were licensed from Beth Israel Deaconess Medical Center and bred and housed in a specific pathogen-free Vivarium at Purinomia Biotech, Inc. All mice were kept in a temperature-controlled room with alternating 12-hour dark light cycles.

Blood and bone marrow granulocytes profiles in healthy hCD39KI mice were evaluated using 8-12 weeks old male and female mice after euthanasia. Samples were processed and analyzed as described below.

Antibody Administration in Healthy hCD39KI Mice

For FIG. 12: To analyze the in vivo effects of PEO22 and its lower-ADCC counterpart (PEOWT22) on blood granulocytes, 8-12 weeks old male healthy hCD39KI mice received either sterile 0.9% saline or 1 mg/kg of PEO22 or PEOWT22 (i.p.) on days 0 and 2. Blood samples were collected through lateral vein nick on day 0 (before antibody injection) and through vena cava on day 3 at the terminal harvest. Samples were processed and analyzed as described below.

For FIG. 13: To analyze the in vivo effect of PEO22 on blood and bone marrow eosinophils, 8-12 weeks old female healthy hCD39KI mice were treated with saline or PEO22 (1 mg/kg, i.p.) every two days for a total of 4 doses. Blood and bone marrow samples were collected from euthanized animals one day after the last dosing, processed and analyzed as described below.

### Mouse Eosinophilic Asthma Model Establishment and Study Design

The mouse eosinophilic asthma model was established as described in the literature (Matsuoka et al., Kurume Medical Journal, 65:37-46 (2018); Chen et al., Scientific Reports, 10:10557 (2020)) with modifications. 8-12 weeks old female hCD39KI mice were used in the studies. Briefly, all mice were sensitized by administration (i.p.) of an ovalbumin (OVA) sensitization solution (100 μg OVA mixed with 0.1 mL of aluminum hydroxide adjuvant) on days 1 and 7. For OVA challenge, mice were anesthetized with isoflurane and administered (i.n.) with a 40 μg/40 μL OVA solution on days 11-25 (daily for 15 consecutive days).

Mice received either saline or 1 mg/kg of PEO22 (i.p.) every two days on days 19, 21, 23 and 25 for a total of 4 doses. One day after the last dose, mice were anesthetized with isoflurane, and blood, bone marrow and lungs were collected and analyzed as described below.

### Mouse Atopic Dermatitis (AD) Model Establishment and Study Design

AD mouse model was induced by repeated epicutaneous (EC) challenge of adhesive-stripped skin with OVA. For that, 8-12 weeks old female hCD39KI mice were first subjected to OVA sensitization by i.p. administration of OVA solution (10 μg of OVA+0.1 mL of aluminum hydroxide adjuvant) on days 1 and 7. On day 14, mice were anesthetized with isoflurane, back's mice skin (around 1 cm in diameter) was shaved, and tissue adhesive liquid (LiquiVet Rapid, Oasis Medical Mettawa) was applied to the shaved skin. After 5 minutes, the adhesive plaque was removed together with the superficial corneal epithelium. OVA challenge was then performed by applying 0.1 mg/mL of OVA solution to the exposed skin. Each mouse was exposed to OVA solution challenge on the same site twice a week for five weeks. 0.5 mL of saline solution was given to each mouse subcutaneously during OVA challenge to prevent allergic reaction and keep animal hydration.

During the last week of OVA challenge, mice received either saline or 1 mg/kg of PEO22 (i.p.) every two days for a total of three doses. Animals were euthanized two days after the last dosing, blood and skin were collected for flow cytometry and histological examination, respectively.

### Mouse Eosinophilic Rhinosinustis Model Establishment and Study Design

Eosinophilic rhinosinustis mouse model was induced by OVA sensitization and OVA/Aspergillus oryzae-derived protease challenges. For that, 8-12 weeks old female hCD39KI mice were first subjected to OVA sensitization by subcutaneous administration of OVA solution (10 μg of OVA+0.1 mL of aluminum hydroxide adjuvant) on days 1 and 7. For OVA/protease challenge, on day 14, mice were anesthetized with isoflurane and administered (i.n.) with a 20 μg/20 μL OVA/protease mixture solution three times a week for 6 weeks.

During the last week of OVA/protease challenge, mice received either saline or 3 mg/kg of PEO22 (i.p.) every two days for a total of four doses. Animals were euthanized one day after the last dosing, blood and nasal cavities tissues were collected for flow cytometry and histological examination, respectively.

### Mouse Bone Marrow Collection, Processing, and Analysis

Mouse femur was collected and extremities were cut with a sterile scissor. Bone marrow cells were flushed by inserting a 25G needle attached to a 1 ml syringe containing cold RPMI-1640 media supplemented with 2% FBS (R2 buffer) into the bone. Cell flush were then passed through a 70 μm strainer, collected in a 5 mL FACS tube on ice, and centrifuged at 3500 rpm for 5 minutes at 8° C. After supernatant removal, remaining cells were resuspended in 1 mL of 1×Lysing buffer (BD Pharm Lyse), mixed and incubated for 3 minutes on ice protected from light. After centrifugation at 3500 rpm for 5 minutes at 8° C., cells were resuspended in 1 mL of R2 buffer, passed through a 70 μm strainer and collected into a new 1.5 mL Eppendorf tube. Cells were then centrifuged again at 3500 rpm for 5 minutes at 8° C. and resuspended with 1 mL of R2 buffer. Afterwards, 50 μL of cell suspension was aliquoted and blocked with 0.5 μL of Fc blocker (BD Biosciences #553142) for 10 minutes at 4° C., followed by antibody panel staining (Table 1) for 15 minutes at 4° C. Lastly, cells were washed once with R2 buffer, resuspended in 100 μL of R2 buffer, stained with 7-AAD (1:20) for 5 minutes on ice, transferred to a FACS tube containing 400 μL of R2 buffer, and analyzed by a Cytek Aurora flow cytometer. Absolute number and percentage (%) of each cell subpopulation (expressed as in relation to CD45⁺ or CD45⁺CD11b⁺ cells) as well as their respective human CD39 expression and other eosinophil cell surface markers (expressed as mean fluorescence intensity-MFI) were determined by using FCS Express 7 software (De Novo Software).

### Mouse Blood Collection, Processing, and Analysis

For mice terminal blood collection: Blood samples were drawn from vena cava after animals were anesthetized with isoflurane gas and submitted to laparotomy.

For mice non-terminal blood collection: Blood samples were collected through lateral vein nick with no anesthesia.

Animal's blood (200 μL) was collected into a 1.5 mL Eppendorf tube containing 1 μL of 10% EDTA and mix. 50 μL of EDTA-containing blood sample was transferred to a new 1.5 mL Eppendorf tube and incubated with 0.5 μL of Fc blocker (BD Biosciences #553142) for 10 minutes at 4° C., followed by antibody panel staining (Table 2) for 15 minutes at 4° C. Red blood cell lysis was then preformed using 1 mL of 1×ACK Lysing Buffer for 10 minutes at room temperature in the dark. Afterwards, cells were centrifuged at 3500 rpm for 5 minutes at 4° C., followed by a second round of red blood cell lysis (using BD Pharm Lyse). White blood cells were then washed with 1 mL of R2 buffer, resuspended in 100 μL of R2 buffer, and stained with 7-AAD (1:20) for 5 minutes on ice. Lastly, cells were transferred to a FACS tube containing 400 μL of R2 buffer and analyzed by a Cytek Aurora flow cytometer. Absolute number and percentage (%) of each granulocyte subpopulation (expressed as in relation to CD45⁺ or CD45⁺CD11b⁺ cells) as well as their respective human CD39 expression and other eosinophil cell surface markers (expressed as mean fluorescence intensity-MFI) were determined by using FCS Express 7 software (De Novo Software).

### Mouse Tissue Collection, Processing, and Analysis

The lungs, skin, and nasal cavities were removed from euthanized mice, fixed into formalin-fixed paraffin embedded (FFPE) blocks, and stained with H&E (Hematoxylin & Eosin) to assess the tissue pathology.

### Human Blood Processing and Analysis

Blood samples from healthy human donors were used for granulocytes analysis. Briefly, 50 μL of samples were aliquoted and incubated with 2.5 μL of human Fc blocker (Biolegend #422302) for 10 minutes at 4° C., followed by antibody panel staining (Table 3) for 15 minutes at 4° C. Red blood cell lysis was then performed using 1 mL of 1×ACK Lysing Buffer for 10 minutes at room temperature in the dark. Afterwards, cells were centrifuged at 3500 rpm for 10 minutes at 4° C. and washed once with 1 mL of R2 buffer. White blood cells were then resuspended in 100 μL of R2 buffer and stained with 7-AAD (1:20) for 5 minutes on ice. Lastly, cells were transferred to a FACS tube containing 400 μL of R2 buffer and analyzed by a Cytek Aurora flow cytometer. Percentage (%) of each granulocyte subpopulation as well as their respective human CD39 expression (expressed as mean fluorescence intensity-MFI) were determined by using FCS Express 7 software (De Novo Software).

### Statistical Analyses

Results are presented as mean (+SEM) and statistical analyses were performed using GraphPad Prism 9 (GraphPad Software, San Diego, CA).

TABLE 1
Detection Antibodies for Mouse Bone Marrow Analysis

| Name | Vendor/Cat# | Concentration |
|---|---|---|
| Rat anti-mouse CD45_Brilliant Violet 510 ™ | BioLegend/ | 1:1000 |
| | 103138 | |
| Rat anti-mouse/human CD11b_Alexa Fluor ® 700 | BioLegend/ | 1:1000 |
| | 101222 | |
| Rat anti-mouse Ly-6G_Brilliant Violet 785 ™ | BioLegend/ | 1:1000 |
| | 127645 | |
| Rat anti-mouse Ly-6C_Brilliant Violet 605 ™ | BioLegend/ | 1:1000 |
| | 128035 | |
| Rat anti-mouse Ly-6G and Ly-6C (Gr-1)_{—} | BD BioSciences/ | 1:1000 |
| Brilliant Violet 480 ™ | 746614 | |
| Rat anti-mouse F4/80_PE/Cyanine 5 | BioLegend/ | 1:1000 |
| | 123112 | |
| Rat anti-mouse CD200R3_PE/Cyanine7 | BioLegend/ | 1:500 |
| | 142212 | |
| Rat anti-mouse CD138_Brilliant Violet 421 ™ | BioLegend/ | 1:500 |
| | 142523 | |
| Rat anti-mouse CD49b (pan-NK cells)_PE | Biolegend/ | 1:500 |
| | 108908 | |
| Armenian Hamster anti-mouse CD11c_Pacific | BioLegend/ | 1:500 |
| Blue ™ | 117322 | |
| Rat anti-mouse CD193 (CCR3)_PE | BioLegend/ | 1:500 |
| | 144505 | |
| Armenian Hamster anti-mouse FcεRIα_APC | BioLegend/ | 1:500 |
| | 134316 | |
| Armenian Hamster anti-mouse | BioLegend/ | 1:500 |
| FcεRIα_PE/Cyanine7 | 134318 | |
| Rabbit anti-human CD39 Clone 8C11_Alexa | Purinomia Biotech Inc./ | 1:500 |
| Fluor ® 647 | NA | |
| Rabbit IgG isotype control (RbNP15)_Alexa | eBioscience ™/ | 1:250 |
| Fluor ® 647 | 51461682 | |
| Rat anti-mouse CD170 (Siglec-F)_Alexa Fluor ® | BioLegend/ | 1:250 |
| 488 | 155524 | |
| Rat anti-mouse CD125 (IL-5Rα)_Brilliant Violet | BD BioSciences/ | 1:250 |
| 711 ™ | 740817 | |
| Rat anti-mouse CD101_PE | Invitrogen/ | 1:250 |
| | 12-1011-82 | |

TABLE 2
Detection Antibodies for Mouse Blood Analysis

| Name | Vendor/Cat# | Concentration |
|---|---|---|
| Rat anti-mouse CD45_Brilliant Violet 510 ™ | BioLegend/ | 1:1000 |
| | 103138 | |
| Rat anti-mouse/human CD11b_Alexa Fluor ® 700 | BioLegend/ | 1:1000 |
| | 101222 | |
| Rat anti-mouse Ly-6G_Brilliant Violet 785 ™ | BioLegend/ | 1:1000 |
| | 127645 | |
| Rat anti-mouse Ly-6C_Brilliant Violet 605 ™ | BioLegend/ | 1:1000 |
| | 128035 | |
| Rat anti-mouse Ly-6G and Ly-6C (Gr-1)_{—} | BD BioSciences/ | 1:1000 |
| Brilliant Violet 480 ™ | 746614 | |
| Rat anti-mouse CD49b (pan-NK cells)_PE | Biolegend/ | 1:500 |
| | 108908 | |
| Rat anti-mouse CD200R3_PE/Cyanine7 | BioLegend/ | 1:500 |
| | 142212 | |
| Armenian Hamster anti-mouse FcεRIα_APC | BioLegend/ | 1:500 |
| | 134316 | |
| Rat anti-mouse CD193 (CCR3)_PE | BioLegend/ | 1:500 |
| | 144505 | |
| Rabbit anti-human CD39 Clone 8C11_Alexa | Purinomia Biotech Inc./ | 1:500 |
| Fluor ® 647 | NA | |
| Rabbit IgG isotype control (RbNP15)_Alexa | eBioscience ™ | 1:250 |
| Fluor ® 647 | 51461682 | |
| Rat anti-mouse CD125 (IL-5Rα)_Brilliant Violet | BD BioSciences/ | 1:250 |
| 711 ™ | 740817 | |
| Rat anti-mouse CD170 (Siglec-F)_Alexa Fluor ® | BioLegend/ | 1:250 |
| 488 | 155524 | |
| Rat anti-mouse CD101_PE | Invitrogen/ | 1:250 |
| | 12-1011-82 | |

TABLE 3
Detection Antibodies for Human Blood Analysis

| Name | Vendor/Cat# | Concentration |
|---|---|---|
| Mouse anti-human CD45_Brilliant Violet 510 ™ | BioLegend/ | 1:20 |
| | 304036 | |
| Mouse anti-human CD16 (FcγRIII)_FITC | BioLegend/ | 1:20 |
| | 360716 | |
| Mouse anti-human CD14_PE-Cyanine5 | ThermoFisher Scientific/ | 1:20 |
| | 15-0149-42 | |
| Mouse anti-human CD24_Brilliant Violet 421 ™ | BioLegend/ | 1:20 |
| | 311122 | |
| Mouse anti-human CD123 (IL-3Rα)_Alexa | BioLegend/ | 1:20 |
| Fluor ® 700 | 306040 | |
| Mouse anti-human HLA-DR_Brilliant Violet | BioLegend/ | 1:20 |
| 650 ™ | 307650 | |
| Mouse anti-human CD3_PE | BioLegend/ | 1:20 |
| | 981004 | |
| Mouse anti-human CD56 (NCAM)_Brilliant | BioLegend/ | 1:20 |
| Violet 605 ™ | 318334 | |
| Mouse anti-human CD4_Pacfic Blue ™ | BioLegend/ | 1:20 |
| | 317429 | |
| Mouse anti-human CD193 (CCR3)_Brilliant | BioLegend/ | 1:20 |
| Violet 711 ™ | 310731 | |
| Mouse anti-human Siglec-8_PE/Cyanine7 | BioLegend/ | 1:20 |
| | 347111 | |
| Mouse anti-human CD11c_Brilliant Violet 785 ™ | BioLegend/ | 1:20 |
| | 301644 | |
| Rabbit anti-human CD39 Clone 8C11_Alexa | Purinomia Biotech Inc. | 1:500 |
| Fluor ® 647 | | |
| Rabbit IgG Isotype Control (RbNP15)_Alexa | eBioscience ™ | 1:250 |
| Fluor ® 647 | 51461682 | |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) on the World Wide Web and/or the National Center for Biotechnology Information (NCBI) on the World Wide Web.

### Equivalents and Scope

The details of one or more embodiments encompassed by the present invention are set forth in the description above. Although the preferred materials and methods have been described above, any materials and methods similar or equivalent to those described herein may be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which this invention belongs. In the case of conflict, the present description provided above will control.

Those skilled in the art will recognize or be able to ascertain using no more than routine experimentation, many equivalents to the specific embodiments encompassed by the present invention described herein. The scope encompassed by the present invention is not intended to be limited to the description provided herein and such equivalents are intended to be encompassed by the appended claims.

The articles “a” and “an” are used herein to refer to one or to more than one (i.e., to at least one) of the grammatical object of the article unless indicated to the contrary or otherwise evident from the context. By way of example, “an element” means one element or more than one element. Claims or descriptions that include “or” between one or more members of a group are considered satisfied if one, more than one, or all of the group members are present in, employed in, or otherwise relevant to a given product or process unless indicated to the contrary or otherwise evident from the context. The present invention includes embodiments in which exactly one member of the group is present in, employed in, or otherwise relevant to a given product or process. The present invention also includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges in different embodiments encompassed by the present invention, to the tenth of the unit of the lower limit of the range, unless the context clearly dictates otherwise.

In addition, it is to be understood that any particular embodiment encompassed by the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions encompassed by the present invention (e.g., any antibiotic, therapeutic or active ingredient; any method of production; any method of use; etc.) may be excluded from any one or more claims, for any reason, whether or not related to the existence of prior art.

It is to be understood that the words which have been used are words of description rather than limitation, and that changes may be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope encompassed by the present invention.

What is claimed is:

1. A method for reducing eosinophil cells or eosinophilic function in a subject comprising administering an anti-CD39 antibody, or antigen-binding fragment thereof to the subject,
wherein the anti-CD39 antibody, or antigen-binding fragment thereof, comprises:
(i) at least one antigen binding domain that binds ectonucleoside triphosphate diphosphohydrolase-1 (CD39) at a site such that the anti-CD39 antibody forms a stable immune complex, and
(ii) an FcγRIIIa binding moiety that binds FcγRIIIa receptor and confers a) antibody-dependent cellular cytotoxicity (ADCC) activity and/or b) antibody-dependent cellular phagocytosis (ADCP) activity against CD39+ cells to the anti-CD39 antibody.

2. The method of claim 1, wherein the eosinophil cells are CD39+eosinophil cells.

3. The method of claim 1 or 2, wherein the CD39+eosinophil cells:
(i) co-express one or more of the cell surface markers selected from the group consisting of CD45, CD11b, Siglec-8, the α subunit of IL-5 receptor (IL-5Rα or CD125), the α subunit of IL-3 receptor (IL-3Rα or CD123), IL-4R, IL-9R, IL-13R, IL-14R, ST2 (IL-33R), PIRA, PIRB, L-selectin, EMR1, CCR3 (CD193), and CRTh2 (CD294);
(ii) are CD45⁺CD11b⁺eosinophil cells; and/or
(iii) are CD45⁺CD11b⁺Siglec-8+eosinophil cells.

4. The method of any one of claims 1-3, wherein the anti-CD39 antibody, or antigen-binding fragment thereof, promotes:
(i) stable immune complex formation when incubated with HCC1739BL cells as characterized by loss of less than 30% of the immune complex after 24 hours, optionally wherein the immune complex formation is detected by fluorescence intensity using a fluorescently labeled secondary antibody;
(ii) depletion of CD39+eosinophils;
(iii) binding to a CD39 epitope having a sequence selected from the group of CD39 amino acid epitope sequences listed in FIG. 30; and/or
(iv) binding to CD39 in a manner that is non-competitive or only partially competitive with monoclonal antibody Clone A1 binding to CD39.

5. The method of claim 4, wherein the anti-CD39 antibody, or antigen-binding fragment thereof, promotes depletion of CD39+eosinophils via ADCC-mediated killing and/or ADCP-mediated killing.

6. The method of claim 4, wherein the anti-CD39 antibody, or antigen-binding fragment thereof, promotes depletion of CD39+eosinophils in the form of an antibody-drug conjugate that is taken up by and is toxic to the CD39+eosinophils.

7. The method of any one of claims 1-6, wherein the FcγRIIIa binding moiety is selected from the group consisting of an Fc domain, an antibody or fragment thereof that binds to FcγRIIIα, and an FcγRIIIa binding peptide.

8. The method of any one of claims 1-7, wherein the antigen binding domain is selected from the group consisting of a Fab, Fab′, F(ab′)₂, Fv or single chain Fv (scFv), Fav, dsFv, sc (Fv)₂, Fde, sdFv, single domain antibody (dAb), and diabodies fragments, optionally wherein the antigen-binding domain is an scFV comprising the sequence of SEQ ID NO: 40.

9. The method of any one of claims 1-8, wherein the anti-CD39 antibody, or antigen-binding fragment, is monoclonal.

10. The method of any one of claims 1-9, wherein the anti-CD39 antibody, or antigen-binding fragment thereof, has a VH domain with an amino acid sequence that can be encoded by a nucleic acid that hybridizes under stringent conditions to the nucleic acid of SEQ ID NO. 1 and a VL domain with an amino acid sequence that can be encoded by a nucleic acid that hybridizes under stringent conditions to the nucleic acid of SEQ ID NO. 3.

11. The method of any one of claims 1-10, wherein the anti-CD39 antibody, or antigen-binding fragment thereof, comprises a heavy chain having CDRs at least 60% identical to the CDRs of SEQ ID NO. 2, 6, 10, 14, 18, 22, 26, 42, 46, 50, or 54, and a light chain having CDRs at least 60% identical to the CDRs of SEQ ID NO. 4, 8, 12, 16, 20, 24, 28, 44, 48, 52, or 56.

12. The method of any one of claims 1-11, wherein the anti-CD39 antibody, or antigen-binding fragment thereof, comprises a variable heavy (VH) chain at least 60% identical to SEQ ID NO. 2, 6, 10, 14, 18, 22, 26, 42, 46, 50, or 54, and a variable light (VL) chain at least 60% identical to SEQ ID NO. 4, 8, 12, 16, 20, 24, 28, 44, 48, 52, or 56.

13. The method of any one of claims 1-12, wherein the anti-CD39 antibody, or antigen-binding fragment thereof, comprises:
(i) a heavy chain variable domain comprising a complementarity determining region (CDR) H1 having an amino acid sequence at least 80% identical to SEQ ID NO. 29, a CDRH2 having an amino acid sequence at least 80% identical to SEQ ID NO. 30, and a CDRH3 having an amino acid sequence at least 80% identical to SEQ ID NO. 31; and
(ii) a light chain variable domain comprising a CDRL1 having an amino acid sequence at least 80% identical to SEQ ID NO. 32, a CDRL2 having an amino acid sequence at least 80% identical to SEQ ID NO. 33, and a CDRL3 having an amino acid sequence at least 80% identical to SEQ ID NO. 34.

14. The method of claim 13, wherein the anti-CD39 antibody, or antigen-binding fragment thereof, comprising
(i) a heavy chain variable domain comprising a CDRH1 having the sequence of SEQ ID NO: 29, a CDRH2 having the amino acid sequence of SEQ ID NO: 30, and a CDRH3 having the sequence of SEQ ID NO: 31; and
(ii) a light chain variable domain comprising a CDRL1 having the sequence of SEQ ID NO: 32, a CDRL2 having the sequence of SEQ ID NO: 33, and a CDRL3 having the sequence of SEQ ID NO: 34.

15. The method of claim 14, wherein the anti-CD39 antibody, or antigen-binding fragment thereof, comprising:
(i) a heavy chain variable domain comprising the sequence of SEQ ID NO: 2; and
(ii) a light chain variable domain comprising the sequence of SEQ ID NO: 4.

16. The method of claim 15, wherein the anti-CD39 antibody, or antigen-binding fragment thereof, comprising:
(i) a heavy chain comprising the sequence of SEQ ID NO: 36; and
(ii) a light chain comprising the sequence of SEQ ID NO: 38.

17. The method of any one of claims 1-12, wherein the anti-CD39 antibody, or antigen-binding fragment thereof, comprises a heavy chain having CDRs selected from the group consisting of CDRs of SEQ ID NO. 6, 10, 14, 18, 22, 26, 42, 46, 50, and 54, and a light chain having CDRs selected from the group consisting of CDRs of SEQ ID NO. 8, 12, 16, 20, 24, 28, 44, 48, 52, and 56, and human framework sequences to form humanized heavy and light chains with an antigen binding site able to specifically bind human CD39.

18. The method of any one of claims 1-17, wherein the anti-CD39 antibody, or antigen-binding fragment thereof, comprises an Fc domain of an IgG1 or IgG3 isotype.

19. The method of any one of claims 1-18, wherein the Fc domain is human.

20. The method of any one of claims 1-19, wherein the anti-CD39 antibody, or antigen-binding fragment thereof, is hypo-fucosylated or afucosylated.

21. The method of any one of claims 1-20, wherein the anti-CD39 antibody, or antigen-binding fragment thereof, is human or is humanized.

22. The method of any one of claims 1-21, wherein the anti-CD39 antibody, or antigen-binding fragment thereof, is a bispecific including at least one additional antigen binding site for an eosinophil antigen.

23. The method of claim 22, wherein the additional antigen binding site binds to one or more of the following targets selected from the group consisting of Siglec-8, IL-5Rα (CD125), IL-3Rα (CD123), IL-4R, IL-9R, IL-13R, IL-14R, ST2 (IL-33R), PIRA, PIRB, L-selectin, EMR1, CCR3 (CD193), and CRTh2 (CD294).

24. The method of claim 22, wherein the additional antigen binding site binds an antigen upregulated on activated eosinophils.

25. The method of claim 22, wherein the additional antigen binding site binds to CD3, CD4, YSTCR, CD9, CD28, CD29, CD40, CD44, CD45, CD45RO, CD48, CD58, CD63 (lysosome-associated membrane protein 3), CD66b (CEACAM8), CD66e (CEACAM5), CD67, CD69, CD80, CD86, C5aR (CD88), CD101, CD122, CD137 (tumor necrosis factor receptor superfamily member 9, induced by lymphocyte activation, 4-1BB), CD274 (programmed death ligand 1), aub integrin (CD41), a2 integrin (CD49b), a4 integrin (CD49d), αL integrin (CD11a), αM integrin (CD11b), αX integrin (CD11c), αD integrin, β2 integrin (CD18), Aminopeptidase N (CD13), FcαRI (CD89), FcγRIII (CD16), FcγRII (CD32), Fc∈RII (CD23), Granulocyte monocyte-colony stimulating factorRα (CD116), HLA-DR, Intercellular adhesion molecule-1 (CD54), Interleukin (IL)-2Rα (CD25), IL-17RA, IL-17RB, Galectin-3, Neuropeptide S receptor, P-selectin glycoprotein ligand-1 (CD162), Semaphorin 7A (CD108), Thymic stromal lymphopoietin protein receptor (TSLPR), activated αM integrin, activated β1 integrin (CD29), activated B2 integrin, activated FcγRII, or activated CRTh2 (CD294).

26. The method of any one of claims 1-25, wherein the anti-CD39 antibody, or antigen-binding fragment thereof, reduces eosinophils.

27. The method of any one of claims 1-26, wherein the anti-CD39 antibody, or antigen-binding fragment thereof, reduces CD39^{high} inducible and/or activated eosinophils, optionally wherein the CD39^{high} inducible and/or activated eosinophils are i) presented in a pathological condition such as asthma, vasculitis, dermatitis, or rhinosinusitis; and/or ii) located in a space selected from the group consisting of blood, bone marrow, lesions, and/or a combination thereof.

28. The method of any one of claims 1-27, wherein an eosinophil-associated disease amenable to therapy with the anti-CD39 antibody is determined according to the presence of CD39^{high} inducible and/or activated eosinophils in a space selected from the group consisting of blood, bone marrow, lesions, and/or a combination thereof.

29. The method of any one of claims 1-28, wherein the subject has a disease or condition that involves unwanted eosinophilic activity.

30. The method of claim 29, wherein the unwanted eosinophilic activity is caused by aberrant activation or overabundance of eosinophils.

31. The method of claim 29 or 30, wherein the disease or condition is an inflammatory disorder or an autoimmune disease.

32. The method of claim 31, wherein the inflammatory disorder is a gastrointestinal inflammatory disorder.

33. The method of claim 32, wherein the gastrointestinal inflammatory disorder is eosinophilic esophagitis and/or Crohn's disease.

34. The method of claim 33, wherein the method further comprises administering to the subject one or more agents selected from the group consisting of glucocorticosteroids, leukotriene antagonists, mast cell stabilizers, immunomodulators, and Proton Pump Inhibitors (PPIs).

35. The method of claim 31, wherein the inflammatory disorder is a chronic inflammatory condition.

36. The method of claim 35, wherein the chronic inflammatory condition is selected from the group consisting of rheumatoid arthritis (RA), autoimmune conditions, inflammatory bowel diseases, non-healing wounds, multiple sclerosis, cancer, atherosclerosis, vasculitis, Sjogren's disease, diabetes, lupus erythematosus, asthma, fibrotic diseases, UV damage, and psoriasis.

37. The method of claim 36, wherein the fibrotic disease is selected from the group consisting of pulmonary fibrosis, liver fibrosis, heart disease, arthrofibrosis, Dupuytren's contracture, keloid fibrosis, mediastinal fibrosis, myelofibrosis, nephrogenic systemic fibrosis, retroperitoneal fibrosis, and scleroderma.

38. The method of claim 37, wherein the pulmonary fibrosis is cystic fibrosis, idiopathic pulmonary fibrosis, or progressive massive fibrosis.

39. The method of claim 37, wherein the liver fibrosis is liver fibrosis, liver cirrhosis, or primary biliary cirrhosis.

40. The method of claim 37, wherein the heart disease is atrial fibrosis, endomyocardial fibrosis, or old myocardial infarction.

41. The method of claim 36, wherein the inflammatory bowel disease is ulcerative colitis or Crohn's disease.

42. The method of claim 29 or 30, wherein the disease or condition is an inflammatory or obstructive airways disease.

43. The method of claim 42, wherein the inflammatory or obstructive airways disease is selected from the group consisting of asthma, acute lung injury (ALI), adult/acute respiratory distress syndrome (ARDS), chronic obstructive pulmonary, airways or lung disease (COPD, COAD or COLD), emphysema, exacerbation of airways hyperreactivity consequent to other drug therapy, bronchitis, and pneumoconiosis.

44. The method of claim 29 or 30, wherein the disease or condition is an inflammatory or allergic condition of the skin.

45. The method of claim 44, wherein the inflammatory or allergic condition of the skin is selected from the group consisting of psoriasis, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, and acne vulgaris.

46. The method of claim 29 or 30, wherein the disease or condition is pulmonary inflammatory diseases, axial spondyloarthropathy, primary biliary cholangitis, allergic rhinitis, chronic pulmonary disease, allergy, or eosinophilia.

47. The method of claim 29 or 30, wherein the disease or condition is drug-induced eosinophilia, such as eosinophilic asthma and hypereosinophilic disorders that are secondary to immune checkpoint inhibitor (ICI) therapies and/or other drugs including but not limited to antimalarials (e.g., pyrimethamine and dapson), penicillins, glycopeptides, cephalosporins, sulphonamides, tetracyclines (especially minocycline), nitrofurantoin, anti-tuberculous therapies, ACE inhibitors, tryptophan, anticonvulsants (e.g., phenytoin, carbamazepine, and phenobarbitone), NSAIDs, gold, H2-receptor antagonists, proton pump inhibitors, aminosalicylates, and chlorpropamide.

48. The method of claim 29 or 30, wherein the disease or condition relates to the respiratory, digestive, cardiovascular, endocrine, integumentary, skeletomuscular, or neurological system, or is a non-oncology hematologic disease.

49. The method of any one of claims 1-30, wherein the disease or condition is tissue graft rejection.

50. The method of claim 49, wherein the tissue graft is autologous or allogeneic.

51. The method of any one of claims 1-50, wherein the subject is a mammal.

52. The method of claim 51, wherein the mammal is a human or a rodent.

53. The method of any one of claims 1-52, wherein the anti-CD39 antibody, or antigen-binding fragment thereof, is administered to the subject with one or more pharmaceutically acceptable excipients, buffers or solutions.

54. The method of any one of claims 1-53, wherein the anti-CD39 antibody, or antigen-binding fragment thereof, is administered to the subject at a dose of 0.01 to 10 mg/kg, optionally wherein the dosing is provided by a continuous slow-releasing delivery platform to avoid/limit antibody-mediated target cytosis (or antigenic modulation or antigen shaving) for optimum ADCC-mediated and/or ADCP-mediated eosinophil cell depletion efficacy.

55. The method of any one of claims 1-54, wherein the anti-CD39 antibody, or antigen-binding fragment thereof, is administered to the subject once or more daily, trice a week, twice a week, once a week, once every two weeks, once every three weeks, or once every four weeks, optionally wherein the administration is once a week.

56. The method of any one of claims 1-55, wherein the anti-CD39 antibody, or antigen-binding fragment thereof, is administered to the subject for a duration of at least 2 to 6 treatment cycles, or is administered to the subject monthly for life-long use.

57. The method of any one of claims 1-56, wherein the anti-CD39 antibody, or antigen-binding fragment thereof, is administered to the subject via parenteral administration, submucosal hydrogel administration, pulmonary, or topical application, wherein the parenteral administration is by subcutaneous, intravenous, or intramuscular administration.

58. A method of treating a disease or condition associated with unwanted eosinophilic activity in a subject comprising administering an anti-CD39 antibody, or antigen-binding fragment thereof to the subject, wherein the anti-CD39 antibody, or antigen-binding fragment thereof, comprises:
(i) at least one antigen binding domain that binds ectonucleoside triphosphate diphosphohydrolase-1 (CD39) at a site such that the anti-CD39 antibody forms a stable immune complex, and
(ii) an FcγRIIIa binding moiety that binds FcγRIIIa receptor and confers a) antibody-dependent cellular cytotoxicity (ADCC) activity and/or b) antibody-dependent cellular phagocytosis (ADCP) activity against CD39+ cells to the anti-CD39 antibody.

59. The method of claim 58, wherein the disease or condition associated with unwanted eosinophilic activity is caused by 1) aberrant activation or eosinophilia, 2) an inflammatory disorder, and/or 3) drug-induced eosinophilia.

60. The method of claim 58 or 59, wherein the eosinophilic activity is from CD39+eosinophil cells, optionally wherein the CD39+eosinophil cells:
(i) co-express one or more of the cell surface markers selected from the group consisting of CD45, CD11b, Siglec-8, the α subunit of IL-5 receptor (IL-5Rα or CD125), the α subunit of IL-3 receptor (IL-3Rα or CD123), IL-4R, IL-9R, IL-13R, IL-14R, ST2 (IL-33R), PIRA, PIRB, L-selectin, EMR1, CCR3 (CD193), and CRTh2 (CD294);
(ii) are CD45⁺CD11b⁺eosinophil cells; and/or
(iii) are CD45⁺CD11b⁺Siglec-8+eosinophil cells.

61. The method of any one of claims 58-60, wherein the anti-CD39 antibody, or antigen-binding fragment thereof, promotes:
(i) stable immune complex formation when incubated with HCC1739BL cells as characterized by loss of less than 30% of the immune complex after 24 hours, optionally wherein the immune complex formation is detected by fluorescence intensity using a fluorescently labeled secondary antibody;
(ii) depletion of CD39+eosinophils;
(iii) binding to a CD39 epitope having a sequence selected from the group of CD39 amino acid epitope sequences listed in FIG. 30; and/or
(iv) binding to CD39 in a manner that is non-competitive or only partially competitive with monoclonal antibody Clone A1 binding to CD39.

62. The method of claim 61, wherein the anti-CD39 antibody, or antigen-binding fragment thereof, promotes depletion of CD39+eosinophils via ADCC-mediated killing and/or ADCP-mediated killing.

63. The method of claim 61, wherein the anti-CD39 antibody, or antigen-binding fragment thereof, promotes depletion of CD39+eosinophils in the form of an antibody-drug conjugate that is taken up by and toxic to the CD39+eosinophils.

64. The method of any one of claims 58-63, wherein the FcγRIIIa binding moiety is selected from the group consisting of an Fc domain, an antibody or fragment thereof that binds to FcγRIIIa, and an FcγRIIIa binding peptide.

65. The method of any one of claims 58-64, wherein the antigen binding domain is selected from the group consisting of a Fab, Fab′, F(ab′)₂, Fv or single chain Fv (scFv), Fav, dsFv, sc (Fv)₂, Fde, sdFv, single domain antibody (dAb), and diabodies fragments, optionally wherein the antigen-binding domain is an scFV comprising the sequence of SEQ ID NO: 40.

66. The method of any one of claims 58-65, wherein the anti-CD39 antibody, or antigen-binding fragment, is monoclonal.

67. The method of any one of claims 58-66, wherein the anti-CD39 antibody, or antigen-binding fragment thereof, has a VH domain with an amino acid sequence that can be encoded by a nucleic acid that hybridizes under stringent conditions to the nucleic acid of SEQ ID NO. 1 and a VL domain with an amino acid sequence that can be encoded by a nucleic acid that hybridizes under stringent conditions to the nucleic acid of SEQ ID NO. 3.

68. The method of any one of claims 58-67, wherein the anti-CD39 antibody, or antigen-binding fragment thereof, comprises a heavy chain having CDRs at least 60% identical to the CDRs of SEQ ID NO. 2, 6, 10, 14, 18, 22, 26, 42, 46, 50, or 54, and a light chain having CDRs at least 60% identical to the CDRs of SEQ ID NO. 4, 8, 12, 16, 20, 24, 28, 44, 48, 52, or 56.

69. The method of any one of claims 58-68, wherein the anti-CD39 antibody, or antigen-binding fragment thereof, comprises a variable heavy (VH) chain at least 60% identical to SEQ ID NO. 2, 6, 10, 14, 18, 22, 26, 42, 46, 50, or 54, and a variable light (VL) chain at least 60% identical to SEQ ID NO. 4, 8, 12, 16, 20, 24, 28, 44, 48, 52, or 56.

70. The method of any one of claims 58-69, wherein the anti-CD39 antibody, or antigen-binding fragment thereof, comprises:
(i) a heavy chain variable domain comprising a complementarity determining region (CDR) H1 having an amino acid sequence at least 80% identical to SEQ ID NO. 29, a CDRH2 having an amino acid sequence at least 80% identical to SEQ ID NO. 30, and a CDRH3 having an amino acid sequence at least 80% identical to SEQ ID NO. 31; and
(ii) a light chain variable domain comprising a CDRL1 having an amino acid sequence at least 80% identical to SEQ ID NO. 32, a CDRL2 having an amino acid sequence at least 80% identical to SEQ ID NO. 33, and a CDRL3 having an amino acid sequence at least 80% identical to SEQ ID NO. 34.

71. The method of claim 70, wherein the anti-CD39 antibody, or antigen-binding fragment thereof, comprising
(i) a heavy chain variable domain comprising a CDRH1 having the sequence of SEQ ID NO: 29, a CDRH2 having the amino acid sequence of SEQ ID NO: 30, and a CDRH3 having the sequence of SEQ ID NO: 31; and
(ii) a light chain variable domain comprising a CDRL1 having the sequence of SEQ ID NO: 32, a CDRL2 having the sequence of SEQ ID NO: 33, and a CDRL3 having the sequence of SEQ ID NO: 34.

72. The method of claim 71, wherein the anti-CD39 antibody, or antigen-binding fragment thereof, comprising:
(i) a heavy chain variable domain comprising the sequence of SEQ ID NO: 2; and
(ii) a light chain variable domain comprising the sequence of SEQ ID NO: 4.

73. The method of claim 72, wherein the anti-CD39 antibody, or antigen-binding fragment thereof, comprising:
(i) a heavy chain comprising the sequence of SEQ ID NO: 36; and
(ii) a light chain comprising the sequence of SEQ ID NO: 38.

74. The method of any one of claims 58-73, wherein the anti-CD39 antibody, or antigen-binding fragment thereof, comprises a heavy chain having CDRs selected from the group consisting of CDRs of SEQ ID NO. 6, 10, 14, 18, 22, 26, 42, 46, 50, and 54, and a light chain having CDRs selected from the group consisting of CDRs of SEQ ID NO. 8, 12, 16, 20, 24, 28, 44, 48, 52, and 56, and human framework sequences to form humanized heavy and light chains with an antigen binding site able to specifically bind human CD39.

75. The method of any one of claims 58-74, wherein the anti-CD39 antibody, or antigen-binding fragment thereof, comprises an Fc domain of an IgG1 or IgG3 isotype.

76. The method of any one of claims 58-75, wherein the Fc domain is human.

77. The method of any one of claims 58-76, wherein the anti-CD39 antibody, or antigen-binding fragment thereof, is hypo-fucosylated or afucosylated.

78. The method of any one of claims 58-77, wherein the anti-CD39 antibody, or antigen-binding fragment thereof, is human or is humanized.

79. The method of any one of claims 58-78, wherein the anti-CD39 antibody, or antigen-binding fragment thereof, is a bispecific including at least one additional antigen binding site for an eosinophil antigen.

80. The method of claim 79, wherein the additional antigen binding site binds to one or more of the following targets selected from the group consisting of Siglec-8, IL-5Rα (CD125), IL-3Rα (CD123), IL-4R, IL-9R, IL-13R, IL-14R, ST2 (IL-33R), PIRA, PIRB, L-selectin, EMR1, CCR3 (CD193), and CRTh2 (CD294).

81. The method of claim 79 or 80, wherein the additional antigen binding site binds an antigen upregulated on activated eosinophils.

82. The method of claim 79-81, wherein the additional antigen binding site binds to CD3, CD4, γδTCR, CD9, CD28, CD29, CD40, CD44, CD45, CD45RO, CD48, CD58, CD63 (lysosome-associated membrane protein 3), CD66b (CEACAM8), CD66e (CEACAM5), CD67, CD69, CD80, CD86, C5aR (CD88), CD101, CD122, CD137 (tumor necrosis factor receptor superfamily member 9, induced by lymphocyte activation, 4-1BB), CD274 (programmed death ligand 1), aub integrin (CD41), a2 integrin (CD49b), a4 integrin (CD49d), αL integrin (CD11a), αM integrin (CD11b), αX integrin (CD11c), αD integrin, β2 integrin (CD18), Aminopeptidase N (CD13), FcαRI (CD89), FcγRIII (CD16), FcγRII (CD32), Fc∈RII (CD23), Granulocyte monocyte-colony stimulating factorRα (CD116), HLA-DR, Intercellular adhesion molecule-1 (CD54), Interleukin (IL)-2Rα (CD25), IL-17RA, IL-17RB, Galectin-3, Neuropeptide S receptor, P-selectin glycoprotein ligand-1 (CD162), Semaphorin 7A (CD108), Thymic stromal lymphopoietin protein receptor (TSLPR), activated αM integrin, activated β1 integrin (CD29), activated β2 integrin, activated FcγRII, or activated CRTh2 (CD294).

83. The method of any one of claims 58-82, wherein the anti-CD39 antibody, or antigen-binding fragment thereof, reduces eosinophils.

84. The method of any one of claims 58-83, wherein the anti-CD39 antibody, or antigen-binding fragment thereof, reduces CD39^{high} inducible and/or activated eosinophils, optionally wherein the CD39^{high} inducible and/or activated eosinophils are i) presented in a pathological condition such as asthma, vasculitis, dermatitis, or rhinosinusitis; and/or ii) located in a space selected from the group consisting of blood, bone marrow, lesions, and/or a combination thereof.

85. The method of any one of claims 58-84, wherein an eosinophil-associated disease amenable to therapy with the anti-CD39 antibody is determined according to the presence of CD39^{high} inducible and/or activated eosinophils in a space selected from the group consisting of blood, bone marrow, lesions, and/or a combination thereof.

86. The method of any one of claims 59-85, wherein the inflammatory disorder is a gastrointestinal inflammatory disorder.

87. The method of claim 86, wherein the gastrointestinal inflammatory disorder is eosinophilic esophagitis and/or Crohn's disease.

88. The method of claim 87, wherein the method further comprises administering to the subject one or more agents selected from the group consisting of glucocorticosteroids, leukotriene antagonists, mast cell stabilizers, immunomodulators, and Proton Pump Inhibitors (PPIs).

89. The method of any one of claims 59-85, wherein the inflammatory disorder is a chronic inflammatory condition.

90. The method of claim 89, wherein the chronic inflammatory condition is selected from the group consisting of rheumatoid arthritis (RA), autoimmune conditions, inflammatory bowel diseases, non-healing wounds, multiple sclerosis, cancer, atherosclerosis, vasculitis, Sjogren's disease, diabetes, lupus erythematosus, asthma, fibrotic diseases, UV damage, and psoriasis.

91. The method of claim 90, wherein the fibrotic disease is selected from the group consisting of pulmonary fibrosis, liver fibrosis, heart disease, arthrofibrosis, Dupuytren's contracture, keloid fibrosis, mediastinal fibrosis, myelofibrosis, nephrogenic systemic fibrosis, retroperitoneal fibrosis, and scleroderma.

92. The method of claim 91, wherein the pulmonary fibrosis is cystic fibrosis, iodiopathic pulmonary fibrosis, or progressive massive fibrosis.

93. The method of claim 91, wherein the liver fibrosis is liver fibrosis, liver cirrhosis, or primary biliary cirrhosis.

94. The method of claim 91, wherein the heart disease is atrial fibrosis, endomyocardial fibrosis, or old myocardial infarction.

95. The method of claim 90, wherein the inflammatory bowel disease is ulcerative colitis or Crohn's disease.

96. The method of any one of claims 59-85, wherein the inflammatory disorder is an inflammatory or obstructive airways disease.

97. The method of claim 96, wherein the inflammatory or obstructive airways disease is selected from the group consisting of asthma, acute lung injury (ALI), adult/acute respiratory distress syndrome (ARDS), chronic obstructive pulmonary, airways or lung disease (COPD, COAD or COLD), emphysema, exacerbation of airways hyperreactivity consequent to other drug therapy, bronchitis, and pneumoconiosis.

98. The method of any one of claims 59-85, wherein the inflammatory disorder is an inflammatory or allergic condition of the skin.

99. The method of claim 98, wherein the inflammatory or allergic condition of the skin is selected from the group consisting of psoriasis, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, and acne vulgaris.

100. The method of any one of claims 59-85, wherein the inflammatory disorder is acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), or osteoarthritis.

101. The method of any one of claims 58-85, wherein the disease or condition is drug-induced eosinophilia, such as eosinophilic asthma and hypereosinophilic disorders that are secondary to immune checkpoint inhibitor (ICI) therapies and/or other drugs including but not limited to antimalarials (e.g., pyrimethamine and dapson), penicillins, glycopeptides, cephalosporins, sulphonamides, tetracyclines (especially minocycline), nitrofurantoin, anti-tuberculous therapies, ACE inhibitors, tryptophan, anticonvulsants (e.g., phenytoin, carbamazepine, and phenobarbitone), NSAIDs, gold, H2-receptor antagonists, proton pump inhibitors, aminosalicylates, and chlorpropamide.

102. The method of any one of claims 58-101, wherein the subject is a mammal.

103. The method of claim 102, wherein the mammal is a human or a rodent.

104. The method of any one of claims 58-103, wherein the anti-CD39 antibody, or antigen-binding fragment thereof, is administered to the subject with one or more pharmaceutically acceptable excipients, buffers or solutions.

105. The method of any one of claims 58-104, wherein the anti-CD39 antibody, or antigen-binding fragment thereof, is administered to the subject at a dose of 0.01 to 10 mg/kg, optionally wherein the dosing is provided by a continuous slow-releasing delivery platform to avoid/limit antibody-mediated target cytosis (or antigenic modulation or antigen shaving) for optimum ADCC-mediated and/or ADCP-mediated eosinophil cell depletion efficacy.

106. The method of any one of claims 58-105, wherein the anti-CD39 antibody, or antigen-binding fragment thereof, is administered to the subject once or more daily, trice a week, twice a week, once a week, once every two weeks, once every three weeks, or once every four weeks, optionally wherein the administration is once a week.

107. The method of any one of claims 58-106, wherein the anti-CD39 antibody, or antigen-binding fragment thereof, is administered to the subject for a duration of at least 2 to 6 treatment cycles, or is administered to the subject monthly for long-term use.

108. The method of any one of claims 58-107, wherein the anti-CD39 antibody, or antigen-binding fragment thereof, is administered to the subject via parenteral administration or submucosal hydrogel administration, pulmonary, or topical application, wherein the parenteral administration is by subcutaneous, intravenous, or intramuscular administration.

109. The method of any one of claims 58-108, wherein the method further comprises administering to the subject at least one additional therapeutic agent.

110. The method of any one of claim 109, wherein the at least one additional therapeutic agent is an anti-inflammatory agent, optionally wherein the anti-inflammatory agent is selected from the group consisting of non-steroidal anti-inflammatory drugs (NSAIDS), corticosteroids, leukotriene modifiers, and cytokine pathway blockers.

111. The method of claim 110, wherein the at least one additional therapeutic agent is administered prior to, concurrently with, and/or subsequently to, administration of the anti-CD39 antibody, or antigen-binding fragment thereof.


SEQUENCE LISTING (56 sequences)

SEQ ID NO: 1 - DNA, 357 bp, synthetic construct
  misc_feature 1..357: note = derived from human variable chain library
  source 1..357: mol_type = other DNA
  CDS 1..357
gaggtgcaactggtggagtctgggggaggtgtggtaaggcctggggggtccctgagactctcctgtgcagcctctggattcaccttcagtagctatgctatgcactgggtccgccaggctccaggcaaggggctggagtgggtggcagttatatcatatgatgtaagcaataaatactacgcagactccgtgaagggccgattcaccatctccagagacaattccaagaacacgctgtatctgcaaatgaacagcctgagagctgaggacacggctgtgtattactgtgcgagatcttactactactactacggtatggacgtctggggccaagggaccacggtcaccgtctcctca

SEQ ID NO: 2 - protein, 119 aa, synthetic construct
  REGION 1..119: note = Synthetic Construct
EVQLVESGGGVVRPGGSLRLSCAASGFTFSSYAMHWVRQAPGKGLEWVAVISYDVSNKYYADSVKGRFTISRDNSKNTLYLQMNSLRAEDTAVYYCARSYYYYYGMDVWGQGTTVTVSS

SEQ ID NO: 3 - DNA, 321 bp, synthetic construct
  misc_feature 1..321: note = Derived from human variable chain library
  source 1..321: mol_type = other DNA
  CDS 1..321
gatgttgtgatgacccagtctccatcctccctgtctgcatctgtaggagacagagtcaccatcacttgccgggcaagtcagagcattagcaggtacttagcctggtaccaacagaaacctggccaggctcccaggctcctcatctatgatgcatccaacagggccactggcatcccagtcaggttcagtggcagtgggtctgggacagacttcactctcaccatcagcagactggagccagaagattttgcagtgtattactgtcagcagtttggtaggtcacctcggacgttcggccaagggacacgactggagattaaa

SEQ ID NO: 4 - protein, 107 aa, synthetic construct
  REGION 1..107: note = Synthetic Construct
DVVMTQSPSSLSASVGDRVTITCRASQSISRYLAWYQQKPGQAPRLLIYDASNRATGIPVRFSGSGSGTDFTLTISRLEPEDFAVYYCQQFGRSPRTFGQGTRLEIK

SEQ ID NO: 5 - DNA, 357 bp, synthetic construct
  misc_feature 1..357: note = derived from rabbit monoclonal
  source 1..357: mol_type = other DNA
  CDS 1..357
cagtcagtgaaggaggccgggggtcgcctggtaacgcctggaggatccctgacactcacctgcacagtctctggattctccctcagtgcgtatggaataagttgggtccgccaggctccagggaagggactggaatggatcggaatcatttatagtagtggtaggacttactacgcgaactgggcgaaaggccgattcaccatctccaaaacctcgtcgaccacggtggatctgaaaatgaccagtctgacaaccgaggacacggccgcctatttctgtgccagatcacgggctggtattagtagtggtgatggttttgattcctggggcccaggcaccctggtcaccgtctcctca

SEQ ID NO: 6 - protein, 119 aa, synthetic construct
  REGION 1..119: note = Synthetic Construct
QSVKEAGGRLVTPGGSLTLTCTVSGFSLSAYGISWVRQAPGKGLEWIGIIYSSGRTYYANWAKGRFTISKTSSTTVDLKMTSLTTEDTAAYFCARSRAGISSGDGFDSWGPGTLVTVSS

SEQ ID NO: 7 - DNA, 330 bp, synthetic construct
  misc_feature 1..330: note = derived from rabbit monoclonal
  source 1..330: mol_type = other DNA
  CDS 1..330
gcccttgtgatgacccagactccatcctccgtgtctgcagctgtgggaggcacagtcaccatcaattgccaggccagtcagaacatttacagcaatttagcctggtatcagcagaaaccagggcagcgtccccagctcctgatctacagggcatccactctggcatctggggtcccatcgcggttcaaaggcagtgcatctgggacagaatacactctcaccatcagcggtgtgcagtgtgacgatgctgccacttactattgtcaacagggttttgatagtagtaacattgataatactttcggcggagggaccgaggtggtggtcaca

SEQ ID NO: 8 - protein, 110 aa, synthetic construct
  REGION 1..110: note = Synthetic Construct
ALVMTQTPSSVSAAVGGTVTINCQASQNIYSNLAWYQQKPGQRPQLLIYRASTLASGVPSRFKGSASGTEYTLTISGVQCDDAATYYCQQGFDSSNIDNTFGGGTEVVVT

SEQ ID NO: 9 - DNA, 345 bp, synthetic construct
  misc_feature 1..345: note = derived from rabbit monoclonal
  source 1..345: mol_type = other DNA
  CDS 1..345
cagtcggtggaggagtccgggggtcgcctggtcacgcctgggacacacctgacactcacctgcacagtctctggattctccctcagtaagagtataataagttgggtccgccaggctccagggaaggggctggaatacatcggaatcattggtagtagtggtagcacatactacgcgaactgggcgaaaggccgattcaccatctccaaaacctcgtcgaccacggtggatctgagaatgaccagtctgacacccgaggacacggccacctatttctgtgccagaggacttctttattctggtaataaatcgtggggcccgggcaccctggtcaccgtctcctca

SEQ ID NO: 10 - protein, 115 aa, synthetic construct
  REGION 1..115: note = Synthetic Construct
QSVEESGGRLVTPGTHLTLTCTVSGFSLSKSIISWVRQAPGKGLEYIGIIGSSGSTYYANWAKGRFTISKTSSTTVDLRMTSLTPEDTATYFCARGLLYSGNKSWGPGTLVTVSS

SEQ ID NO: 11 - DNA, 330 bp, synthetic construct
  misc_feature 1..330: note = derived from rabbit monoclonal
  source 1..330: mol_type = other DNA
  CDS 1..330
gccattgatatgacccagactccatcctccgtgtctgcagctgtgggaggcacagtcaccatcaactgccagtccagtcagagtgttttactgaacaaccaattatcctggtttcagcagaaaccagggcagcctcccaagctcctgatctatgatgcatccactctggaatctggggtcccatctcggttcacaggcagtggatctgggacacagttcactctcaccatcagcgacctggagtgtgacgatgctgccacttactattgtttaggcggttatagtgggaacctttatgctttcggcggagggaccgaggtgctagtcaaa

SEQ ID NO: 12 - protein, 110 aa, synthetic construct
  REGION 1..110: note = Synthetic Construct
AIDMTQTPSSVSAAVGGTVTINCQSSQSVLLNNQLSWFQQKPGQPPKLLIYDASTLESGVPSRFTGSGSGTQFTLTISDLECDDAATYYCLGGYSGNLYAFGGGTEVLVK

SEQ ID NO: 13 - DNA, 351 bp, synthetic construct
  misc_feature 1..351: note = derived from rabbit monoclonal
  source 1..351: mol_type = other DNA
  CDS 1..351
cagtcggtggaggagtccgggggtcgcctggtcacgcctgggacacccctgacactcacctgcacagtctctggattctccctcagtagctatgcaataagttgggtccgccaggctccagggaaggggctcgaatatatcgcgatcattaatagttatggtaccacatactacgcgagctgggcgaaaggccgagtcaccatctccaaaacctcgagcacggtggatctgaaaatctccagtccgacaaccgaggacacggccacctatttctgtgccagaggcgatagttatggtagtggtgttggtttgggcttgtggggcccaggcaccctggtcaccgtctcctca

SEQ ID NO: 14 - protein, 117 aa, synthetic construct
  REGION 1..117: note = Synthetic Construct
QSVEESGGRLVTPGTPLTLTCTVSGFSLSSYAISWVRQAPGKGLEYIAIINSYGTTYYASWAKGRVTISKTSSTVDLKISSPTTEDTATYFCARGDSYGSGVGLGLWGPGTLVTVSS

SEQ ID NO: 15 - DNA, 330 bp, synthetic construct
  misc_feature 1..330: note = derived from rabbit monoclonal
  source 1..330: mol_type = other DNA
  CDS 1..330
gcctatgatatgacccagactccagcctctgtggaggtagctgtgggaggcacagtcaccatcaagtgccaggccagtcagaacatttacagcaatttagcctggtatcagcagaaaccagggcagcgtcccaagctcctgatctacagggcatccagtctggcatctggggtcccgtcgcggttcagtggcagtggatctgggacagagttcactctcaccatcagcggtgtgcagtgtgacgatgctgccacttactactgtcaacagggttttagtagtaataatgttgataatactttcggcggagggaccgaggtggtggtcaaa

SEQ ID NO: 16 - protein, 110 aa, synthetic construct
  REGION 1..110: note = Synthetic Construct
AYDMTQTPASVEVAVGGTVTIKCQASQNIYSNLAWYQQKPGQRPKLLIYRASSLASGVPSRFSGSGSGTEFTLTISGVQCDDAATYYCQQGFSSNNVDNTFGGGTEVVVK

SEQ ID NO: 17 - DNA, 357 bp, synthetic construct
  misc_feature 1..357: note = derived from rabbit monoclonal
  source 1..357: mol_type = other DNA
  CDS 1..357
cagtcggtggaggagtccgggggtcgcctggtcacgcctgggacacccctgacactcacctgcaccgtctccggattctccctcagtagctatgcaatgagctgggtccgccaggctccagggaaggggctggaatacatcggaatcattagtagtagtggtagcacatactacgcgagctgggcgaaaggccgattcaccatctccaaaacctcgaccacggtggatctgaaaatctccagtccgacaaccgaggacacggccacctatttctgtgccagagatcgtgttatttatagtattggtccgtattattttaatttgtggggcccaggcaccctggtcaccgtctcctca

SEQ ID NO: 18 - protein, 119 aa, synthetic construct
  REGION 1..119: note = Synthetic Construct
QSVEESGGRLVTPGTPLTLTCTVSGFSLSSYAMSWVRQAPGKGLEYIGIISSSGSTYYASWAKGRFTISKTSTTVDLKISSPTTEDTATYFCARDRVIYSIGPYYFNLWGPGTLVTVSS

SEQ ID NO: 19 - DNA, 330 bp, synthetic construct
  misc_feature 1..330: note = derived from rabbit monoclonal
  source 1..330: mol_type = other DNA
  CDS 1..330
gcctatgatatgacccagactccatcctccgtgtctgcaactgtgggaggcacagtcaccatcaattgccaggccagtgagatcatttatagcaatttagcctggtatcagcagaaaccagggcagcctcccaagctcctgatctatggcgcatccactctggcatctggggtcccatcgcggttcaaaggcagtggatctgggacagagtacactctcaccatcagcgacctgcagtgtgacgatgctgccacttactactgtcaacagagttttagtagtaataatgttgggaatattttcggcggagggaccgaggtggtggtcaaa

SEQ ID NO: 20 - protein, 110 aa, synthetic construct
  REGION 1..110: note = Synthetic Construct
AYDMTQTPSSVSATVGGTVTINCQASEIIYSNLAWYQQKPGQPPKLLIYGASTLASGVPSRFKGSGSGTEYTLTISDLQCDDAATYYCQQSFSSNNVGNIFGGGTEVVVK

SEQ ID NO: 21 - DNA, 351 bp, synthetic construct
  misc_feature 1..351: note = derived from rabbit monoclonal
  source 1..351: mol_type = other DNA
  CDS 1..351
cagtcggtggaggagtccgggggtcgcctggtcacgcctgggacacccctgacactcacctgcacagcctctggattctccctcagtacccatgcaataaactgggtccgccaggctccagggaaggggctggaatggatcgggatcacttatgctagtggtaggacatattacgcgagctgggcgaaaggccgattcaccatctccaaaacctcgaccacggtggatctgaaaatcaccagtccgacaaccgaggacacggccacctatttctgtgccagaaatggggctgatgaaacattttactactttgacttgtggggcccaggcaccctggtcaccgtctcctca

SEQ ID NO: 22 - protein, 117 aa, synthetic construct
  REGION 1..117: note = Synthetic Construct
QSVEESGGRLVTPGTPLTLTCTASGFSLSTHAINWVRQAPGKGLEWIGITYASGRTYYASWAKGRFTISKTSTTVDLKITSPTTEDTATYFCARNGADETFYYFDLWGPGTLVTVSS

SEQ ID NO: 23 - DNA, 330 bp, synthetic construct
  misc_feature 1..330: note = derived from rabbit monoclonal
  source 1..330: mol_type = other DNA
  CDS 1..330
gcctatgatatgacccagactccagcctccgtggaggcagctgtgggaggcacagtcaccatcaagtgccaggccagtcagaatattaatacttggttatcctggtatcagcagaaggcagggcagcctcccaagctcctgatctacagggcatccactctggcatctggggtctcatcgcggttcaaaggcagtggatctgggacacagttcactctcaccatcagcggcgtggagtgtgccgatgctgccacttactactgtcaacaatatgatgctagtattaatattgataatgctttcggcggagggaccgaggtggtggtcaaa

SEQ ID NO: 24 - protein, 110 aa, synthetic construct
  REGION 1..110: note = Synthetic Construct
AYDMTQTPASVEAAVGGTVTIKCQASQNINTWLSWYQQKAGQPPKLLIYRASTLASGVSSRFKGSGSGTQFTLTISGVECADAATYYCQQYDASINIDNAFGGGTEVVVK

SEQ ID NO: 25 - DNA, 351 bp, synthetic construct
  misc_feature 1..351: note = derived from rabbit monoclonal
  source 1..351: mol_type = other DNA
  CDS 1..351
cagtcggtggaggagtccgggggtcgcctggtcacgcctgggacacccctgacactcacctgcacagtctctggaatcgacctcagtagcaatgcaatgagctgggtccgccaggctccagggaaggggctggaatatatcggaattattaggaataatgatatcacatactacgcgagctgggcgaaaggccgattcaccatctccaaaacctcgaccacggtggatctgataatcaccagtccgacaaccgaggacacggccacctatttctgtgccagagggggtggttcttacagtattgtcttctggaacttatggggcccaggcaccctggtcaccgtctcctca

SEQ ID NO: 26 - protein, 117 aa, synthetic construct
  REGION 1..117: note = Synthetic Construct
QSVEESGGRLVTPGTPLTLTCTVSGIDLSSNAMSWVRQAPGKGLEYIGIIRNNDITYYASWAKGRFTISKTSTTVDLIITSPTTEDTATYFCARGGGSYSIVFWNLWGPGTLVTVSS

SEQ ID NO: 27 - DNA, 330 bp, synthetic construct
  misc_feature 1..330: note = derived from rabbit monoclonal
  source 1..330: mol_type = other DNA
  CDS 1..330
gcctatgatatgacccagactccagcctctgtggaggtagctgtgggaggcacagtcaccatcaattgccaggccagtgagaggatttatagcaatttagcctggtatcagcagaaaccagggcagcgtcccaaactcctgatctattatgcatccactctggcatctggggtctcatcgcggttcaaaggcagtggatctgggacacagttcactctcaccatcagcggcgtgcagtgtgccgatgctgccacttactactgtcagcagggttatagtaataataatgttgacaatactttcggcggagggaccgaggtggtggtcaga

SEQ ID NO: 28 - protein, 110 aa, synthetic construct
  REGION 1..110: note = Synthetic Construct
AYDMTQTPASVEVAVGGTVTINCQASERIYSNLAWYQQKPGQRPKLLIYYASTLASGVSSRFKGSGSGTQFTLTISGVQCADAATYYCQQGYSNNNVDNTFGGGTEVVVR

SEQ ID NO: 29 - protein, 8 aa, synthetic construct
  REGION 1..8: note = Derived from human VH domain
GFTFSSYA

SEQ ID NO: 30 - protein, 8 aa, synthetic construct
  REGION 1..8: note = Derived from human VH domain
ISYDVSNK

SEQ ID NO: 31 - protein, 12 aa, synthetic construct
  REGION 1..12: note = Derived from human VH domain
ARSYYYYYGMDV

SEQ ID NO: 32 - protein, 6 aa, synthetic construct
  REGION 1..6: note = Derived from human VL domain
QSISRY

SEQ ID NO: 33 - skipped

SEQ ID NO: 34 - protein, 9 aa, synthetic construct
  REGION 1..9: note = Derived from human VL domain
QQFGRSPRT

SEQ ID NO: 35 - DNA, 1350 bp, synthetic construct
  misc_feature 1..1350: note = Derived from human variable domain and human Ig domain
  source 1..1350: mol_type = other DNA
  CDS 1..1350
gaggtgcaactggtggagtctgggggaggtgtggtaaggcctggggggtccctgagactctcctgtgcagcctctggattcaccttcagtagctatgctatgcactgggtccgccaggctccaggcaaggggctggagtgggtggcagttatatcatatgatgtaagcaataaatactacgcagactccgtgaagggccgattcaccatctccagagacaattccaagaacacgctgtatctgcaaatgaacagcctgagagctgaggacacggctgtgtattactgtgcgagatcttactactactactacggtatggacgtctggggccaagggaccacggtcaccgtctcctcagcctccactaagggcccatccgtgttcccactggcaccctctagtaagagcacatctgggggtactgccgctctgggatgtctggtgaaggattacttcccagagccagtcaccgtgtcctggaacagcggggccctgacttccggtgtccatacctttccagctgtgctgcagtcatccggcctgtacagcctgagctctgtggtcaccgtccccagttcatccctgggaacacagacttatatctgcaacgtgaatcacaagccatccaatacaaaagtcgacaagaaagtggaacccaagagctgtgataaaacccatacatgccccccttgtcctgctccagagctgctgggaggaccatccgtgttcctgtttccacccaagcctaaagacactctgatgatttctcgaacccccgaagtcacatgcgtggtcgtggacgtgtcccacgaggatcctgaagtcaagttcaactggtacgtggatggcgtcgaggtgcataatgccaagacaaaaccacgagaggaacagtacaacagtacctatcgtgtcgtgtcagtcctgacagtgctgcaccaggactggctgaacgggaaggaatataagtgcaaagtgagcaataaggcactgcccgcccctatcgagaaaacaatttctaaggctaaaggacagcctagggaaccacaggtgtacactctgcctccatcacgggacgagctgacaaagaaccaggtcagtctgacttgtctggtgaaagggttctatccttctgatatcgccgtggagtgggaaagtaatggtcagccagagaacaattacaagaccacaccccctgtcctggactctgatgggagtttctttctgtattccaagctgaccgtggataaaagccggtggcagcagggtaatgtctttagttgttcagtgatgcacgaggcactgcacaatcactacacccagaaatcactgtcactgtcaccaggtaaatga

SEQ ID NO: 36 - protein, 449 aa, synthetic construct
  REGION 1..449: note = Synthetic Construct
EVQLVESGGGVVRPGGSLRLSCAASGFTFSSYAMHWVRQAPGKGLEWVAVISYDVSNKYYADSVKGRFTISRDNSKNTLYLQMNSLRAEDTAVYYCARSYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 645 bp, synthetic construct
  misc_feature 1..645: note = Derived from human variable domain and human Ig domain
  source 1..645: mol_type = other DNA
  CDS 1..645
gatgttgtgatgacccagtctccatcctccctgtctgcatctgtaggagacagagtcaccatcacttgccgggcaagtcagagcattagcaggtacttagcctggtaccaacagaaacctggccaggctcccaggctcctcatctatgatgcatccaacagggccactggcatcccagtcaggttcagtggcagtgggtctgggacagacttcactctcaccatcagcagactggagccagaagattttgcagtgtattactgtcagcagtttggtaggtcacctcggacgttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 38 - protein, 214 aa, synthetic construct
  REGION 1..214: note = Synthetic Construct
DVVMTQSPSSLSASVGDRVTITCRASQSISRYLAWYQQKPGQAPRLLIYDASNRATGIPVRFSGSGSGTDFTLTISRLEPEDFAVYYCQQFGRSPRTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 732 bp, synthetic construct
  misc_feature 1..732: note = Derived from human variable domains
  source 1..732: mol_type = other DNA
  CDS 1..732
gatgttgtgatgacccagtctccatcctccctgtctgcatctgtaggagacagagtcaccatcacttgccgggcaagtcagagcattagcaggtacttagcctggtaccaacagaaacctggccaggctcccaggctcctcatctatgatgcatccaacagggccactggcatcccagtcaggttcagtggcagtgggtctgggacagacttcactctcaccatcagcagactggagccagaagattttgcagtgtattactgtcagcagtttggtaggtcacctcggacgttcggccaagggacacgactggagattaaaggcggatcctctaggtcaagttccagcggcggcggtggcagcggaggcggcggtgaggtgcaactggtggagtctgggggaggtgtggtaaggcctggggggtccctgagactctcctgtgcagcctctggattcaccttcagtagctatgctatgcactgggtccgccaggctccaggcaaggggctggagtgggtggcagttatatcatatgatgtaagcaataaatactacgcagactccgtgaagggccgattcaccatctccagagacaattccaagaacacgctgtatctgcaaatgaacagcctgagagctgaggacacggctgtgtattactgtgcgagatcttactactactactacggtatggacgtctggggccaagggaccacggtcaccgtctcctca

SEQ ID NO: 40 - protein, 244 aa, synthetic construct
  REGION 1..244: note = Synthetic Construct
DVVMTQSPSSLSASVGDRVTITCRASQSISRYLAWYQQKPGQAPRLLIYDASNRATGIPVRFSGSGSGTDFTLTISRLEPEDFAVYYCQQFGRSPRTFGQGTRLEIKGGSSRSSSSGGGGSGGGGEVQLVESGGGVVRPGGSLRLSCAASGFTFSSYAMHWVRQAPGKGLEWVAVISYDVSNKYYADSVKGRFTISRDNSKNTLYLQMNSLRAEDTAVYYCARSYYYYYGMDVWGQGTTVTVSS

SEQ ID NO: 41 - DNA, 351 bp, synthetic construct
  misc_feature 1..351: note = chimeric sequences
  source 1..351: mol_type = other DNA
  CDS 1..351
cagtcggtggaggagtccgggggtcgcctggtcacgcctgggacacccctgacactcacctgcacagtctctggaatcgacctcagtaacaatgcaatgagctgggtccgccaggctccagggaaggggctggaatatatcggaatcattaggagtagtggtagtacatattacgcgaactgggcaaaaggccggttcaccatctccaaaacctcgaccacggtggatctgataatcaccagtccgacaaccgaggacacggccacctatttctgtgccagagggggtggttcttacagtattgtcttctggaacttgtggggcccaggcaccctggtcaccgtctcctca

SEQ ID NO: 42 - protein, 117 aa, synthetic construct
  REGION 1..117: note = Synthetic Construct
QSVEESGGRLVTPGTPLTLTCTVSGIDLSNNAMSWVRQAPGKGLEYIGIIRSSGSTYYANWAKGRFTISKTSTTVDLIITSPTTEDTATYFCARGGGSYSIVFWNLWGPGTLVTVSS

SEQ ID NO: 43 - DNA, 333 bp, synthetic construct
  misc_feature 1..333: note = chimeric sequences
  source 1..333: mol_type = other DNA
  CDS 1..333
gcctatgatatgacccagactccagcctctgtggaggtagctgtgggaggcacagtcaccatcaattgccaggccagtgagaggatttatagcaatttagcctggtatcagcagaaaccagggcagcgtcccaagctcctgatctattatacatccactctggcatctggggtctcatcgcggttcaaaggcagtggatctgggacacagttcactctcaccatcagcggcgtggagtgtgccgatgctgccacttactactgtcaacagggttatagtagtagtaatgttgacaatactttcggcggagggaccgaggtggtggtcaaaggt

SEQ ID NO: 44 - protein, 111 aa, synthetic construct
  REGION 1..111: note = Synthetic Construct
AYDMTQTPASVEVAVGGTVTINCQASERIYSNLAWYQQKPGQRPKLLIYYTSTLASGVSSRFKGSGSGTQFTLTISGVECADAATYYCQQGYSSSNVDNTFGGGTEVVVKG

SEQ ID NO: 45 - DNA, 363 bp, synthetic construct
  misc_feature 1..363: note = chimeric sequences
  source 1..363: mol_type = other DNA
  CDS 1..363
ggcgagcagcagctggtggagagcggcggaggcctggtgcagcctggaggaagcctgaggctgagctgcgccgtgtccggcttcagcctgagcagctacgccatcagctgggtgaggcaggcccccggaaagggcctggagtacatcgccatcatcaacagctacggcaccacctactacgccagctgggccaagggcagagtgaccatctccaaggattcctccaagaacaccgtgtacctgcagatgggctccctgagagccgaggatatggccgtgtacttttgcgccagaggcgattcctacggctccggcgtgggcctgggcctgtggggacctggaaccctggtgacagtgtcctcc

SEQ ID NO: 46 - protein, 121 aa, synthetic construct
  REGION 1..121: note = Synthetic Construct
GEQQLVESGGGLVQPGGSLRLSCAVSGFSLSSYAISWVRQAPGKGLEYIAIINSYGTTYYASWAKGRVTISKDSSKNTVYLQMGSLRAEDMAVYFCARGDSYGSGVGLGLWGPGTLVTVSS

SEQ ID NO: 47 - DNA, 333 bp, synthetic construct
  misc_feature 1..333: note = chimeric sequences
  source 1..333: mol_type = other DNA
  CDS 1..333
ggagactaccagatgacacagtcccctagcaccctgtccgcctccgtgggcgacagagtgacaatcacctgtcaggcctcccagaatatctacagcaatctggcctggtaccagcagaagcctggcaagaggcccaagctgctgatctacagagccagctccctggcctccggcgtgccatctagattttccggctccggcagcggcacagagtttaccctgacaatcagcagcctgcagcccgatgatttcgccacctactactgtcagcagggcttcagcagcaataatgtggacaatacatttggcggcggcacaaaggtggagatcaag

SEQ ID NO: 48 - protein, 111 aa, synthetic construct
  REGION 1..111: note = Synthetic Construct
GDYQMTQSPSTLSASVGDRVTITCQASQNIYSNLAWYQQKPGKRPKLLIYRASSLASGVPSRFSGSGSGTEFTLTISSLQPDDFATYYCQQGFSSNNVDNTFGGGTKVEIK

SEQ ID NO: 49 - DNA, 354 bp, synthetic construct
  misc_feature 1..354: note = chimeric sequences
  source 1..354: mol_type = other DNA
  CDS 1..354
ggcgagcagcagctggtggagagcggcggaggcctggtgcagcctggaggaagcctgaggctgagctgcgccgtgtccggcttttccctgagcaagagcatcatcagctgggtgaggcaggcccctggcaagggcctggagtacatcggcatcatcggcagcagcggctccacctactacgccaactgggccaagggcagattcacaatctccaaggactcctccaagaataccgtgtacctgcagatgggctccctgagggccgaggatatggccgtgtacttttgtgccagaggcctgctgtactccggcaataagtcctggggccccggcacactggtgaccgtgagctcc

SEQ ID NO: 50 - protein, 118 aa, synthetic construct
  REGION 1..118: note = Synthetic Construct
GEQQLVESGGGLVQPGGSLRLSCAVSGFSLSKSIISWVRQAPGKGLEYIGIIGSSGSTYYANWAKGRFTISKDSSKNTVYLQMGSLRAEDMAVYFCARGLLYSGNKSWGPGTLVTVSS

SEQ ID NO: 51 - DNA, 333 bp, synthetic construct
  misc_feature 1..333: note = chimeric sequences
  source 1..333: mol_type = other DNA
  CDS 1..333
ggcgacatcgtgatgacccagtcccccgattccctggccgtgtccctgggcgagagagccacaatcaattgtcagtcctcccagagcgtgctgctgaacaatcagctgtcctggttccagcagaagcctggccagcctcccaagctgctgatctacgacgcctccacactggagtccggcgtgcccgataggttcagcggctccggcagcggcaccgactttaccctgaccatctccagcctgcaggccgaggatgtggccgtgtactactgcctgggcggctacagcggcaacctgtacgcctttggcggcggcaccaaggtggagatcaag

SEQ ID NO: 52 - protein, 111 aa, synthetic construct
  REGION 1..111: note = Synthetic Construct
GDIVMTQSPDSLAVSLGERATINCQSSQSVLLNNQLSWFQQKPGQPPKLLIYDASTLESGVPDRFSGSGSGTDFTLTISSLQAEDVAVYYCLGGYSGNLYAFGGGTKVEIK

SEQ ID NO: 53 - DNA, 369 bp, synthetic construct
  misc_feature 1..369: note = chimeric sequences
  source 1..369: mol_type = other DNA
  CDS 1..369
ggcgagcagcagctggtggagtccggcggaggcctggtgcagccaggaggaagcctgaggctgtcctgtgccgtgagcggcttctccctgagctcctacgccatgagctgggtgaggcaggcccccggaaagggcctggagtacatcggcatcatcagcagcagcggcagcacatactacgccagctgggccaagggcaggttcacaatcagcaaggattcctccaagaatacagtgtacctgcagatgggctccctgagggccgaggacatggccgtgtacttctgtgccagagacagggtcatctattccatcggcccttactacttcaacctgtggggccccggcacactggtgacagtgtccagc

SEQ ID NO: 54 - protein, 123 aa, synthetic construct
  REGION 1..123: note = Synthetic Construct
GEQQLVESGGGLVQPGGSLRLSCAVSGFSLSSYAMSWVRQAPGKGLEYIGIISSSGSTYYASWAKGRFTISKDSSKNTVYLQMGSLRAEDMAVYFCARDRVIYSIGPYYFNLWGPGTLVTVSS

SEQ ID NO: 55 - DNA, 333 bp, synthetic construct
  misc_feature 1..333: note = chimeric sequences
  source 1..333: mol_type = other DNA
  CDS 1..333
ggcgattaccagatgacacagtccccctcctccctgagcgcctccgtgggagatagggtgaccatcacatgccaggccagcgagatcatctacagcaatctggcctggtaccagcagaagcccggcaagccccccaagctgctgatctacggcgcctccacactggccagcggcgtgcctagcagattcagcggcagcggctccggcaccgattacaccctgacaatctccagcctgcagcctgaggattttgccacatactactgtcagcagtccttcagctccaataacgtgggcaacatcttcggcggcggcacaaaggtggagatcaag

SEQ ID NO: 56 - protein, 111 aa, synthetic construct
  REGION 1..111: note = Synthetic Construct
GDYQMTQSPSSLSASVGDRVTITCQASEIIYSNLAWYQQKPGKPPKLLIYGASTLASGVPSRFSGSGSGTDYTLTISSLQPEDFATYYCQQSFSSNNVGNIFGGGTKVEIK